(19) United States
(12) Patent Application Publication
(10) Pub. No.: US 2014/0357518 A1
(43) Pub. Date: Dec. 4, 2014

(54) Methods and Compositions for the Treatment and Diagnosis of Thyroid Cancer

(71) Applicant: Oncocyte Corporation, Alameda, CA (US)

(72) Inventors: Karen Chapman, Mill Valley, CA (US); Joseph Wagner, San Ramon, CA (US); Michael West, Mill Valley, CA (US); Markus Daniel Lacher, Lafayette, CA (US); Jennifer Lorie Kidd, Alameda, CA (US)

(73) Assignee: Oncocyte Corporation, Alameda, CA (US)

(21) Appl. No.: 14/371,215

(22) PCT Filed: Jan. 11, 2013
(86) PCT No.: PCT/US2013/021286

§ 371 Date: Jul. 9, 2014

Related U.S. Application Data
(60) Provisional application No. 61/585,823, filed on Jan. 12, 2012.

(51) Int. Cl. C12Q 1/68; G01N 33/574

(52) U.S. Cl. CPC C12Q 1/6886; G01N 33/57407; C12Q 2600/158
USPC 506/9; 435/6.12; 435/7.1

(57) ABSTRACT

The invention provides methods, compositions and kits for the detection and treatment of thyroid cancer.

This application claims priority to U.S. Provisional Application No. 61/585,823 filed on Jan. 12, 2012 the entire contents of which is hereby incorporated by reference.

### FIELD OF THE INVENTION

The field of the invention relates to cancer and the diagnosis and treatment of cancer.

### BACKGROUND

Early detection of cancer can impact treatment outcomes and disease progression. Typically, cancer detection relies on diagnostic information obtained from biopsy, x-rays, CAT scans, NMR and the like. These procedures may be invasive, time consuming and expensive. Moreover, they have limitations with regard to sensitivity and specificity. There is a need in the field of cancer diagnostics for a highly specific, highly sensitive, rapid, inexpensive, and relatively non-invasive method of diagnosing cancer. Various embodiments of the invention described below meet this need as well as other needs existing in the field of diagnosing and treating cancer.

### SUMMARY OF THE INVENTION

Embodiments of the disclosure provide methods of diagnosis, prognosis and treatment of cancer, e.g. thyroid cancer. Other embodiments provide compositions relating to the diagnosis, prognosis and treatment of cancer, such as thyroid cancer. In certain embodiments one or more of the markers disclosed herein, e.g. SEQ ID NOS: 1-29, may be used in the diagnosis, prognosis and treatment of thyroid cancer as disclosed infra. In some embodiments one or more of the markers disclosed infra may be used to distinguish a malignant thyroid tumor from a benign thyroid tumor using the methods described below. In some embodiments the invention provides a method of distinguishing a thyroid follicular adenoma from a thyroid follicular carcinoma.

In certain embodiments the invention provides a method of detecting thyroid cancer in a subject comprising a) obtaining a sample from a subject; b) contacting the sample obtained from the subject with one or more agents that detect one or more markers expressed by a thyroid cancer cell c) contacting a non-cancerous cell with the one or more agents from b); and d) comparing the expression level of the marker in the sample obtained from the subject with the expression level in the non-cancerous cell, wherein a higher level of expression of the marker in the sample compared to the non-cancerous cell indicates that the subject has thyroid cancer. Suitable markers include the genes encoded for by SEQ ID NOS: 1-29.

In some embodiments the invention provides a method of detecting thyroid cancer in a subject comprising a) obtaining a sample from a subject b) contacting the sample obtained from the subject with one or more agents that detect expression of one or more of the markers encoded by genes chosen from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a complement thereof; c) contacting a non-cancerous cell with the one or more agents from b); and d) comparing the expression level of one or more of the markers encoded by genes chosen from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B AHNAK2, CYTOKERATINE19, or a complement thereof in the non-cancerous cell with the sample obtained from the subject, wherein a higher level of expression in the sample of one or more of the markers encoded by genes chosen from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233 NMU KIAA1324, CCDC85A, CRABP2, C14orf78, TNFRSF11B, AHNAK2, CYTOKERATINE19 or a complement thereof in the sample obtained from the subject compared to the non-cancerous cell indicates that the subject has thyroid cancer.

In other embodiments the invention provides a method of detecting thyroid cancer in a subject comprising a) obtaining a sample from a subject b) contacting the sample obtained from the subject with one or more agents that detect expression of a panel of markers encoded by the genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a complement thereof, c) contacting a non-cancerous cell, with the one or more agents from b); and d) comparing the expression level of the panel of markers encoded for by the genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a complement thereof in the sample obtained from the subject with the expression level of the panel of markers encoded for by the genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a complement thereof; in the non-cancerous cell, wherein a higher level of expression of the panel of markers encoded for by genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233 NMU KIAA1324, CCDC85A, CRABP2, C14orf78, TNFRSF11B, AHNAK2, CYTOKERATINE19 or a complement thereof in the sample compared to the non-cancerous cell indicates that the subject has thyroid cancer.

In further embodiments the invention provides a method of detecting thyroid cancer cells in a sample comprising a) obtaining a sample b) contacting the sample obtained in a) with one or more agents that detect expression of one or more of the markers encoded by genes chosen from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a complement thereof; c) contacting a non-cancerous cell with the one or more agents from b); and d) comparing the expression level of one or more of the markers encoded by genes chosen from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19 or a complement thereof in the sample obtained in a) with the expression level of one or more of the markers encoded by genes chosen from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a complement thereof; in the non-cancerous cell, wherein a higher level of expression of one or more of the markers encoded by genes chosen from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19 or a complement thereof, in the sample compared to the non-cancerous cell indicates that the sample contains thyroid cancer cells. The sample may be an in vitro sample or an in vivo sample, or derived from an in vivo sample.

With regard to the embodiments described in the preceding paragraphs, the sample may be any sample as described infra, for example, a bodily fluid, such as blood, serum or urine. The sample may be a cellular sample or the extract of a cellular sample. The sample may be a tissue sample. Nucleic acids and/or proteins may be isolated from the sample. Nucleic acids such as RNA may be transcribed into cDNA. The agent may be one or more molecules that bind specifically to one or more proteins expressed by the cancer cell or one or more nucleic acids expressed by the cell. For example, the agent may be a protein such as an antibody that binds specifically to the protein expressed by one of the marker genes identified infra. The agent may be one or more nucleic acids that hybridize to a nucleic acid expressed by the cancer cell. The nucleic acid expressed by the cancer cell may be an RNA molecule, e.g. an mRNA molecule. The nucleic acid molecule that hybridizes to the nucleic acid expressed by the cancer cell may be a DNA molecule, such as a DNA probe.

In still other embodiments the invention provides a composition of matter useful in distinguishing a thyroid cancer cell from a non-cancerous cell comprising one or more molecules that specifically bind to a molecule expressed at higher levels by a thyroid cancer cell compared to a non-cancer cell. As an example, the composition may comprise a protein, that binds to one or more molecules expressed by the thyroid cancer cell at higher levels compared to the non-cancer cell. As another example, the composition may comprise a nucleic acid, e.g. a DNA molecule such as an oligonucloetide that binds to one or more molecules such as an mRNA molecule or a cDNA molecule reverse transcribed from an mRNA molecule expressed by the thyroid cancer cell at higher levels compared to the non-cancer cell.

In still other embodiments the invention provides a composition of matter useful in distinguishing a thyroid malignant tumor cell from a thyroid benign tumor cell comprising one or more molecules that specifically bind to a molecule expressed at higher levels by a thyroid malignant tumor cell compared to a thyroid benign tumor cell. As an example, the composition may comprise a protein, that binds to one or more molecules expressed by the thyroid malignant tumor cell at higher levels compared to the thyroid benign tumor cell. As another example, the composition may comprise a nucleic acid, e.g. a DNA molecule such as an oligonucloetide that binds to one or more molecules such as an mRNA molecule or a cDNA molecule reverse transcribed from an mRNA molecule expressed by the thyroid malignant tumor cell at higher levels compared to the thyroid benign tumor cell. Suitable molecules include agents that bind to one or more of the nucleic acids, or proteins encoded for by those nucleic acids described infra that are expressed at higher levels in malignant thyroid tumors compared to benign thyroid tumors.

In still other embodiments the invention provides a composition of matter useful in distinguishing a thyroid malignant tumor cell from a thyroid benign tumor cell comprising one or more molecules that specifically bind to a molecule expressed at higher levels by a thyroid benign tumor cell compared to a thyroid malignant tumor cell. As an example, the composition may comprise a protein, that binds to one or more molecules expressed by the thyroid benign tumor cell at higher levels compared to the thyroid malignant tumor cell. As another example, the composition may comprise a nucleic acid, e.g. a DNA molecule such as an oligonucloetide that binds to one or more molecules such as an mRNA molecule or a cDNA molecule reverse transcribed from an mRNA molecule expressed by the thyroid benign tumor cell at higher levels compared to the thyroid malignant tumor cell. Suitable molecules include agents that bind to one or more of the nucleic acids, or proteins encoded for by those nucleic acids described infra that are expressed at higher levels in benign thyroid tumors compared to malignant thyroid tumors.

In some embodiments the invention provides a composition of matter comprising one or more proteins, such as an antibody, that specifically binds to a molecule expressed by a thyroid cancer cell chosen from the markers encoded by the SEQ ID NOS: 1-29. The molecule expressed by the thyroid cancer cell may be expressed by the cancer cell at a level that is higher than the level expressed by a non-cancerous cell.

In some embodiments the invention provides a composition of matter comprising one or more proteins, such as an antibody, that specifically binds to a molecule expressed by a thyroid cancer cell chosen from the markers encoded by the genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19. The molecule expressed by the thyroid cancer cell may be expressed by the cancer cell at a level that is higher than the level of the same marker expressed by a non-cancerous cell.

In further embodiments the invention provides a composition of matter comprising a plurality of proteins, such as a plurality antibodies, that specifically binds to a panel of molecules expressed by a thyroid cancer cell wherein the panel of markers comprises molecule encoded by the genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf18, TNFRSF11B, AHNAK2, CYTOKERATINE19 or a complement thereof. The panel of markers may be expressed at a level that is higher than the level of the panel of markers in a non-cancerous cell.

In further embodiments the invention provides a composition of matter comprising a plurality of proteins, such as a plurality antibodies, that specifically binds to a panel of molecules expressed by a thyroid cancer cell wherein the panel of markers comprises molecule encoded by the genes TGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a complement thereof. The panel of markers may be expressed at a level that is higher than the level of the panel of markers in a non-cancerous cell.

In certain embodiments the invention provides a composition of matter comprising a protein, such as an antibody, that specifically binds to a molecule expressed by an thyroid cancer cell chosen from a molecule encoded by one or more of the genes chosen from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19 or a complement thereof. The molecule expressed by the thyroid cancer cell may be expressed by the thyroid cancer cell at level that is higher than the level expressed by a non-cancerous cell.

In other embodiments the invention provides a composition of matter comprising a nucleic acid that specifically binds to a molecule, such as an mRNA molecule, expressed by a thyroid cancer cell wherein the molecule is chosen from a marker encoded for by the genes listed in SEQ ID NOS: 1-29. The molecule expressed by the thyroid cancer cell may be expressed by the thyroid cancer cell at level that is higher than the level expressed by a non-cancerous cell.

In other embodiments the invention provides a composition of matter comprising a nucleic acid that specifically binds to a molecule, such as an mRNA molecule, expressed by a thyroid cancer cell wherein the molecule is chosen from a marker encoded for by the genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI13L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19. The molecule expressed by the thyroid cancer cell may be expressed by the cancer cell at level that is higher than the level expressed by a non-cancerous cell.

In still further embodiments the invention provides a method of determining if a thyroid cancer in a subject is advancing comprising a) measuring the expression level of one or more markers associated with thyroid cancer at a first time point; b) measuring the expression level of the one or more markers measured in a) at a second time point, wherein the second time point is subsequent to the first time point; and c) comparing the expression level measured in a) and b), wherein an increase in the expression level of the one or more markers in b) compared to a) indicates that the subject's thyroid cancer is advancing. Suitable markers include those markers encoded for by the genes provided in SEQ ID NOS: 1-29.

In some embodiments the invention provides a method of determining if a thyroid cancer in a subject is advancing comprising a) measuring the expression level of the panel of markers encoded for by the genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 at a first time point; b) measuring the expression level of the markers measured in a) at a second time point, wherein the second time point is subsequent to the first time point; and c) comparing the expression level measured in a) and b), wherein an increase in the expression level of the markers at the second time point compared to the first time point indicates that the subject's thyroid cancer is advancing.

In some embodiments the invention provides antigens (i.e. cancer-associated polypeptides) associated with thyroid cancer as targets for diagnostic and/or therapeutic antibodies. In some embodiments, the antigen may be chosen from a protein encoded by, a gene listed in SEQ ID NOS: 1-29, a fragment thereof, or a combination of proteins encoded by a gene listed in SEQ ID NOS 1-29.

In some embodiments the invention provides antigens (i.e. cancer-associated polypeptides) associated with thyroid cancer as targets for diagnostic and/or therapeutic antibodies. In some embodiments, the antigen may include a panel of proteins encoded by the genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19 or a fragment thereof.

In yet other embodiments the invention provides a method of eliciting an immune response to a thyroid cancer cell comprising contacting a subject with a protein or protein fragment that is expressed by a thyroid cancer cell thereby eliciting an immune response to the thyroid cancer cell. As an example the subject may be contacted intravenously or intramuscularly with protein or protein fragment.

In further embodiments the invention provides a method of eliciting an immune response to a thyroid cancer cell comprising contacting a subject with one or more proteins or protein fragments that is encoded by a gene chosen from the genes listed in SEQ ID NOS: 1-29, thereby eliciting an immune response to a thyroid cancer cell. As an example the subject may be contacted with the protein or the protein fragment intravenously or intramuscularly.

In further embodiments the invention provides a method of eliciting an immune response to a thyroid cancer cell comprising contacting a subject with one or more proteins or protein fragments that is encoded by a gene chosen from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19 or a fragment thereof thereby eliciting an immune response to a thyroid cancer cell. As an example the subject may be contacted with the protein or the protein fragment intravenously or intramuscularly.

In yet other embodiments the invention provides a kit for detecting thyroid cancer cells in a sample. The kit may comprise one or more agents that detect expression of any the cancer associated sequences disclosed infra e.g. SEQ ID NOS 1-29. The agents may bind to one or more of the cancer associated sequences disclosed infra. The kit may include agents that are proteins and/or nucleic acids for example. In one embodiment the kit provides a plurality of agents. The agents may be able to detect the panel of markers encoded by the genes comprising IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19 or a complement thereof.

In still other embodiments the invention provides a kit for detecting thyroid cancer in a sample comprising a plurality of agents that specifically bind to a molecule encoded for by one or more of the genes chosen from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19.

In other embodiments the invention provides a kit for detection of thyroid cancer in a sample obtained from a subject. The kit may comprise one or more agents that bind specifically to a molecule expressed specifically by a thyroid cancer cell, e.g. one or more of the markers encoded for by SEQ ID NOS; 1-29. The kit may comprise one or more containers and instructions for determining if the sample is positive for cancer. The kit may optionally contain one or more multiwell plates, a detectable substance such as a dye, a radioactively labeled molecule, a chemiluminescently labeled molecule and the like. The detectable substance may be linked to the agent that specifically binds to a molecule expressed by a thyroid cancer cell. The kit may further contain a positive control (e.g. one or more thyroid cancer cells; or specific known quantities of the molecule expressed by the thyroid cancer cell) and a negative control (e.g. a tissue or cell sample that is non-cancerous).

In some embodiments the invention provides a kit for the detection of thyroid cancer comprising one or more agents that specifically bind one or more markers encoded by genes chosen from a gene disclosed infra., e.g., IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19. The agent may be a protein, such as an antibody. Alternatively, the agent may be a nucleic such as a DNA molecule or an RNA molecule. The kit may comprise one or more containers and instructions for determining if the sample is positive for cancer. The kit may optionally contain one or more multiwell plates, a detectable substance such as a dye, a radioactively labeled molecule, a chemiluminescently labeled molecule and the like. The detectable substance may be linked to the agent that specifically binds the one or more markers disclosed infra. The kit may further contain a positive control (e.g. one or more thyroid cancer cells; or specific known quantities of the molecule expressed by the thyroid cancer cell) and a negative control (e.g. a tissue or cell sample that is non-cancerous). As an example the kit may take the form of an ELISA or a DNA microarray. In some embodiments the kit may include one or more antibodies suitable for use in a fluorescent activated cell sorter, e.g. use in flow cytometry.

Some embodiments are directed to a method of treating thyroid cancer in a subject, the method comprising administering to a subject in need thereof a therapeutic agent modulating the activity of a thyroid cancer associated protein, wherein the cancer associated protein is encoded by gene listed in SEQ ID NOS: 1-29, homologs thereof, combinations thereof, or a fragment thereof. In some embodiments, the therapeutic agent binds to the cancer associated protein. In some embodiments, the therapeutic agent is an antibody. In some embodiments, the antibody may be a monoclonal antibody or a polyclonal antibody. In some embodiments, the antibody is a humanized or human antibody. In some embodiments the antibody may be conjugated with a drug or a toxin.

In some embodiments, a method of treating thyroid cancer in a subject may comprise administering to a subject in need thereof a therapeutic agent that modulates the expression of one or more genes chosen from those listed in SEQ ID NOS: 1-29, fragments thereof, homologs thereof, and/or complements thereof.

In further embodiments, the invention provides a method of treating thyroid cancer may comprise a gene knockdown of one or more genes listed in SEQ ID NOS: 1-29, fragments thereof, homologs thereof, and or compliments thereof.

In still other embodiments, the present invention provides methods of screening a drug candidate for activity against thyroid cancer, the method comprising: (a) contacting a cell that expresses one or more thyroid cancer associated genes chosen from those listed in SEQ ID NOS: 1-29 with a drug candidate; (b) detecting an effect of the drug candidate on expression of the one or more thyroid cancer associated genes in the cell from a); and (c) comparing the level of expression of one or more of the genes recited in a) in the absence of the drug candidate to the level of expression of the one or more genes recited in a) in the presence of the drug candidate; wherein a decrease in the expression of the thyroid cancer associated gene in the presence of the drug candidate indicates that the candidate has activity against thyroid cancer.

In some embodiments, the present invention provides methods of visualizing a thyroid tumor comprising a) targeting one or more thyroid cancer associated proteins with a labeled molecule that binds specifically to the cancer tumor, wherein the thyroid cancer associated protein is selected from a protein encoded for by one or more genes chosen from those listed in SEQ ID NOS: 1-29; and b) detecting the labeled molecule, wherein the labeled molecule visualizes the tumor. Visualization may be done in vivo, or in vitro. The tumor may be a malignant thyroid tumor or a benign thyroid tumor.

In yet other embodiments the invention provides methods of visualizing a thyroid cancer tumor comprising a) targeting one or more thyroid cancer associated genes, e.g. one or more genes encoded for by SEQ ID NOS: 1-29, with a labeled molecule, such as a nucleic acid that binds specifically to the cancer tumor genes chosen from those listed in SEQ ID NOS: 1-29; and b) detecting the labeled molecule, wherein the labeled molecule visualizes the tumor. Visualization may be done in vivo, or in vitro.

### DESCRIPTION OF DRAWINGS

For a fuller understanding of the nature and advantages of the present invention, reference should be had to the following detailed description taken in connection with the accompanying drawings, in which:

FIG. 1A shows the expression of IGSF1 in normal cells and tissues versus thyroid tumors.

FIG. 1B shows the expression of IGSF1 relative to 3-Actin on Tissue Scan Thyroid I Array.

FIG. 2 shows the expression of IGSF21 in normal cells and tissues versus thyroid tumors.

FIG. 3A shows the expression of TM7SF4 in normal cells and tissues versus thyroid tumors.

FIG. 3B shows the expression of TM7SF4 relative to β-Actin on Tissue Scan Thyroid I Array.

FIG. 4 shows the expression of FLJ30058 in normal cells and tissues versus thyroid tumors.

FIG. 5 shows the expression of CITED1 in normal cells and tissues versus thyroid tumors.

FIG. 6 shows the expression of ZCCHC12 in normal cells and tissues versus thyroid tumors.

FIG. 7 shows the expression of CLDN16 in normal cells and tissues versus thyroid tumors.

FIG. 8 shows the expression of FN1 in normal cells and tissues versus thyroid tumors.

FIG. 9 shows the expression of SERPINA1 in normal cells and tissues versus thyroid tumors.

FIG. 10 shows the expression of STK32A in normal cells and tissues versus thyroid tumors.

FIG. 11 shows the expression of UNQ9433 in normal cells and tissues versus thyroid tumors.

FIG. 12 shows the expression of BC030766 in normal cells and tissues versus thyroid tumors.

FIG. 13 shows the expression of AK023519 in normal cells and tissues versus thyroid tumors.

FIG. 14 shows the expression of SLC34A2 in normal cells and tissues versus thyroid tumors.

FIG. 15 shows the expression of BX538295 in normal cells and tissues versus thyroid tumors.

FIG. 16 shows the expression of IGFL2 in normal cells and tissues versus thyroid tumors.

FIG. 17 shows the expression of CHI3L1 in normal cells and tissues versus thyroid tumors.

FIG. 18 shows the expression of CYP24A1 in normal cells and tissues versus thyroid tumors.

FIG. 19 shows the expression of IGSF1 in normal cells and tissues versus thyroid tumors.

FIG. 20 shows the expression of CHI3L1 in normal cells and tissues versus thyroid tumors.

FIG. 21 shows the expression of TM7SF4 in normal cells and tissues versus thyroid tumors.

FIG. 22 shows the expression of ZCCHC12 in normal cells and tissues versus thyroid tumors.

FIG. 23 shows the expression of SFTPB in normal cells and tissues versus thyroid tumors.

FIG. 24 shows the expression of NMU in normal cells and tissues versus thyroid tumors.

FIG. 25 shows the expression of PLAG1 in normal cells and tissues versus thyroid tumors.

FIG. 26 shows the expression of FLJ30058 in normal cells and tissues versus thyroid tumors.

FIG. 27 shows the expression of IGSF 1 in benign thyroid tumor cells and tissues versus malignant thyroid tumors.

FIG. 28 shows the expression of CHI3L1 in benign thyroid tumor cells and tissues versus malignant thyroid tumors.

FIG. 29 shows the expression of ZCCH12 in benign thyroid tumor cells and tissues versus malignant thyroid tumors.

FIG. 30 shows the expression of NMU in benign thyroid tumor cells and tissues versus malignant thyroid tumors.

FIG. 31 shows the expression of PLAG1 in benign thyroid tumor cells and tissues versus malignant thyroid tumors.

FIG. 32 shows the expression of FLJ30058 in benign thyroid tumor cells and tissues versus malignant thyroid tumors.

FIG. 33 shows the expression of SLCO4C1 in benign thyroid tumor cells and tissues versus malignant thyroid tumors.

FIG. 34 shows a composite of 8 markers for thyroid cancer using a binary cutoff setting sensitivity to 100%.

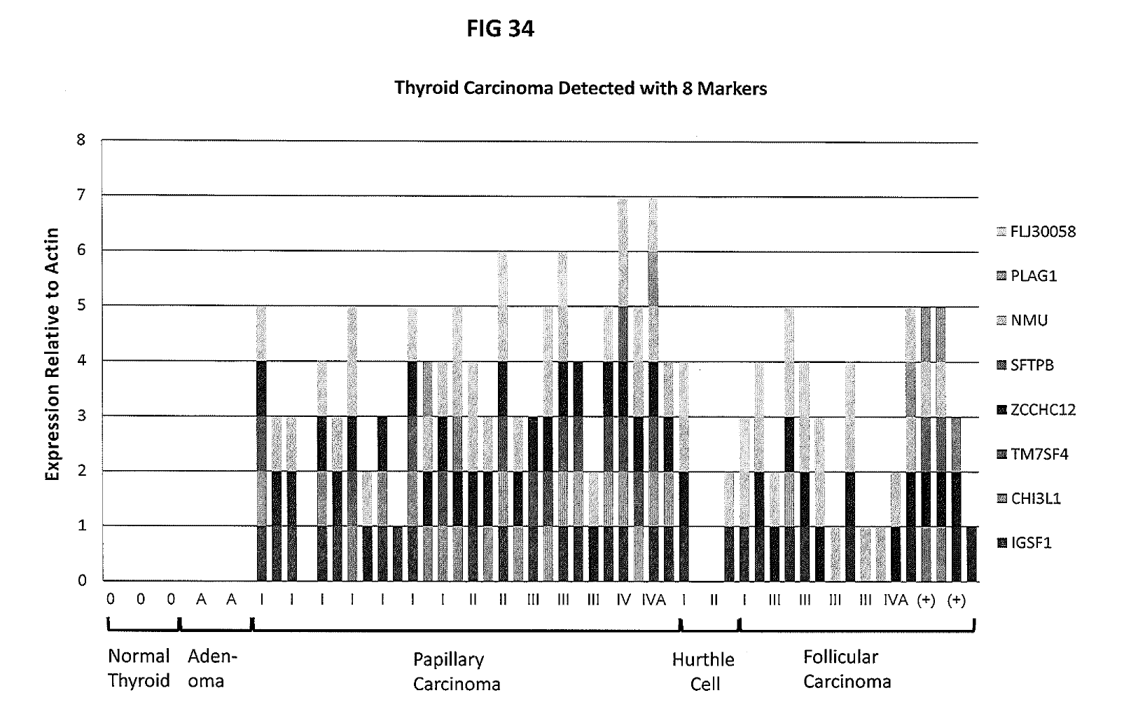

FIG. 35 shows that AHNAK2 protein is expressed in thyroid carcinoma cells.

FIG. 36 shows that Cytokeratine 19 protein is expressed in thyroid carcinoma cells.

FIG. 37 shows that FLJ30058 protein is expressed in thyroid carcinoma cells.

FIG. 38 shows TNFRSF11B mRNA expression levels in benign and malignant human thyroid samples as measured by an LDA Assay. Sample identification numbers in parentheses indicate samples assessed in both Examples 23 and 24.

FIG. 39 shows C14orf78 (AHNAK2) mRNA expression levels in benign and malignant human thyroid samples as measured by an LDA Assay. Sample identification numbers in parentheses indicate samples assessed in both Examples 23 and 24.

FIG. 40 shows PLAG1 mRNA expression levels in benign and malignant human thyroid samples as measured by an LDA Assay. Sample identification numbers in parentheses indicate samples assessed in both Examples 23 and 24.

FIG. 41 shows CRABP2 mRNA expression levels in benign and malignant human thyroid samples as measured by an LDA Assay. Sample identification numbers in parentheses indicate samples assessed in both Examples 23 and 24.

FIG. 42 shows CCDC85A mRNA expression levels in benign and malignant human thyroid samples as measured by an LDA Assay. Sample identification numbers in parentheses indicate samples assessed in both Examples 23 and 24.

FIG. 43 shows FLJ30058 (ARHGAP36) mRNA expression levels in benign and malignant human thyroid samples as measured by an LDA Assay. Sample identification numbers in parentheses indicate samples assessed in both Examples 23 and 24.

FIG. 44 shows KIAA1324 mRNA expression levels in benign and malignant human thyroid samples as measured by an LDA Assay. Sample identification numbers in parentheses indicate samples assessed in both Examples 23 and 24.

FIG. 45 shows NMU mRNA expression levels in benign and malignant human thyroid samples as measured by an LDA Assay (LDA-Exp. I). Sample identification numbers in parentheses indicate samples assessed in both Examples 23 and 24.

FIG. 46 shows that a six marker panel distinguishes between adenoma and carcinoma with 100% sensitivity and specificity of 91%.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. Nothing herein is to be construed as an admission that the invention is not entitled to antedate such disclosure by virtue of prior invention.

As used herein, the singular forms “a,” “an,” and “the” include plural reference unless the context clearly dictates otherwise. Thus, for example, reference to a “therapeutic” is a reference to one or more therapeutics and equivalents thereof known to those skilled in the art, and so forth.

As used herein, the term “about” means plus or minus 10% of the numerical value of the number with which it is being used. Therefore, about 50% means in the range of 45% to 55%.

“Administering,” when used in conjunction with a therapeutic, means to administer a therapeutic directly into or onto a target tissue or to administer a therapeutic to a patient whereby the therapeutic treats the tissue to which it is targeted. Thus, as used herein, the term “administering,” when used in conjunction with a therapeutic, can include, but is not limited to, providing the therapeutic into or onto the target tissue; providing the therapeutic systemically to a patient by, e.g., intravenous injection whereby the therapeutic reaches the target tissue; providing the therapeutic in the form of the encoding sequence thereof to the target tissue (e.g., by so-called gene-therapy techniques). “Administering” a composition may be accomplished by oral administration, intravenous Injection, intraperitoneal injection, intramuscular injection, subcutaneous injection, transdermal diffusion or electrophoresis, local injection, extended release delivery devices including locally implanted extended release devices such as bioerodible or reservoir-based implants, as protein therapeutics or as nucleic acid therapeutic via gene therapy vectors, topical administration, or by any of these methods in combination with other known techniques. Such combination techniques include, without limitation, heating, radiation and ultrasound.

“Agent” as used herein refers to a molecule that specifically binds to a cancer associated sequence or a molecule encoded for by a cancer associated sequence or a receptor that binds to a molecule encoded for by a cancer associated sequence. Examples of agents include nucleic acid molecules, such as DNA and proteins, such as antibodies. The agent may be linked with a label or detectible substance as described infra. The agent may be linked with a therapeutic agent or a toxin.

The term “amplify” as used herein means creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases, or any combination thereof.

The term “animal,” “patient” or “subject” as used herein includes, but is not limited to, humans, non-human primates and non-human vertebrates such as wild, domestic and farm animals including any mammal, such as cats, dogs, cows, sheep, pigs, horses, rabbits, rodents such as mice and rats. In some embodiments, the term “subject,” “patient” or “animal” refers to a male. In some embodiments, the term “subject,” “patient” or “animal” refers to a female.

The term “antibody”, as used herein, means an immunoglobulin or a part thereof, and encompasses any polypeptide comprising an antigen-binding site regardless of the source, method of production, or other characteristics. The term includes for example, polyclonal, monoclonal, monospecific, polyspecific, humanized, single-chain, chimeric, synthetic, recombinant, hybrid, mutated, and CDR-grafted antibodies. A part of an antibody can include any fragment which can bind antigen, for example, an Fab, F (ab′)₂, Fv, scFv.

The term “biological sources” as used herein refers to the sources from which the target polynucleotides or proteins or peptide fragments may be derived. The source can be of any form of “sample” as described infra, including but not limited to, cell, tissue or fluid. “Different biological sources” can refer to different cells/tissues/organs of the same individual, or cells/tissues/organs from different individuals of the same species, or cells/tissues/organs from different species.

The term “capture reagent” refers to a reagent, for example an antibody or antigen binding protein, capable of binding a target molecule or analyte to be detected in a sample.

The term “gene expression result” refers to a qualitative and/or quantitative result regarding the expression of a gene or gene product. Any method known in the art may be used to quantitate a gene expression result. The gene expression result can be an amount or copy number of the gene, the RNA encoded by the gene, the mRNA encoded by the gene, the protein product encoded by the gene, or any combination thereof. The gene expression result can also be normalized or compared to a standard. The gene expression result can be used, for example, to determine if a gene is expressed, overexpressed, or differentially expressed in two or more samples by comparing the gene expression results from 2 or more samples or one or more samples with a standard or a control.

The term “homology,” as used herein, refers to a degree of complementarity. There may be partial homology or complete homology. The word “identity” may substitute for the word “homology.” A partially complementary nucleic acid sequence that at least partially inhibits an identical sequence from hybridizing to a target nucleic acid is referred to as “substantially homologous.” The inhibition of hybridization of the completely complementary nucleic acid sequence to the target sequence may be examined using a hybridization assay (Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

As used herein, the term “hybridization” or “hybridizing” refers to hydrogen bonding, which may be Watson-Crick, Hoogsteen or reversed Hoogsteen hydrogen bonding between complementary nucleoside or nucleotide bases. For example, adenine and thymine are complementary nucleobases which pair through the formation of hydrogen bonds. “Complementary,” as used herein in reference to nucleic acid molecules refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that a nucleic acid sequence need not be 100% complementary to that of its target nucleic acid to be specifically hybridizable. A nucleic acid compound is specifically hybridizable when there is binding of the molecule to the target, and there is a sufficient degree of complementarity to avoid non-specific binding of the molecule to non-target sequences under conditions in which specific binding is desired, i.e., under physiological conditions in the case of in vivo assays or therapeutic treatment, and in the case of in vitro assays, under conditions in which the assays are performed.

The term “inhibiting” includes the administration of a compound of the present disclosure to prevent the onset of the symptoms, alleviating the symptoms, or eliminating the disease, condition or disorder. The term “inhibiting” may also refer to lowering the expression level of gene, such as a gene encoding a cancer associated sequence. Expression level of RNA and/or protein may be lowered.

The term “label” and/or detectible substance refer to a composition capable of producing a detectable signal indicative of the presence of the target polynucleotide or a polypeptide or protein in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by a device or method, such as, but not limited to, a spectroscopic, photochemical, biochemical, immunochemical, electrical, optical, chemical detection device or any other appropriate device. In some embodiments, the label may be detectable visually without the aid of a device. The term “label” is used to refer to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

A “microarray” is a linear or two-dimensional array of, for example, discrete regions, each having a defined area, formed on the surface of a solid support. The density of the discrete regions on a microarray is determined by the total numbers of target polynucleotides to be detected on the surface of a single solid phase support, preferably at least about 50/cm² more preferably at least about 100/cm², even more preferably at least about 500/cm², and still more preferably at least about 1,000/cm². As used herein, a DNA microarray is an array of oligonucleotide primers placed on a chip or other surfaces used to identify, amplify, detect, or clone target polynucleotides. Since the position of each particular group of primers in the array is known, the identities of the target polynucleotides can be determined based on their binding to a particular position in the microarray.

As used herein, the term “naturally occurring” refers to sequences or structures that may be in a form normally found in nature. “Naturally occurring” may include sequences in a form normally found in any animal.

The use of “nucleic acid,” “polynucleotide” or “oligonucleotide” or equivalents herein means at least two nucleotides covalently linked together. In some embodiments, an oligonucleotide is an oligomer of 6, 8, 10, 12, 20, 30 or up to 100 nucleotides. In some embodiments, an oligonucleotide is an oligomer of at least 6, 8, 10, 12, 20, 30, 40, 50, 60, 70, 80, 90, 100, 150, 200, 300, 400, or 500 nucleotides. A “polynucleotide” or “oligonucleotide” may comprise DNA, RNA, PNA or a polymer of nucleotides linked by phosphodiester and/or any alternate bonds.

As used herein, the term “optional” or “optionally” refers to embodiments where the subsequently described structure, event or circumstance may or may not occur, and that the description includes instances where the event occurs and instances where it does not.

The phrases “percent homology,” “% homology,” “percent identity,” or “% identity” refer to the percentage of sequence similarity found in a comparison of two or more amino acid or nucleic acid sequences. Percent identity can be determined electronically, e.g., by using the MEGALIGN program (LASERGENE software package, DNASTAR). The MEGALIGN program can create alignments between two or more sequences according to different methods, e.g., the Clustal Method. (Higgins, D. G. and P. M. Sharp (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be calculated by the Clustal Method, or by other methods known in the art, such as the Jotun Hein Method. (See, e.g., Hein, J. (1990) Methods Enzymol. 183:626-645.) Identity between sequences can also be determined by other methods known in the art, e.g., by varying hybridization conditions.

By “pharmaceutically acceptable”, it is meant the carrier, diluent or excipient must be compatible with the other ingredients of the formulation and not deleterious to the recipient thereof.

“Recombinant protein,” as used herein, means a protein made using recombinant techniques, for example, but not limited to, through the expression of a recombinant nucleic acid as depicted infra. A recombinant protein may be distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises about 50-75%, about 80%, or about 90%. In some embodiments, a substantially pure protein comprises about 80-99%, 85-99/%, 90-99%, 95-99%, or 97-99% by weight of the total protein. A recombinant protein can also include the production of a cancer associated protein from one organism (e.g. human) in a different organism (e.g. yeast, E. coli, or the like) or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed herein.

As used herein, the term “sample” refers to composition that is being tested or treated with a reagent, agent, capture reagent, binding partner and the like. Samples may be obtained from subjects. In some embodiments, the sample may be blood, plasma, serum, or any combination thereof. A sample may be derived from blood, plasma, serum, or any combination thereof. Other typical samples include, but are not limited to, any bodily fluid obtained from a mammalian subject, tissue biopsy, sputum, lymphatic fluid, blood cells (e.g., peripheral blood mononuclear cells), tissue or fine needle biopsy samples, urine, peritoneal fluid, colostrums, breast milk, fetal fluid, fecal material, tears, pleural fluid, or cells therefrom. The sample may be processed in some manner before being used in a method described herein, for example a particular component to be analyzed or tested according to any of the methods described infra. One or more molecules, such as proteins or nucleic acids may be analyzed in a sample to determine the level of expression. One or more molecules may be isolated from a sample for example prior to analysis to determine the expression level.

As used herein, a polynucleotide “derived from” a designated sequence refers to a polynucleotide sequence which is comprised of a sequence of approximately at least about 6 nucleotides, preferably at least about 8 nucleotides, more preferably at least about 10-12 nucleotides, and even more preferably at least about 15-20 nucleotides corresponding to a region of the designated nucleotide sequence. “Corresponding” means homologous to or complementary to the designated sequence. Preferably, the sequence of the region from which the polynucleotide is derived is homologous to or complementary to a sequence that is unique to a cancer associated gene.

As used herein, the term “tag,” “sequence tag” or “primer tag sequence” refers to an oligonucleotide with specific nucleic acid sequence that serves to identify a batch of polynucleotides bearing such tags therein. Polynucleotides from the same biological source are covalently tagged with a specific sequence tag so that in subsequent analysis the polynucleotide can be identified according to its source of origin. The sequence tags also serve as primers for nucleic acid amplification reactions.

The term “support” refers to conventional supports such as beads, particles, dipsticks, fibers, filters, membranes, and silane or silicate supports such as glass slides.

As used herein, the term “therapeutic” or “therapeutic agent” means an agent that can be used to treat, combat, ameliorate, prevent or improve an unwanted condition or disease of a patient. In part, embodiments of the present disclosure are directed to the treatment of cancer or the decrease in proliferation of cells. In some embodiments, the term “therapeutic” or “therapeutic agent” may refer to any molecule that associates with or affects the target marker or cancer associated sequence disclosed infra, its expression or its function. In various embodiments, such therapeutics may include molecules such as, for example, a therapeutic cell, a therapeutic peptide, a therapeutic gene, a therapeutic compound, or the like, that associates with or affects the target marker or cancer associated sequence disclosed infra, its expression or its function.

A “therapeutically effective amount” or “effective amount” of a composition is a predetermined amount calculated to achieve the desired effect, i.e., to inhibit, block, or reverse the activation, migration, metastasis, or proliferation of cells. In some embodiments, the effective amount is a prophylactic amount. In some embodiments, the effective amount is an amount used to medically treat the disease or condition. The specific dose of a composition administered according to this invention to obtain therapeutic and/or prophylactic effects will, of course, be determined by the particular circumstances surrounding the case, including, for example, the composition administered, the route of administration, and the condition being treated. It will be understood that the effective amount administered will be determined by the physician in the light of the relevant circumstances including the condition to be treated, the choice of composition to be administered, and the chosen route of administration. A therapeutically effective amount of composition of this invention is typically an amount such that when it is administered in a physiologically tolerable excipient composition, it is sufficient to achieve an effective systemic concentration or local concentration in the targeted tissue.

The terms “treat,” “treated,” or “treating” as used herein can refer to both therapeutic treatment or prophylactic or preventative measures, wherein the object is to prevent or slow down (lessen) an undesired physiological condition, symptom, disorder or disease, or to obtain beneficial or desired clinical results. In some embodiments, the term may refer to both treating and preventing. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “tissue” refers to any aggregation of similarly specialized cells that are united in the performance of a particular function.

Cancer Associated Sequences

In some embodiments, the present disclosure provides for nucleic acid and protein sequences that are associated with cancer, herein termed “cancer associated” or “CA” sequences. In some embodiments the present disclosure provides nucleic acids and proteins sequences associated with benign thyroid tumors. In some embodiments, the present disclosure provides nucleic acid and protein sequences that are associated with thyroid cancers or carcinomas such as, without limitation, carcinoma, any malignant thyroid neoplasm, papillary thyroid cancer, follicular thyroid cancer, medullary thyroid cancer, anaplastic thyroid cancer, lymphoma, squamous cell carcinoma, papillary microsarcoma, or a combination thereof. The method of diagnosing may comprise measuring the level of expression of a cancer associated marker disclosed herein. The method may further comprise comparing the expression level of the cancer associated sequence with a standard and/or a control. The standard may be from a sample known to contain thyroid cancer cells. The control may include known thyroid cancer cells and/or non-cancerous cells, such as non-cancer cells derived from thyroid tissue or a sample containing a benign thyroid tumor.

Cancer associated sequences may include those that are up-regulated (i.e. expressed at a higher level), as well as those that are down-regulated (i.e. expressed at a lower level), in cancers. Cancer associated sequences can also include sequences that have been altered (i.e., translocations, truncated sequences or sequences with substitutions, deletions or insertions, including, but not limited to, point mutations) and show either the same expression profile or an altered profile. In some embodiments, the cancer associated sequences are from humans; however, as will be appreciated by those in the art, cancer associated sequences from other organisms may be useful in animal models of disease and drug evaluation; thus, other cancer associated sequences may be useful, including those obtained from any subject, such as, without limitation, sequences from vertebrates, including mammals, such as rodents (rats, mice, hamsters, guinea pigs, etc.), primates, and farm animals (including sheep, goats, pigs, cows, horses, etc.). Cancer associated sequences from other organisms may be obtained using the techniques outlined herein.

Examples of cancer associated sequences include SEQ ID NOS: 1-29.

In some embodiments the invention provides one or more markers that are expressed at higher levels in malignant thyroid tumor cells, e.g., follicular carcinoma cells compared to benign thyroid tumor cells. As an example one or more of the following markers are express at higher levels in malignant follicular carcinoma tumor cells compared to follicular adenoma tumor cells: C14orf78, PLAG1, CRABP2, FLJ30058, NMU. In other embodiments the invention provides one or more markers that are expressed at higher levels in benign thyroid tumors compared to malignant thyroid tumors. An example of a marker that may be expressed at higher level in an adenoma cell compared to a carcinoma cell includes TNFRSF11B and KIAA1324.

In some embodiments, the cancer associated sequences are nucleic acids. As will be appreciated by those skilled in the art and as described herein, cancer associated sequences of embodiments herein may be useful in a variety of applications including diagnostic applications to detect nucleic acids or their expression levels in a subject, therapeutic applications or a combination thereof. Further, the cancer associated sequences of embodiments herein may be used in screening applications; for example, generation of biochips comprising nucleic acid probes to the cancer associated sequences.

A nucleic acid of the present disclosure may include phosphodiester bonds, although in some cases, as outlined below (for example, in antisense applications or when a nucleic acid is a candidate drug agent), nucleic acid analogs may have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripla 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996),). Other analog nucleic acids include those with positive backbones (Denpcy et al., Proc. Natl. Acad. Set. USA 92:6097 (1995); non-ionic backbones (U.S. Pat. Nos. 5,386,023, 5,637,684, 5,602,240, 5,216,141 and U.S. Pat. No. 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp. 169-176). Several nucleic acid analogs are described in Rawls, C & E News Jun. 2, 1997 page 35. These modifications of the ribose-phosphate backbone may be done for a variety of reasons, for example to increase the stability and half-life of such molecules in physiological environments for use in anti-sense applications or as probes on a biochip.

As will be appreciated by those skilled in the art, such nucleic acid analogs may be used in some embodiments of the present disclosure. In addition, mixtures of naturally occurring nucleic acids and analogs can be made; alternatively, mixtures of different nucleic acid analogs, and mixtures of naturally occurring nucleic acids and analogs may be made.

In some embodiments, the nucleic acids may be single stranded or double stranded or may contain portions of both double stranded or single stranded sequence. As will be appreciated by those skilled in the art, the depiction of a single strand also defines the sequence of the other strand; thus the sequences described herein also includes the complement of the sequence. The nucleic acid may be DNA, both genomic and cDNA, RNA, or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus, for example, the subject units of a peptide nucleic acid, each containing a base, are referred to herein as a nucleoside.

In some embodiments, cancer associated sequences may include both nucleic acid and amino acid sequences. In some embodiments, the cancer associated sequences may include sequences having at least about 60% homology with the disclosed sequences. In some embodiments, the cancer associated sequences may have at least about 65%, about 70%, about 75%, about 80%, about 85%, about 90%, about 95%, about 97%, about 99%, about 99.8% homology with the disclosed sequences. In some embodiments, the cancer associated sequences may be “mutant nucleic acids”. As used herein, “mutant nucleic acids” refers to deletion mutants, insertions, point mutations, substitutions, translocations.

In some embodiments, the cancer associated sequences may be recombinant nucleic acids. By the term “recombinant nucleic acid” herein refers to nucleic acid molecules, originally formed in vitro, in general, by the manipulation of nucleic acid by polymerases and endonucleases, in a form not normally found in nature. Thus a recombinant nucleic acid may also be an isolated nucleic acid, in a linear form, or cloned in a vector formed in vitro by ligating DNA molecules that are not normally joined, are both considered recombinant for the purposes of this invention. It is understood that once a recombinant nucleic acid is made and reintroduced into a host cell or organism, it can replicate using the in vivo cellular machinery of the host cell rather than in vitro manipulations; however, such nucleic acids, once produced recombinantly, although subsequently replicated in vivo, are still considered recombinant or isolated for the purposes of the invention. As used herein, a “polynucleotide” or “nucleic acid” is a polymeric form of nucleotides of any length, either ribonucleotides or deoxyribonucleotides. This term includes double- and single-stranded DNA and RNA. It also includes known types of modifications, for example, labels which are known in the art, methylation, “caps”, substitution of one or more of the naturally occurring nucleotides with an analog, internucleotide modifications-such as, for example, those with uncharged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

The use of microarray analysis of gene expression allows the identification of host sequences associated with thyroid cancer. These sequences may then be used in a number of different ways, including diagnosis, prognosis, screening for modulators (including both agonists and antagonists), antibody generation (for immunotherapy and imaging), etc. However, as will be appreciated by those skilled in the art, sequences that are identified in one type of cancer may have a strong likelihood of being involved in other types of cancers as well. Thus, while the sequences outlined herein are initially identified as correlated with thyroid cancers, they may also be found in other types of cancers as well.

Some embodiments described herein may be directed to the use of cancer associated sequences for diagnosis and treatment of thyroid cancer. In some embodiments, the cancer associated sequence may be selected from: IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf18, TNFRSF11B, AHNAK2, CYTOKERATINE19 or a combination thereof. In some embodiments, these cancer associated sequences may be associated with thyroid cancers including, without limitation, carcinoma, any malignant thyroid neoplasm, papillary thyroid cancer, follicular thyroid cancer, medullary thyroid cancer, anaplastic thyroid cancer, lymphoma, squamous cell carcinoma, papillary microsarcoma, or a combination thereof.

In some embodiments, the cancer associated sequences may be DNA sequences encoding the above mRNA or the cancer associated protein or cancer associated polypeptide expressed by the above mRNA or homologs thereof. In some embodiments, the cancer associated sequence may be a mutant nucleic acid of the above disclosed sequences. In some embodiments, the homolog may have at least about 60%, at least about 65%, at least about 70%, at least about 75%, at least about 80%, at least about 85%, at least about 90%, at least about 95%, at least about 97%, at least about 98%, at least about 99%, at least about 99.5% identity with the disclosed polypeptide sequence.

In some embodiments, an isolated nucleic acid comprises at least 10, 12, 15, 20 or 30 contiguous nucleotides of a sequence selected from the group consisting of the cancer associated polynucleotide sequences disclosed in SEQ ID NOS 1-29.

In some embodiments, the polynucleotide, or its complement or a fragment thereof, further comprises a detectable label, is attached to a solid support, is prepared at least in part by chemical synthesis, is an antisense fragment, is single stranded, is double stranded or comprises a microarray.

In some embodiments, the invention provides an isolated polypeptide, encoded within an open reading frame of a cancer associated sequence selected from the polynucleotide sequences shown in SEQ ID NOS 1-29, or its complement. In some embodiments, the invention provides an isolated polypeptide, wherein said polypeptide comprises the amino acid sequence encoded by a polynucleotide selected from the group consisting of sequences disclosed in SEQ ID NOS 1-29. In some embodiments, the invention provides an isolated polypeptide, wherein said polypeptide comprises the amino acid sequence encoded by a cancer associated polypeptide as described infra.

In some embodiments, the invention further provides an isolated polypeptide, comprising the amino acid sequence of an epitope of the amino acid sequence of a cancer associated polypeptide disclosed infra. The polypeptide or fragment thereof may be attached to a solid support. In some embodiments the invention provides an isolated antibody (monoclonal or polyclonal) or antigen binding fragment thereof, that binds to such a polypeptide. The isolated antibody or antigen binding fragment thereof may be attached to a solid support. The isolated antibody or antigen binding fragment thereof may further comprise a detectable substance.

Some embodiments also provide for antigens (e.g., cancer-associated polypeptides) associated with a variety of cancers as targets for diagnostic and/or therapeutic antibodies, e.g. thyroid cancer. These antigens may also be useful for drug discovery (e.g., small molecules) and for further characterization of cellular regulation, growth, and differentiation.

Methods of Detecting and Diagnosing Thyroid Cancer

In some embodiments the invention provides a method to distinguish between a benign thyroid tumor and a malignant thyroid tumor. The method may comprise contacting a sample with an agent that binds to one or more genes or gene products that is expressed differentially between a benign thyroid tumor and a malignant thyroid tumor. A benign thyroid tumor may include a follicular adenoma. A malignant thyroid tumor may include a follicular carcinoma or a papillary carcinoma. In some embodiments the gene encoding the marker is expressed at higher levels in a malignant tumor compared to a benign tumor. Thus, in some embodiments one or more of the genes encoding the markers PLAG1, CRABP2, FLJ30058, NMU, CCDC85A may be used in the method to distinguish between a benign thyroid tumor and a malignant thyroid tumor. In other embodiments the gene encoding the marker is expressed at higher levels in benign thyroid tumor cells compared to malignant thyroid tumor cells. Thus, in some embodiments the one or more genes encoding the markers TNFRSF11B and KIAA1324 may be used to distinguish between a malignant thyroid tumor and a benign thyroid tumor.

Where the marker is expressed at higher levels in a malignant thyroid tumor compared to a benign thyroid tumor the invention provides a method of distinguishing between a benign thyroid tumor and a malignant thyroid tumor comprising obtaining a sample from a subject; b) contacting the sample obtained from the subject with one or more agents that detect one or more markers expressed by a thyroid tumor cell c) contacting a benign thyroid tumor cell and/or a malignant thyroid tumor cell with the one or more agents from b); and d) comparing the expression level of the marker in the sample obtained from the subject with the expression level in the benign tumor cell and/or the malignant tumor cell, wherein 1) a higher level of expression of the marker in the sample compared to the benign tumor cell indicates that the subject has a malignant thyroid tumor; 2) a level of expression equal to or greater than the level of expression in the malignant thyroid tumor indicates the subject has a malignant thyroid tumor; 3) a level of expression equal to or less than the level of expression in the non-malignant thyroid tumor indicates the subject has a benign thyroid tumor; 4) a level of expression less than the level of expression in the malignant thyroid tumor indicates the subject has a benign thyroid tumor; and 5) equal to or less than the expression level in the benign tumor cell indicates the subject has a benign tumor. Any one or more of the results recited above (i.e. 1-5) may be used to distinguish between a malignant tumor and a benign tumor. Suitable markers include the genes described infra.

Where the marker is expressed at higher levels in a benign thyroid tumor compared to a malignant thyroid tumor the invention provides a method of distinguishing between a benign thyroid tumor and a malignant thyroid tumor comprising obtaining a sample from a subject; b) contacting the sample obtained from the subject with one or more agents that detect one or more markers expressed by a thyroid tumor cell c) contacting a benign thyroid tumor cell and/or a malignant thyroid tumor cell with the one or more agents from b); and d) comparing the expression level of the marker in the sample obtained from the subject with the expression level in the benign tumor cell and/or the malignant tumor cell, wherein 1) a higher level of expression of the marker in the sample compared to the benign tumor cell indicates that the subject has a benign thyroid tumor; 2) a level of expression equal to or greater than the level of expression in the malignant thyroid tumor indicates the subject has a benign thyroid tumor; 3) a level of expression equal to or less than the level of expression in the non-malignant thyroid tumor indicates the subject has a benign thyroid tumor; 4) a level of expression less than the level of expression in the malignant thyroid tumor indicates the subject has a benign thyroid tumor; and 5) equal to or less than the expression level in the benign tumor cell indicates the subject has a benign tumor. Any one or more of the results recited above (i.e. 1-5) may be used to distinguish between a malignant tumor and a benign tumor. Suitable markers include the genes described infra.

In some embodiments, the method of detecting or diagnosing thyroid cancer may comprise assaying gene expression of a subject in need thereof. In some embodiments, detecting a level of a cancer associated sequence may comprise techniques such as, but not limited to, PCR, mass spectroscopy, microarray, gel electrophoresis, western blots, Southern blots, northern blots, immune-precipitation, immune-cytochemistry, flow cytometry, affinity chromatography, hybridization using one more probes that specifically bind a nucleic acid encoding a cancer associated sequence disclosed infra. Information relating to expression of the receptor can also be useful in determining therapies aimed at up or down-regulating the cancer associated sequence's signaling using agonists or antagonists.

In some embodiments, a method of diagnosing thyroid cancer may comprise detecting a level of the cancer associated protein in a subject. In some embodiments, a method of screening for cancer may comprise detecting a level of the cancer associated protein. In some embodiments, the cancer associated protein is encoded by a nucleotide sequence selected from a sequence disclosed in SEQ ID NOS 1-29, a fragment thereof or a complementary sequence thereof. In some embodiments, a method of detecting cancer in a sample may comprise contacting the sample obtained from a subject with an antibody that specifically binds the protein. In some embodiments, the antibody may be a monoclonal antibody or a polyclonal antibody. In some embodiments, the antibody may be a humanized or a recombinant antibody. Antibodies can be made that specifically bind to this region using known methods and any method is suitable. In some embodiments, the antibody specifically binds to one or more of a molecule, such as protein or peptide, encoded for by one or more cancer associated sequences disclosed infra.

In some embodiments, the antibody binds to an epitope from a protein encoded by the nucleotide sequence disclosed in SEQ ID NOS: 1-29 with an antibody against the protein. In some embodiments, the epitope is a fragment of the protein sequence encoded by the nucleotide sequence of any of the cancer associated sequences disclosed infra. In some embodiments, the epitope comprises about 1-10, 1-20, 1-30, 3-10, or 3-15 residues of the cancer associated sequence. In some embodiments, the epitope is not linear.

In some embodiments, the antibody binds to the regions described herein or a peptide with at least 90, 95, or 99% homology or identity to the region. In some embodiments, the fragment of the regions described herein is 5-10 residues in length. In some embodiments, the fragment of the regions (e.g. epitope) described herein are 3-5 residues in length. The fragments are described based upon the length provided. In some embodiments, the epitope is about 3, 4, 5, 6, 7, 8, 9, 10, 11, 12, 13, 14, 15, or 20 residues in length.

In some embodiments, the sequence to which the antibody binds may include both nucleic acid and amino acid sequences. In some embodiments, the sequence to which the antibody binds may include sequences having at least about 60% homology with the disclosed sequences. In some embodiments, the sequence to which the antibody binds may have at least about 65%, about 70%, about 75%, about 80%, about 85%, about 90%, about 95%, about 97%, about 99%, about 99.8% homology with the disclosed sequences. In some embodiments, the sequences may be referred to as “mutant nucleic acids” or “mutant peptide sequences.”

In some embodiments, a subject can be diagnosed with thyroid cancer by detecting the presence of a cancer associated sequence (e.g. SEQ ID NOS: 1-29) in a sample obtained from a subject. In some embodiments, the method comprises detecting the presence or absence of a cancer associated sequence selected from sequences disclosed in SEQ ID NOS 1-29, wherein the absence of the cancer associated sequence indicates that absence of thyroid cancer. In some embodiments, the method further comprises treating the subject diagnosed with thyroid cancer with an antibody that binds to a cancer associated sequence disclosed infra and inhibits the growth or progression of the thyroid cancer. As discussed, thyroid cancer may be detected in any type of sample, including, but not limited to, serum, blood, tumor tissue and the like. The sample may be any type of sample as it is described herein.

Any suitable assay may be used to screen for the presence, absence or expression level of one or more proteins encoded for by a cancer associated sequence described infra. In some embodiments the assay may be for example an ELISA, a radio-immuno assay, a western blot, a flow cytometry assay and the like.

In some embodiments, the method of diagnosing a subject with thyroid cancer comprises obtaining a sample and detecting the presence of a cancer associated sequence selected from sequences disclosed in SEQ ID NOS: 1-29, wherein the presence of the cancer associated sequence indicates the subject has thyroid cancer. In some embodiments, detecting the presence of a cancer associated sequence selected from sequences disclosed infra comprises contacting the sample with an antibody or other type of capture reagent or specific binding partner that specifically binds to the cancer associated sequence's protein and detecting the presence or absence of the binding to the cancer associated sequence's protein in the sample.

In some embodiments, the present disclosure provides a method of diagnosing thyroid cancer, or a neoplastic condition in a subject, the method comprising obtaining a cancer associated sequence gene expression result of a cancer associated sequence selected from sequences disclosed infra from a sample derived from a subject; and diagnosing thyroid cancer or a neoplastic condition in the subject based on the cancer associated sequence gene expression result, wherein the subject is diagnosed as having thyroid cancer or a neoplastic condition if the cancer associated sequence is expressed at a level that is 1) higher than a negative control such a non-cancerous thyroid tissue or cell sample and/or 2) higher than or equivalent to the expression level of the cancer associated sequence in a standard or positive control wherein the standard or positive control is known to contain thyroid cancer cells.

Some embodiments are directed to a biochip comprising one or more nucleic acid sequences which encodeone or more cancer associated proteins. In some embodiments, a biochip comprises a nucleic acid molecule which encodes at least a portion of a cancer associated protein. In some embodiments, the cancer associated protein is encoded by a sequence selected from SEQ ID NOS 1-29, homologs thereof, combinations thereof, or a fragment thereof. In some embodiments, the nucleic acid molecule specifically hybridizes with a nucleic acid sequence selected from SEQ ID NOS 1-29. In some embodiments, the biochip comprises a first and second nucleic molecule wherein the first nucleic acid molecule specifically hybridizes with a first sequence selected from cancer associated sequences disclosed infra and the second nucleic acid molecule specifically hybridizes with a second sequence selected from cancer associated sequences disclosed infra, wherein the first and second sequences are not the same sequence. In some embodiments, the present invention provides methods of detecting or diagnosing cancer, such as thyroid cancer, comprising detecting the expression of a nucleic acid sequence selected from a sequence disclosed in SEQ ID NOS: 1-29, wherein a sample is contacted with a biochip comprising a sequence selected from sequences disclosed in SEQ ID NOS: 1-29, homologs thereof, combinations thereof; or a fragment thereof.

Also provided herein is a method for diagnosing or determining the propensity to cancers, for example thyroid cancer, by measuring the expression level of one or more of the cancer associated sequences disclosed infra in a sample and comparing the expression level of the one or more cancer associated sequences in the sample with expression level of the same cancer associated sequences in a non-cancerous cell. A higher level of expression of one or more of the cancer associated sequences disclosed infra compared to the non-cancerous cell indicates a propensity for the development of cancer, e.g., thyroid cancer.

In some embodiments, the invention provides a method for detecting a cancer associated sequence with the expression of a polypeptide in a test sample, comprising detecting a level of expression of at least one polypeptide such as, without limitation, a cancer associated protein encoded for by a sequence disclosed infra, or a fragment thereof. In some embodiments, the method comprises comparing the level of expression of the polypeptide in the test sample with a level of expression of polypeptide in a normal sample, i.e. a non-cancerous sample, wherein an altered level of expression of the polypeptide in the test sample relative to the level of polypeptide expression in the normal sample is indicative of the presence of cancer in the test sample. In some embodiments, the polypeptide expression is compared to a cancer sample, wherein the level of expression is at least the same as the cancer is indicative of the presence of cancer in the test sample. In some embodiments the test sample is compared to a normal, e.g. a non-cancerous sample where an expression level in the test sample that is greater than that found in the normal sample indicates the presence of cancer in the test sample. In some embodiments, the sample is a cell sample. In some embodiments the sample is a tissue sample. In some embodiments the sample is a bodily fluid. Examples of suitable bodily fluids, include, but are not limited to, blood, serum, saliva or urine. In some embodiments the sample is a blood sample. In some embodiments the sample is a serum sample. In some embodiments the sample is a urine sample.

In some embodiments, the invention provides a method for detecting cancer by detecting the presence of an antibody in a test serum sample. In some embodiments, the antibody recognizes a polypeptide or an epitope of a cancer associated sequence disclosed herein. In some embodiments, the method comprises detecting a level of an antibody against an antigenic polypeptide such as, without limitation, a cancer associated protein such as a protein encoded for by a cancer associated sequence disclosed infra, or an antigenic fragment thereof. In some embodiments, the method comprises comparing the level of the antibody in the test sample with a level of the antibody in the control sample, wherein an altered level of antibody in said test sample relative to the level of antibody in the control sample is indicative of the presence of cancer in the test sample. In some embodiments, the control sample is a sample derived from a non-cancerous sample e.g. blood or serum obtained from a subject that is cancer free. In some embodiments, the control is derived from a cancer sample, and, therefore, in some embodiments, the method comprises comparing the levels of binding and/or the amount of antibody in the sample, wherein when the levels or amount are the same as the cancer control sample is indicative of the presence of cancer in the test sample.

In some embodiments, a method for diagnosing cancer or a neoplastic condition comprises a) determining the expression of one or more genes comprising a nucleic acid sequence selected from the group consisting of the human genomic and mRNA sequences described in SEQ ID NOS: 1-29, in a first sample type (e.g. tissue, bodily fluid, etc.) of a first individual; and b) comparing said expression of said gene(s) from a second normal sample type from said first individual or a second unaffected individual; wherein a difference in said expression indicates that the first individual has cancer. In some embodiments, the expression is increased as compared to the normal sample.

In some embodiments, the invention also provides a method for detecting presence or absence of cancer cells in a subject. In some embodiments, the method comprises contacting one or more cells from the subject with an antibody as described herein. The antibody may be conjugated to a detectible substance. In some embodiments the antibody that binds to a protein encoded for by a cancer associated sequence disclosed infra may bind to a second antibody wherein the second antibody is conjugated to a detectible substance. In some embodiments the antibody that binds to a protein encoded for by a cancer associated sequence disclosed infra is bound to a solid support. In some embodiments, the method comprises detecting a complex of a cancer associated protein and the antibody, wherein detection of the complex indicates with the presence of cancer cells in the subject. The complex may include a detectable substance as described infra. The complex may include a solid support, such as bead, a chip, a magnet, a multiwell plate and the like.

In some embodiments, the present disclosure provides methods of detecting cancer in a test sample, comprising: (i) detecting a level of activity of at least one polypeptide that is a gene product; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample, wherein said gene product is a product of a gene selected from one or more of the cancer associated sequences provided infra.

Capture Reagents and Specific Binding Partners

The invention provides for specific binding partners and capture reagents that bind specifically to cancer associated sequences disclosed infra and the polypeptides or proteins encoded for by those sequences. The capture reagents and specific binding partners may be used in diagnostic assays as disclosed infra and/or in therapeutic methods described infra as well as in drug screening assays disclosed infra. Capture reagents include for example nucleic acids and proteins. Suitable proteins include antibodies.

As used herein, the term “specifically binds” or “specifically binding” means binding that is measurably different from a non-specific interaction. Specific binding can be measured, for example, by determining binding of a molecule compared to binding of a control molecule, which generally is a molecule of similar structure that does not have binding activity. For example, specific binding is indicated if the molecule has measurably higher affinity for cells expressing a protein encoded for by a cancer associated sequence disclosed infra than for cells that do not express the same protein encoded for by the cancer associated sequences disclosed infra. Specificity of binding can be determined, for example, by competitive inhibition of a known binding molecule.

The term “specifically binding,” as used herein, includes both low and high affinity specific binding. Specific binding can be exhibited, for example, by a low affinity homing molecule having a Kd of at least about 10⁻⁴ M. Specific binding also can be exhibited by a high affinity homing molecule, for example, a homing molecule having a Kd of at least about 10⁻⁵ M. Such a molecule can have, for example, a Kd of at least about 10⁻⁶ M, at least about 10⁻⁷ M, at least about 10⁻⁴ M, at least about 10⁻⁹ M, at least about 10⁻¹⁰ M, or can have a Kd of at least about 10⁻¹ M or 10¹² M or greater. Both low and high affinity homing molecules are useful and are encompassed by the invention. Low affinity homing molecules are useful in targeting, for example, multivalent conjugates. High affinity homing molecules are useful in targeting, for example, multivalent and univalent conjugates.

In some embodiments the specific binding partner or capture reagent is an antibody. Binding in IgG antibodies, for example, is generally characterized by an affinity of at least about 10⁻⁷ M or higher, such as at least about 10⁻⁸ M or higher, or at least about 10⁻⁹ M or higher, or at least about 10⁻¹⁰ or higher, or at least about 10⁻¹ M or higher, or at least about 10⁻¹² M or higher. The term is also applicable where, e.g., an antigen-binding domain is specific for a particular epitope that is not carried by numerous antigens, in which case the antibody or antigen binding protein carrying the antigen-binding domain will generally not bind other antigens. In some embodiments, the capture reagent has a Kd equal or less than 10⁻⁹ M, 10⁻¹⁰ M, or 10⁻¹¹ M for its binding partner (e.g. antigen). In some embodiments, the capture reagent has a Ka greater than or equal to 10⁹ M⁻¹ for its binding partner. Capture reagent can also refer to, for example, antibodies. Intact antibodies, also known as immunoglobulins, are typically tetrameric glycosylated proteins composed of two light (L) chains of approximately 25 kDa each, and two heavy (H) chains of approximately 50 kDa each. Two types of light chain, termed lambda and kappa, exist in antibodies. Depending on the amino acid sequence of the constant domain of heavy chains, immunoglobulins are assigned to five major classes: A, D, E, G, and M, and several of these may be further divided into subclasses (isotypes), e.g., IgG, IgG2, IgG3, IgG4, IgA1, and IgA2. Each light chain is composed of an N-terminal variable (V) domain (VL) and a constant (C) domain (CL). Each heavy chain is composed of an N-terminal V domain (VH), three or four C domains (CHs), and a hinge region. The CH domain most proximal to VH is designated CHI. The VH and VL domains consist of four regions of relatively conserved sequences named framework regions (FR1, FR2, FR3, and FR4), which form a scaffold for three regions of hypervariable sequences (complementarity determining regions, CDRs). The CDRs contain most of the residues responsible for specific interactions of the antibody or antigen binding protein with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is the greatest source of molecular diversity within the antibody or antigen binding protein-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, Eds. Harlow et al., 1988. One of skill in the art will recognize that each subunit structure, e.g., a CH, VH, CL, VL, CDR, and/or FR structure, comprises active fragments. For example, active fragments may consist of the portion of the VH, VL, or CDR subunit that binds the antigen, i.e., the antigen-binding fragment, or the portion of the CH subunit that binds to and/or activates an Fc receptor and/or complement.

Non-limiting examples of binding fragments encompassed within the term “antigen-specific antibody” used herein include: (i) an Fab fragment, a monovalent fragment consisting of the VL, VH, CL and CHI domains; (ii) an F(ab′)2 fragment, a bivalent fragment comprising two Fab fragments linked by a disulfide bridge at the hinge region; (iii) an Fd fragment consisting of the VH and CH 1 domains; (iv) an Fv fragment consisting of the VL and VH domains of a single arm of an antibody, (v) a dAb fragment, which consists of a VH domain; and (vi) an isolated CDR. Furthermore, although the two domains of the Fv fragment, VL and VH, are coded for by separate genes, they may be recombinantly joined by a synthetic linker, creating a single protein chain in which the VL and VH domains pair to form monovalent molecules (known as single chain Fv (scFv)). The most commonly used linker is a 15-residue (Gly₄Ser)₃ peptide, but other linkers are also known in the art. Single chain antibodies are also intended to be encompassed within the terms “antibody or antigen binding protein,” or “antigen-binding fragment” of an antibody. The antibody can also be a polyclonal antibody, monoclonal antibody, chimeric antibody, antigen-binding fragment, Fc fragment, single chain antibodies, or any derivatives thereof.

Antibodies can be obtained using conventional techniques known to those skilled in the art, and the fragments are screened for utility in the same manner as intact antibodies. Antibody diversity is created by multiple germline genes encoding variable domains and a variety of somatic events. The somatic events include recombination of variable gene segments with diversity (D) and joining (J) gene segments to make a complete VH domain, and the recombination of variable and joining gene segments to make a complete VL domain. The recombination process itself is imprecise, resulting in the loss or addition of amino acids at the V (D) J junctions. These mechanisms of diversity occur in the developing B cell prior to antigen exposure. After antigenic stimulation, the expressed antibody genes in B cells undergo somatic mutation. Based on the estimated number of germline gene segments, the random recombination of these segments, and random VH-VL pairing, up to 1.6×10⁷ different antibodies may be produced (Fundamental Immunology, 3rd ed. (1993), ed. Paul, Raven Press, New York, N.Y.). When other processes that contribute to antibody diversity (such as somatic mutation) are taken into account, it is thought that upwards of 1×10 different antibodies may be generated (Immunoglobulin Genes, 2nd ed. (1995), eds. Jonio et al., Academic Press, San Diego, Calif.). Because of the many processes involved in generating antibody diversity, it is unlikely that independently derived monoclonal antibodies with the same antigen specificity will have identical amino acid sequences.

Antibody or antigen binding protein molecules capable of specifically interacting with the antigens, epitopes, or other molecules described herein may be produced by methods well known to those skilled in the art. For example, monoclonal antibodies can be produced by generation of hybridomas in accordance with known methods. Hybridomas formed in this manner can then be screened using standard methods, such as enzyme-linked immunosorbent assay (ELISA) and Biacore analysis, to identify one or more hybridomas that produce an antibody that specifically interacts with a molecule or compound of interest. As an alternative to preparing monoclonal antibody-secreting hybridomas, a monoclonal antibody to a polypeptide of the present disclosure may be identified and isolated by screening a recombinant combinatorial immunoglobulin library (e.g., an antibody phage display library) with a polypeptide of the present disclosure to thereby isolate immunoglobulin library members that bind to the polypeptide. Techniques and commercially available kits for generating and screening phage display libraries are well known to those skilled in the art. Additionally, examples of methods and reagents particularly amenable for use in generating and screening antibody or antigen binding protein display libraries can be found in the literature.

Examples of chimeric antibodies include, but are not limited to, humanized antibodies. The antibodies described herein can also be human antibodies. In some embodiments, the capture reagent comprises a detection reagent. The detection reagent can be any reagent that can be used to detect the presence of the capture reagent binding to its specific binding partner. The capture reagent can comprise a detection reagent directly or the capture reagent can comprise a particle that comprises the detection reagent. In some embodiments, the capture reagent and/or particle comprises a color, colloidal gold, radioactive tag, fluorescent tag, or a chemiluminescent substrate. The particle can be, for example, a viral particle, a latex particle, a lipid particle, or a fluorescent particle.

The capture reagents (e.g. antibody) of the present disclosure can also include an anti-antibody, i.e. an antibody that recognizes another antibody but is not specific to an antigen, such as, but not limited to, anti-IgG, anti-IgM, or ant-IgE antibody. This non-specific antibody can be used as a positive control to detect whether the antigen specific antibody is present in a sample.

Nucleic acid capture reagents include DNA, RNA and PNA molecules for example. The nucleic acid may be about 5 nucleotides long, about 10 nucleotides long, about 15 nucleotides long, about 20 nucleotides long, about 25 nucleotides long, about 30 nucleotides long, about 35 nucleotides long about 40 nucleotides long. The nucleic acid may be greater than 30 nucleotides long. The nucleic acid may be less than 30 nucleotides long.

Treatment of Thyroid Cancer

In some embodiments, thyroid cancers expressing one of the cancer associated sequences disclosed infra may be treated by antagonizing the cancer associated sequence's activity. In some embodiments, a method of treating thyroid cancer may comprise administering a therapeutic such as, without limitation, antibodies that antagonize the ligand binding to the cancer associated sequence, small molecules that inhibit the cancer associated sequence's expression or activity, siRNAs directed towards the cancer associated sequence, or the like.

In some embodiments, a method of treating cancer (e.g. thyroid or other types of cancer) comprises detecting the presence of a cancer associated sequence's receptor and administering a cancer treatment. The treatment may specifically bind to the cancer associated sequence's receptor. The cancer treatment may be any cancer treatment or one that is specific to the inhibiting the action of a cancer associated sequence. For example, various cancers are tested to determine if a specific molecule is present before giving a cancer treatment. In some embodiments, therefore, a sample would be obtained from the patient and tested for the presence of a cancer associated sequence or the overexpression of a cancer associated sequence as described herein. In some embodiments, if a cancer associated sequence is found to be overexpressed then a thyroid cancer treatment or therapeutic is administered to the subject. The thyroid cancer treatment may be a conventional non-specific treatment, such as chemotherapy, or the treatment may comprise a specific treatment that only targets the activity of the cancer associated sequence or the receptor to which the cancer associated sequence binds. These treatments can be, for example, an antibody that specifically binds to the cancer associated sequence and inhibits its activity. The treatment may be a nucleic acid that downregulates or silences the expression of the cancer associated sequence.

Some embodiments herein describe method of treating cancer or a neoplastic condition comprising administering an antibody against the cancer associated sequence to a subject. In some embodiments, the antibody may be monoclonal or polyclonal. In some embodiments, the antibody may be humanized or recombinant. In some embodiments, the antibody may neutralize biological activity of the cancer associated sequence by binding to and/or interfering with the cancer associated sequence's receptor. In some embodiments the antibody may bind to site on the protein encoded for by the cancer associated DNA sequence that is not the receptor. In some embodiments, administering the antibody may be to a biological fluid or tissue, such as, without limitation, blood, urine, serum, tumor tissue, or the like.

In some embodiments, a method of treating cancer may comprise administering an agent that interferes with the synthesis, secretion, receptor binding or receptor signaling of cancer associated proteins or its receptors. In some embodiments, the cancer may be selected from, including, without limitation, carcinoma, any malignant thyroid neoplasm, papillary thyroid cancer, follicular thyroid cancer, medullary thyroid cancer, anaplastic thyroid cancer, lymphoma, squamous cell carcinoma, papillary microsarcoma, or a combination thereof.

In some embodiments, the cancer cell may be targeted specifically with a therapeutic based upon the differentially expressed gene or gene product. For example, in some embodiments, the differentially expressed gene product may be an enzyme, which can convert an anti-cancer prodrug into its active form. Therefore, in normal cells, where the differentially expressed gene product is not expressed or expressed at significantly lower levels, the prodrug may be either not activated or activated in a lesser amount, and may be, therefore less toxic to normal cells. Therefore, the cancer prodrug may, in some embodiments, be given in a higher dosage so that the cancer cells can metabolize the prodrug, which will, for example, kill the cancer cell, and the normal cells will not metabolize the prodrug or not as well, and, therefore, be less toxic to the patient. An example of this is where tumor cells overexpress a metalloprotease, which is described in Atkinson et al., British Journal of Pharmacology (2008) 153, 1344-1352. Using proteases to target cancer cells Is also described in Carl et al., PNAS, Vol. 77, No. 4, pp. 2224-2228, April 1980. For example, doxorubicin or other type of chemotherapeutic can be linked to a peptide sequence that is specifically cleaved or recognized by the differentially expressed gene product. The doxorubicin or other type of chemotherapeutic is then cleaved from the peptide sequence and is activated such that it can kill or inhibit the growth of the cancer cell whereas in the normal cell the chemotherapeutic is never internalized into the cell or is not metabolized as efficiently, and is, therefore, less toxic.

In some embodiments, a method of treating thyroid cancer may comprise gene knockdown of one or more cancer associated sequences described herein. Gene knockdown refers to techniques by which the expression of one or more of an organism's genes is reduced, either through genetic modification (a change in the DNA of one of the organism's chromosomes such as, without limitation, chromosomes encoding cancer associated sequences) or by treatment with a reagent such as a short DNA or RNA oligonucleotide with a sequence complementary to either an mRNA transcript or a gene. In some embodiments, the oligonucleotide used may be selected from RNase-H competent antisense, such as, without limitation, ssDNA oligonucleotides, ssRNA oligonucleotides, phosphorothioate oligonucleotides, or chimeric oligonucleotides; RNase-independent antisense, such as morpholino oligonucleotides, 2′-O-methyl phosphorothioate oligonucleotides, locked nucleic acid oligonucleotides, or peptide nucleic acid oligonucleotides; RNAi oligonucleotides, such as, without limitation, siRNA duplex oligonucleotides, or shRNA oligonucleotides; or any combination thereof. In some embodiments, a plasmid may be introduced into a cell, wherein the plasmid expresses either an antisense RNA transcript or an shRNA transcript. The oligo introduced or transcript expressed may interact with the target mRNA (ex. sequences disclosed in Table 1) by complementary base pairing (a sense-antisense interaction).

The specific mechanism of silencing may vary with the oligo chemistry. In some embodiments, the binding of a oligonucleotide described herein to the active gene or its transcripts may cause decreased expression through blocking of transcription, degradation of the mRNA transcript (e.g. by small interfering RNA (siRNA) or RNase-H dependent antisense) or blocking either mRNA translation, pre-mRNA splicing sites or nuclease cleavage sites used for maturation of other functional RNAs such as miRNA (e.g. by Morpholino oligonucleotides or other RNase-H independent antisense). For example, RNase-H competent antisense oligonucleotides (and antisense RNA transcripts) may form duplexes with RNA that are recognized by the enzyme RNase-H, which cleaves the RNA strand. As another example, RNase-independent oligonucleotides may bind to the mRNA and block the translation process. In some embodiments, the oligonucleotides may bind in the 5′-UTR and halt the initiation complex as it travels from the 5′-cap to the start codon, preventing ribosome assembly. A single strand of RNAi oligonucleotides may be loaded into the RISC complex, which catalytically cleaves complementary sequences and inhibits translation of some mRNAs bearing partially-complementary sequences. The oligonucleotides may be introduced into a cell by any technique including, without limitation, electroporation, microinjection, salt-shock methods such as, for example, CaCl2 shock; transfection of anionic oligo by cationic lipids such as, for example, Lipofectamine; transfection of uncharged oligonucleotides by endosomal release agents such as, for example, Endo-Porter; or any combination thereof. In some embodiments, the oligonucleotides may be delivered from the blood to the cytosol using techniques selected from nanoparticle complexes, virally-mediated transfection, oligonucleotides linked to octaguanidinium dendrimers (Morpholino oligonucleotides), or any combination thereof.

In some embodiments, a method of treating thyroid cancer may comprise treating a subject with a suitable reagent to knockdown or inhibit expression of a gene encoding the mRNA disclosed in SEQ ID NOS: 1-29, or a combination thereof. In other embodiments the invention provides for the in vitro knockdown of the expression of one or more of the genes disclosed in SEQ ID NOS: 1-29, for example in an in vitro culture of cells or cells obtained from a sample obtained from a subject.

In some embodiments, the cancers treated by modulating the activity or expression of sequences disclosed in Table 1, Table 2 and or SEQ ID NOS: 1-29 or the gene product thereof.

In some embodiments, a method of treating cancer comprises administering an antibody (e.g. monoclonal antibody, human antibody, humanized antibody, recombinant antibody, chimeric antibody, and the like) that specifically binds to a cancer associated protein that is expressed on a cell surface. In some embodiments, the antibody binds to an extracellular domain of the cancer associated protein. In some embodiments, the antibody binds to a cancer associated protein differentially expressed on a cancer cell surface relative to a normal cell surface, or, in some embodiments, to at least one human cancer cell line. In some embodiments, the antibody is linked to a therapeutic agent or a toxin.

In some embodiments, implementation of an immunotherapy strategy for treating, reducing the symptoms of; or preventing cancer or neoplasms, (e.g., a vaccine) may be achieved using many different techniques available to the skilled artisan.

Immunotherapy or the use of antibodies for therapeutic purposes has been used in recent years to treat cancer. Passive immunotherapy involves the use of monoclonal antibodies in cancer treatments. See, for example, Cancer: Principles and Practice of Oncology, 6 Th Edition (2001) Chapt. 20 pp. 495-508. Inherent therapeutic biological activity of these antibodies include direct inhibition of tumor cell growth or survival, and the ability to recruit the natural cell killing activity of the body's immune system. These agents may be administered alone or in conjunction with radiation or chemotherapeutic agents. Alternatively, antibodies may be used to make antibody conjugates where the antibody is linked to a toxic agent and directs that agent to the tumor by specifically binding to the tumor.

Screening for Cancer Therapeutics

The invention provides for screening assays to determine if a candidate molecule has an inhibitory effect on the growth and or metastasis of thyroid cancer cells.

Suitable candidates include proteins, peptides, nucleic acids such as DNA, RNA shRNA sm RNA and the like, small molecules including small organic molecules and small inorganic molecules. A small molecule may include molecules less than 50 kd.

In some embodiments, a method of identifying an anti-cancer agent is provided, wherein the method comprises contacting a candidate agent to a sample; and determining the cancer associated sequence's activity in the sample. In some embodiments, the candidate agent is identified as an anti-cancer agent if the cancer associated sequence's activity is reduced in the sample after the contacting. In other embodiments the candidate agent reduces the expression level of one or more cancer associated sequences disclosed infra.

In some embodiments, the candidate agent is an antibody. In some embodiments, the method comprises contacting a candidate antibody that binds to the cancer associated sequence with a sample, and assaying for the cancer associated sequence's activity, wherein the candidate antibody is identified as an anti-cancer agent if the cancer associated sequence activity is reduced in the sample after the contacting. A cancer associated sequence's activity can be any activity of the cancer associated sequence. An example of an activity may include inhibiting enzymatic activity either of the cancer associated sequence itself or of an enzyme that interacts with or is modulated by the cancer associated sequence either at the nucleic acid level or the protein level.

In some embodiments, the present disclosure provides methods of identifying an anti-cancer (e.g. thyroid cancer) agent comprising contacting a candidate agent to a cell sample; and determining activity of a cancer associated sequence, wherein the candidate agent is identified as an anti-cancer agent if the cancer associated sequence's activity is reduced in the cell sample after the contacting. In some embodiments, the present disclosure provides methods of identifying an anti-cancer agent, the method comprising contacting a candidate agent that binds to a cancer associated sequence selected from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a combination thereof with a cell sample, and assaying for the cancer associated sequence's activity or expression level, wherein the candidate antibody is identified as an anti-cancer agent if the cancer associated sequence's activity is reduced in the cell sample after the contacting.

In some embodiments, a method of screening drug candidates includes comparing the level of expression of the cancer-associated sequence in the absence of the drug candidate to the level of expression in the presence of the drug candidate.

Some embodiments are directed to a method of screening for a therapeutic agent capable of binding to a cancer-associated sequence (nucleic acid or protein), the method comprising combining the cancer-associated sequence and a candidate therapeutic agent, and determining the binding of the candidate agent to the cancer-associated sequence.

Further provided herein is a method for screening for a therapeutic agent capable of modulating the activity of a cancer-associated sequence. In some embodiments, the method comprises combining the cancer-associated sequence and a candidate therapeutic agent, and determining the effect of the candidate agent on the bioactivity of the cancer-associated sequence. An agent that modulates the bioactivity of a cancer associated sequence may be used as a therapeutic agent capable of modulating the activity of a cancer-associated sequence.

In certain embodiments the invention provides a method of screening for anticancer activity comprising: (a) contacting a cell that expresses a cancer associated gene selected from one or more cancer associated sequences disclosed infra, homologs thereof, combinations thereof, or fragments thereof with an anticancer drug candidate; (b) detecting an effect of the anticancer drug candidate on an expression of the cancer associated sequence in the cell (either at the nucleic acid or protein level); and (c) comparing the level of expression in the absence of the drug candidate to the level of expression in the presence of the drug candidate; wherein an effect on the expression of the cancer associate polynucleotide indicates that the candidate has anticancer activity. For example the drug candidate may lower the expression level of the cancer associated sequence in the cell.

In some embodiments, a method of evaluating the effect of a candidate cancer drug may comprise administering the drug to a patient and removing a cell sample from the patient. The expression profile of the cell is then determined. In some embodiments, the method may further comprise comparing the expression profile of the patient to an expression profile of a healthy individual. In some embodiments, the expression profile comprises measuring the expression of one or more or any combination thereof of the sequences disclosed herein. In some embodiments, where the expression profile of one or more or any combination thereof of the sequences disclosed herein is modified (increased or decreased) the candidate cancer drug is said to be effective.

In some embodiments, the invention provides a method of screening for anticancer activity comprising: (a) providing a cell that expresses a cancer associated gene that encodes a nucleic acid sequence selected from the group consisting of the cancer associated sequences shown in SEQ ID NOS 1-29, or fragment thereof, (b) contacting the cell, which can be derived from a cancer cell with an anticancer drug candidate; (c) monitoring an effect of the anticancer drug candidate on an expression of the cancer associated sequence in the cell sample, and optionally (d) comparing the level of expression in the absence of said drug candidate to the level of expression in the presence of the drug candidate.

Suitable drug candidates include, but are not limited to an inhibitor of transcription, a G-protein coupled receptor antagonist, a growth factor antagonist, a serine-threonine kinase antagonist, a tyrosine kinase antagonist. In some embodiments, where the candidate modulates the expression of the cancer associated sequence the candidate is said to have anticancer activity. In some embodiments, the anticancer activity is determined by measuring cell growth. In some embodiments, the candidate inhibits or retards cell growth and is said to have anticancer activity. In some embodiments, the candidate causes the cell to die, and thus, the candidate is said to have anticancer activity.

In some embodiments, the present invention provides a method of screening for activity against thyroid cancer. In some embodiments, the method comprises contacting a cell that overexpresses a cancer associated gene which is complementary to a cancer associated sequence selected from cancer associated sequences disclosed infra, homologs thereof, combinations thereof, or fragments thereof with a thyroid cancer drug candidate. In some embodiments, the method comprises detecting an effect of the thyroid cancer drug candidate on an expression of the cancer associated polynucleotide in the cell or an effect on the cell's growth or viability. In some embodiments, the method comprises comparing the level of expression, cell growth, or viability in the absence of the drug candidate to the level of expression, cell growth, or viability in the presence of the drug candidate; wherein an effect on the expression of the cancer associated polynucleotide, cell growth, or viability indicates that the candidate has activity against a thyroid cancer cell that overexpresses a cancer associated gene, wherein said gene comprises a sequence that is a sequence selected from sequences disclosed in SEQ ID NOS: 1-29, or complementary thereto, homologs thereof, combinations thereof, or fragments thereof. In some embodiments, the drug candidate may include, for example, a transcription inhibitor, a G-protein coupled receptor antagonist, a growth factor antagonist, a serine-threonine kinase antagonist, or a tyrosine kinase antagonist.

Methods of Identifying Thyroid Cancer Markers

The pattern of gene expression in a particular living cell may be characteristic of its current state. Nearly all differences in the state or type of a cell are reflected in the differences in RNA levels of one or more genes. Comparing expression patterns of uncharacterized genes may provide clues to their function. High throughput analysis of expression of hundreds or thousands of genes can help in (a) identification of complex genetic diseases, (b) analysis of differential gene expression over time, between tissues and disease states, and (c) drug discovery and toxicology studies. Increase or decrease in the levels of expression of certain genes correlate with cancer biology. For example, oncogenes are positive regulators of tumorigenesis, while tumor suppressor genes are negative regulators of tumorigenesis. (Marshall, Cell, 64: 313-406 (1991); Weinberg, Science, 254: 1138-1146 (1991)). Accordingly, some embodiments herein provide for polynucleotide and polypeptide sequences involved in cancer and, in particular, in oncogenesis.

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes. Carcinogenesis is fundamentally driven by somatic cell evolution (i.e. mutation and natural selection of variants with progressive loss of growth control). The genes that serve as targets for these somatic mutations are classified as either protooncogenes or tumor suppressor genes, depending on whether their mutant phenotypes are dominant or recessive, respectively.

Some embodiments of the invention are directed to cancer associated sequences (“target markers”). Some embodiments are directed to methods of identifying novel target markers useful in the diagnosis and treatment of cancer wherein expression levels of mRNAs, mRNAs, proteins, or protein post translational modifications including but not limited to phosphorylation and sumoylation are compared between five categories of cell types: (1) immortal pluripotent stem cells (such as embryonic stem (“ES”) cells, induced pluripotent stem (“iPS”) cells, and germ-line cells such as embryonal carcinoma (“EC”) cells) or gonadal tissues; (2) ES, iPS, or EC-derived clonal embryonic progenitor (“EP”) cell lines, (3) nucleated blood cells including but not limited to CD34+ cells and CD133+ cells; (4) normal mortal somatic adult-derived tissues and cultured cells including: skin fibroblasts, vascular endothelial cells, normal non-lymphoid and non-cancerous tissues, and the like, and (5) malignant cancer cells including cultured cancer cell lines or human tumor tissue. mRNAs, miRNAs, or proteins that are generally expressed (or not expressed) in categories 1, 3, and 5, or categories 1 and 5 but not expressed (or expressed) in categories 2 and 4 are candidate targets for cancer diagnosis and therapy. Some embodiments herein are directed to human applications, non-human veterinary applications, or a combination thereof.

In some embodiments, a method of identifying a target marker comprises the steps of: 1) obtaining a molecular profile of the mRNAs, miRNAs, proteins, or protein modifications of immortal pluripotent stem cells (such as embryonic stein (“ES”) cells, induced pluripotent stem (“iPS”) cells, and germ-line cells such as embryonal carcinoma (“EC”) cells); 2) ES, iPS, or EC-derived clonal embryonic progenitor (“EP”) cell lines malignant cancer cells including cultured cancer cell lines or human tumor tissues, and comparing those molecules to those present in mortal somatic cell types such as cultured clonal human embryonic progenitors, cultured somatic cells from fetal or adult sources, or normal tissue counterparts to malignant cancer cells. Target markers that are shared between pluripotent stem cells such as hES cells and malignant cancer cells, but are not present in a majority of somatic cell types may be candidate diagnostic markers and therapeutic targets.

Cancer associated sequences of embodiments herein are disclosed, for example, in SEQ ID NOS 1-29. These sequences were extracted from fold-change and filter analysis. Expression of cancer associated sequences in normal and thyroid tumor tissues is disclosed infra.

Once expression is determined, the gene sequence results may be further filtered by considering fold-change in cancer cell lines vs. normal tissue; general specificity; secreted or not, level of expression in cancer cell lines; and signal to noise ratio.

It will be appreciated that there are various methods of obtaining expression data and uses of the expression data. For example, the expression data that can be used to detect or diagnose a subject with cancer can be obtained experimentally. In some embodiments, obtaining the expression data comprises obtaining the sample and processing the sample to experimentally determine the expression data. The expression data can comprise expression data for one or more of the cancer associated sequences described herein. The expression data can be experimentally determined by, for example, using a microarray or quantitative amplification method such as, but not limited to, those described herein. In some embodiments, obtaining expression data associated with a sample comprises receiving the expression data from a third party that has processed the sample to experimentally determine the expression data.

Detecting a level of expression or similar steps that are described herein may be done experimentally or provided by a third-party as is described herein. Therefore, for example, “detecting a level of expression” may refer to experimentally measuring the data and/or having the data provided by another party who has processed a sample to determine and detect a level of expression data.

The comparison of gene expression on an mRNA level using Illumina gene expression microarrays hybridized to RNA probe sequences may be used. For example samples may be prepared from diverse categories of cell types: 1) human embryonic stem (“ES”) cells, or gonadal tissues 2) ES, iPS, or EC-derived clonal embryonic progenitor (“EP”) cell lines, 3) nucleated blood cells including but not limited to CD34+ cells and CD133+ cells; 4) Normal mortal somatic adult-derived tissues and cultured cells including: skin fibroblasts, vascular endothelial cells, normal non-lymphoid and non-cancerous tissues, and the like, and 5) malignant cancer cells including cultured cancer cell lines or human tumor tissue and filters was performed to detect genes that are generally expressed (or not expressed) in categories 1, 3, and 5, or categories 1 and 5 but not expressed (or expressed) in categories 2 and 4. Therapies in these cancers based on this observation would be based on reducing the expression of the above referenced transcripts up-regulated in cancer, or otherwise reducing the expression of the gene products.

Techniques for Analyzing Samples

Any technique known in the art may be used to analyze a sample according to the methods disclosed infra such as methods of detecting or diagnosing cancer in a sample or identifying a new cancer associated sequence. Exemplary techniques are provided below.

Gene Expression Assays: Measurement of the gene expression levels may be performed by any known methods in the art, including but not limited to quantitative PCR, or microarray gene expression analysis, bead array gene expression analysis and Northern analysis. The gene expression levels may be represented as relative expression normalized to the ADPRT (Accession number NM_{—}001618.2), GAPD (Accession number NM_{—}002046.2), or other housekeeping genes known in the art. In the case of microarrayed probes of mRNA expression, the gene expression data may also be normalized by a median of medians method. In this method, each array gives a different total intensity. Using the median value is a robust way of comparing cell lines (arrays) in an experiment. As an example, the median was found for each cell line and then the median of those medians became the value for normalization. The signal from the each cell line was made relative to each of the other cell lines.

RNA extraction: Cells of the present disclosure may be incubated with 0.05% trypsin and 0.5 mM EDTA, followed by collecting in DMEM (Gibco, Gaithersburg, Md.) with 0.5% BSA. Total RNA may be purified from cells using the RNeasy Mini kit (Qiagen, Hilden, Germany).

Isolation of total RNA and miRNA from cells: Total RNA or samples enriched for small RNA species may be isolated from cell cultures that undergo serum starvation prior to harvesting RNA to approximate cellular growth arrest observed in many mature tissues. Cellular growth arrest may be performed by changing to medium containing 0.5% serum for 5 days, with one medium change 2-3 days after the first addition of low serum medium. RNA may be harvested according to the vendor's instructions for Qiagen RNEasy kits to isolate total RNA or Ambion mirVana kits to isolate RNA enriched for small RNA species. The RNA concentrations may be determined by spectrophotometry and RNA quality may be determined by denaturing agarose gel electrophoresis to visualize 28S and 18S RNA. Samples with clearly visible 28S and 18S bands without signs of degradation and at a ratio of approximately 2:1, 28S:18S may be used for subsequent miRNA analysis.

Assay for miRNA in samples isolated from human cells: The miRNAs may be quantitated using a Human Panel TaqMan MicroRNA Assay from Applied Biosystems, Inc. This is a two-step assay that uses stem-loop primers for reverse transcription (RT) followed by real-time TaqMan®. The assay includes two steps, reverse transcription (RT) and quantitative PCR. Real-time PCR may be performed on an Applied Biosystems 7500 Real-Time PCR System. The copy number per cell may be estimated based on the standard curve of synthetic mir-16 miRNA and assuming a total RNA mass of approximately 15 pg/cell.

The reverse transcription reaction may be performed using 1× cDNA archiving buffer, 3.35 units MMLV reverse transcriptase, 5 mM each dNTP, 1.3 units AB RNase inhibitor, 2.5 nM 330-plex reverse primer (RP), 3 ng of cellular RNA in a final volume of 5 μl. The reverse transcription reaction may be performed on a BioRad or MJ thermocycler with a cycling profile of 20° C. for 30 sec; 42° C. for 30 sec; 50° C. for 1 see, for 60 cycles followed by one cycle of 85° C. for 5 min.

Real-Time PCR.

Two microlitres of 1:400 diluted Pre-PCR product may be used for a 20 ul reaction. All reactions may be duplicated. Because the method is very robust, duplicate samples may be sufficient and accurate enough to obtain values for miRNA expression levels. TaqMan universal PCR master mix of ABI may be used according to manufacturer's suggestion. Briefly, Ix TaqMan Universal Master Mix (ABI), 1 uM Forward Primer, 1 uM Universal Reverse Primer and 0.2 uM TaqMan Probe may be used for each real-time PCR. The conditions used may be as follows: 95° C. for 10 min, followed by 40 cycles at 95° C. for 15 s, and 60° C. for 1 min. All the reactions may be run on ABI Prism 7000 Sequence Detection System.

Microarray Hybridization and Data Processing.

cDNA samples and cellular total RNA (5 μg in each of eight individual tubes) may be subjected to the One-Cycle Target Labeling procedure for biotin labeling by in vitro transcription (IVT) (Affymetrix, Santa Clara, Calif.) or using the illumina Total Prep RNA Labelling kit. For analysis on Affymetix gene chips, the cRNA may be subsequently fragmented and hybridized to the Human Genome U133 Plus 2.0 Array (Aftymetrix) according to the manufacturer's instructions. The microarray image data may be processed with the GeneChip Scanner 3000 (Affymetrix) to generate CEL data. The CEL data may be then subjected to analysis with dChip softvare, which has the advantage of normalizing and processing multiple datasets simultaneously. Data obtained from the eight nonamplified controls from cells, from the eight independently amplified samples from the diluted cellular RNA, and from the amplified cDNA samples from 20 single cells may be normalized separately within the respective groups, according to the program's default setting. The model based expression indices (MBEI) may be calculated using the PM/MM difference mode with log-2 transformation of signal intensity and truncation of low values to zero. The absolute calls (Present, Marginal and Absent) may be calculated by the Affymetrix Microarray Software 5.0 (MAS 5.0) algorithm using the dChip default setting. The expression levels of only the Present probes may be considered for all quantitative analyses described below. The GEO accession number for the microarray data is GSE4309. For analysis on Illumina Human HT-12 v4 Expression Bead Chips, labeled cRNA may be hybridized according to the manufacturer's instructions.

Calculation of Coverage and Accuracy.

A true positive is defined as probes called Present in at least six of the eight nonamplified controls, and the true expression levels are defined as the log-averaged expression levels of the Present probes. The definition of coverage is (the number of truly positive probes detected in amplified samples)/(the number of truly positive probes). The definition of accuracy is (the number of truly positive probes detected in amplified samples)/(the number of probes detected in amplified samples). The expression levels of the amplified and nonamplified samples may be divided by the class interval of 20.5 (20, 20.5, 21, 21.5 . . . ), where accuracy and coverage are calculated. These expression level bins may be also used to analyze the frequency distribution of the detected probes.

Analysis of Gene Expression Profiles of Cells:

The unsupervised clustering and class neighbor analyses of the microarray data from cells may be performed using GenePattern software (http://www.broad.mit.edu/cancer/soflware/genepattern/), which performs the signal-to-noise ratio analysis/T-test in conjunction with the permutation test to preclude the contribution of any sample variability, including those from methodology and/or biopsy, at high confidence. The analyses may be conducted on the 14,128 probes for which at least 6 out of 20 single cells provided Present calls and at least 1 out of 20 samples provided expression levels >20 copies per cell. The expression levels calculated for probes with Absent/Marginal calls may be truncated to zero. To calculate relative gene expression levels, the Ct values obtained with Q-PCR analyses may be corrected using the efficiencies of the individual primer pairs quantified either with whole human genome (BD Biosciences) or plasmids that contain gene fragments. The relative expression levels may be further transformed into copy numbers with a calibration line calculated using the spike RNAs included in the reaction mixture (log₁₀[expression level]=1.05×log₁₀[copy number]+4.65). The Chi-square test for independence may be performed to evaluate the association of gene expressions with Gata4, which represents the difference between cluster 1 and cluster 2 determined by the unsupervised clustering and which is restricted to PE at later stages. The expression levels of individual genes measured with Q-PCR may be classified Into three categories: high (>100 copies per cell), middle (10-100 copies per cell), and low (<10 copies per cell). The Chi-square and P-values for independence from Gata4 expression may be calculated based on this classification. Chi squared is defined as follows: χ2=ΣΣ(n fij−fi fj)²/n fi fj, where i and j represent expression level categories (high, middle or low) of the reference (Gata4) and the target gene, respectively; fi, fj, and fij represent the observed frequency of categories i, j and ij, respectively; and n represents the sample number (n=24). The degrees of freedom may be defined as (r−1)×(c−1), where r and c represent available numbers of expression level categories of Gata4 and of the target gene, respectively.

Generating an Immune Response Against Thyroid Cancer

In some embodiments, antigen presenting cells (APCs) may be used to activate T lymphocytes in vivo or ex vivo, to elicit an immune response against cells expressing a cancer associated sequence. APCs are highly specialized cells and may include, without limitation, macrophages, monocytes, and dendritic cells (DCs). APCs may process antigens and display their peptide fragments on the cell surface together with molecules required for lymphocyte activation. In some embodiments, the APCs may be dendritic cells. DCs may be classified into subgroups, including, e.g., follicular dendritic cells, Langerhans dendritic cells, and epidermal dendritic cells. In other embodiments the invention provides a method of eliciting an antibody response to one or more of the cancer associated sequences disclosed infra. The method may comprise administering a protein or a peptide fragment encoded by one or more of the cancer associated sequences disclosed infra to a subject.

Some embodiments are directed to the use of cancer associated polypeptides and polynucleotides encoding a cancer associated sequence, a fragment thereof; or a mutant thereof, and antigen presenting cells (such as, without limitation, dendritic cells), to elicit an immune response against cells expressing a cancer-associated polypeptide sequence, such as, without limitation, cancer cells, in a subject. In some embodiments, the method of eliciting an immune response against cells expressing a cancer associated sequence comprises (1) isolating a hematopoletic stem cell, (2) genetically modifying the cell to express a cancer associated sequence, (3) differentiating the cell into DCs; and (4) administering the DCs to the subject (e.g., human patient). In some embodiments, the method of eliciting an immune response includes (1) isolating DCs (or isolation and differentiation of DC precursor cells), (2) pulsing the cells with a cancer associated sequence, and; (3) administering the DCs to the subject. These approaches are discussed in greater detail, infra. In some embodiments, the pulsed or expressing DCs may be used to activate T lymphocytes ex vivo. These general techniques and variations thereof may be within the skill of those in the art (see, e.g., WO97/29182; WO 97/04802; WO 97/22349; WO 96/23060; WO 98/01538; Hsu et al., 1996, Nature Med. 2:52-58), and that still other variations may be discovered in the future. In some embodiments, the cancer associated sequence is contacted with a subject to stimulate an immune response. In some embodiments, the immune response is a therapeutic immune response so as to treat a subject as described infra. In some embodiments, the immune response is a prophylactic immune response. For example, the cancer associated sequence can be contacted with a subject under conditions effective to stimulate an immune response. The cancer associated sequence can be administered as, for example, a DNA molecule (e.g. DNA vaccine), RNA molecule, or polypeptide, or any combination thereof. Administering a sequence to stimulate an immune response was known, but the identity of which sequences to use was not known prior to the present disclosure. Any sequence or combination of sequences disclosed herein or a homolog thereof can be administered to a subject to stimulate an immune response.

In some embodiments, dendritic cell precursor cells are isolated for transduction with a cancer associated sequence, and induced to differentiate into dendritic cells. The genetically modified DCs express the cancer associated sequence, and may display peptide fragments on the cell surface.

In some embodiments, the cancer associated sequence expressed comprises a sequence of a naturally occurring protein. In some embodiments, the cancer associate sequence does not comprise a naturally occurring sequence. As already noted, fragments of naturally occurring proteins may be used; in addition, the expressed polypeptide may comprise mutations such as deletions, insertions, or amino acid substitutions when compared to a naturally occurring polypeptide, so long as at least one peptide epitope can be processed by the DC and presented on a MHC class I or II surface molecule. In some embodiments, it may be desirable to use sequences other than “wild type,” in order to, for example, increase antigenicity of the peptide or to increase peptide expression levels. In some embodiments, the introduced cancer associated sequences may encode variants such as polymorphic variants (e.g., a variant expressed by a particular human patient) or variants characteristic of a particular cancer (e.g., a cancer in a particular subject).

In some embodiments, a cancer associated sequence may be introduced (transduced) into DCs or stem cells in any of a variety of standard methods, including transfection, recombinant vaccinia viruses, adeno-associated viruses (AAVs), retroviruses, etc.

In some embodiments, the transformed DCs of the invention may be introduced into the subject (e.g., without limitation, a human patient) where the DCs may induce an immune response. Typically, the immune response Includes a cytotoxic T-lymphocyte (CTL) response against target cells bearing antigenic peptides (e.g., In a MHC class I/peptide complex). These target cells are typically cancer cells.

In some embodiments, when the DCs are to be administered to a subject, they may preferably isolated from, or derived from precursor cells from, that subject (i.e., the DCs may administered to an autologous subject). However, the cells may be infused into HLA-matched allogeneic or HLA-mismatched allogeneic subject. In the latter case, immunosuppressive drugs may be administered to the subject.

In some embodiments, the cells may be administered in any suitable manner. In some embodiments, the cell may be administered with a pharmaceutically acceptable carrier (e.g., saline). In some embodiments, the cells may be administered through intravenous, intra-articular, intramuscular, intradermal, intraperitoneal, or subcutaneous routes. Administration (i.e., immunization) may be repeated at time intervals. Infisions of DC may be combined with administration of cytokines that act to maintain DC number and activity (e.g., GM-CSF, IL-12).

In some embodiments, the dose administered to a subject may be a dose sufficient to induce an immune response as detected by assays which measure T cell proliferation, T lymphocyte cytotoxicity, and/or effect a beneficial therapeutic response in the patient over time, e.g., to inhibit growth of cancer cells or result in reduction in the number of cancer cells or the size of a tumor.

In some embodiments, DCs are obtained (either from a patient or by in vitro differentiation of precursor cells) and pulsed with antigenic peptides having a cancer associated sequence. The pulsing results in the presentation of peptides onto the surface MHC molecules of the cells. The peptide/MHC complexes displayed on the cell surface may be capable of inducing a MHC-restricted cytotoxic T-lymphocyte response against target cells expressing cancer associated polypeptides (e.g., without limitations, cancer cells).

In some embodiments, cancer associated sequences used for pulsing may have at least about 6 or 8 amino acids and fewer than about 30 amino acids or fewer than about 50 amino acid residues in length. In some embodiments, an immunogenic peptide sequence may have from about 8 to about 12 amino acids. In some embodiments, a mixture of human protein fragments may be used; alternatively a particular peptide of defined sequence may be used. The peptide antigens may be produced by de novo peptide synthesis, enzymatic digestion of purified or recombinant human peptides, by purification of the peptide sequence from a natural source (e.g., a subject or tumor cells from a subject), or expression of a recombinant polynucleotide encoding a human peptide fragment.

In some embodiments, the amount of peptide used for pulsing DC may depend on the nature, size and purity of the peptide or polypeptide. In some embodiments, an amount of from about 0.05 ug/ml to about 1 mg/ml, from about 0.05 ug/ml to about 500 ug/ml, from about 0.05 ug/ml to about 250 ug/ml, from about 0.5 ug/ml to about 1 mg/ml, from about 0.5 ug/ml to about 500 ug/ml, from about 0.5 ug/ml to about 250 ug/ml, or from about 1 ug/ml to about 100 ug/ml of peptide may be used. After adding the peptide antigen(s) to the cultured DC, the cells may then be allowed sufficient time to take up and process the antigen and express antigen peptides on the cell surface in association with either class I or class II MHC. In some embodiments, the time to take up and process the antigen may be about 18 to about 30 hours, about 20 to about 30 hours, or about 24 hours.

Numerous examples of systems and methods for predicting peptide binding motifs for different MHC Class I and II molecules have been described. Such prediction could be used for predicting peptide motifs that will bind to the desired MHC Class I or II molecules. Examples of such methods, systems, and databases that those of ordinary skill in the art might consult for such purpose include:
I. Peptide Binding Motifs for MHC Class I and 11 Molecules; William E. Biddison, Roland Martin, Current Protocols in Immunology, Unit 11 (DOI: 10.1002/0471 142735.ima01is36; Online Posting Date: May, 2001).

Reference 1 above, provides an overview of the use of peptide-binding motifs to predict interaction with a specific MHC class I or H allele, and gives examples for the use of MHC binding motifs to predict T-cell recognition.

Table 3 provides an exemplary result for a HLA peptide motif search at the NIH Center for Information Technology website, BioInformatics and Molecular Analysis Section.

TABLE 3
exemplary result for HLA peptide motif search

| User Parameter and Scoring Information: | | | Explicit number |
|---|---|---|---|
| Method selected to mimic the number of | | | |
| results | | | |
| Number of results requested | | | 20 |
| HLA molecule type selected | | | A_0201 |
| Length selected for subsequences to be | | | 9 |
| scored | | | |
| Echoing mode selected for input sequence | | | Y |
| Echoing format | | | Numbered lines |
| Length of user's input peptide sequence | | | 369 |
| Number of subsequence scores calculated | | | 361 |
| Number of top-scoring subsequences | | | 20 |
| reported back in scoring output table | | | |
| | | | Score (estimate of |
| | | | half time of |
| | | | disassociation of a |
| | Scoring Results | Subsequence residue | molecule containing |
| Rank | Start Position | listing | this subsequence |
| 1 | 310 | SLLKFLAKV (SEQ | 2249.173 |
| | | ID NO: 32) | |
| 2 | 183 | MLLVFGIDV (SEQ | 1662.432 |
| | | ID NO: 33) | |
| 3 | 137 | KVTDLVQFL (SEQ | 339.313 |
| | | ID NO: 34) | |
| 4 | 254 | GLYDGMMEHL | 315.870 |
| | | (SEQ ID NO: 35) | |
| 5 | 228 | ILILSIIFI (SEQ ID | 224.357 |
| | | NO: 36) | |
| 6 | 296 | FLWGPRAHA (SEQ | 189.678 |
| | | ID NO: 37) | |
| 7 | 245 | VIWEALNMM (SEQ | 90.891 |
| | | ID NO: 38) | |
| 8 | 308 | KMSILKFLA (SEQ | 72.836 |
| | | ID NO: 39) | |
| 9 | 166 | KNYEDHFPL (SEQ | 37.140 |
| | | ID NO: 40) | |
| 10 | 201 | FVLVTSLGL (SEQ | 31.814 |
| | | ID NO: 41) | |
| 11 | 174 | ILFSEASEC (SEQ | 31.249 |
| | | ID NO: 42) | |
| 12 | 213 | GMLSDVQSM | 30.534 |
| | | (SEQ ID NO: 43) | |
| 13 | 226 | ILILILSII (SEQ ID | 16.725 |
| | | NO: 44) | |
| 14 | 225 | GILILILSI (SEQ ID | 12.208 |
| | | NO: 45) | |
| 15 | 251 | NMMGLYDGM | 9.758 |
| | | (SEQ ID NO: 46) | |
| 16 | 88 | QIACSSPSV (SEQ | 9.563 |
| | | ID NO: 47) | |
| 17 | 66 | LIPSTPEEV (SEQ | 7.966 |
| | | ID NO: 48) | |
| 18 | 220 | SMPKTGILI (SEQ | 7.535 |
| | | ID NO: 49) | |
| 19 | 233 | IIFIEGYCT (SEQ ID | 6.445 |
| | | NO: 50) | |
| 20 | 247 | WEALNMGL (SEQ | 4.395 |
| | | ID NO: 51) | |

One skilled in the art of peptide-based vaccination may determine which peptides would work best in individuals based on their HLA alleles (e.g., due to “MHC restriction”). Different HLA alleles will bind particular peptide motifs (usually 2 or 3 highly conserved positions out of 8-10) with different energies which can be predicted theoretically or measured as dissociation rates. Thus, a skilled artisan may be able to tailor the peptides to a subject's HLA profile.

In some embodiments, the present disclosure provides methods of eliciting an immune response against cells expressing a cancer associated sequence comprising contacting a subject with a cancer associated sequence under conditions effective to elicit an immune response in the subject, wherein said cancer associated sequence comprises a sequence or fragment thereof a gene selected from one or more of the cancer associated sequences provided infra.

Transfecting Cells with Cancer Associated Sequences

Cells may be transfected with one or more of the cancer associated sequences disclosed infra. Transfected cells may be useful in screening assays, diagnosis and detection assays. Transfected cells expressing one or more cancer associated sequence disclosed herein may be used to obtain isolated nucleic acids encoding cancer associated sequences and/or isolated proteins or peptide fragments encoded by one or more cancer associated sequences.

Electroporation may be used to introduce the cancer associated nucleic acids described herein into mammalian cells (Neumann, E. et al. (1982) EMBO J. 1, 841-845), plant and bacterial cells, and may also be used to introduce proteins (Marrero, M. B. et al. (1995) J. Biol. Chem. 270, 15734-15738; Nolkrantz, K. et al. (2002) Anal. Chem. 74, 4300-4305; Rul, M. et al. (2002) Life Sci. 71, 1771-1778). Cells (such as the cells of this invention) suspended in a buffered solution of the purified protein of interest are placed in a pulsed electrical field. Briefly, high-voltage electric pulses result in the formation of small (nanometer-sized) pores in the cell membrane. Proteins enter the cell via these small pores or during the process of membrane reorganization as the pores close and the cell returns to its normal state. The efficiency of delivery may be dependent upon the strength of the applied electrical field, the length of the pulses, temperature and the composition of the buffered medium. Electroporation is successful with a variety of cell types, even some cell lines that are resistant to other delivery methods, although the overall efficiency is often quite low. Some cell lines may remain refractory even to electroporation unless partially activated.

Microinjection may be used to introduce femtoliter volumes of DNA directly into the nucleus of a cell (Capecchi, M. R. (1980) Cell 22, 470-488) where it can be integrated directly into the host cell genome, thus creating an established cell line bearing the sequence of interest. Proteins such as antibodies (Abarzua, P. et al. (1995) Cancer Res. 55, 3490-3494; Theiss, C. and Meller, K. (2002) Exp. Cell Res. 281, 197-204) and mutant proteins (Naryanan, A. et al. (2003) J. Cell Sci. 116, 177-186) can also be directly delivered into cells via microinjection to determine their effects on cellular processes firsthand. Microinjection has the advantage of introducing macromolecules directly into the cell, thereby bypassing exposure to potentially undesirable cellular compartments such as low-pH endosomes.

Several proteins and small peptides have the ability to transduce or travel through biological membranes independent of classical receptor-mediated or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. In some embodiments, protein transduction domains (PTDs) from these proteins may be fused to other macromolecules, peptides or proteins such as, without limitation, a cancer associated polypeptide to successfully transport the polypeptide into a cell (Schwarze, S. R. et al. (2000) Trends Cell Biol. 10, 290-295). Exemplary advantages of using fusions of these transduction domains is that protein entry is rapid, concentration-dependent and appears to work with difficult cell types (Fenton, M. et al. (1998) J. Immunol. Methods 212, 41-48).

In some embodiments, liposomes may be used as vehicles to deliver oligonucleotides, DNA (gene) constructs and small drug molecules into cells (Zabner, J. et al. (1995) J. Biol. Chem. 270, 18997-19007; Felgner, P. L. et al. (1987) Proc. Natl. Acad. Sci. USA 84, 7413-7417). Certain lipids, when placed in an aqueous solution and sonicated, form closed vesicles consisting of a circularized lipid bilayer surrounding an aqueous compartment. The vesicles or liposomes of embodiments herein may be formed in a solution containing the molecule to be delivered. In addition to encapsulating DNA in an aqueous solution, cationic liposomes may spontaneously and efficiently form complexes with DNA, with the positively charged head groups on the lipids interacting with the negatively charged backbone of the DNA. The exact composition and/or mixture of cationic lipids used can be altered, depending upon the macromolecule of interest and the cell type used (Felgner, J. H. et al. (1994) J. Biol. Chem. 269, 2550-2561). The cationic liposome strategy has also been applied successfully to protein delivery (Zelphati, O. et al. (2001) J. Biol. Chem. 276, 35103-35110). Because proteins are more heterogeneous than DNA, the physical characteristics of the protein, such as its charge and hydrophobicity, may influence the extent of its interaction with the cationic lipids.

Pharmaceutical Compositions and Modes of Administration

Modes of administration for a therapeutic (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of therapeutic to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the therapeutic of the present disclosure and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present disclosure. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions of the present disclosure can be formulated for parenteral administration by injection, e.g., by bolus injection or continuous infusion. The compositions can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compositions can be formulated readily by combining the therapeutic with pharmaceutically acceptable carriers well known in the art. Such carriers enable the therapeutic of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active therapeutic doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active therapeutic can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the pharmaceutical compositions can take the form of, e.g., tablets or lozenges formulated in a conventional manner.

For administration by inhalation, the therapeutic for use according to the present disclosure is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the therapeutic and a suitable powder base such as lactose or starch.

The compositions of the present disclosure can also be formulated in rectal compositions such as suppositories or retention enemas, e.g., containing conventional suppository bases such as cocoa butter or other glycerides.

In addition to the formulations described previously, the therapeutic of the present disclosure can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present disclosure, for example, can be applied to a plaster, or can be applied by transdermal, therapeutic systems that are consequently supplied to the organism.

Pharmaceutical compositions can include suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compositions of the present disclosure can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component may include one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearylfiimarate, fatty acid, fatty alcohol, fatty acid ester, glycerylbehenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethyklene fatty alcohol ether, polyethoxylated sterol, polyethoxylated castor oil, polyethoxylated vegetable oil, or sodium chloride.

Kits

Also provided by the invention are kits and systems for practicing the subject methods, as described above, such components configured to diagnose cancer in a subject, treat cancer in a subject, detect cancer in a sample, or perform basic research experiments on cancer cells (e.g., either derived directly from a subject, grown in vitro or ex vivo, or from an animal model of cancer. The various components of the kits may be present in separate containers or certain compatible components may be pre-combined into a single container, as desired.

In some embodiments, the invention provides a kit for diagnosing the presence of cancer in a test sample, said kit comprising at least one polynucleotide that selectively hybridizes to a cancer associated polynucleotide sequence shown in SEQ ID NOS 1-29, or its complement. The kit may include a protein or a peptide that binds to one or more of the cancer associated sequences described infra, e.g. IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19. The kit may include one or more a probes such as one or more oligonucleotides that bind to one or more of the cancer associated sequences disclosed infra, e.g. IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19. In another embodiment the invention provides an electronic library comprising a cancer associated polynucleotide, a cancer associated polypeptide, or fragment thereof, disclosed infra. In some embodiments the kit may include one or more capture reagents or specific binding partners of one or more cancer associated sequences disclosed infra.

The subject systems and kits may also include one or more other reagents for performing any of the subject methods. The reagents may include one or more matrices, solvents, sample preparation reagents, buffers, desalting reagents, enzymatic reagents, denaturing reagents, probes, polynucleotides, vectors (e.g., plasmid or viral vectors), etc., where calibration standards such as positive and negative controls may be provided as well. As such, the kits may include one or more containers such as vials or bottles, with each container containing a separate component for carrying out a sample processing or preparing step and/or for carrying out one or more steps for producing a normalized sample according to the present disclosure.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control samples and reagents, e.g., two or more control samples for use in testing the kit.

Additional Embodiments of the Invention

In some embodiments, the methods comprise targeting a marker that is expressed at abnormal levels in thyroid tumor tissue in comparison to normal somatic tissue. In some embodiments, the marker may comprise a sequence disclosed herein or in Table 1, a complement thereof, or a combination thereof. In some embodiments, the marker may be selected from a sequence encoding IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 a homolog thereof, a fragment thereof, a complement thereof or a combination thereof. In some embodiments, the marker may comprise a sequence selected from SEQ ID NOs: 1-29, a fragment thereof, a complement thereof, or a combination thereof or is encoded by the same. In some embodiments, the methods for the treatment of thyroid cancer and related pharmaceutical preparations and kits are provided. Some embodiments are directed to methods of treating thyroid cancer comprising administering a composition including a therapeutic that affects the expression, abundance or activity of a target marker. In some embodiments, the target marker may include a sequence described herein or in Table 1, a complement thereof, or any combination thereof. In some embodiments, the target marker may comprise a sequence selected from SEQ ID NOs: 1-29, a fragment thereof, a complement thereof, or a combination thereof.

Some embodiments provide methods of detecting thyroid cancer comprising detecting a level of a target marker associated with the cancer. In some embodiments, the target marker may include a sequence described herein or in Table 1, a complement thereof or any combination thereof. In some embodiments, the marker may be selected from a sequence selected from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11 AHNAK2, CYTOKERATINE19B, a fragment thereof, a complement thereof or a combination thereof. In some embodiments, the marker may be selected from SEQ ID NOs: 1-29, a fragment thereof, a complement thereof, or a combination thereof.

Some embodiments herein provide antigens (i.e., cancer-associated polypeptides) associated with thyroid cancer as targets for diagnostic and/or therapeutic antibodies. In some embodiments, the antigen may be selected from a sequence selected from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 a fragment thereof, a complement thereof or a combination thereof. In some embodiments, the antigen may be encoded by a sequence selected from SEQ ID NOs: 1-29, a fragment thereof, a complement thereof, or a combination thereof. In some embodiments, these antigens may be useful for drug discovery (e.g., small molecules) and for further characterization of cellular regulation, growth, and differentiation.

Some embodiments describe a method of diagnosing thyroid cancer in a subject, the method comprising: (a) determining the expression of one or more genes or gene products or homologs thereof; and (b) comparing the expression of the one or more nucleic acid sequences from a second normal sample from the first subject or a second unaffected subject, wherein a difference in the expression indicates that the first subject has thyroid cancer, wherein the gene or the gene product is referred to as a gene selected from: IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 a gene described in Table 1 or 2 (with sequences incorporated by reference via the accession number), a fragment thereof, a complement thereof or a combination thereof.

Some embodiments describe a method of eliciting an immune response against cells expressing a cancer associated sequence comprising contacting a subject with a cancer associated sequence under conditions effective to elicit an immune response in the subject, wherein the cancer associated sequence comprises a sequence or fragment thereof selected from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 a sequence described in Table 1 or Table 2 (including but not limited to the sequences described in the accession files), or a combination thereof.

Some embodiments describe a method of detecting thyroid cancer in a test sample, comprising: (i) detecting a level of activity of at least one polypeptide that is a gene product; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of thyroid cancer in the test sample, wherein the gene product is a product of a gene selected from: IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf18, TNFRSF11B, AHNAK2, CYTOKERATINE19 a sequence described in Table 1 or Table 2, or a combination thereof.

Some embodiments herein are directed to a method of treating thyroid cancer in a subject, the method comprising administering to a subject in need thereof a therapeutic agent modulating the activity of a cancer associated protein, wherein the cancer associated protein is encoded by a nucleic acid comprising a nucleic acid sequence selected from a sequence described herein or in Table 1, homologs thereof, combinations thereof, or a fragment thereof. In some embodiments, the therapeutic agent binds to the cancer associated protein. In some embodiments, the therapeutic agent is an antibody. In some embodiments, the antibody may be a monoclonal antibody or a polyclonal antibody. In some embodiments, the antibody is a humanized or human antibody. In some embodiments, a method of treating thyroid cancer may comprise gene knockdown of a gene selected from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 a sequence described in Table 1 or Table 2, or a combination thereof. In some embodiments, a method of treating thyroid cancer may comprise treating cells to knockdown or inhibit expression of a gene encoding an mRNA of IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 a sequence described in Table 1 or Table 2, a fragment thereof, a complement thereof, or a combination thereof. In some embodiments, the cancer is thyroid cancer. In some embodiments, the thyroid cancer is selected from carcinoma, any malignant thyroid neoplasm, papillary thyroid cancer, follicular thyroid cancer, medullary thyroid cancer, anaplastic thyroid cancer, lymphoma, squamous cell carcinoma, papillary microsarcoma, or a combination thereof. The methods disclosed herein may also be used for diagnosis and treatment of other cancers and other conditions in which cells have become immortalized.

In some embodiments, a method of diagnosing a subject with thyroid cancer comprises obtaining a sample and detecting the presence of a cancer associated sequence selected from a sequence described herein or in Table 1, a fragment thereof or a complement thereof wherein the presence of the cancer associated sequence indicates the subject has thyroid cancer or a sequence that specifically hybridizes with a gene selected from the group of IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf178, TNFRSF11B, AHNAK2, CYTOKERATINE19 a sequence described in Table 1 or Table 2 (sequence incorporated by reference via the accession number), a fragment thereof a complement thereof, or a combination thereof. In some embodiments, detecting the presence of a cancer associated sequence comprises contacting the sample with an antibody or other type of capture reagent that specifically binds to the cancer associated sequence's protein and detecting the presence or absence of the binding to the cancer associated sequence's protein in the sample. In some embodiments, the thyroid cancer is selected from carcinoma, any malignant thyroid neoplasm, papillary thyroid cancer, follicular thyroid cancer, medullary thyroid cancer, anaplastic thyroid cancer, lymphoma, squamous cell carcinoma, papillary microsarcoma, or a combination thereof. The methods disclosed herein may also be used for diagnosis and treatment of other conditions in which cells have become immortalized.

In some embodiments, the present invention provides methods of treating cancer in a subject, the method comprising administering to a subject in need thereof a therapeutic agent that modulates the activity of IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 a sequence described in Table 1 or Table 2 (sequence incorporated by reference via the accession number), a fragment thereof a complement thereof, or a combination thereof, wherein the therapeutic agent treats the cancer in the subject. In some embodiments, the cancer is thyroid cancer. In some embodiments, the thyroid cancer is selected from carcinoma, any malignant thyroid neoplasm, papillary thyroid cancer, follicular thyroid cancer, medullary thyroid cancer, anaplastic thyroid cancer, lymphoma, squamous cell carcinoma, papillary microsarcoma, or a combination thereof. The methods disclosed herein may also be used for treatment of other cancers and other conditions in which cells have become immortalized.

In some embodiments, the present invention provides methods of diagnosing thyroid cancer in a subject, the method comprising determining the expression of a gene disclosed in Table 1 or a gene selected from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 a fragment thereof, a complement thereof, or a combination thereof from a sample; and diagnosing thyroid cancer in the subject based on the expression, wherein the subject is diagnosed as having thyroid cancer if the gene is overexpressed.

In some embodiments, the present invention provides methods of detecting thyroid cancer in a test sample, the method comprising: (i) detecting a level of an antibody, wherein the antibody binds to an antigenic polypeptide encoded by a nucleic acid sequence comprising a sequence encoding IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 a sequence described in Table 1 or Table 2 (sequence incorporated by reference via the accession number), a homolog thereof, a fragment thereof, a complement thereof, or a combination thereof; and (ii) comparing the level of the antibody in the test sample with a level of the antibody in a control sample, wherein an altered level of antibody in the test sample relative to the level of antibody in the control sample is indicative of the presence of thyroid cancer in the test sample. In some embodiments, the nucleic acid sequence may be selected from SEQ ID NOs: 1-29, a fragment thereof, a complement thereof, or a combination thereof.

In some embodiments, the present invention provides methods of detecting thyroid cancer in a test sample, comprising: (i) detecting a level of activity of at least one polypeptide that is encoded by a nucleic acid comprising a nucleic acid sequence encoding IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 a sequence described in Table 1 or Table 2 (sequence incorporated by reference via the accession number), a homolog thereof, a fragment thereof, a complement thereof, or a combination thereof; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of thyroid cancer in the test sample. In some embodiments, the nucleic acid sequence may be selected from SEQ ID NOs: 1-29, a fragment thereof, a complement thereof, or a combination thereof.

In some embodiments, the present invention provides methods of detecting thyroid cancer in a test sample, the method comprising: (I) detecting a level of expression of at least one polypeptide that is encoded by a nucleic acid comprising a nucleic acid sequence encoding IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19, or a sequence disclosed in Table 1, homologs thereof, combinations thereof, or a fragment thereof; and (ii) comparing the level of expression of the polypeptide in the test sample with a level of expression of polypeptide in a normal sample, wherein an altered level of expression of the polypeptide in the test sample relative to the level of polypeptide expression in the normal sample is indicative of the presence of thyroid cancer in the test sample. In some embodiments, the nucleic acid sequence may be selected from SEQ ID NOs: 1-29, a fragment thereof, a complement thereof, or a combination thereof.

In some embodiments, the present invention provides methods of detecting thyroid cancer in a test sample, the method comprising: (I) detecting a level of expression of a nucleic acid sequence comprising a nucleic acid sequence encoding IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19or a sequence disclosed in Table 1, homologs thereof, mutant nucleic acids thereof, combinations thereof, or a fragment thereof; and (ii) comparing the level of expression of the nucleic acid sequence in the test sample with a level of expression of nucleic acid sequence in a normal sample, wherein an altered level of expression of the nucleic acid sequence in the test sample relative to the level of nucleic acid sequence expression in the normal sample is indicative of the presence of thyroid cancer in the test sample. In some embodiments, the nucleic acid sequence may be selected from SEQ ID NOs: 1-29, a fragment thereof, a complement thereof, or a combination thereof.

In some embodiments, the present invention provides methods of screening for activity against thyroid cancer, the method comprising: (a) contacting a cell that expresses a cancer associated gene comprising a sequence encoding IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19or a sequence disclosed in Table 1, a complement thereof, homologs thereof, combinations thereof, or fragments thereof with a cancer drug candidate; (b) detecting an effect of the cancer drug candidate on an expression of the cancer associated polynucleotide in the cell; and (c) comparing the level of expression in the absence of the drug candidate to the level of expression in the presence of the drug candidate; wherein an effect on the expression of the cancer associate polynucleotide indicates that the candidate has activity against thyroid cancer. In some embodiments, the cancer associated gene comprises a sequence selected from SEQ ID NOs: 1-29, a fragment thereof, a complement thereof, or a combination thereof.

In some embodiments, the present invention provides methods of screening for activity against thyroid cancer, the method comprising: (a) contacting a cell that overexpresses a cancer associated gene comprising a sequence encoding IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19or a sequence disclosed in Table 1, a complement thereof, homologs thereof, combinations thereof, or fragments thereof with a cancer drug candidate; (b) detecting an effect of the cancer drug candidate on an expression of the cancer associated polynucleotide in the cell or an effect on cell growth or viability; and (c) comparing the level of expression, cell growth, or viability in the absence of the drug candidate to the level of expression, cell growth, or viability in the presence of the drug candidate; wherein an effect on the expression of the cancer associated polynucleotide, cell growth, or viability indicates that the candidate has activity against the thyroid cancer cell that overexpresses a cancer associated gene comprising a sequence encoding IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a sequence disclosed in Table 1, a complement thereof, homologs thereof, combinations thereof, or fragments thereof. In some embodiments, the cancer associated gene comprises a sequence selected from SEQ ID NOs: 1-29, a fragment thereof, a complement thereof, or a combination thereof.

In some embodiments, the present invention provides methods of diagnosing thyroid cancer in a subject, the method comprising: a) determining the expression of one or more nucleic acid sequences, wherein the one or more nucleic acid sequences comprises a sequence encoding IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19 or a sequence disclosed in Table 1, homologs thereof, combinations thereof, or fragments thereof in a first sample of a first subject; and b) comparing the expression of the one or more nucleic acid sequences from a second normal sample from the first subject or a second unaffected subject, wherein a difference in the expression of a sequence disclosed herein or in Table 1 indicates that the first subject has thyroid cancer. In some embodiments, the sequence may be selected from SEQ ID NOs: 1-29, a fragment thereof, a complement thereof, or a combination thereof.

In some embodiments, the present invention provides methods of diagnosing thyroid cancer in a subject, the method comprising: a) determining the expression of one or more genes or gene products or homologs thereof in a subject; and b) comparing the expression of the one or more genes or gene products or homologs thereof in the subject to the expression of one or more genes or gene products or homologs there of from a normal sample from the subject or a normal sample from an unaffected subject, wherein a difference in the expression indicates that the subject has thyroid cancer, wherein the one or more genes or gene products comprises a sequence encoding IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI13L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19 or a sequence disclosed in Table 1, homologs thereof, combinations thereof, or fragments thereof. In some embodiments, the sequence may be selected from SEQ ID NOs: 1-29, a fragment thereof, a complement thereof, or a combination thereof.

In some embodiments, the present invention provides methods of detecting cancer in a test sample, comprising: (i) detecting a level of activity of at least one polypeptide; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample, wherein the polypeptide is a gene product of a sequence disclosed in Table 1 or is a gene product of IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a sequence disclosed in Table 1, homologs thereof, combinations thereof, or fragments thereof. In some embodiments, the polypeptide is encoded by a sequence selected from SEQ ID NOs: 1-29, a fragment thereof, a complement thereof, or a combination thereof.

In some embodiments, the present invention provides methods of diagnosing thyroid cancer in a subject, the method comprising: obtaining one or more gene expression results for one or more sequences, wherein the one or more sequences comprises a sequence encoding IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B, AHNAK2, CYTOKERATINE19 or a sequence disclosed in Table 1, homologs thereof, combinations thereof, or fragments thereof, from a sample derived from a subject; and diagnosing cancer in the subject based on the one or more gene expression results, wherein the subject is diagnosed as having cancer if one or more genes is overexpressed. In some embodiments, the sequence may be selected from SEQ ID NOs: 1-29, a fragment thereof, a complement thereof, or a combination thereof.

In some embodiments, the present invention provides methods of diagnosing a subject with thyroid cancer or as a person suspected of having thyroid cancer by determining the amount of protein in a subject of IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU, KIAA1324, CCDC85A, CRABP2, C14orf78, TNFRSF11B AHNAK2, CYTOKERATINE19, or a protein product of a sequence disclosed in Table 1, homologs thereof, combinations thereof, or fragments thereof. The amount of protein can be determined in a sample, such as but not limited to, serum, blood, or urine.

In some embodiments, the present invention provides methods of utilizing the promoter sequences of genes disclosed herein including: IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B AHNAK2, CYTOKERATINE19, or a sequence disclosed in Table 1, homologs thereof, combinations thereof, or fragments thereof, to express a transgene that results in the destruction or inhibits the growth, migration, or angiogenesis, of tumors and cells residing in tumors. By nonlimiting example, said promoter and transgene sequence may be expressed in exogenous cells such as perivascular cells, including mesenchymal stein cells, pericytes, RGS5 positive pericytes, or dispose stromal fraction cells that are introduced into the tumor or tumor site after the removal of the tumor, or into the blood circulation such that the exogenous cells activate the transgene subsequent to inhabiting the tumor site.

In some embodiments, the present invention provides methods of visualizing a tumor in a subject comprising targeting a cancer associated protein with a labeled molecule, wherein the cancer associated protein is selected from a protein described herein, and detecting the labeled molecule, wherein the labeled molecule visualizes the tumor in the subject. The protein may be selected from IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19or a protein product of a sequence disclosed in Table 1, homologs thereof, combinations thereof, or fragments thereof. In some embodiments, the protein may be encoded by a sequence selected from SEQ ID NOs: 1-29, a fragment thereof, a complement thereof, or a combination thereof.

### Example 1

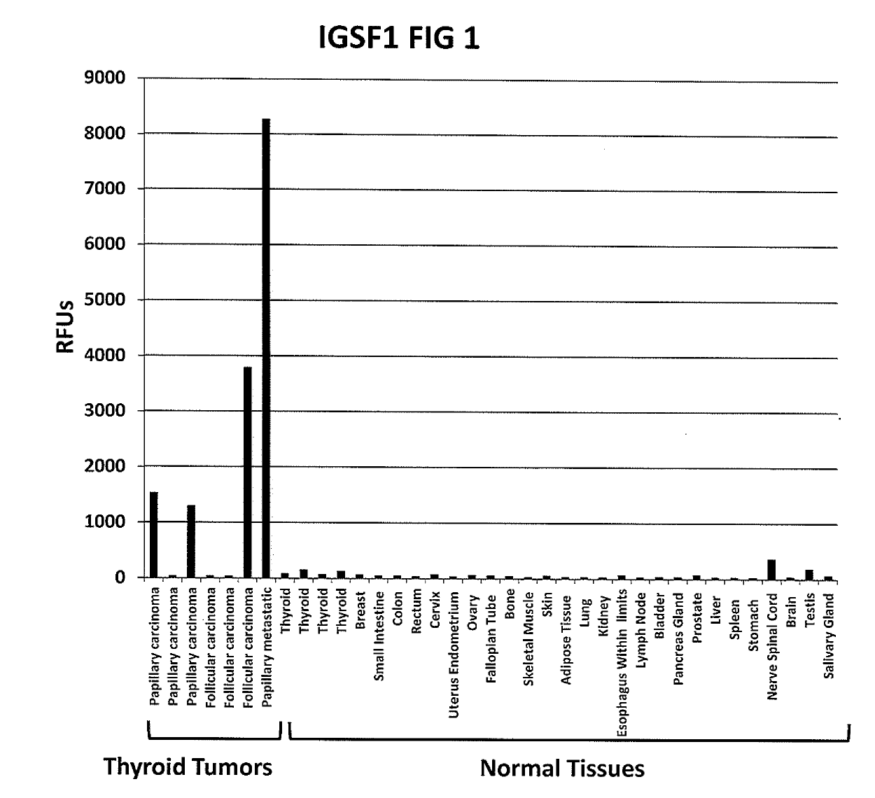

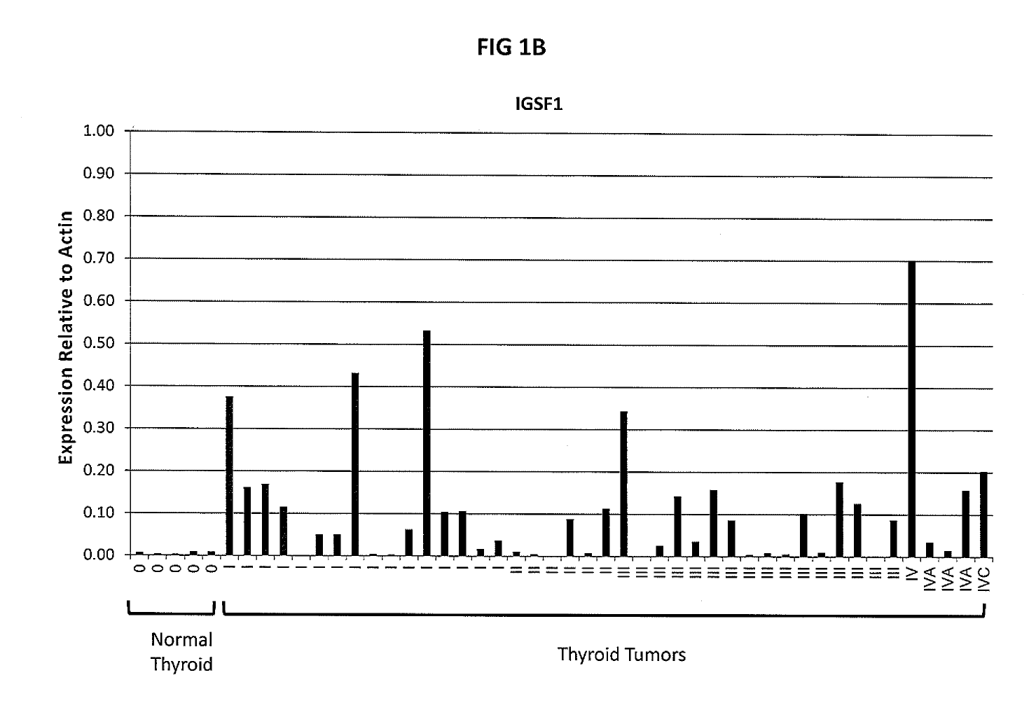

IGSF1: IGSF1 (Accession number NM_{—}001555.2) encodes Homo sapiens immunoglobulin superfamily, member 1. It is disclosed here that IGSF1 is a novel marker for thyroid tumors. As shown in FIG. 1A, IGSF1 expression was assayed by Illumina microarray, a probe specific for IGSF1 (probe sequence CCCTGCAAGTCAGCCCCATCTGCTGTTCCTGGTCTCTAATCACCTOAGC (SEQ ID NO: 52); Illumina probe ID ILMN_{—}1679299) detected strong gene expression (>400 RFUs) in thyroid gland tumor papillary carcinoma, thyroid gland follicular carcinoma and metastatic papillary thyroid carcinoma. In contrast, expression of IGSF1 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<400 RFUs). The specificity of elevated IGSF1 expression in malignant tumors of thyroid origin shown herein demonstrates that IGSF1 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

qPCR with primers recognizing IGSF1 can be used to distinguish between normal thyroid and malignant thyroid tumors as is shown in FIG. 1B. This figure shows qPCR for IGSF1 using OriGene TissueScan Thyroid Cancer cDNA arrays. As FIG. 1B shows, most malignant thyroid tumors are positive for IGSF1 via this qPCR assay, whereas the normal thyroid tissue is negative. Thus, a qPCR assay for IGSF1 may be used to distinguish between normal thyroid and thyroid tumors. Further, the qPCR assay may be used to correlate the marker with patient outcome or susceptibility to particular therapeutic approaches.

Therapeutics that target IGSF1 can be identified using the methods described herein and therapeutics that target IGSF1 include, but are not limited to, antibodies that modulate the activity of IGSF1. The manufacture and use of antibodies are described herein.

### Example 2

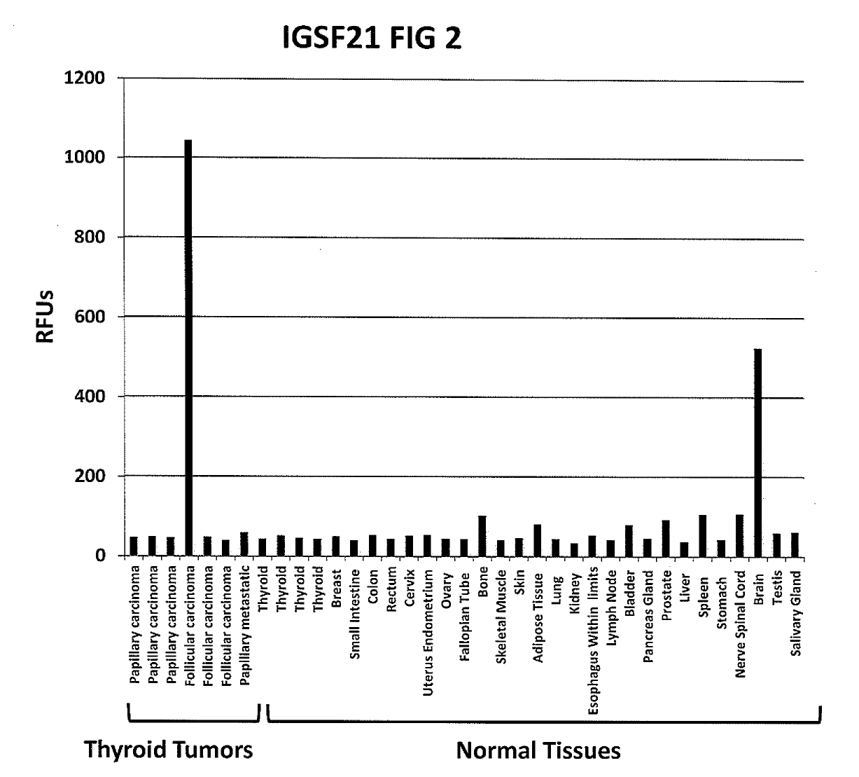

IGSF21: IGSF21 (Accession number NM_{—}032880.2) encodes Homo sapiens immunoglobin superfamily, member 21. It is disclosed here that IGSF21 is a novel marker for thyroid tumors. As shown in FIG. 2, IGSF21 expression was assayed by Illumina microarray, a probe specific for IGSF21 (probe sequence ACCTTGGTGCTCGCCCTGACAGTGATTCTGGAGCTGACGTGAAGGCACCC(SEQ ID NO: 53); Illumina probe ID ILMN_{—}1730039) detected strong gene expression (>600 RFUs) in thyroid gland follicular carcinoma. In contrast, expression of IGSF21 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<600 RFUs). The specificity of elevated IGSF21 expression in malignant tumors of thyroid origin shown herein demonstrates that IGSF21 is a marker for the diagnosis of thyroid cancer (e.g. including but not limited to thyroid follicular carcinomas) and is a target for therapeutic intervention in thyroid cancer. The marker may be detected in urine as well as sera.

Therapeutics that target IGSF21 can be identified using the methods described herein and therapeutics that target IGSF21 include, but are not limited to, antibodies that modulate the activity of IGSF21. The manufacture and use of antibodies are described herein.

### Example 3

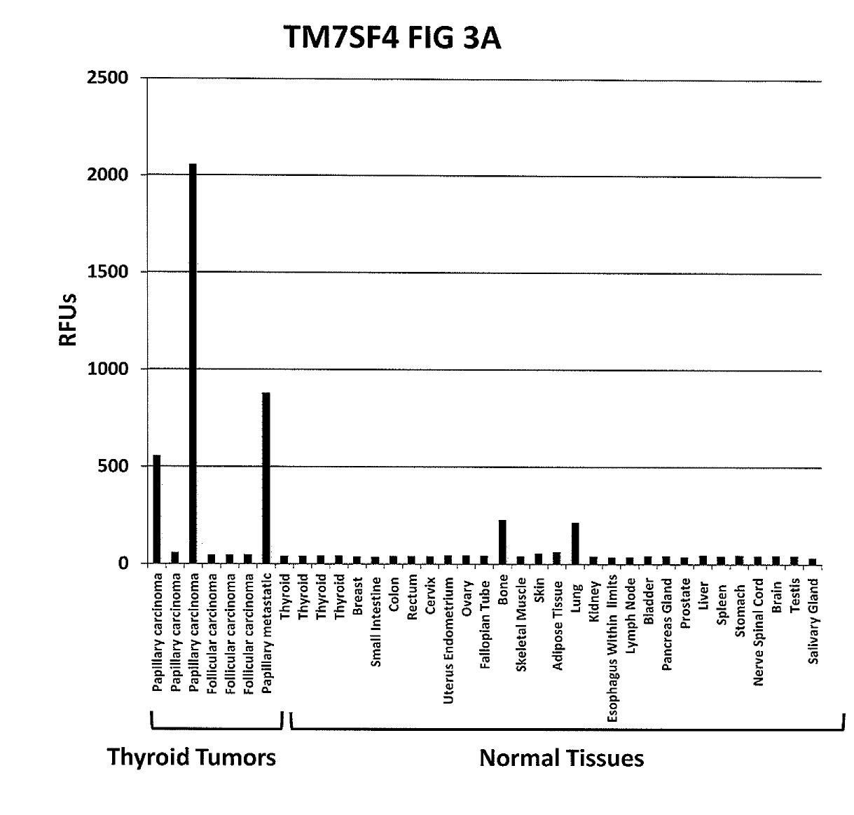

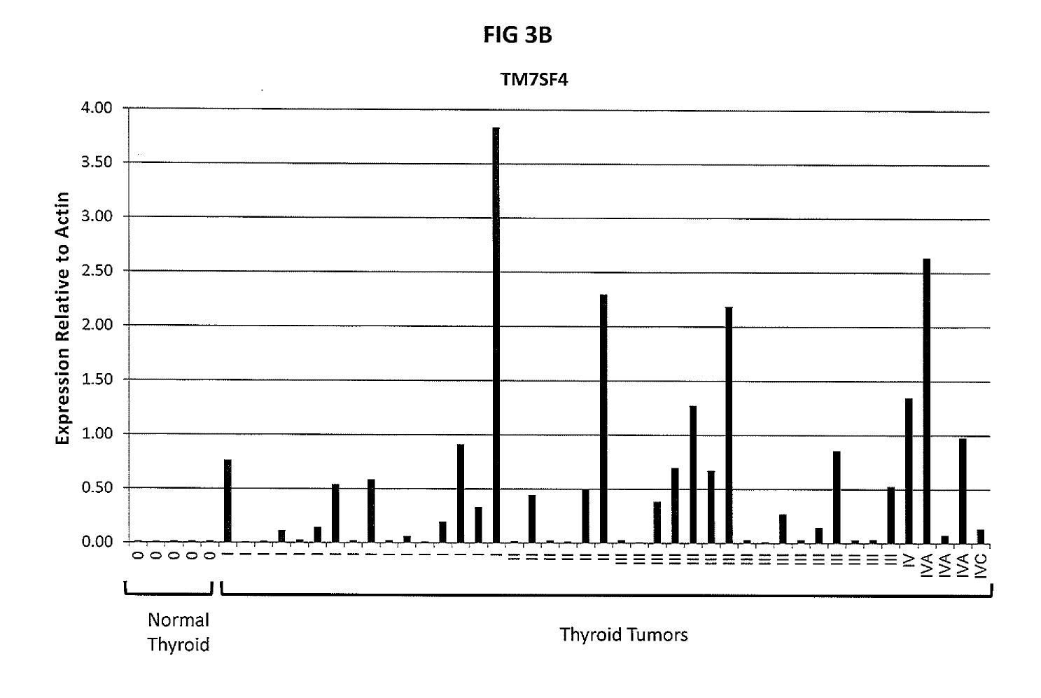

TM7SF4: TM7SF4 (Accession number NM_{—}030788.2) encodes Homo sapiens transmembrane 7 superfamily member 4. It is disclosed here that TM7SF4 is a novel marker for thyroid tumors. As shown in FIG. 3A, TM7SF4 expression was assayed by Illumina microarray, a probe specific for TM7SF4 (probe sequence GCAGCACCTGGTTATGCCTCCTITCATCTCAAAGCCAAAGAGCTGCCAGG(SEQ ID NO: 54); Illumina probe ID ILMN_{—}1793730) detected strong gene expression (>300 RFUs) in thyroid gland tumor papillary carcinoma and metastatic papillary thyroid carcinoma. In contrast, expression of TM7SF4 in a wide variety of normal tissues including normal thyroid, kidney, breast, colon, rectum, cervix, endomnetrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<300 RFUs). The specificity of elevated TM7SF4 expression in malignant tumors of thyroid origin shown herein demonstrates that TM7SF4 is a marker for the diagnosis of thyroid cancer (e.g. including but not limited to thyroid papillary carcinomas and metastatic thyroid tumors), and is a target for therapeutic intervention in thyroid cancer. The marker may be detected in urine as well as sera.

qPCR with primers recognizing TM7SF4 can be used to distinguish between normal thyroid and malignant thyroid tumors as is shown in FIG. 3B. This figure shows qPCR for TM7SF4 using OriGene TissueScan Thyroid Cancer cDNA arrays. As FIG. 3B shows, most malignant thyroid tumors are positive for TM7SF4 via this qPCR assay, whereas the normal thyroid tissue is negative. Thus, a qPCR assay for TM7SF4 may be used to distinguish between normal thyroid and thyroid tumors. Further, the qPCR assay may be used to correlate the marker with patient outcome or susceptibility to particular therapeutic approaches.

Therapeutics that target TM7SF4 can be identified using the methods described herein and therapeutics that target TM7SF4 include, but are not limited to, antibodies that modulate the activity of TM7SF4. The manufacture and use of antibodies are described herein.

### Example 4

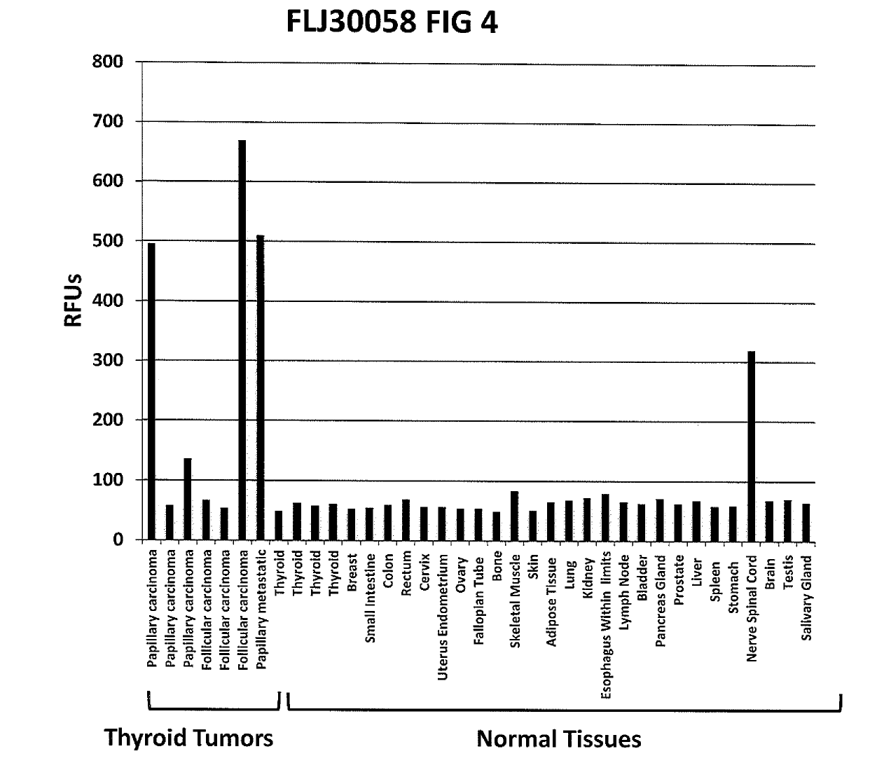

FLJ30058: FLJ30058 (Accession number NM_{—}144967.2) encodes Homo sapiens hypothetical protein FLJ30058. It is disclosed here that FLJ30058 is a novel marker for thyroid tumors. As shown in FIG. 4, FLJ30058 expression was assayed by Illumina microarray, a probe specific for FLJ30058 (probe sequence GTACAGTTTGCTCAGGTCACGCCAACAGGGAAACCTCAAGTOTAGGTCT(SEQ ID NO: 55); Illumina probe ID ILMN_{—}1705466) detected strong gene expression (>400 RFUs) in thyroid gland tumor papillary carcinoma, thyroid gland follicular carcinoma and metastatic papillary thyroid carcinoma. In contrast, expression of FLJ30058 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<400 RFUs). The specificity of elevated FLJ30058 expression in malignant tumors of thyroid origin shown herein demonstrates that FLJ30058 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target FLJ30058 can be identified using the methods described herein and therapeutics that target FLJ30058 include, but are not limited to, antibodies that modulate the activity of FLJ30058. The manufacture and use of antibodies are described herein.

### Example 5

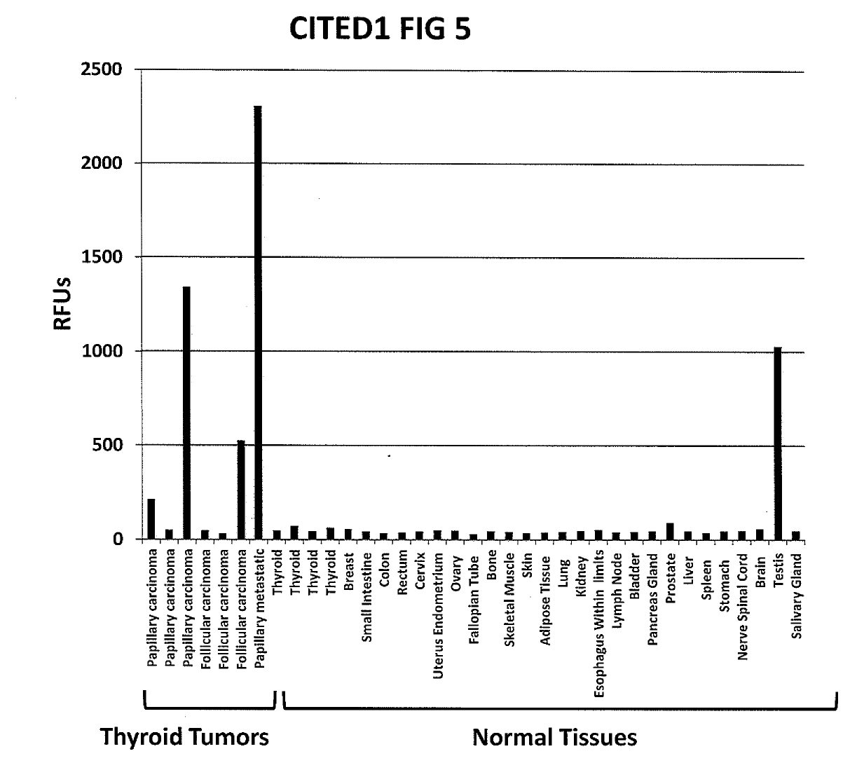

CITED1: CITED1 (Accession number NM_{—}004143.2) encodes Homo sapiens Cbp/p300-interactlng transactivator, with Glu/Asp-rich carboxy-terminal domain. It is disclosed here that CITED1 is a novel marker for thyroid tumors. As shown in FIG. 5, CITED1 expression was assayed by lllumina microarray, a probe specific for CITED1 (probe sequence GCTCCCACTAGTTCCTCGGGATCTCCAATAGGCTCTCCTACAACCACCCC (SEQ ID NO: 56); Illumina probe ID ILMN_{—}1691641) detected strong gene expression (>200 RFUs) in thyroid gland tumor papillary carcinoma, thyroid gland follicular carcinoma and metastatic papillary thyroid carcinoma. In contrast, expression of CITED1 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<200 RFUs), with the exception of testis (1032 RFUs). The specificity of elevated CITED1 expression in malignant tumors of thyroid origin shown herein demonstrates that CITED1 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target CITED1 can be identified using the methods described herein and therapeutics that target CITED1 include, but are not limited to, antibodies that modulate the activity of CITED1. The manufacture and use of antibodies are described herein.

### Example 6

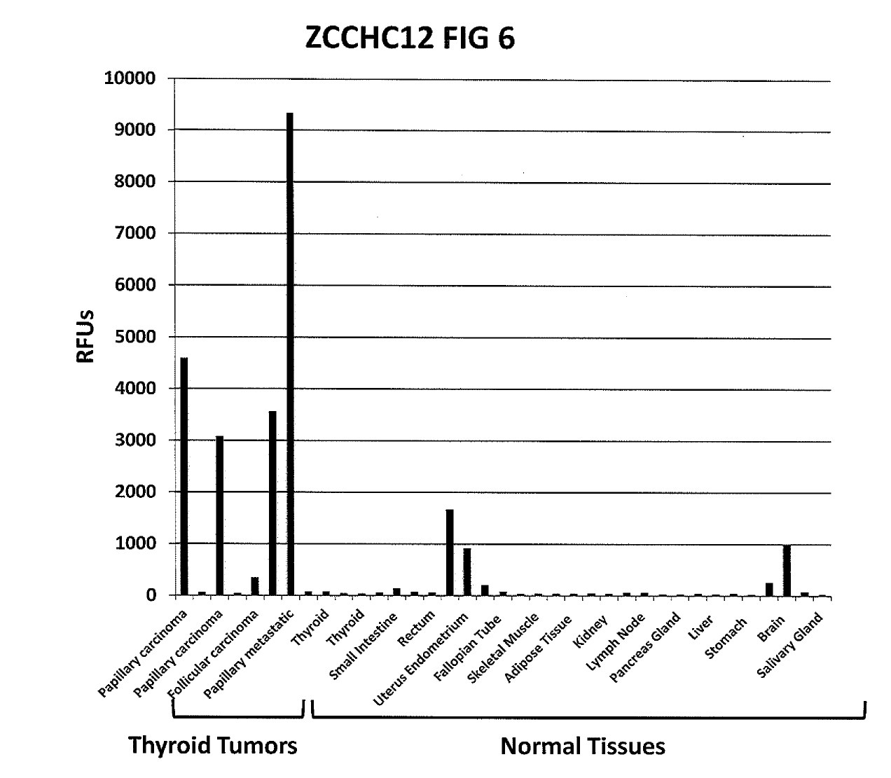

ZCCHC12: ZCCHC12 (Accession number NM_{—}173798.2) encodes Homo sapiens zinc finger, CCHC domain containing 12. It is disclosed here that ZCCHC12 is a novel marker for thyroid tumors. As shown in FIG. 6, ZCCHC12 expression was assayed by Illumina microarray, a probe specific for ZCCHC12 (probe sequence CCCTGCAGCCTACGGGTCTGTITICTGTTGTGTGCCCAITCCTTGACAGC(SEQ ID NO: 57); Illumina probe ID ILMN_{—}1679984) detected strong gene expression (>3000 RFUs) in thyroid gland tumor papillary carcinoma, thyroid gland follicular carcinoma and metastatic papillary thyroid carcinoma. In contrast, expression of ZCCHC12 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<3000 RFUs). The specificity of elevated ZCCHC12 expression in malignant tumors of thyroid origin shown herein demonstrates that ZCCHC12 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target ZCCHC12 can be identified using the methods described herein and therapeutics that target ZCCHC12 include, but are not limited to, antibodies that modulate the activity of ZCCHC12. The manufacture and use of antibodies are described herein.

### Example 7

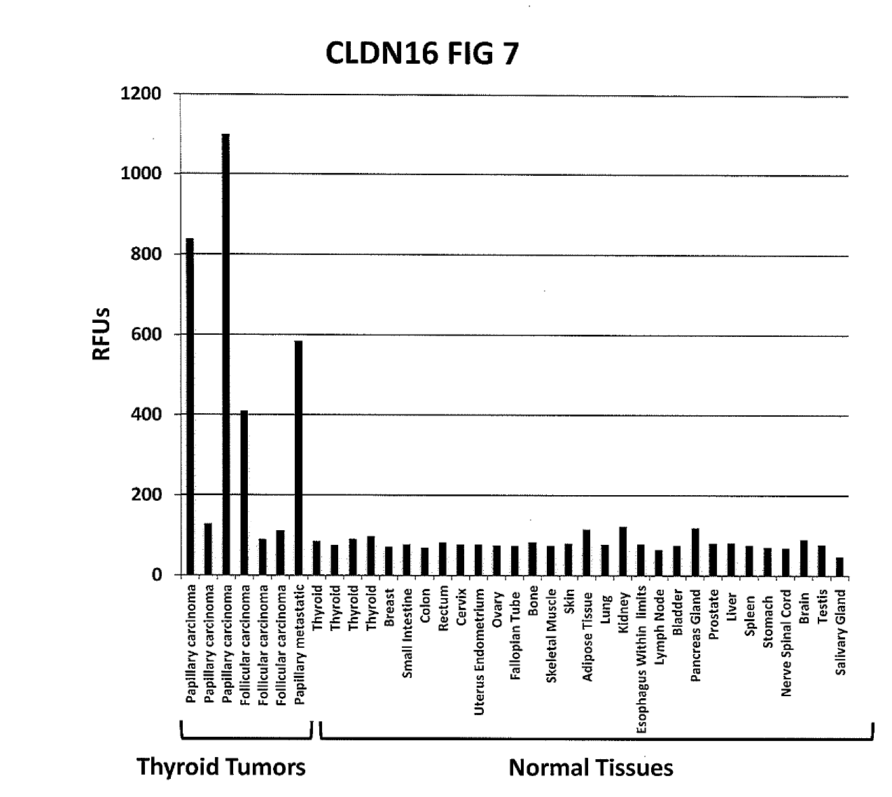

CLDN16: CLDN16 (Accession number NM_{—}006580.2) encodes Homo sapiens claudin 16. It is disclosed here that CLDN16 is a novel marker for thyroid tumors. As shown in FIG. 7, CLDN16 expression was assayed by Illumina microarray, a probe specific for CLDN16 (probe sequence CAGCCCCTCGCACAGAGACGGCCAAAATGTATGCTGTAGACACAAGGGTG(SEQ ID NO: 58); Illumina probe ID ILMN_{—}1707670) detected strong gene expression (>125 RFUs) in thyroid gland tumor papillary carcinoma, thyroid gland follicular carcinoma and metastatic papillary thyroid carcinoma. In contrast, expression of CLDN16 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<125 RFUs). The specificity of elevated CLDN16 expression in malignant tumors of thyroid origin shown herein demonstrates that CLDN16 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target CLDN16 can be identified using the methods described herein and therapeutics that target CLDN16 include, but are not limited to, antibodies that modulate the activity of CLDN16. The manufacture and use of antibodies are described herein.

### Example 8

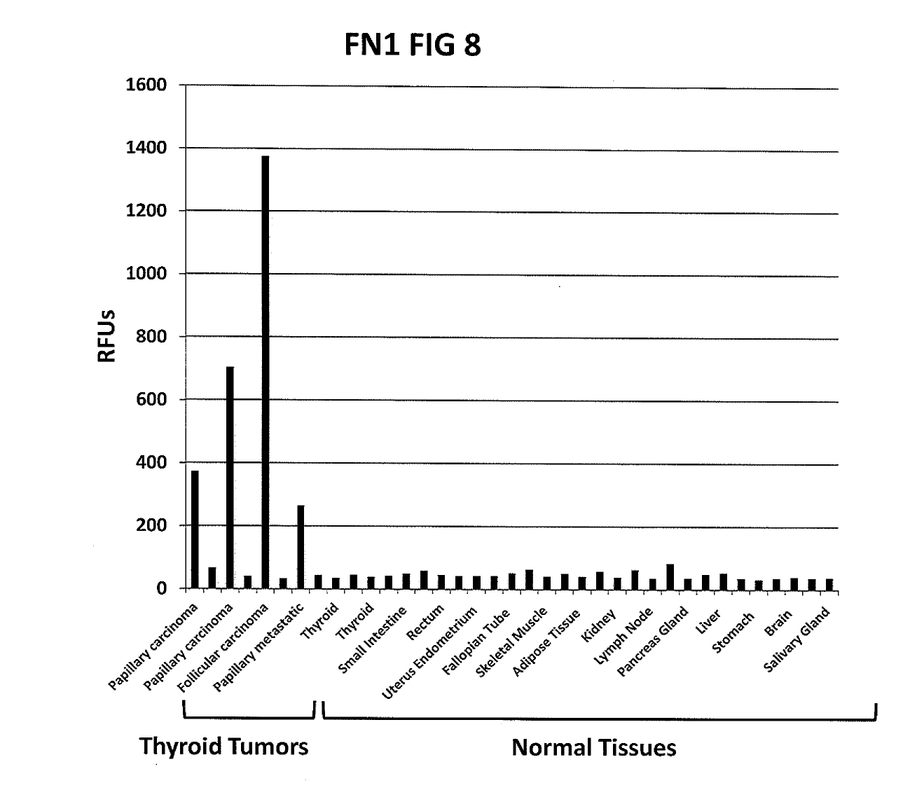

FN1: FN1 (Accession number NM_{—}002026.2) encodes Homo sapiens fibronectin 1. It is disclosed here that FN1 is a novel marker for thyroid tumors. As shown in FIG. 8, FN1 expression was assayed by illumina microarray, a probe specific for FN1 (probe sequence GCAGGTGGAAGTGTGATCCCGTCGACCAATGCCAGGATCAGAGACTGGG (SEQ ID NO: 59); Illumina probe ID ILMN_{—}1778237) detected strong gene expression (>100 RFUs) in thyroid gland tumor papillary carcinoma, thyroid gland follicular carcinoma and metastatic papillary thyroid carcinoma. In contrast, expression of FN1 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<100 RFUs). The specificity of elevated FN1 expression in malignant tumors of thyroid origin shown herein demonstrates that FN1 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target FN1 can be identified using the methods described herein and therapeutics that target FN1 include, but are not limited to, antibodies that modulate the activity of FN1. The manufacture and use of antibodies are described herein.

### Example 9

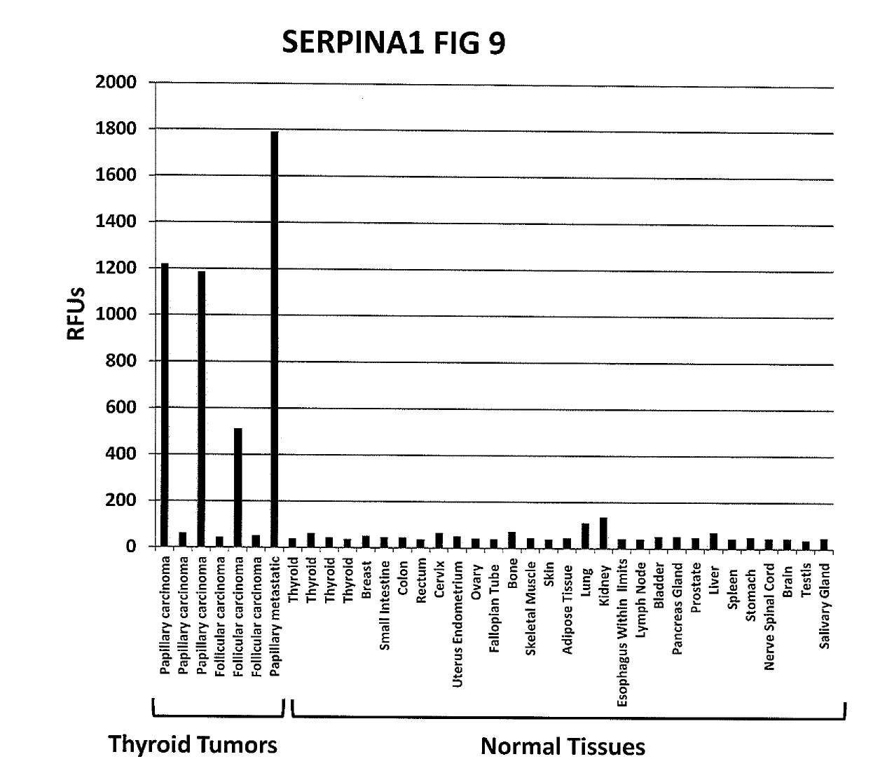

SERPINA1: SERPINA1 (Accession number NM_{—}000295.3) encodes Homo sapiens serpin peptidase inhibitor, clade A (alpha-1 antiproteinase, antitrypsin), member 1. It is disclosed here that SERPINA1 is a novel marker for thyroid tumors. As shown in FIG. 9, SERPINA1 expression was assayed by Illumina microarray, a probe specific for SERPINA1 (probe sequence AGTGGACTTAGCCCCTGTITGCTCCTCCGATAACTGGGTGACCTTGGTT (SEQ ID NO: 60); Illumina probe ID ILMN_{—}1764980) detected strong gene expression (>150 RFUs) in thyroid gland tumor papillary carcinoma, thyroid gland follicular carcinoma and metastatic papillary thyroid carcinoma. In contrast, expression of SERPINA1 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<150 RFUs). The specificity of elevated SERPINA1 expression in malignant tumors of thyroid origin shown herein demonstrates that SERPINA1 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target SERPINA1 can be identified using the methods described herein and therapeutics that target SERPINA1 include, but are not limited to, antibodies that modulate the activity of SERPINA1. The manufacture and use of antibodies are described herein.

### Example 10

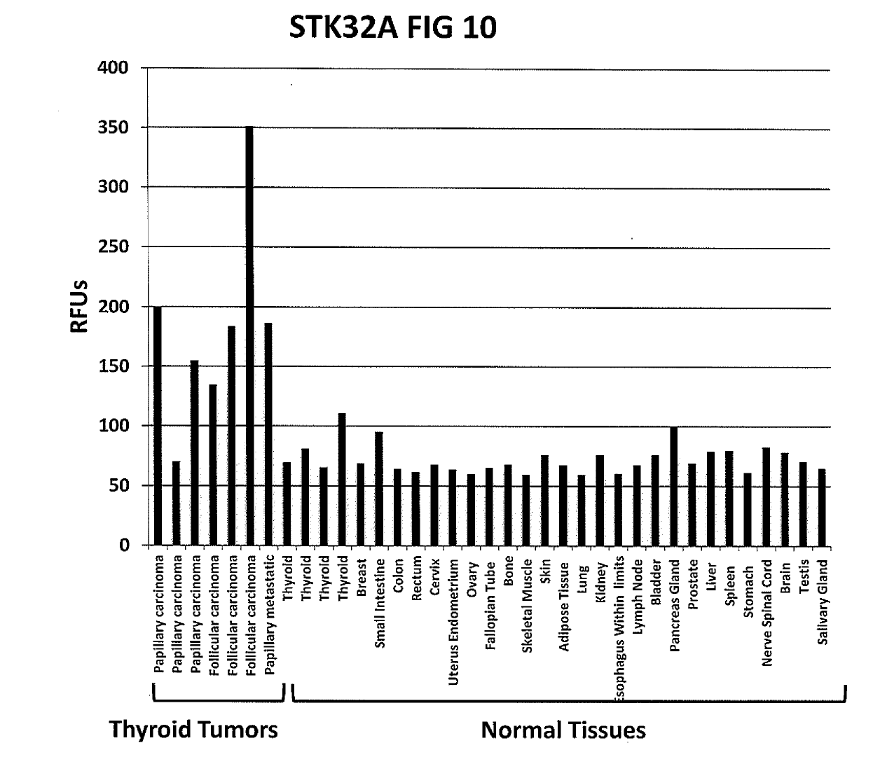

STK32A: STK32A (Accession number NM_{—}145001.2) encodes Homo sapiens serine/threonine kinase 32A. It is disclosed here that STK32A is a novel marker for thyroid tumors. As shown in FIG. 10, STK32A expression was assayed by Illumina microarray, a probe specific for STK32A (probe sequence GGTCATGGCCCTGGACTACCTGCAGAACCAGCGCATCATTCACAGGGATA(SEQ ID NO: 61); Illumina probe ID ILMN_{—}1756612) detected strong gene expression (>120 RFUs) in thyroid gland tumor papillary carcinoma, thyroid gland follicular carcinoma and metastatic papillary thyroid carcinoma. In contrast, expression of STK32A in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<120 RFUs). The specificity of elevated STK32A expression in malignant tumors of thyroid origin shown herein demonstrates that STK32A is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target STK32A can be identified using the methods described herein and therapeutics that target STK32A include, but are not limited to, antibodies that modulate the activity of STK32A. The manufacture and use of antibodies are described herein.

### Example 11

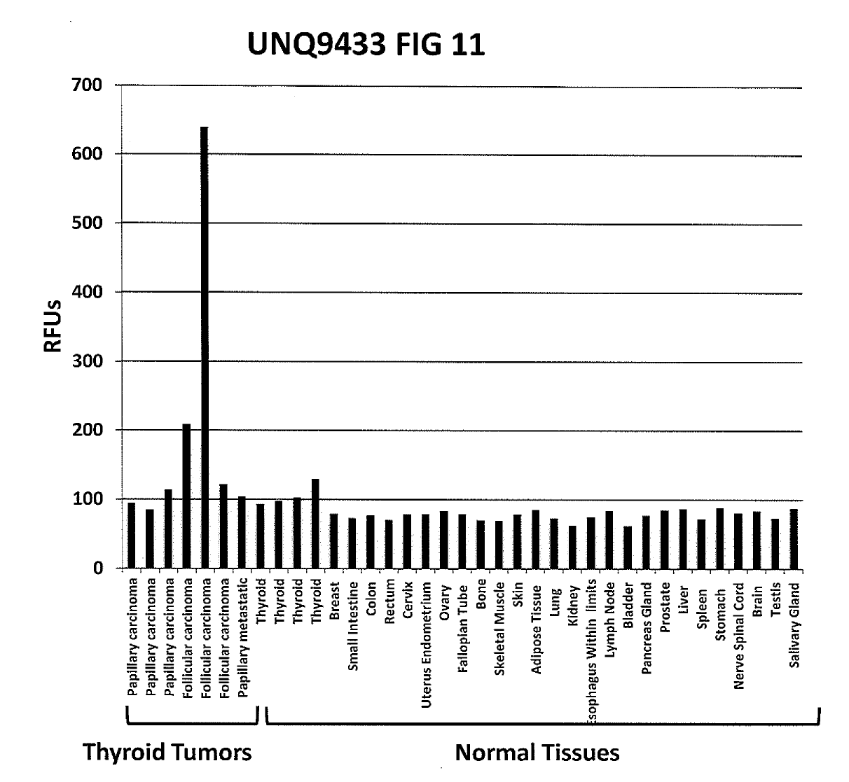

UNQ9433: UNQ9433 (Accession number NM_{—}207413.1) encodes Homo sapiens RPLK9433 (UNQ9433). It is disclosed here that UNQ9433 is a novel marker for thyroid tumors. As shown in FIG. 11, UNQ9433 expression was assayed by Illumina microarray, a probe specific for UNQ9433 (probe sequence AGACTTCCCAGAAATAACTGGTITAGCTGTTTCCTGTCATAGAATGGAGTC (SEQ ID NO: 62); Illumina probe ID ILMN_{—}2091217) detected strong gene expression (>140 RFUs) in thyroid gland follicular carcinoma. In contrast, expression of UNQ9433 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<140 RFUs). The specificity of elevated UNQ9433 expression in malignant tumors of thyroid origin shown herein demonstrates that UNQ9433 is a marker for the diagnosis of thyroid cancer (e.g. Including but not limited to thyroid follicular carcinomas), and is a target for therapeutic intervention in thyroid cancer. The marker may be detected in urine as well as sera.

Therapeutics that target UNQ9433 can be identified using the methods described herein and therapeutics that target UNQ9433 include, but are not limited to, antibodies that modulate the activity of UNQ9433. The manufacture and use of antibodies are described herein.

### Example 12

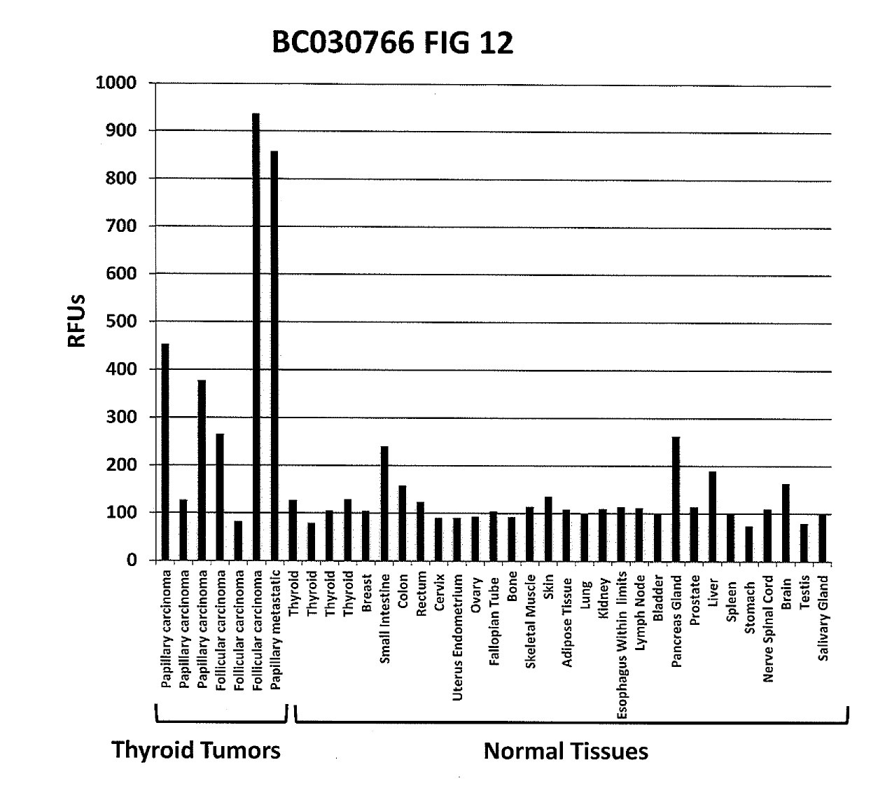

BC030766: BC030766 (Accession number BC030766) encodes Homo sapiens cDNA clone IMAGE:4811759. It is disclosed here that BC030766 is a novel marker for thyroid tumors. As shown in FIG. 12, BC030766 expression was assayed by Illumina microarray, a probe specific for BC030766 (probe sequence CTCTGGCTGCAGTTAAATGGTCTGCATITGCTCTGGCTITCAGGCC (SEQ ID NO: 63); Illumina probe ID ILMN_{—}1904578) detected strong gene expression (>200 RFUs) in thyroid gland tumor papillary carcinoma, thyroid gland follicular carcinoma and metastatic papillary thyroid carcinoma. In contrast, expression of BC030766 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<200 RFUs). The specificity of elevated BC030766 expression in malignant tumors of thyroid origin shown herein demonstrates that BC030766 is a marker for the diagnosis of thyroid cancer (e.g. Including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target BC030766 can be identified using the methods described herein and therapeutics that target BC030766 include, but are not limited to, antibodies that modulate the activity of BC030766. The manufacture and use of antibodies are described herein.

### Example 13

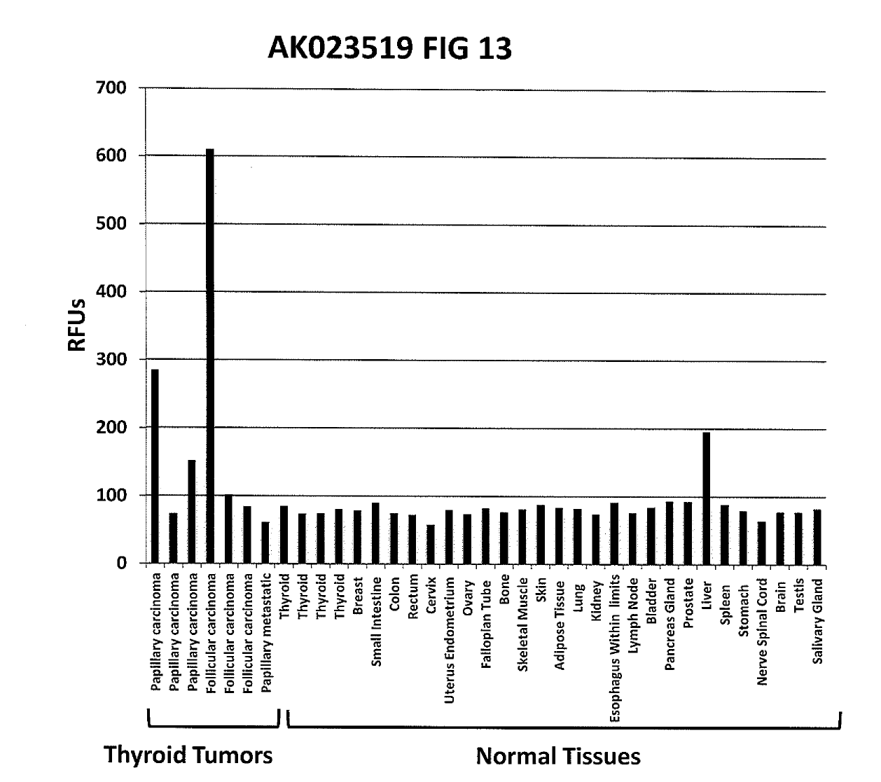

AK023519: AK023519 (Accession number AK023519) encodes Homo sapiens cDNA FLJ13457 fis, clone PLACE1003343. It is disclosed here that AK023519 is a novel marker for thyroid tumors. As shown in FIG. 13, AK023519 expression was assayed by Illumina microarray, a probe specific for AK023519 (probe sequence CAGAGTCTCCGGGCCTTGGTAATTCCTAGACCACAGCACCATGCATTAGG (SEQ ID NO: 64); Illumina probe ID ILMN_{—}1913510) detected strong gene expression (>200 RFUs) in thyroid gland tumor papillary carcinoma and thyroid gland follicular carcinoma. In contrast, expression of AK023519 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<200 RFUs). The specificity of elevated AK023519 expression in malignant tumors of thyroid origin shown herein demonstrates that AK023519 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target AK023519 can be identified using the methods described herein and therapeutics that target AK023519 include, but are not limited to, antibodies that modulate the activity of AK023519. The manufacture and use of antibodies are described herein.

### Example 14

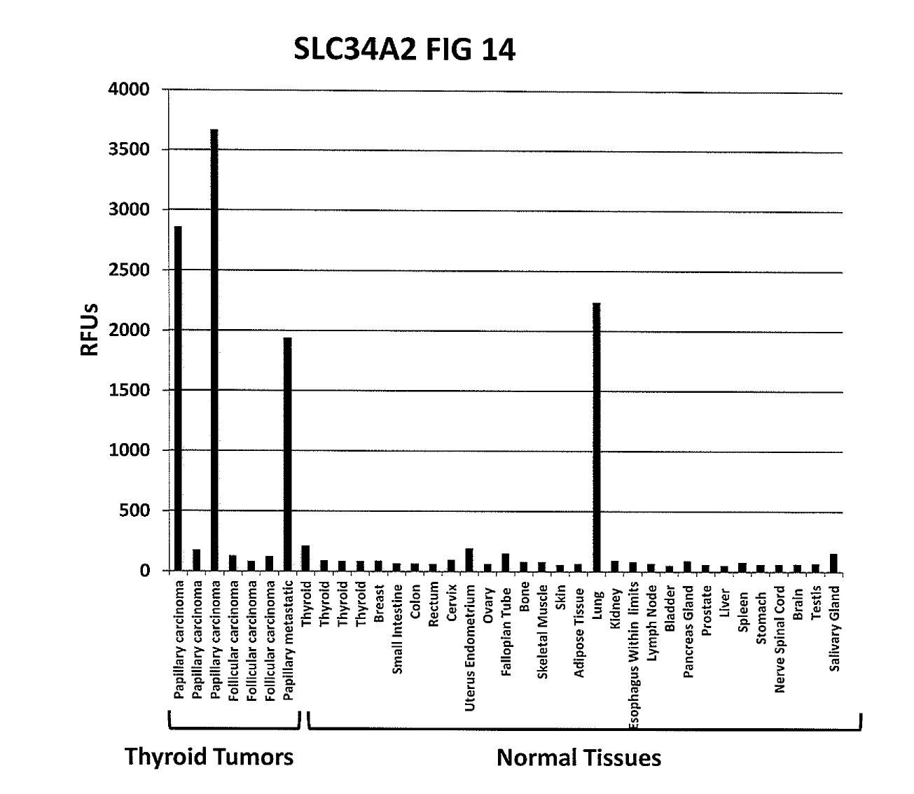

SLC34A2: SLC34A2 (Accession number NM_{—}006424.2) encodes Homo sapiens solute carrier family 34 (sodium phosphate), member 2. It is disclosed here that SLC34A2 is a novel marker for thyroid tumors. As shown in FIG. 14, SLC34A2 expression was assayed by Illumina microarray, a probe specific for SLC34A2 (probe sequence ATCTAGGAAAGGAGGAGTGGGTGTAGCCGTGCAGCAAGATTGGGGCCTCC (SEQ ID NO: 65); Illumina probe ID ILMN_{—}2184109) detected strong gene expression (>2300 RFUs) In thyroid gland tumor papillary carcinoma. In contrast, expression of SLC34A2 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<2300 RFUs). The specificity of elevated SLC34A2 expression in malignant tumors of thyroid origin shown herein demonstrates that SLC34A2 is a marker for the diagnosis of thyroid cancer (e.g. including but not limited to thyroid papillary carcinomas), and is a target for therapeutic intervention in thyroid cancer. The marker may be detected in urine as well as sera.

Therapeutics that target SLC34A2 can be identified using the methods described herein and therapeutics that target SLC34A2 include, but are not limited to, antibodies that modulate the activity of SLC34A2. The manufacture and use of antibodies are described herein.

### Example 15

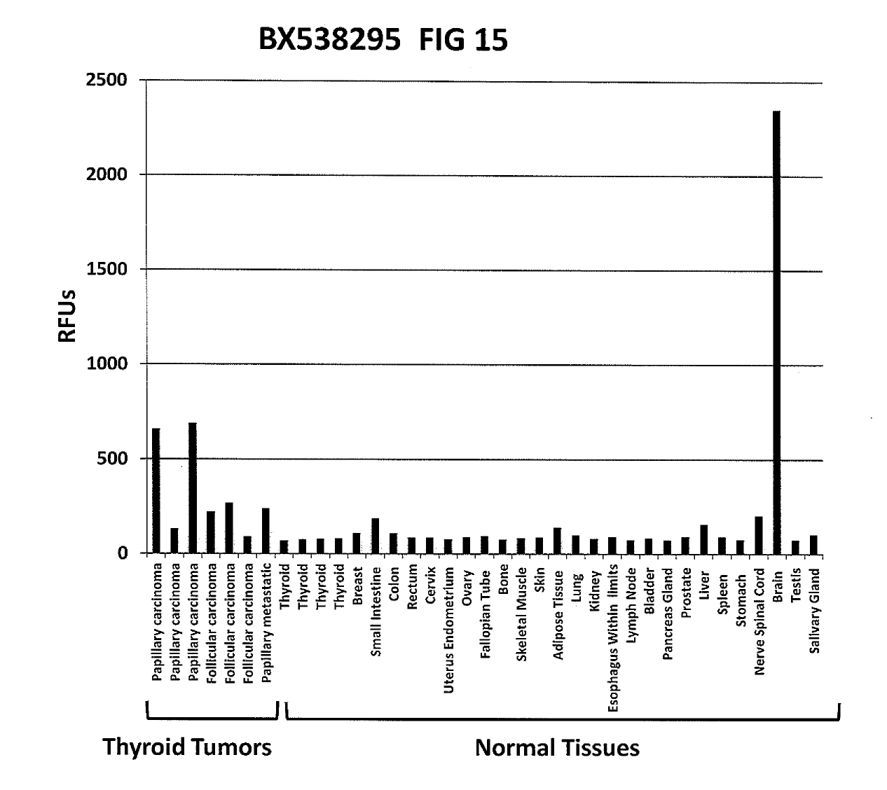

BX538295: BX538295 (Accession number BX538295) encodes Homo sapiens mRNA; cDNA DKFZp686N1644 (from clone DKFZp686N1644). It is disclosed here that BX538295 is a novel marker for thyroid tumors. As shown in FIG. 15, BX538295 expression was assayed by Illumina microarray, a probe specific for BX538295 (probe sequence TCTGGCTTACAGGGGAACACAACTATCCACAAGTGGCCTrTAGTGCTCT (SEQ ID NO: 66); Illumina probe ID ILMN_{—}1861270) detected strong gene expression (>240 RFUs) in thyroid gland tumor papillary carcinoma, thyroid gland follicular carcinoma and metastatic papillary thyroid carcinoma. In contrast, expression of BX538295 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, testis, thyroid, and salivary gland was generally low (<240 RFUs), with the exception of brain (2353 RFUs). The specificity of elevated BX538295 expression in malignant tumors of thyroid origin shown herein demonstrates that BX538295 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target BX538295 can be identified using the methods described herein and therapeutics that target BX538295 include, but are not limited to, antibodies that modulate the activity of BX538295. The manufacture and use of antibodies are described herein.

### Example 16

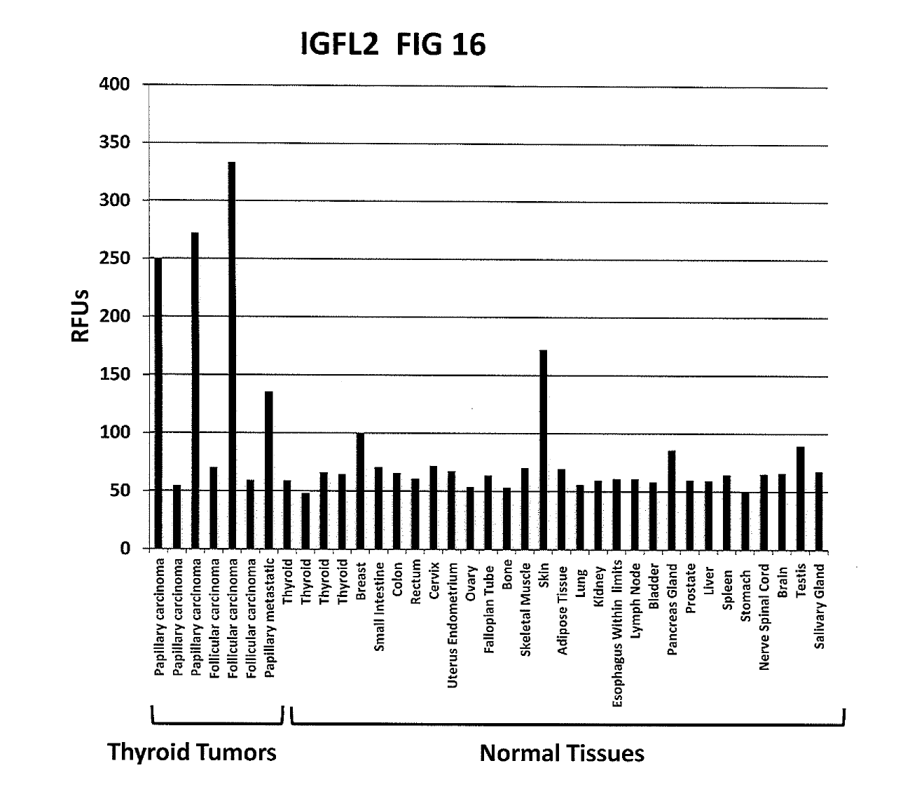

IGFL2: IGFL2 (Accession number NM_{—}001555.2) encodes Homo sapiens IGF-like family member 2. It is disclosed here that IGFL2 is a novel marker for thyroid tumors. As shown in FIG. 16, IGFL2 expression was assayed by Illumina microarray, a probe specific for IGFL2 (probe sequence GCTGGCTCCTGCTTATGTGTCAGTCTGTCTCCTCCTCTTGTGTCCAAGGG (SEQ ID NO: 67); Illumina probe ID ILMN_{—}1790227) detected strong gene expression (>180 RFUs) in thyroid gland tumor papillary carcinoma and thyroid gland follicular carcinoma. In contrast, expression of IGFL2 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<180 RFUs). The specificity of elevated IGFL2 expression in malignant tumors of thyroid origin shown herein demonstrates that IGFL2 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target IGFL2 can be identified using the methods described herein and therapeutics that target IGFL2 include, but are not limited to, antibodies that modulate the activity of IGFL2. The manufacture and use of antibodies are described herein.

### Example 17

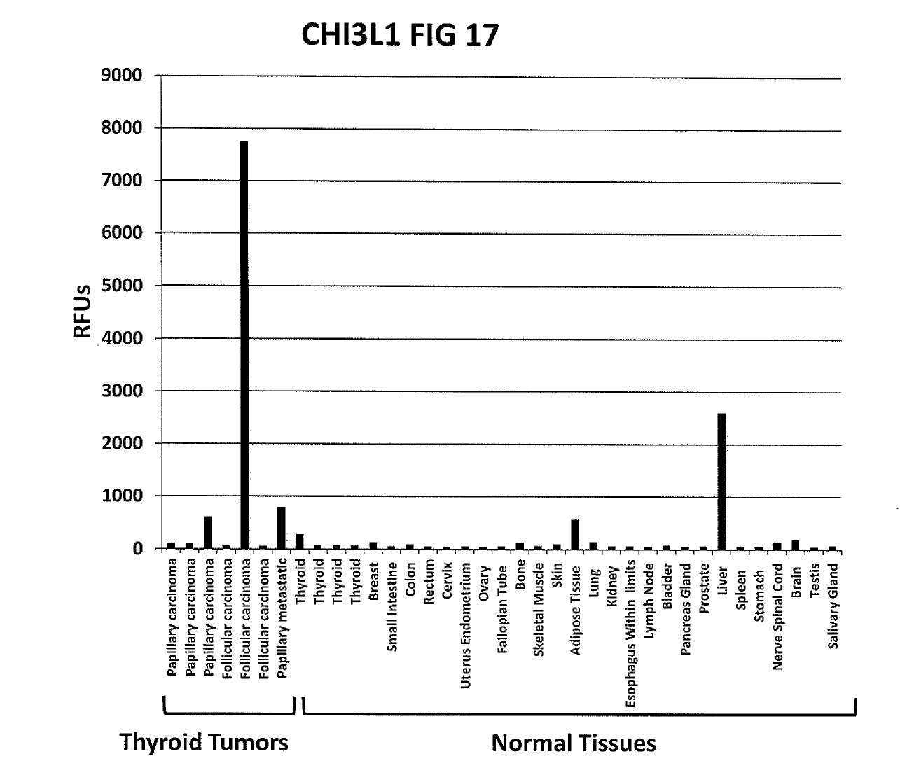

CHI3L1: CHI3L1 (Accession number NM_{—}001276.2) encodes Homo sapiens chitinase 3-like 1 (cartilage glycoprotein-39). It is disclosed here that CHI3L1 is a novel marker for thyroid tumors. As shown in FIG. 17, CHI3L1 expression was assayed by Illumina microarray, a probe specific for CHI3L1 (probe sequence GGGATGGGGCTGTGOGGATAGTGAGGCATCGCAATGTAAGACTCGGGATT (SEQ ID NO: 68); lllumina probe ID ILMN_{—}3307868) detected strong gene expression (>600 RFUs) in thyroid gland tumor papillary carcinoma, thyroid gland follicular carcinoma and metastatic papillary thyroid carcinoma. In contrast, expression of CHI3L1 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, spleen, stomach, spinal cord, brain, testis, thyroid, and salivary gland was generally low (<600 RFUs), with the exception of liver (2605 RFUs). The specificity of elevated CHI3L1 expression in malignant tumors of thyroid origin shown herein demonstrates that CHI3L1 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target CHI3L1 can be identified using the methods described herein and therapeutics that target CHI3L1 include, but are not limited to, antibodies that modulate the activity of CHI3L1. The manufacture and use of antibodies are described herein.

### Example 18

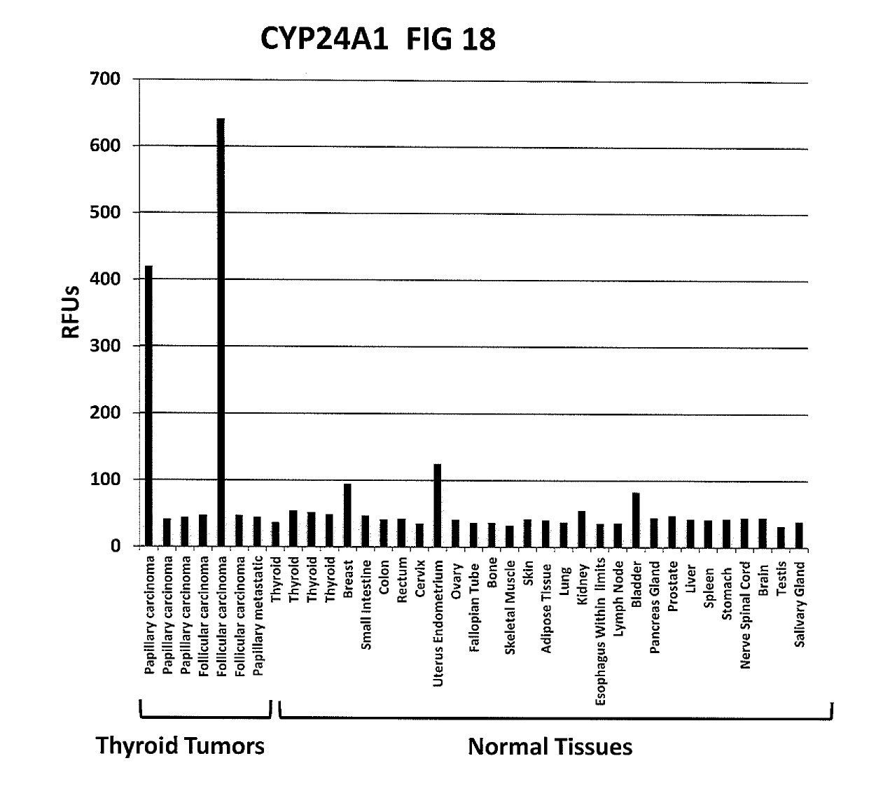

CYP24A1: CYP24A1 (Accession number NM_{—}000782.3) encodes Homo sapiens cytochrome P450, family 24, subfamily A, polypeptide 1. It is disclosed here that CYP24A1 is a novel marker for thyroid tumors. As shown in FIG. 18, CYP24A1 expression was assayed by illumina microarray, a probe specific for CYP24A1 (probe sequence GATITAGGATCTGTGGTGCAGGGCAATGITCAAAGTTTAGTCACAGCTT (SEQ ID NO: 69); Illumina probe ID ILMN_{—}1685663) detected strong gene expression (>200 RFUs) in thyroid gland tumor papillary carcinoma and thyroid gland follicular carcinoma. In contrast, expression of CYP24A1 in a wide variety of normal tissues including normal thyroid, kidney, breast, colon, rectum, cervix, endometrium, ovary, fallopian tube, bone, skeletal muscle, skin, adipose tissue, lung, thyroid, esophagus, lymph node, bladder, pancreas, prostate, liver, spleen, stomach, spinal cord, brain, testis, thyroid, and salivary gland was generally low (<200 RFUs). The specificity of elevated CYP24A1 expression in malignant tumors of thyroid origin shown herein demonstrates that CYP24A1 is a marker for the diagnosis of thyroid cancer (e.g. including but not limited to thyroid papillary carcinomas, thyroid follicular carcinomas and metastatic thyroid tumors), and is a target for therapeutic intervention in thyroid cancer. The marker may be detected in urine as well as sera.

Therapeutics that target CYP24A1 can be identified using the methods described herein and therapeutics that target CYP24A1 include, but are not limited to, antibodies that modulate the activity of CYP24A1. The manufacture and use of antibodies are described herein.

### Example 19

qPCR was performed as described below for the following genes: IGSF1; CHI3L; TM7SF4; ZCCHC12; SFTPB; NMU; PLAG1; and FLJ30058.

PCR primers were designed to be specific for the gene transcript of interest using the Standard Nucleotide BLAST program (NCBI) and to span at least one exon junction. Primers were chosen to have Tms of 58-63° C. calculated with the Breslauer equation, deltaG values >25 Kcal/mol and displaying no self-complementarity using Oligo Cale software. Primers were ordered salt-free purified from the manufacturer (Eurofins MWG) (See Tables for primer sequence and parameters).

RNA was derived from commercial sources (Asterand; OriGene) and cDNA prepared using the SuperScript III First-Strand Synthesis System for RT-PCR (Invitrogen Cat. No. 18080-051) following the random hexamer protocol. Initial validation of primers assessed three major criteria: robustness, linearity and specificity. Acceptance criteria for absolute value robustness was that the final 2̂delta Ct value after subtracting housekeeping genes (GAPDH and GUSB) Ct values >1. Robustness in terms of differentiating disease from benign or normal samples required >2Ct difference of known positive over negative samples, as determined previously by microarray analysis (Illumina). To assess linearity, primers were used to amplify ten-fold dilutions of cDNA. Only primers exhibiting at or near the expected 3.3 Ct shift upon ten-fold dilution of template proceeded for further testing. Specificity was determined both by gel electrophoresis and from observing a single Tm generated from melting curve analysis on the instrument. PCR products were run on a 2% agarose gel and only those generating a single band of expected size passed validation.

Protocols of initial primer validation differed from external validation performed on OriGene TissueScan qPCR arrays chiefly in terms of volume and cDNA target.

PCR Protocol for Initial Primer Validation:

| Reagent | 1 Rx (μL) | Final Conc |
|---|---|---|
| 2X Power SYBR Green Master Mix | 10.0 | 1X |
| (Invitrogen Cat #4368706) | | |
| 100 μM F Primer (Eurofins MWG) | 0.20 | 1 μM |
| 100 μM R Primer (Eurofins MWG) | 0.20 | 1 μM |
| 10 or 1 ng/μL cDNA Template | 1.00 | |
| Molecular Biology grade H₂O | 18.6 | |
| (Cellgro Cat No 46-000-CM) | | |
| | 20.0 | |
| | Thermoprogram used on | |
| PCR Instruments | both Instruments: | |
| ABI 7500 Real Time PCR System | Activation | 50° C. 2:00 |
| ABI 7900HT Sequence Detection System | Denature | 95° C. 10:00 |
| | 40 Cycles | 95° C. 0:15 |
| | | 60° C. 1:00 |
| | Dissociation | 95° C. 0:15 |
| | | 60° C. 0:15 |
| | | 95° C. 0:15 |

Protocol for OriGene TissueScan Arrays:

| Reagent | 1 Rx (μL) | Final Cont |
|---|---|---|
| 2X Power SYBR Green Master Mix | 15.0 | 1X |
| (Invitrogen Cat #4368706) | | |
| 100 μM F Primer (Eurofins MWG) | 0.30 | 1 μM |
| 100 μM R Primer (Eurofins MWG) | 0.30 | 1 μM |
| Molecular Biology grade H₂O | 14.4 | |
| (Cellgro Cat No 46-000-CM) | | |
| | 30.0 | |
| PCR instruments | Thermoprogram used: | |
| ABI 7500 Real Time PCR System | Activation | 50° C. 2:00 |
| | Denature | 95° C. 10:00 |
| | 42 Cycles | 95° C. 0:15 |
| | | 60° C. 1:00 |
| | | (72° C. 0:10) |
| | | Used with amplicons |
| | | >120 bp |
| | Dissociation | 95° C. 0:15 |
| | | 60° C. 0:15 |
| | | 95° C. 0:15 |

Primers used are provided in Tables 4 and 5 below:

TABLE 4

| Gene | | | |
|---|---|---|---|
| Marker | Forward Primer | Forward Primer Sequence | Accession # |
| IGSF1 | JK1132-IGSF1-F | GGGCCTTCAACTACCATCCCAC (SEQ ID NO: 70) | NM_001555.2 |
| CHI3L1 | JK1140-CHI3L1-F | CCCTGTCTAGGTAGCTGGCAC (SEQ ID NO: 71) | NM_001276.2 |
| TM7SF4 | JK1144-TM7SF4-F | AGAGAAACCTGACGCAGGGAGC (SEQ ID NO: 72) | NM_030788.2 |
| ZCCHC12 | JK1158-ZCCHC12-F | TCCACCAGCGGAGCACAGGCC (SEQ ID NO: 73) | NM_173798.2 |
| SFTPB | JK1156-SFTPB-F | CTCTGTGGCCCAGGCACTGC (SEQ ID NO: 74) | NM_000542.2 |
| NMU | JK1210-NMU-F | TCTTTTCTGTCCATTGATTCTCAGCCTC (SEQ ID NO: 75) | NM_006681.2 |
| PLAG1 | JK1218-PLAG1-F | CGGTGTAGAGGCGGCGGAC (SEQ ID NO: 76) | NM_002655.2 |
| FLJ30058 | FLJ30058-ML-F1 | CACAACCCCGACCGCAGGAC (SEQ ID NO: 77) | NM_144967.2 |
| SLCO4C1 | JK1240-SLCO4C1-F | TTATGGCCGGTACTCCTATAACTGTGTC (SEQ ID NO: 78) | NM_180991.4 |

TABLE 5

| Gene | | | |
|---|---|---|---|
| Marker | Reverse Primer | Reverse Primer Sequence | Accession # |
| IGSF1 | JK1133-IGSF1-R | GGCACCAAAGCGTGATGTTCTCC (SEQ ID NO: 79) | NM_001555.2 |
| CHI3L1 | JK1141-CHI3L1-R | TATGCAGAGCAGCACTGGAGC (SEQ ID NO: 80) | NM_001276.2 |
| TM7SF4 | JK1145-TM7SF4-R | GCTATGATTGATGGCAGAAACCAGC (SEQ ID NO: 81) | NM_030788.2 |
| ZCCHC12 | JK1159-ZCCHC12-R | TGCCTTCCTATCTCAGCAGGGGAC (SEQ ID NO: 82) | NM_173798.2 |
| SFTPB | JK1157-SFTPB-R | ACACTCTTGGCATAGGTCATCGGC (SEQ ID NO: 83) | NM_000542.2 |
| NMU | JK1211-NMU-R | CTCTCATGCAGGTGAGGAACGAGC (SEQ ID NO: 84) | NM_006681.2 |
| PLAG1 | JK1219-PLAG1-R | ACTGATGGAAAAAGCCTCAGACTTTGATC (SEQ ID NO: 85) | NM_002655.2 |
| FLJ30058 | FLJ30058-ML-R1 | ACAGGAAATGTCTGGCCACGAGT (SEQ ID NO: 86) | NM_144967.2 |
| SLCO4C1 | JK1241-SLCO4C1-R | TCTGTGGCTGATGGAGGTGGTTTATAC (SEQ ID NO: 87) | NM_180991.4 |

Initial validation experiments were performed using RNA derived from commercial sources (Asterand, Detroit, Mich.; OriGene, Rockville, Md.) and prepared into cDNA using the SuperScript III First-Strand Synthesis System for RT-PCR (Life Technologies, Carlsbad, Calif.) following the random hexamer protocol. The samples were amplified in quantitative reverse-transcriptase PCR (qRT-PCR) reactions with 1 uM final concentration of each of the forward and reverse primers (Eurofins MWG Huntsville, Ala.) using the Power SYBR Green Master Mix Kit (Life Technologies, Carlsbad, Calif.) following the manufacturer's instructions. Sample input was between 3 to 10 ng of cDNA in a final reaction volume of 20 uL. The real-time PCR instruments used were the ABI 7500 Real Time PCR System or the ABI 7900HT Sequence Detection System with the thermoprogram set for 50° C. for 2 minutes, then 95° C. for 10 minutes, followed by 40 cycles of 95° C. for 15 seconds and 60° C. for 1 minute. Dissociation analysis was immediately performed using 95° C. for 15 seconds, 60° C. for 15 seconds and 95° C. for 15 seconds.

Primers demonstrating good correlation and specificity for cancer, as well as exhibiting robustness and linear dose response to sample input proceeded for further testing. TissueScan qPCR arrays (OriGene, Rockville, Md.) were used to test larger number of cDNA samples. The lyophilized cDNA in each well of the array was mixed with 1 uM final concentration of each of the forward and reverse primers using the Power SYBR Green Master Mix Kit (Life Technologies, Carlsbad, Calif.) in a final reaction volume of 30 uL. The real-time PCR instrument used was the ABI 7500 Real Time PCR System with the thermoprogram set for 50° C. for 2 minutes, then 95° C. for 10 minutes, followed by 40 cycles of 95° C. for 15 seconds and 60° C. for 1 minute. Dissociation analysis was immediately performed using 95° C. for 15 seconds, 60° C. for 15 seconds and 95° C. for 15 seconds.

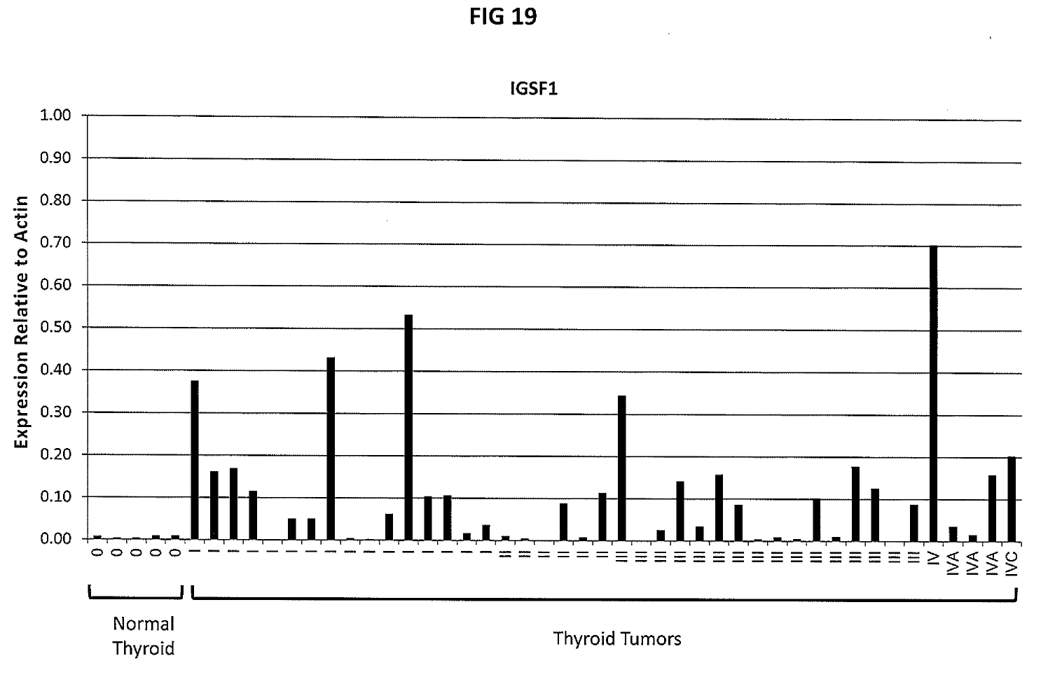

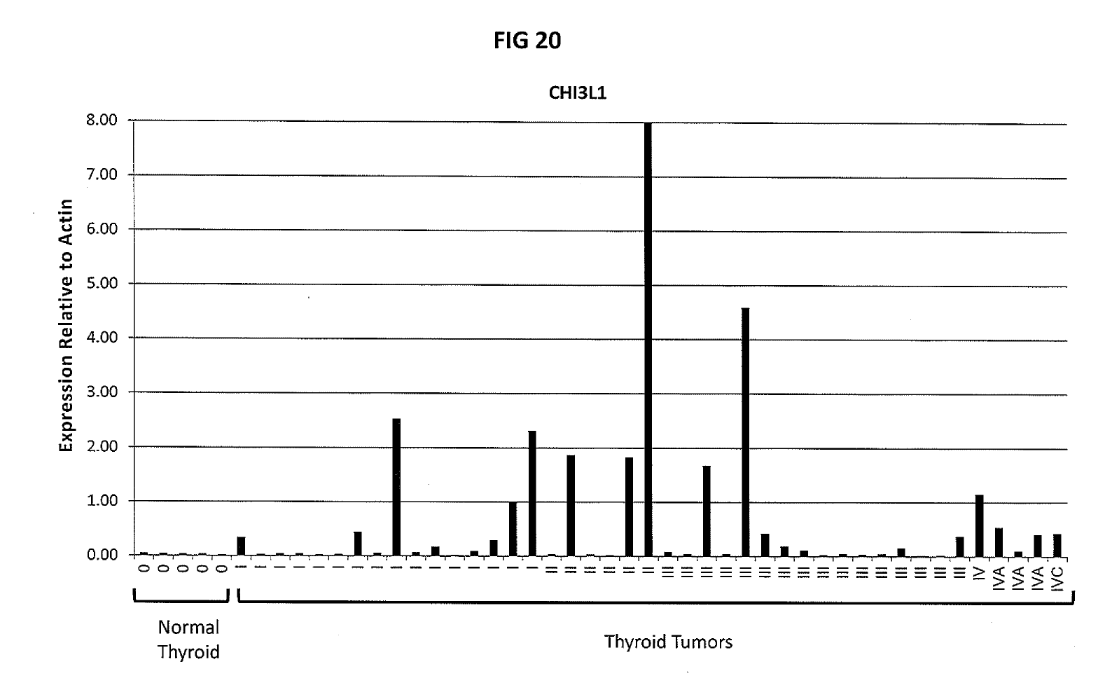

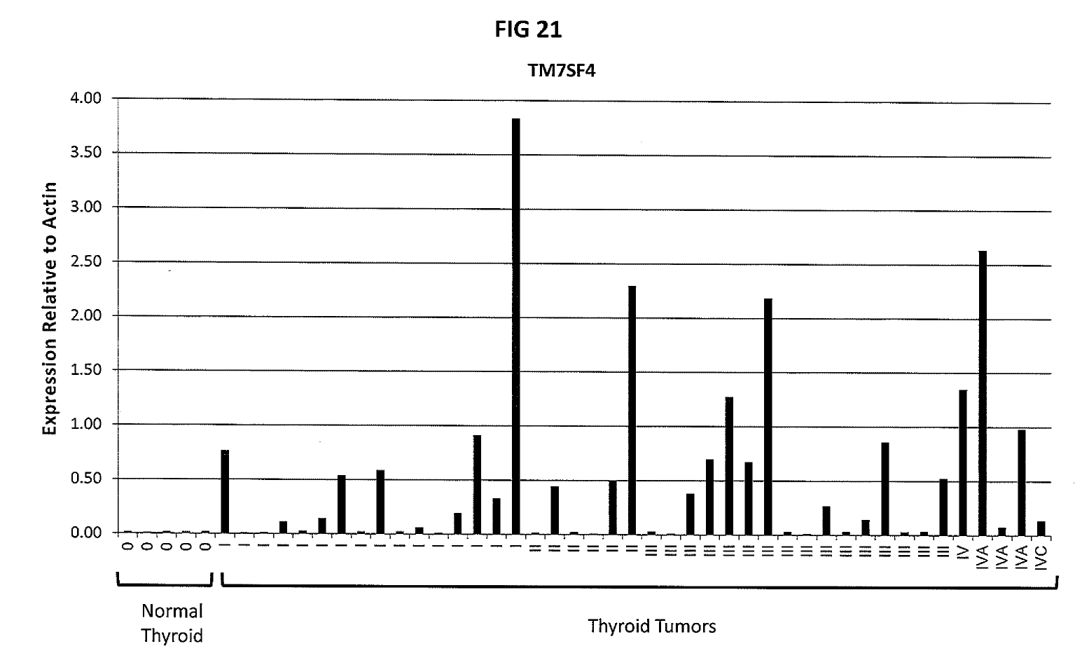

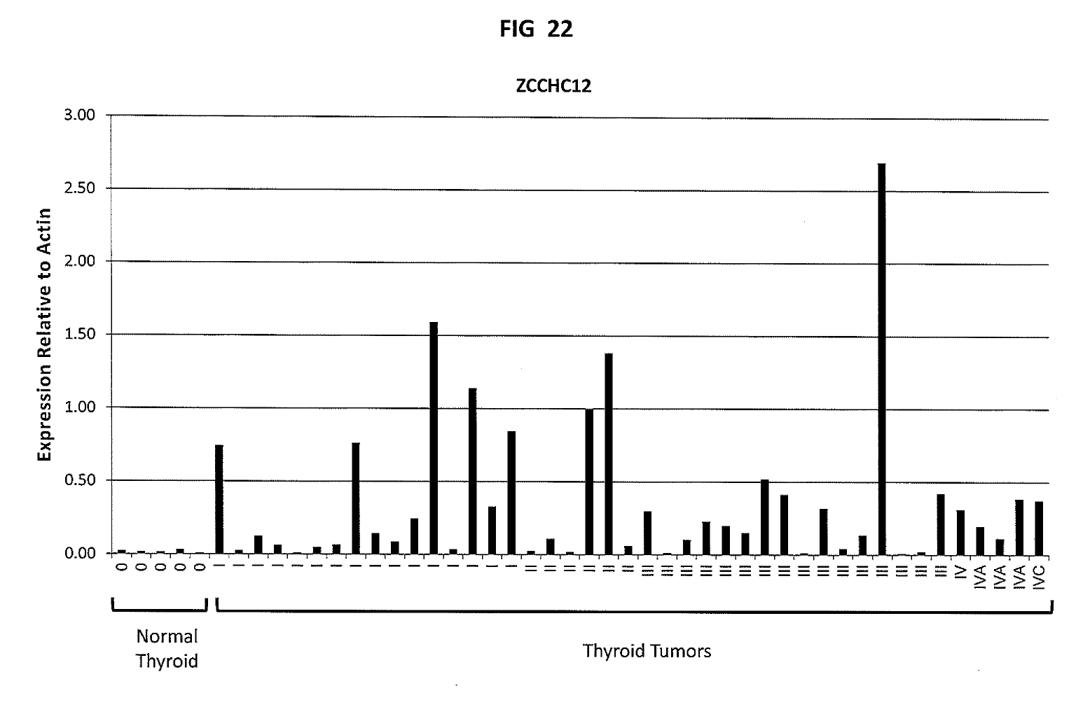

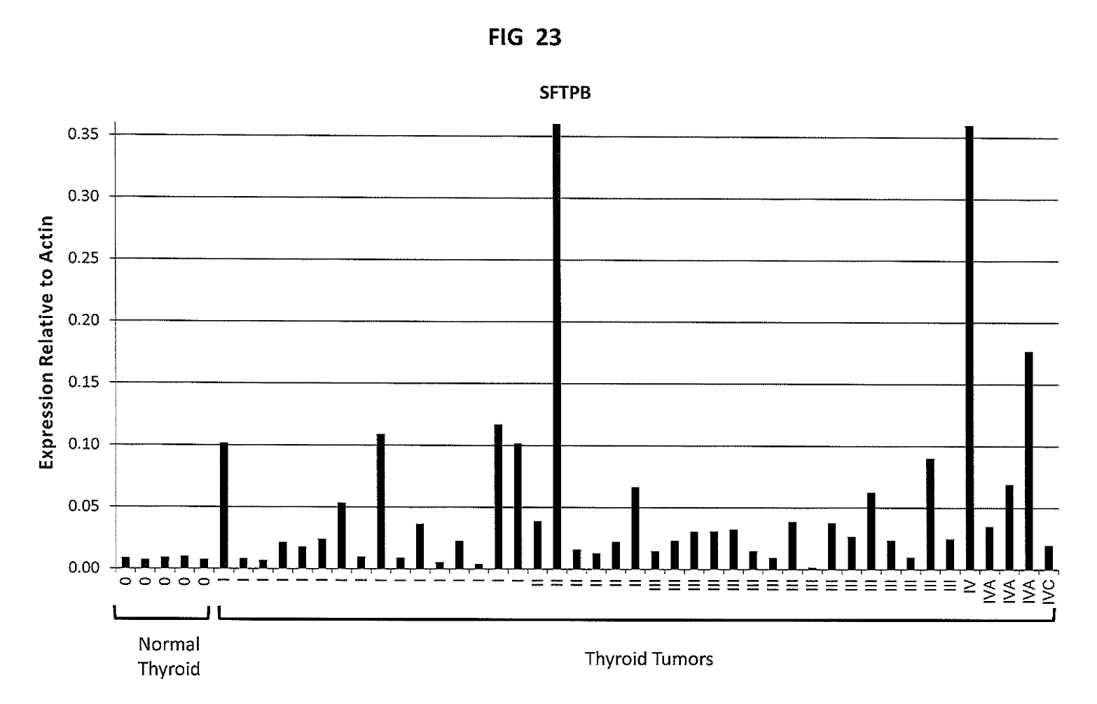

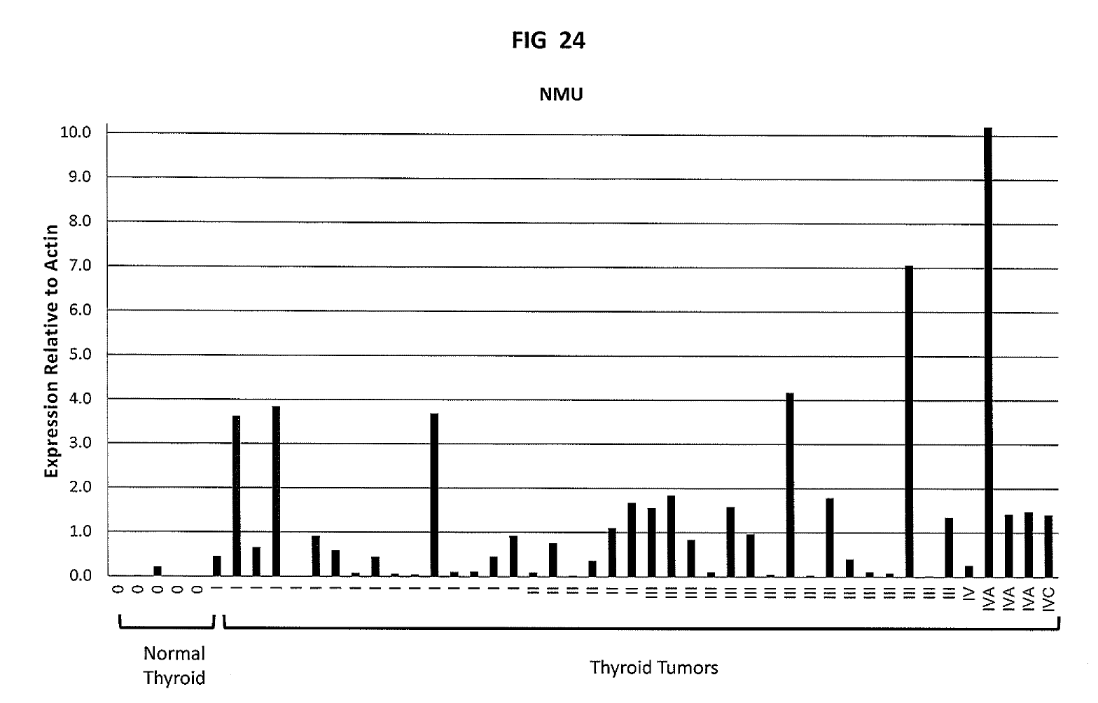

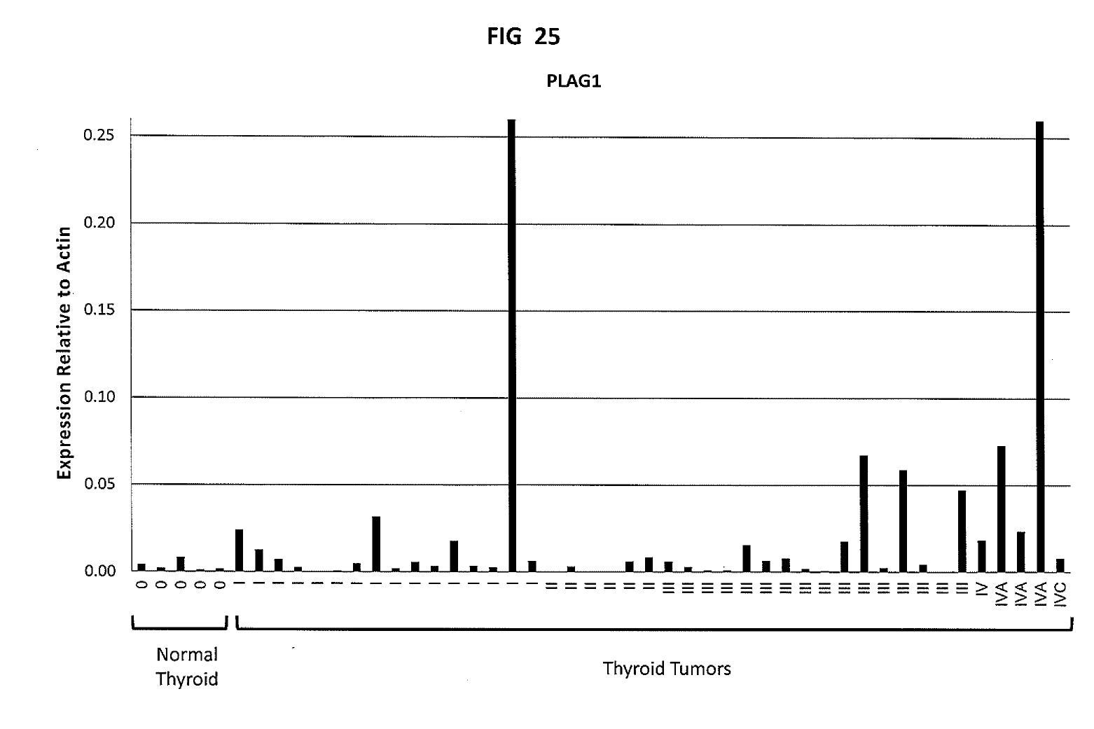

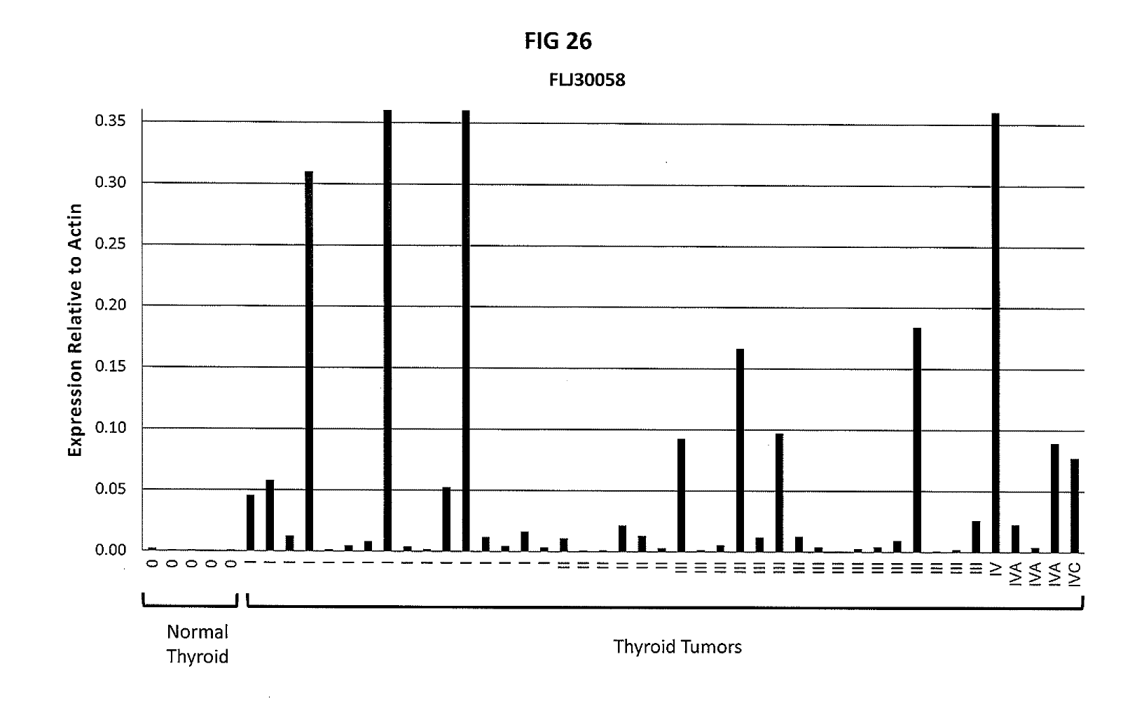

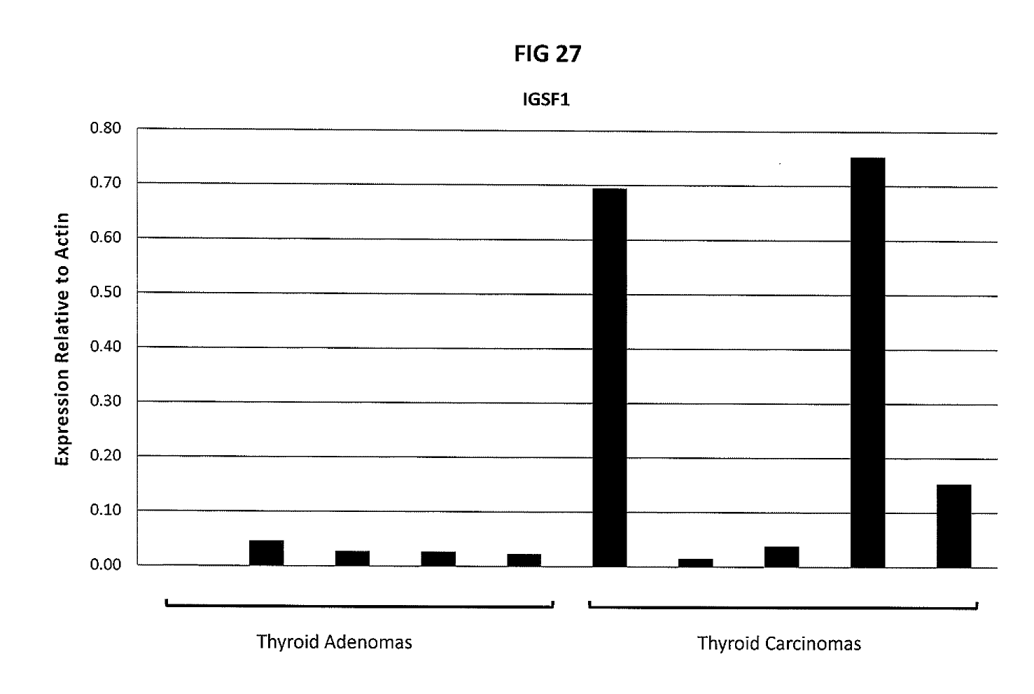

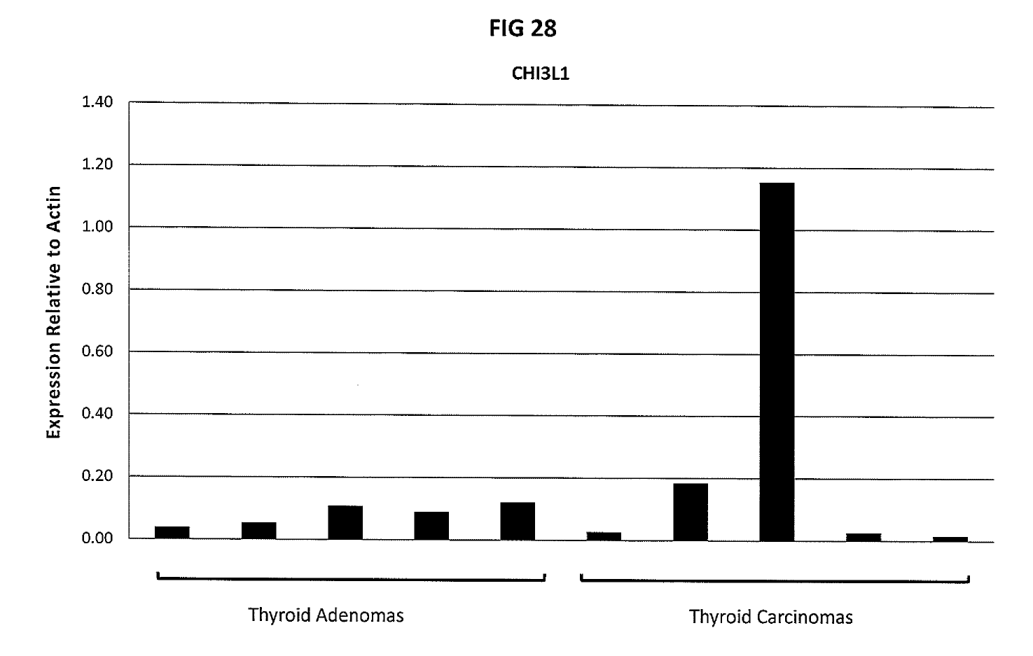

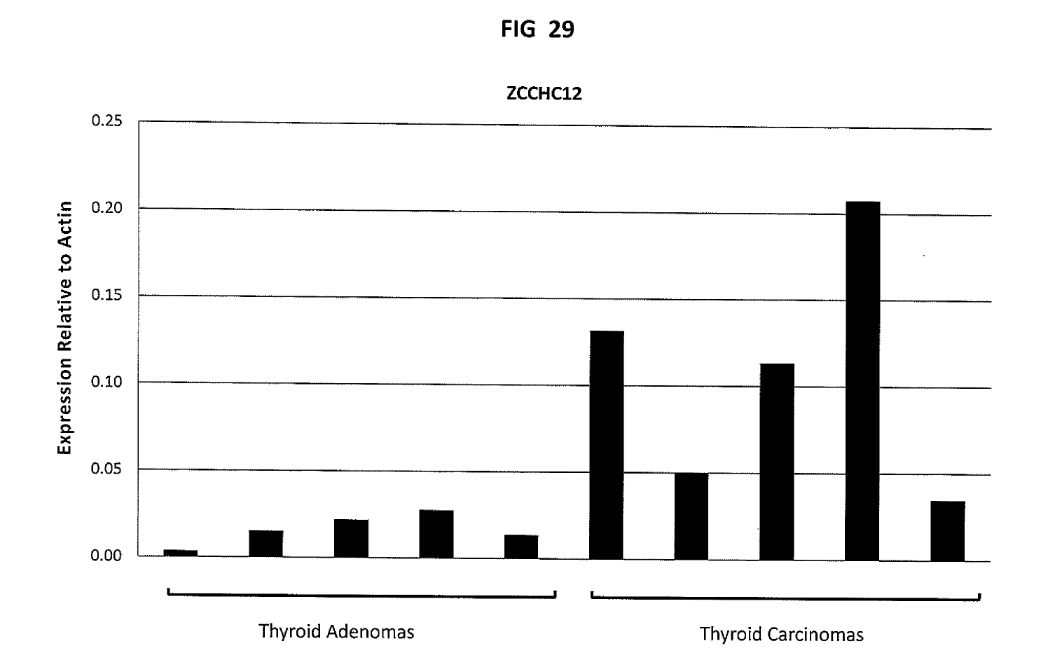

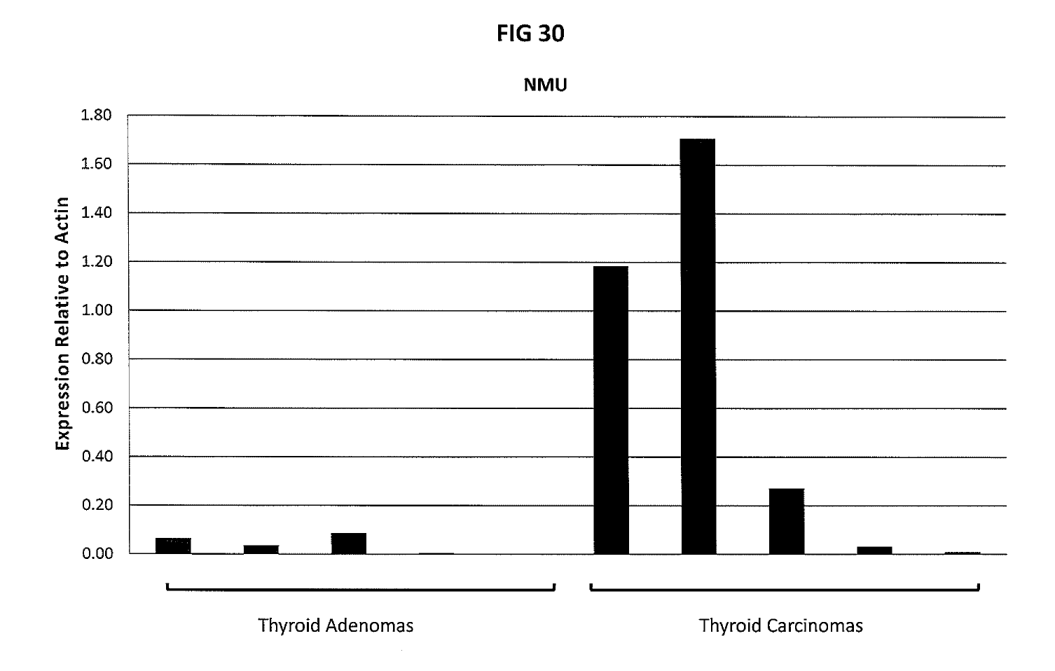

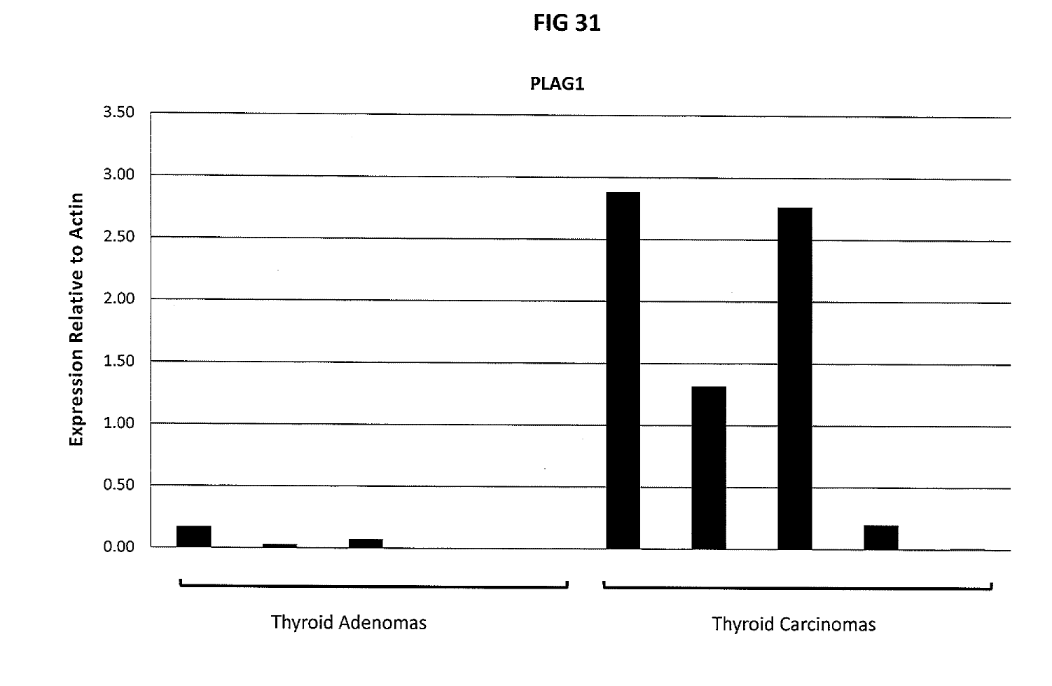

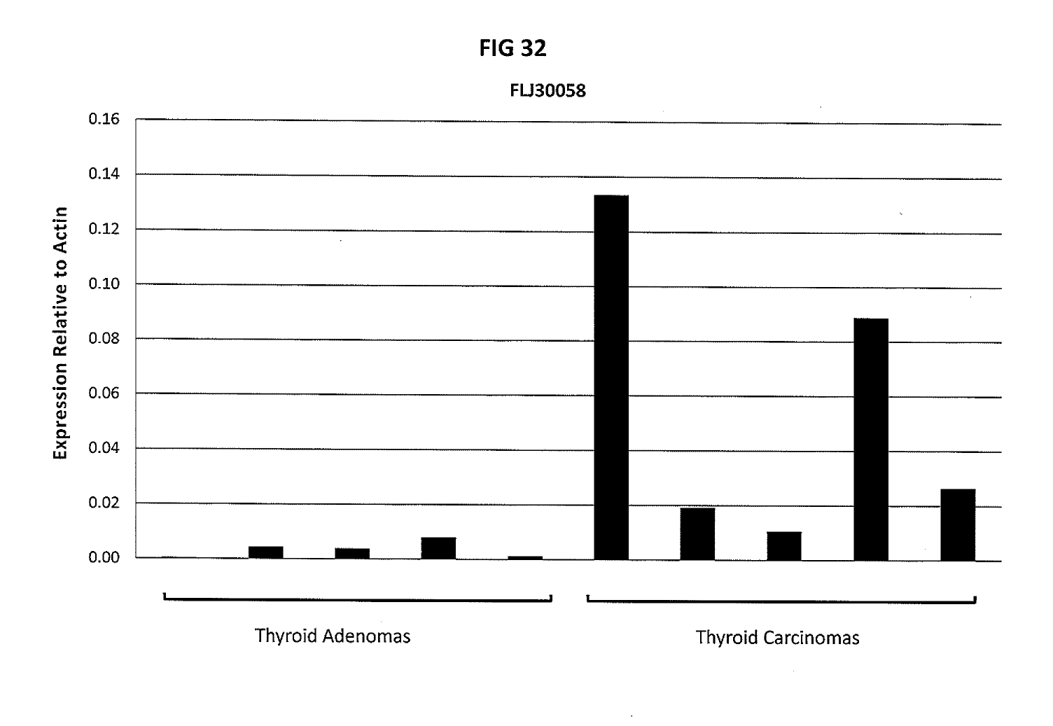

The results are shown in FIGS. 19-26 (the X axis shows results for normal tissue and stages 1-IV for thyroid cancer. The bars on the far right of the figure represent the breakdown of stage IV cancers into sub-types. The results indicate that IGSF1, CHI3L1, TM7SF4, ZCCHC12, SFTPB, NMU, PLAG1, FLJ30058 are all expressed at elevated levels in thyroid cancer compared to normal thyroid tissue. FIGS. 27-32 show a comparison of expression levels between benign thyroid tumors (thyroid adenomas) and malignant thyroid tumors (thyroid carcinomas). The results indicate that IGSF1, CHI3L1, ZCCHC12, NMU, PLAG1, FLJ30058, AND SLCO4C1 are all expressed at elevated levels in malignant thyroid tumors relative to benign thyroid tumors.

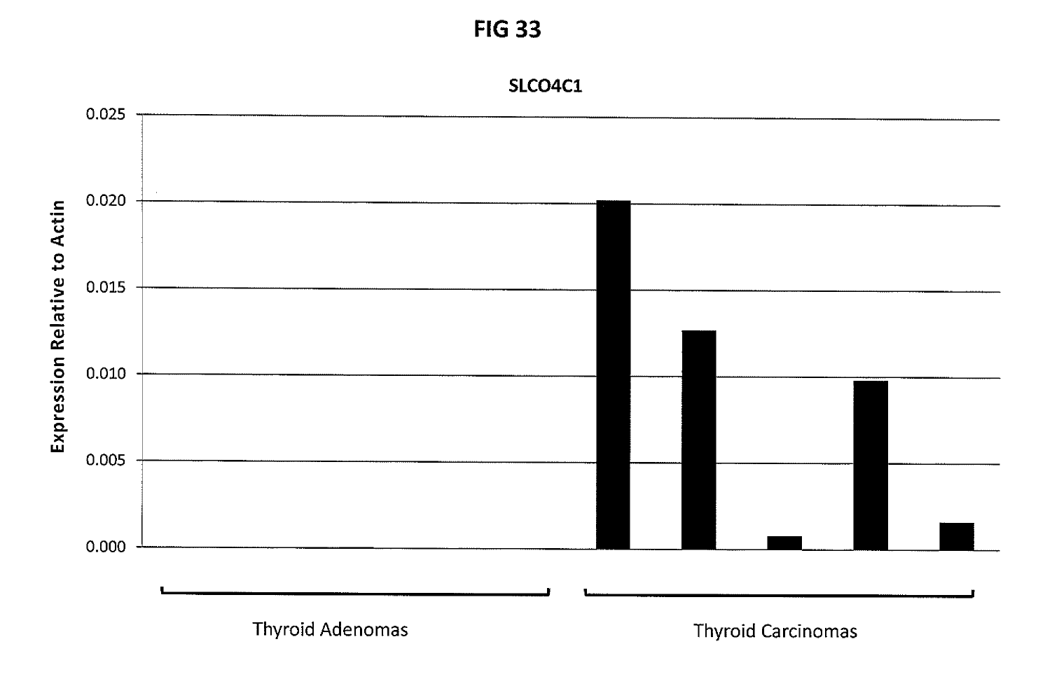

FIG. 33 shows a composite where 8 markers were analyzed using a binary cutoff to obtain 100% specificity using 8 markers. Using this binary cutoff sensitivity was 87% when the number of positive markers was 2 or greater.

### Example 20

### Immunofluorescence Microscopy

Paraffin embedded tissue sections were obtained from Asterand (Detroit, Mich.). These specimens included: Normal thyroid tissue (donors with no history of cancer), and thyroid follicular carcinoma. Prior to the staining with antibodies, the sections were dewaxed in xylene and rehydrated in cycles of ethanol (100%, 95%, 70%) followed by a wash in distilled water. Antigen retrieval was performed in epitope retrieval buffer (IHC World #IW-1100) by incubating the slides at 95 IC 40 minutes using an IHC-Steamer Set (IHC World #IW-1102). Immunostaining was performed using a polyclonal rabbit anti-human AHNAK2 antibody (Novus Biologicals #NBP1-88428) at a 1:200 dilution. The primary antibody was detected using an Alexa Fluor 594 Donkey anti-rabbit IgG (Life Sciences #A21207) at a 1:200 dilution.

Vectashield mounting medium with DAPI was used to preserve the stained samples (Vector Laboratories #H-1200). Images were taken with an exposure time of 400 milliseconds using a Nikon Eclipse TE2000-U at a magnification of 10,000 and an X-Cite 120 fluorescence illumination system (Lumen Dynamics).

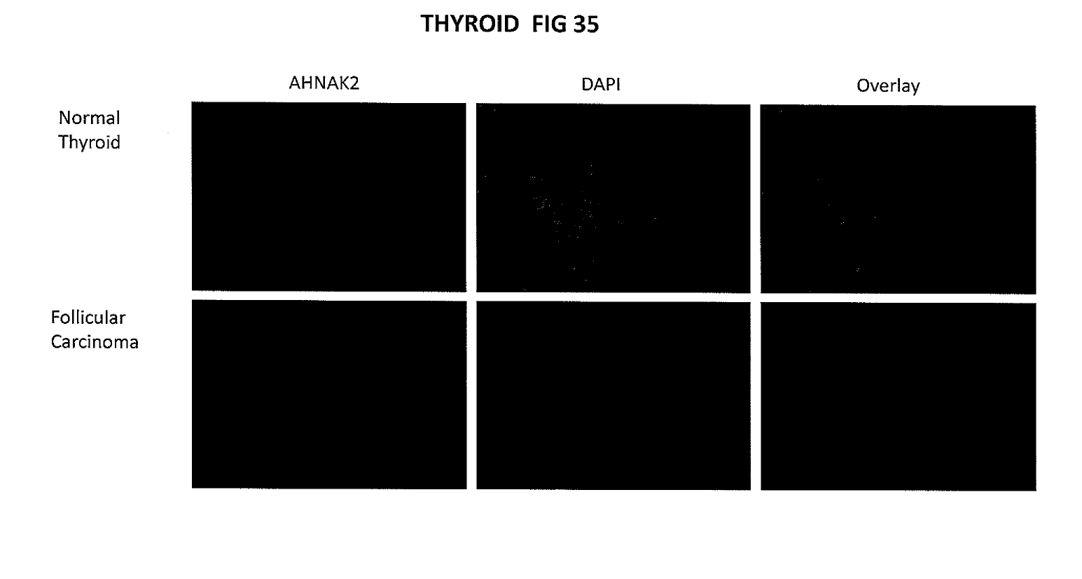

The results are shown in FIG. 35 and indicate that AHNAK2 (C14Oorf78) protein is expressed in thyroid carcinoma cells.

### Example 21

### Immunofluorescence Microscopy

Paraffin embedded tissue sections of thyroid follicular carcinoma were obtained from Asterand (Detroit, Mich.). Prior to the staining with antibodies, the sections were dewaxed in xylene and rehydrated in cycles of ethanol (100%, 95%, 70%) followed by a wash in distilled water. Antigen retrieval was performed in epitope retrieval buffer (LHC World #IW-1100) by incubating the slides at 95° C. 40 minutes using an IHC-Steamer Set (IHC World #IW-1102). Immunostaining was performed using a polyclonal rabbit anti-human Cytokeratine 19 antibody (Abcam #Ab15463) at a 1:100 dilution. The primary antibody was detected using an Alexa Fluor 594 Donkey anti-rabbit IgG (Life Sciences #A21207) at a 1:200 dilution.

Vectashield mounting medium with DAPI was used to preserve the stained samples (Vector Laboratories #H-1200). Images were taken with an exposure time of 400 milliseconds using a Nikon Eclipse TE2000-U at a magnification of 10,000 and an X-Cite 120 fluorescence illumination system (Lumen Dynamics).

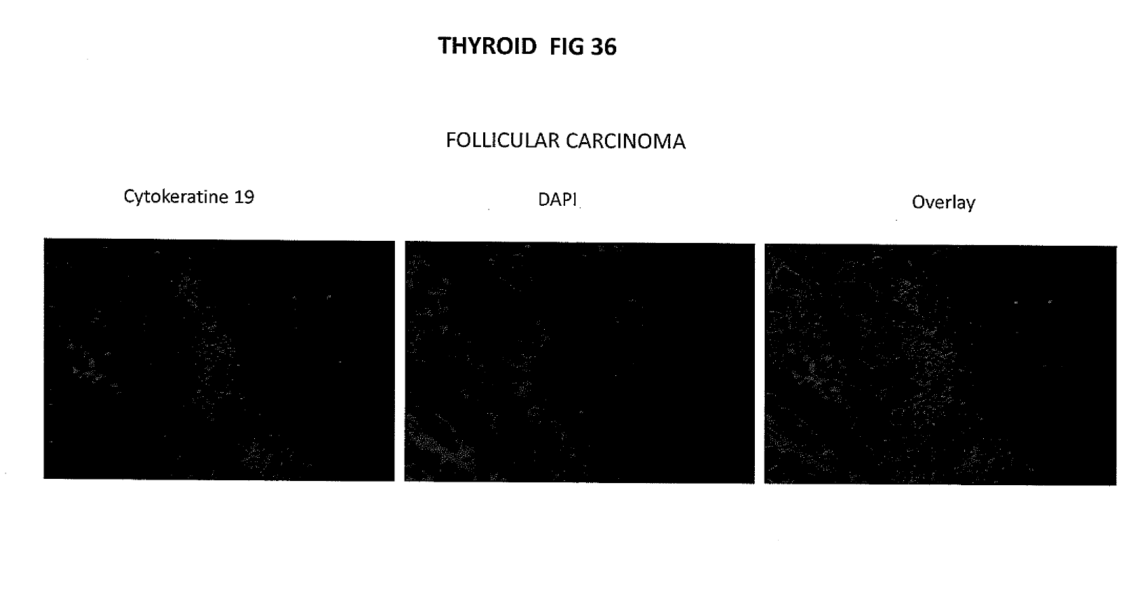

The results are shown in FIG. 36 and indicate that Cytokeratine 19 protein is expressed in thyroid carcinoma cells.

### Example 22

### Immunofluorescence Microscopy

Paraffin embedded tissue sections of thyroid follicular carcinoma were obtained from Asterand (Detroit, Mich.). Prior to the staining with antibodies, the sections were dewaxed in xylene and rehydrated in cycles of ethanol (100%, 95%, 70%) followed by a wash in distilled water. Antigen retrieval was performed in epitope retrieval buffer (IHC World #IW-1100) by incubating the slides at 95° C. 40 minutes using an IHC-Steamer Set (HC World #IW-1102). Immunostaining was performed using a polyclonal rabbit anti-human FLJ30058 antibody (Abcam #Ab127532) at a 1:100 dilution. The primary antibody was detected using an Alexa Fluor 594 Donkey anti-rabbit IgG (Life Sciences #A21207) at a 1:200 dilution.

Vectashield mounting medium with DAPI was used to preserve the stained samples (Vector Laboratories #H-1200). Images were taken with an exposure time of 400 milliseconds using a Nikon Eclipse TE2000-U at a magnification of 10,000 and an X-Cite 120 fluorescence illumination system (Lumen Dynamics).

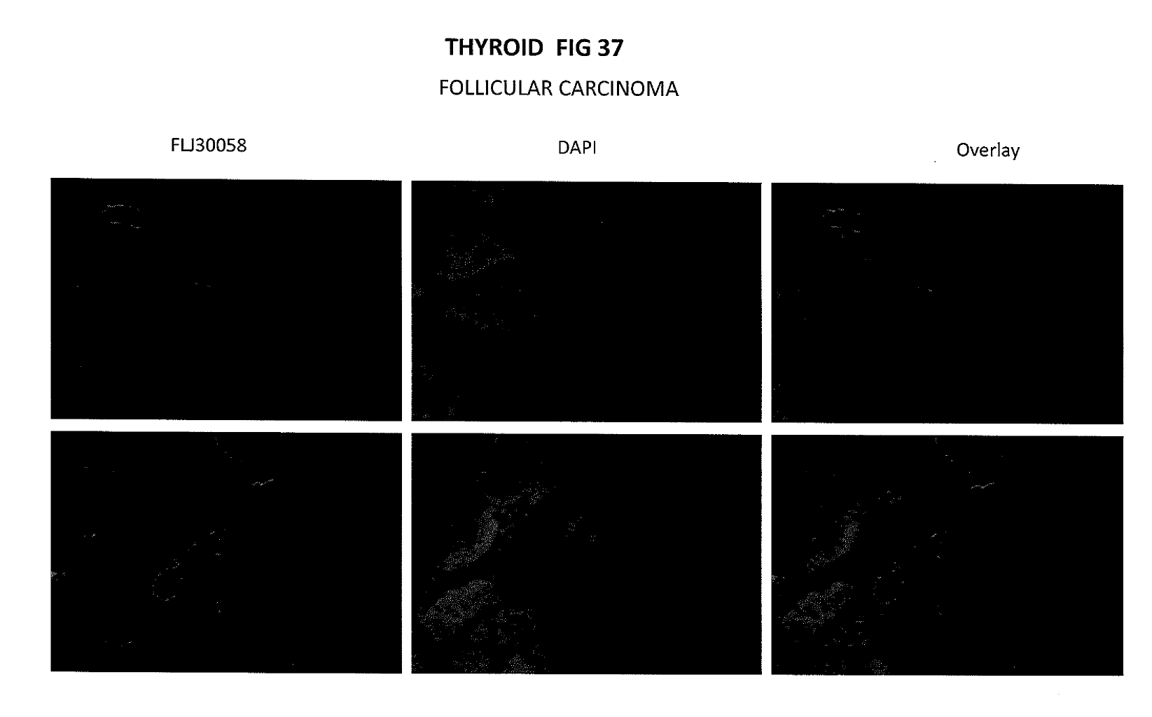

The results are shown in FIG. 37 and indicate that FLJ30058 protein is expressed in thyroid carcinoma cells.

### Example 23

FlexScript™ Ligation-Dependent Amplification (LDA) Assay (Luminex Corporation, Austin Tex.) was used according to the manufacturer's instructions. RNA was reverse-transcribed. Then, two probes per target were hybridized to adjacent regions on the complementary DNA (cDNA), and ligated with a thermostable ligase. Probe-probe pairs were PCR-amplified using universal primers binding to 5′ extensions of the probes (choosing a cycle number at which reactions were expected to be in the dynamic range, i.e. in the exponential amplification phase), and treated with lambda exonuclease to remove one of the strands. The remaining (biotinylated) strands were then hybridized to unique oligonucleotides attached to Luminex microspheres, incubated with streptavidin-phycoerythrin (PE), and quantified based on PE fluorescence. NMU, TNFRSF11B (FLJ30058), CRABP2, PLAG1, CCDC85A, C14orf18 (AHNAK2), and KIAA1324 expression levels were measured with LDA probes containing Illumina probe sequences as transcript-specific regions (25 bp of a 50 bp Illumina probe per LDA probe with two LDA probes covering the entire 50 bp Illumina probe). Data of both experiments (Example 23 and Example 24 below) was normalized to GAPDH mRNA expression.

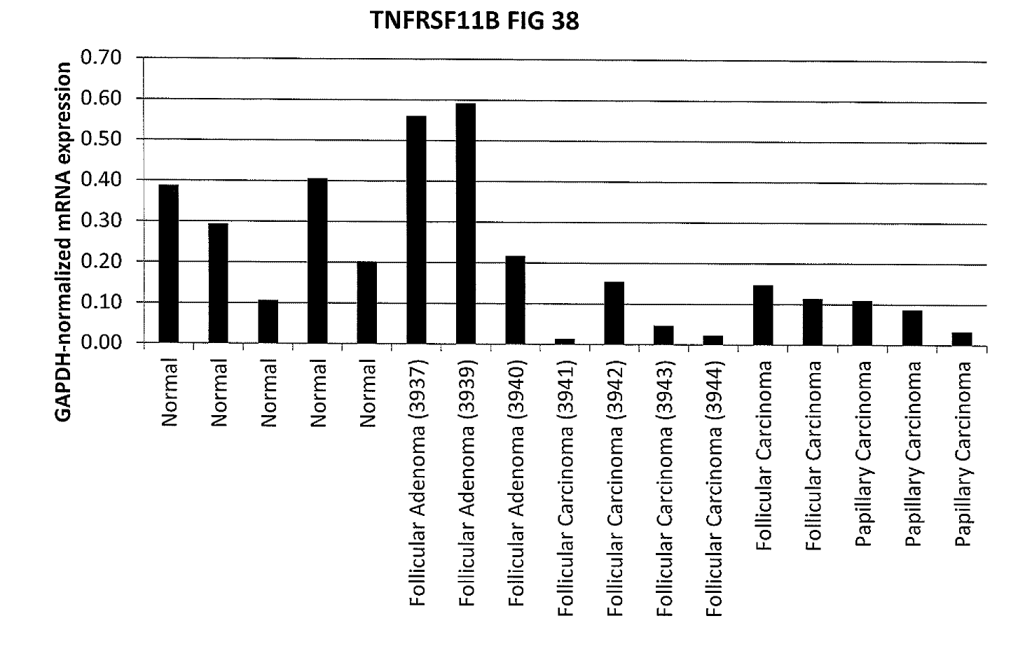

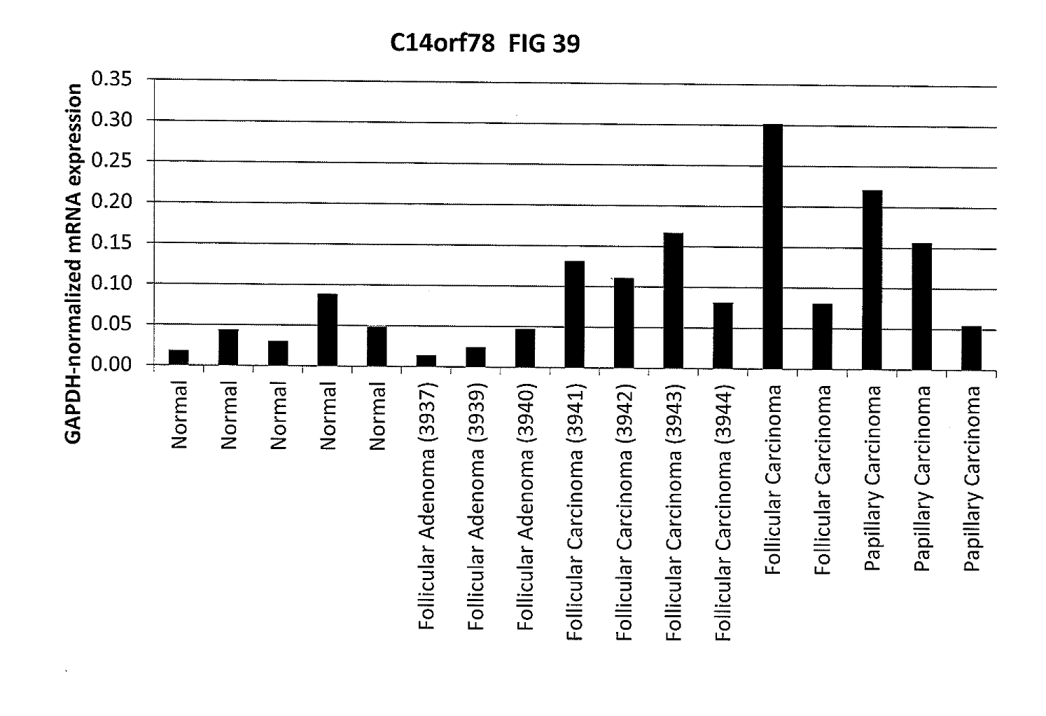

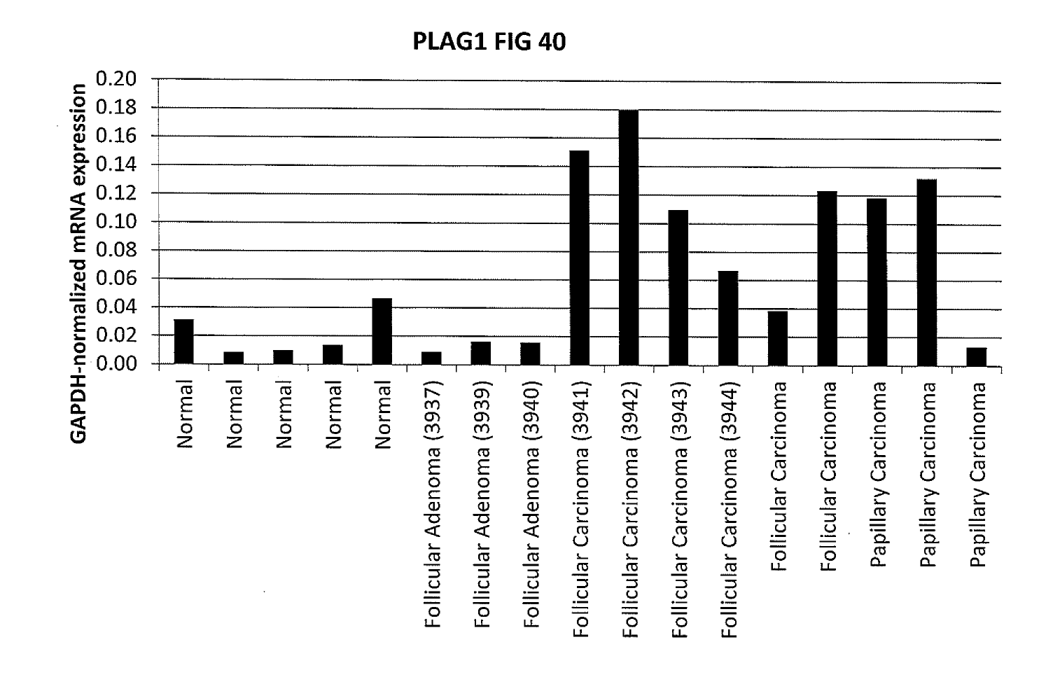

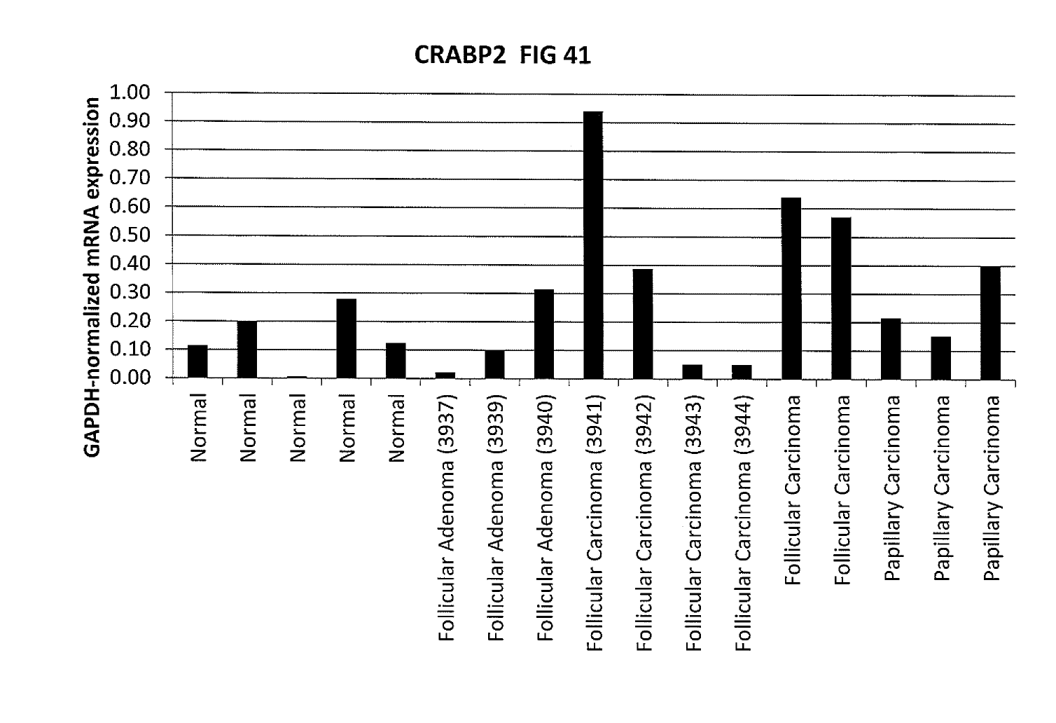

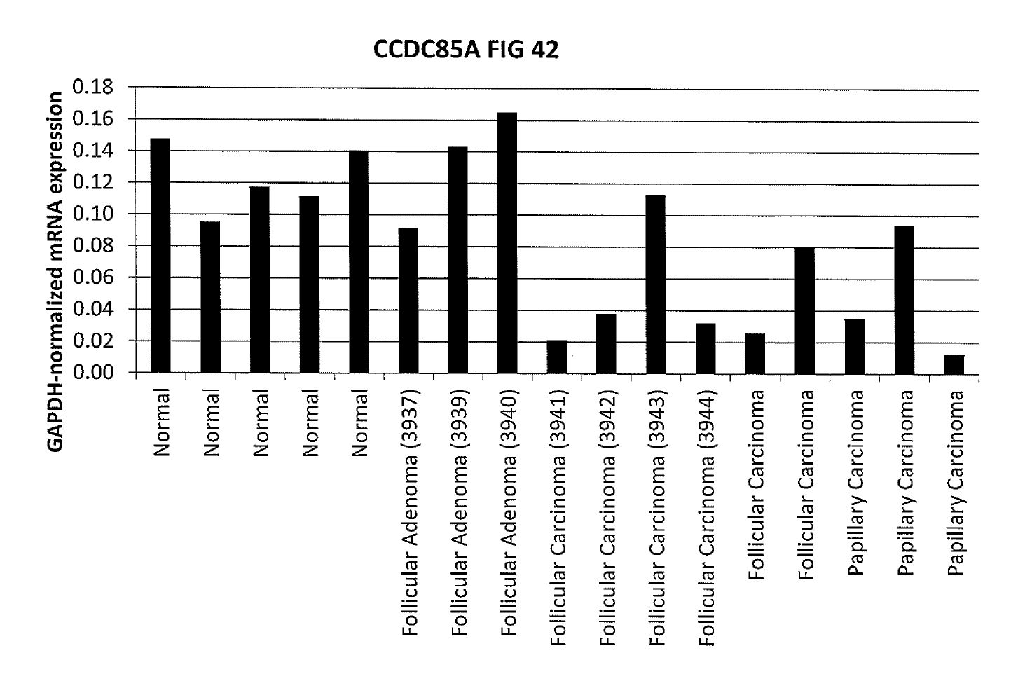

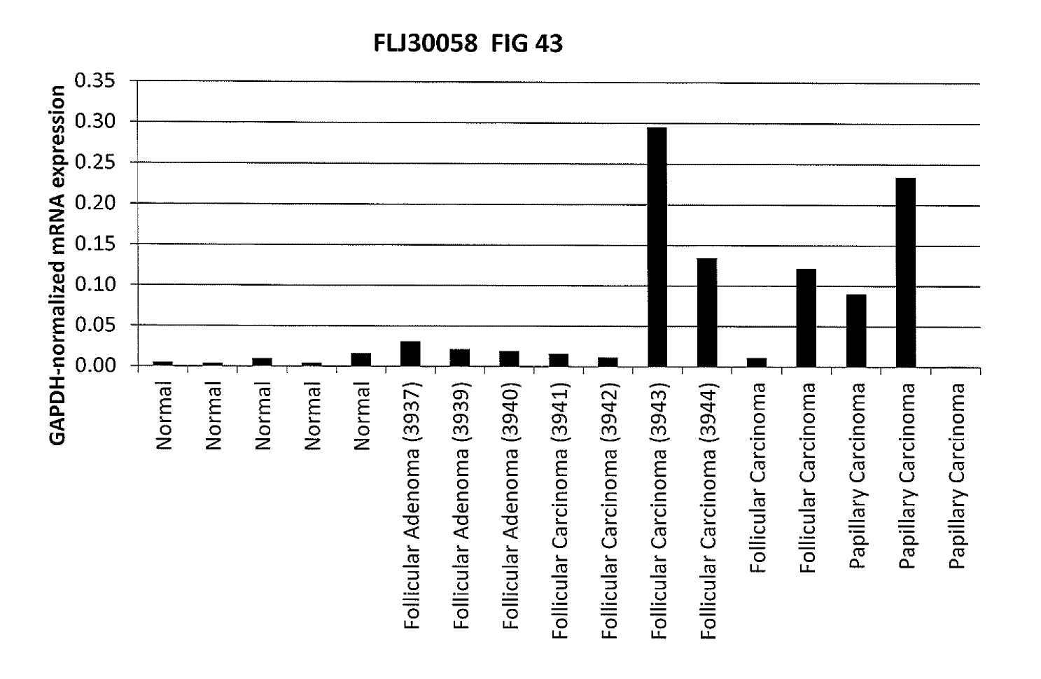

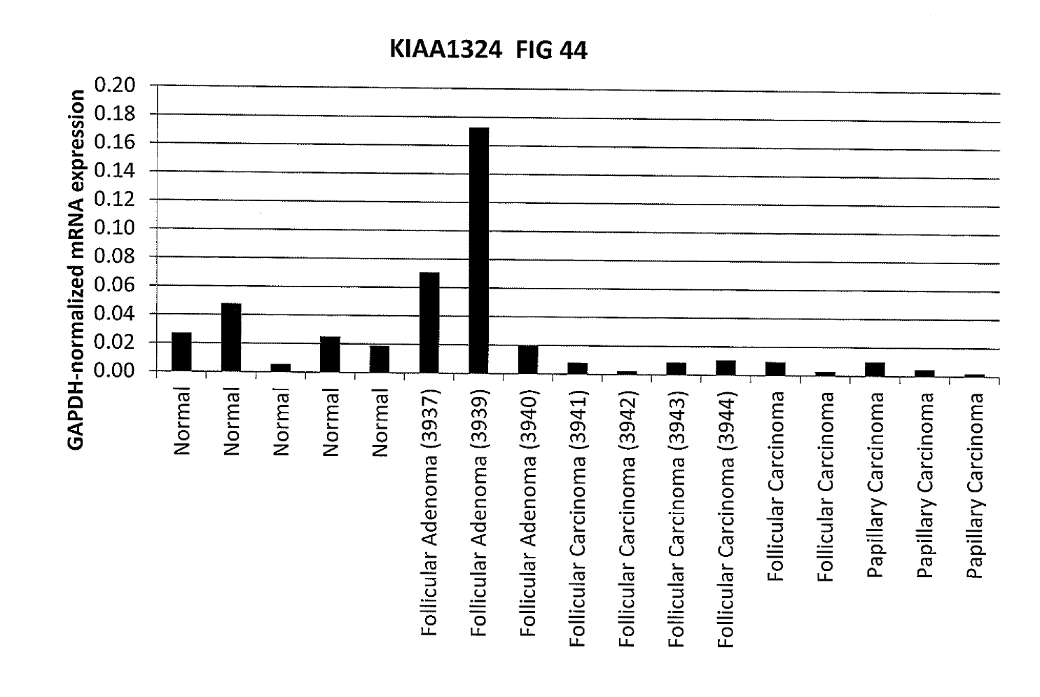

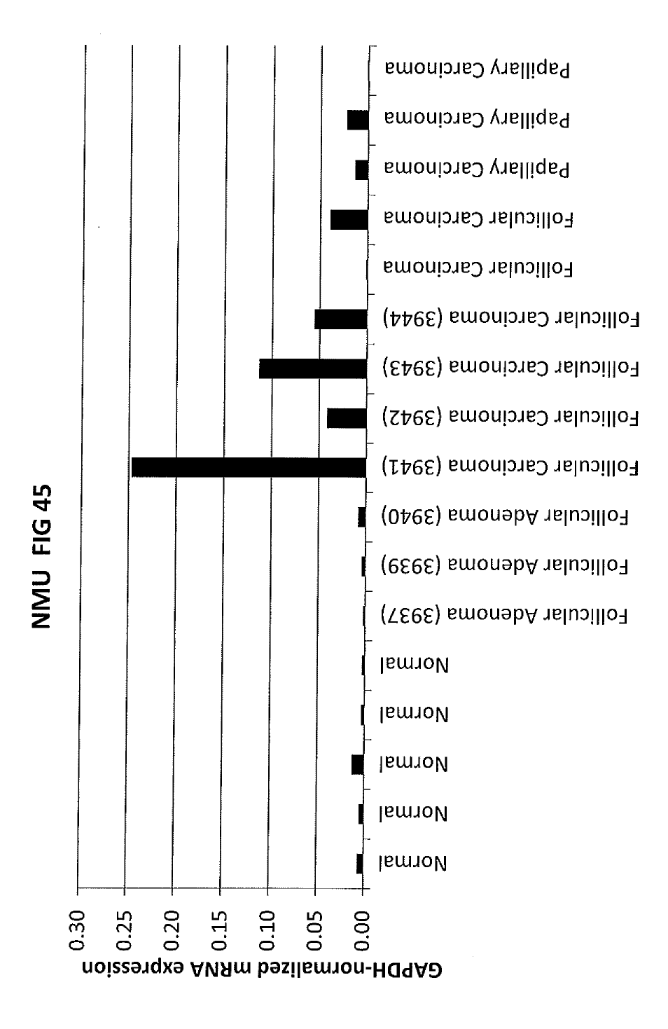

The results are shown in FIGS. 38-45 and Indicate that C14orf78, PLAG1, CRABP2, FLJ30058, NMU are all expressed at elevated levels in carcinomas relative to normal tissue. The results further indicate that TNSFRSF11B and KiAA1324 are both elevated in benign follicular adenomas compared to malignant follicular carcinomas.

### Example 24

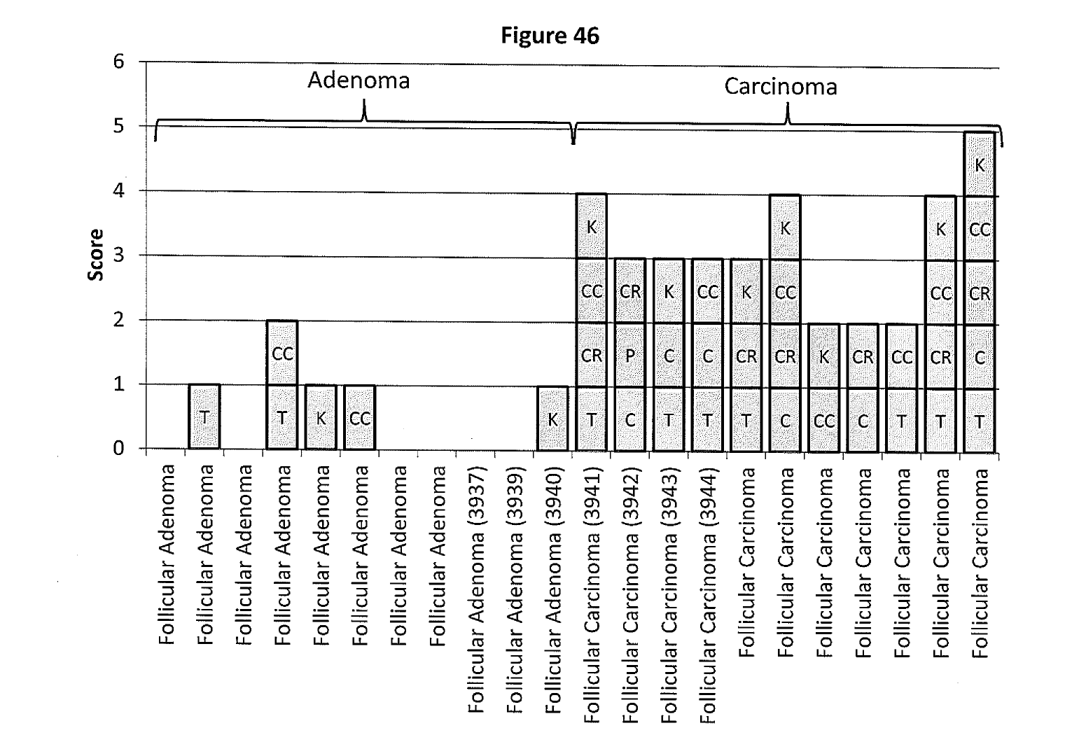

The data were analyzed to discriminate between follicular adenoma versus carcinoma. Expression levels of six markers in benign and malignant human thyroid samples as assessed by an LDA assay were analyzed. The markers analyzed along with their accession numbers and corresponding Illiumina Probe sequences are provided in the Table below. The analysis is presented in FIG. 46. Bars indicate overall scores composed of the scores for individual markers. Binary analysis was used. Expression levels passing a threshold were assigned a score of 1, otherwise of 0. The thresholds for markers predicted to be UP-regulated in carcinomas (C14orf78, PLAG1, CRABP2) are the highest expression levels measured among the adenoma samples. Thus, samples with expression levels higher than the highest value observed among all adenoma samples received positive scores. The thresholds for markers predicted to be DOWN-regulated in carcinomas (TNFRSF11B, CCDC85A, KIAA1324) were selected as the expression levels of the adenoma samples at the twentieth percentile ranked by expression level. Samples BELOW this threshold received a positive score. With a criterion as “minimal total score of 2”, the 6-marker assay has a sensitivity of 100% (11/11 carcinomas identified), and a specificity of 91% (10/11 adenomas identified). Letters within bars indicate samples contributing to the overall score (C=C14orf/8, P=PLAG1, CR=CRABP2, T=TNFRSF11B, CC=CCDC8SA, K=KIAA1324). Sample identification numbers in parentheses indicate samples assessed in both Example 23 and this example).

TABLE 6

| Gene Symbol | Accession No. (NCBI) |
|---|---|
| NMU | NM_006681.1 |
| ARHGAP36 (FLJ30058) | NM_144967.3 |
| TNFRSF11B | NM_002546.3 |
| CRABP2 | NM_001878.2 |
| PLAG1 | NM_002655.1 |
| CCDC85A | NM_001080433.1 |
| AHNAK2 (C14orf78) | NM_138420.2 |
| KIAA1324 | NM_020775.4 |

Illumina Probe Sequence
GCTGCAGCTCGTTCCTCACCTGCATGAGAGAAGAATGAAGAGATTCAGAG (SEQ ID NO: 88)
GTACAGTTTTGCTCAGGTCACGCCAACAGGGAAACCTCAAGTGTAGGTCT (SEQ ID NO: 89)
ATGGCGACCAAGACACCTTGAAGGGCCTAATGCACGCACTAAAGCACTCA (SEQ ID NO: 90)
CCAAGTCAGCAGTCCTAGCCCCAAACCAGCCCAGAGCAGGGTCTCTCTAA (SEQ ID NO: 91)
ACCCAGCTTTTGTCCACAAGGTGACTGTAACTCAGAATGGAAAGTGGGCT (SEQ ID NO: 92)
GCTTTTGTGCCTGCAGCAGAGCCTGCAGAAGCTAATACAAGGGACACTGG (SEQ ID NO: 93)
CACACTGTGGCCCCTGAGTCCCCTAATGTACACGCTGCAGCCAGAATGCA (SEQ ID NO: 94)
GCATAGCACCTTTGCAAGCCTGCGGCGATTTGGGTGCCAGCATCCTGCAA (SEQ ID NO: 95)

HOMO SAPIENS IMMUNOGLOBULIN SUPERFAMILY, MEMBER 1 (IGSF1),
TRANSCRIPT VARIANT 1, MRNA (NM_001555.2)
SEQ ID NO: 1

| | |
|---|---|
| 1 | gggcagtttg ctgcatctgg aggagctcac tggagaatct ccaacatcgg agcgggcctt |
| 61 | caactaccat cccaccacct gctgaggaga aaaattcttc aagactcaga gcacacagcc |
| 121 | agcaccagag gccccatgac cctggacaga ccaggggagg gggccaccat gctgaagaca |
| 181 | ttcactgttt tgctcttttg cattcggatg agtctgggta tgacatcgat agtgatggac |
| 241 | cctcaaccgg agttgtggat agagtccaac tacccccagg ccccttggga gaacatcacg |
| 301 | ctttggtgcc gaagcccctc tcggatatca agcaagttcc tgctgctgaa ggataagaca |
| 361 | cagatgacct ggatccgccc ttcccacaag accttccaag tttcattcct tataggtgcc |
| 421 | cttactgagt ccaatgcagg tctttaccgg tgctgctact ggaaggagac aggctggtca |
| 481 | aagcccagta aagttctaga gttggaggca ccaggccaac tgcccaagcc catcttctgg |
| 541 | attcaggctg agacccccgc tcttcctggg tgtaatgtta acatcctctg ccatggctgg |
| 601 | ctgcaggatt tggtattcat gctgtttaaa gagggatatg cagagcctgt ggattaccaa |
| 661 | gtcccaactg ggacaatggc catattctcc attgacaacc tgacacctga ggatgaaggg |
| 721 | gtttacatct gccgcactca tatccagatg ctccccaccc tgtggtcaga gcccagcaac |
| 781 | cccctgaagc tggttgtagc aggactctac cccaaaccaa ctttgacagc ccatcctggg |
| 841 | cccatcatgg cacctggaga aagcctgaat ctcaggtgcc aagggccaat ctatggaatg |
| 901 | acctttgctc taatgagggt tgaagacttg gagaagtcct tttaccacaa gaagacaata |
| 961 | aaaaatgagg caaatttctt cttccagtct ttgaagatcc aagatactgg acattacctc |
| 1021 | tgtttttact atgacgcatc atatagaggt tcactcctta gtgatgtcct gaaaatctgg |
| 1081 | gtaactgaca ctttccccaa gacctggcta cttgctcggc ccagtgctgt ggtccaaatg |
| 1141 | ggtcagaatg tgagcctacg gtgtcgagga ccagtggatg gagtgggtct tgcactctat |
| 1201 | aagaaaggag aagacaaacc acttcaattt ttggatgcca ccagcatcga tgacaacaca |
| 1261 | tcattcttcc tcaacaatgt aacctacagt gatactggca tctatagctg ccactatctt |
| 1321 | ctcacctgga agacctccat taggatgcca tcacacaaca ctgtggagct tatggttgta |
| 1381 | gataagcccc ccaaaccctc cctgtcagct tggccaagca ctgtgttcaa gctaggaaag |
| 1441 | gccatcaccc ttcagtgccg agtatctcat ccagtactgg aattttctct ggaatgggaa |
| 1501 | gaaagagaaa cattccaaaa attctcagta aacggagact tcatcatcag taatgttgac |
| 1561 | gggaaaggca cagggaccta cagttgcagc tatcgcgtag agacacatcc taacatctgg |
| 1621 | tcacatcgca gtgagcccct gaagctgatg gggccagcag gctatctcac ctggaattac |
| 1681 | gttctgaatg aagctatcag gttgtctcta atcatgcagc ttgttgcctt gctgttggta |
| 1741 | gtgctgtgga taaggtggaa gtgtcggaga ctcagaatca gagaagcctg gttgctggga |
| 1801 | acagctcaag gggtcaccat gctcttcata gtcacggccc ttctctgctg tggactgtgc |
| 1861 | aatggggtat tgatagaaga gactgaaata gtcatgccaa cccctaagcc tgagctgtgg |
| 1921 | gcagagacca actttcctct ggccccgtgg aagaacttaa ccctctggtg cagaagccct |
| 1981 | tctggctcaa ctaaggagtt tgtgttgctg aaggatggga ccgggtggat cgccactcgc |
| 2041 | ccggcctcag agcaggtccg ggctgccttc ccccttggcg ccctgaccca gagccacacc |
| 2101 | gggagctacc actgccattc atgggaggag atggctgtat cggagcccag tgaggcactt |
| 2161 | gagctggtgg ggacagacat cctccccaaa cctgtcattt ctgcttcccc cacaatccgg |
| 2221 | ggccaggaac tacaactccg gtgcaaagga tggctggcag gcatggggtt tgctctgtat |
| 2281 | aaggagggag agcaagaacc tgtccagcaa cttggtgctg ttggaagaga agccttcttt |
| 2341 | acaatccaga gaatggagga taaagacgaa ggcaattaca gctgccgcac tcacactgaa |
| 2401 | aaacgcccct tcaagtggtc tgagcccagt gagccgctgg agcttgtcat aaaagaaatg |
| 2461 | taccctaagc ccttcttcaa gacatgggcc agccctgtgg tcacccctgg tgcccgagtg |
| 2521 | actttcaatt gctccacccc ccaccagcat atgagcttta ttctttacaa agatggaagt |
| 2581 | gaaatagcat ccagtgacag gtcctgggca agtccggggg ccagtgcagc tcactttcta |
| 2641 | atcatttcgg tgggcattgg tgatggaggg aattacagct gccgatatta tgacttttct |
| 2701 | atctggtctg agcccagcga ccctgtggag ctcgtggtga cagaattcta ccccaaaccc |
| 2761 | actctcctgg cacagccagg tcctgtggtg tttcctggga agagtgtgat cctgcgctgc |
| 2821 | caagggactt tccagggcat gaggttcgcc ctcttgcagg agggagccca tgttccctta |
| 2881 | cagtttcgga gtgtctcagg gaactcagct gacttccttc tccacactgt tggagcagag |
| 2941 | gactctggga actatagctg tatctactat gagacaacca tgtcaaacag ggggtcatat |
| 3001 | ctcagtatgc cccttatgat ctgggtgact gacacattcc ctaagccatg gttgtttgct |
| 3061 | gagcccagtt ctgtggttcc catggggcag aatgttactc tctggtgccg agggccggtc |
| 3121 | catggagtag gatacattct gcacaaagaa ggagaagcca cttcaatgca gctctgggga |
| 3181 | tccaccagta atgacggggc attccccatc accaatatat ctggtactag catggggcgt |
| 3241 | tacagctgct gctaccaccc tgactggacc agttctatca agatacaacc tagcaacacc |
| 3301 | ctggaactcc tagtcacagg cttactcccc aaacccagcc tattagccca gcctggtccc |
| 3361 | atggtggccc ctggcgaaaa tatgactctt cagtgtcaag gggaactgcc agactcaaca |
| 3421 | tttgtcctgt tgaaggaggg ggctcaggag cctttagagc aacagaggcc aagtgggtac |
| 3481 | agggctgact tctggatgcc agcagtgaga ggtgaagact ctgggatcta tagctgtgtt |
| 3541 | tattatttgg actctactcc ctttgcagct tcaaatcaca gtgactccct ggagatctgg |
| 3601 | gtgactgata agccccctaa accctctctg tcagcctggc ccagcaccat gttcaagtta |
| 3661 | gggaaggaca tcacccttca gtgccgagga cccctgccag gtgttgaatt tgtcctagaa |
| 3721 | catgatggag aagaagcacc tcagcagttt tcagaggatg gagactttgt catcaacaac |
| 3781 | gtagaaggaa aaggcattgg aaactacagc tgcagctacc gcctccaggc ctaccctgat |
| 3841 | atctggtcag agcctagtga tcccctggag ctggtggggg cagcagggcc tgttgctcag |
| 3901 | gagtgcactg tagggaacat tgtccgaagt agcctaatcg tggtggttgt tgtagccttg |
| 3961 | ggggtagtgc tagccataga gtggaagaag tggcctcgac tgcgaaccag aggctcagag |
| 4021 | acagacggaa gagaccagac cattgccctt gaagagtgta accaagaagg agaaccaggc |
| 4081 | acccctgcca attctccttc atcaacctct cagagaatct ctgtggaact gcccgttcca |
| 4141 | atataataat ctcctccttt acaagagctt tcctctcctc tctcttgctc tcagagacct |
| 4201 | ataaatccaa ccagttaccc tgcaagtcag ccccatctgc tgttccttgg tctctaatca |
| 4261 | cctgagctgg gtaaagggga ttctgggagt tgagagctct gccagggtga gatgtttcct |
| 4321 | gaagagaggt tccccacccc tgtaactcct cactgtactg atttactggc gcatgaaatt |
| 4381 | ctattaaaaa tgcattcttc tgaataaaaa gagtattcac tatttaactt caaaaaaaaa |
| 4441 | aaaaaaaaaa aaaaaaaaaa aaaaaaaaaa aaaaaaaaaa aaaaaaaaaa aa |

HOMO SAPIENS IMMUNOGLOBIN SUPERFAMILY, MEMBER 21 (IGSF21), MRNA
(NM_032880.2)
SEQ ID NO: 2

| | |
|---|---|
| 1 | aggagggggg tgctcgcgcc gccgggagag gcgagcgcga ggcagagagc gcgattcggc |
| 61 | tccaaactcc ggcgctgcag ccgatcggac tctgggccgc ggtgggcacc gcgcgcagct |
| 121 | agggagccga gaaccgcggc gagccccgag gacgcccaga gcgcgagggt cgctgcgcct |
| 181 | cgcagagccg gagccgagtc gagccgggcg cccgggctgc ctggccgcgg cggcatgggg |
| 241 | gcgcccccgc ggctctccgc gctgcccgcc accgcctcgg ccagtggccg gaggcaggag |
| 301 | cgcgtctgag cccatggcga ggggacccgc cgccaccgcc tccacccccg ccgccccgcc |
| 361 | accgccgcca gctcccgggc accatgcgaa ccgccccgag cctccgccgc tgcgtctgcc |
| 421 | tgctgctcgc cgcgatcctg gacctggcgc gcggctacct gacagtcaac attgagcctc |
| 481 | tcccccctgt ggtggctgga gacgccgtga ctttgaagtg taacttcaag acagatgggc |
| 541 | gcatgcggga gatcgtgtgg taccgggtga cggatggtgg caccatcaag caaaagatct |
| 601 | tcaccttcga cgccatgttc tccaccaact actcacacat ggagaactac cgcaagcgag |
| 661 | aggacctggt gtaccagtcc actgtgaggc tgcccgaggt ccggatctca gacaatggtc |
| 721 | cctatgagtg ccatgtgggc atctacgacc gcgccaccag ggagaaggtg gtcctggcat |
| 781 | caggcaacat cttcctcaac gtcatggctc ctcccacctc cattgaagtg gtggctgctg |
| 841 | acacaccagc ccccttcagc cgctaccaag cccagaactt cacgctggtc tgcatcgtgt |
| 901 | ctggaggaaa accagcaccc atggtttatt tcaaacgaga tggggaacca atcgacgcag |
| 961 | tgcccctatc agagccacca gctgcgagct ccggccccct acaggacagc aggcccttcc |
| 1021 | gcagccttct gcaccgtgac ctggatgaca ccaagatgca gaagtcactg tccctcctgg |
| 1081 | acgccgagaa ccggggtggg cgaccctaca cggagcgccc ctcccgtggc ctgaccccag |
| 1141 | atcccaacat cctcctccag ccaaccacag agaacatacc agagacggtc gtgagccgtg |
| 1201 | agtttccccg ctgggtccac agcgccgagc ccacctactt cctgcgccac agccgcaccc |
| 1261 | cgagcagtga cggcactgtg gaagtacgtg ccctgctcac ctggaccctc aacccacaga |
| 1321 | tcgacaacga ggccctcttc agctgcgagg tcaagcaccc agctctgtcg atgcccatgc |
| 1381 | aggcagaggt cacgctggtt gcccccaaag gacccaaaat tgtgatgacg cccagcagag |
| 1441 | cccgggtagg ggacacagtg aggattctgg tccatgggtt tcagaacgga gtcttcccgg |
| 1501 | agcccatgtt cacgtggacg cgggttggga gccgcctcct ggacggcagc gctgagttcg |
| 1561 | acgggaagga gctggtgctg gagcgggttc ccgccgagct caatggctcc atgtatcgct |
| 1621 | gcaccgccca gaacccactg ggctccaccg acacgcacac ccggctcatc gtgtttgaaa |
| 1681 | acccaaatat cccaagagga acggaggact ctaatggttc cattggcccc actggtgccc |
| 1741 | ggctcacctt ggtgctcgcc ctgacagtga ttctggagct gacgtgaagg cacccgcccc |
| 1801 | ggccactcca tcaggcactg acatctccgc gaccggtttt catttctttt ctaaactatt |
| 1861 | tccagtcttg ttcttagtct ctttccatct gtgtcttggc ttcttcagtc ggtttaatta |
| 1921 | aaacaaacag aacaattttc ccc |

HOMO SAPIENS TRANSMEMBRANE 7 SUPERFAMILY MEMBER 4 (TM7SF4), MRNA.
(NM_030788.2)
SEQ ID NO: 3

| | |
|---|---|
| 1 | gcatttctgc attcgaagaa gaatctgaga gaaacctgac gcagggagca tgggtatctg |
| 61 | gacctcaggc actgatatct tcctaagtct ttgggagatt tacgtgtctc caagaagccc |
| 121 | cggatggatg gactttatcc agcatttggg agtttgctgt ttggttgctc ttatttcagt |
| 181 | gggcctcctg tctgtggccg cctgctggtt tctgccatca atcatagcgg ccgctgcctc |
| 241 | ctggattatc acgtgtgttc tgctgtgttg ctccaagcat gcacgatgtt ttattcttct |
| 301 | tgtctttctc tcttgtggcc tgcgtgaagg caggaatgct ttgattgcag ctggcacagg |
| 361 | gatcgtcatc ttgggacacg tagaaaatat ttttcacaac tttaaaggtc tcctagatgg |
| 421 | tatgacttgc aacctaaggg caaagagctt ttccatacat tttccacttt tgaaaaaata |
| 481 | tattgaggca attcagtgga tttatggcct tgccactcca ctaagtgtat ttgatgacct |
| 541 | tgtttcttgg aaccagaccc tggcagtctc tcttttcagt cccagccatg tcctggaggc |
| 601 | acagctaaat gacagcaaag gggaagtcct gagcgtcttg taccagatgg caacaaccac |
| 661 | agaggtgttg tcctccctgg gtcagaagct acttgccttt gcagggcttt cgctcgtcct |
| 721 | gcttggcact ggcctcttca tgaagcgatt tttgggccct tgtggttgga agtatgaaaa |
| 781 | catctacatc accagacaat ttgttcagtt tgatgaaagg gagagacatc aacagaggcc |
| 841 | ctgtgtgctc ccgctgaata aggaggaaag gaggaagtat gtcatcatcc cgactttctg |
| 901 | gccgactcct aaagaaagga aaaacctggg gctgtttttc ctccccatac ttatccatct |
| 961 | ctgcatctgg gtgctgtttg cagctgtaga ttatctgctg tatcggctca ttttctcagt |
| 1021 | gagcaagcag tttcaaagct tgccagggtt tgaggttcac ttgaaactgc acggagagaa |
| 1081 | acaaggaact caagatatta tccatgattc ttcctttaat atatctgtgt ttgaacccaa |
| 1141 | ctgtatccca aaaccaaaat tccttctatc tgagacctgg gttcctctca gtgttattct |
| 1201 | tttgatatta gtgatgctgg gactgttgtc ctctatcctt atgcaactta aaatcctggt |
| 1261 | gtcagcatct ttctacccca gcgtggagag gaagcgcatc caatatctgc atgcaaagct |
| 1321 | gcttaaaaaa agatcaaagc agccgctggg agaagtcaaa agacggctga gtctctatct |
| 1381 | tacaaagatt catttctggc ttccagtcct gaaaatgatt aggaagaagc aaatggacat |
| 1441 | ggcaagtgca gacaagtcat gagagacccc gactactcct cagccacatc gcaccaacaa |
| 1501 | ttctcttcag gtctaggatg gcagtcacta ttcatgccgg ataatagaga actatgtgac |
| 1561 | gcagtcctct caggagtctg agtttacaga gccaacttgc agcacctggt tatgcctcct |
| 1621 | ttcatctcaa agccaaagag ctgccaggta aatggttatg tggtctatgt tccaaacaaa |
| 1681 | ccacatgatc ttgcctgtgt cacaatgtaa caagactcta gctgggtccc ctggtgatga |
| 1741 | gtttcagcat agaataatgt tcaaggaaaa gaaaacgaaa acagtttaaa tctctaccac |
| 1801 | agcctcacaa gcaaatgcta aggggaacat acatgtaaaa agccagcaaa ctatcttcaa |
| 1861 | actcttccgt ccttaatgtc ttccatggct attgccccca caatggtctc ttttctccct |
| 1921 | gctcccttat taaagaactc tttctgaaac cc |

HOMO SAPIENS HYPOTHETICAL PROTEIN FLJ30058 (FLJ30058), MRNA (NM_144967.2)
SEQ ID NO: 4

| | |
|---|---|
| 1 | atgctcgagg tgtccggtga ccgaaacact cttagctggg tgcagggtgc ggctcagggc |
| 61 | gtggtcaccg ggggctactt agggccgccg gtgcggggga cgacgcaaag gttaactgcg |
| 121 | agctgccggg cactcagcgc gggtcatggc gtggatactg gactgccttt tcgcctcggc |
| 181 | ctttgagccc cgcccccgcc gtgcaaaaac aaccagaggc tgctctgctt gagggtgaag |
| 241 | ccgcctccca gttttccctc cccctctacc cccaccccca tagttctctc caccaggtcc |
| 301 | agtgacaatt ggatgatgca gccttgataa tcatccgatt ccagaatggg tggctgcatt |
| 361 | ccttttctga aggcagcaag ggcactgtgc cccagaatca tgcccccttt gctgttgttg |
| 421 | tccgccttca tttttttagt gagtgtcttg ggaggagccc caggacacaa ccccgaccgc |
| 481 | aggacgaaga tggtatcgat acacagcctc tctgagctgg agcgtctgaa gctgcaagag |
| 541 | actgcttacc acgaactcgt ggccagacat ttcctgtccg aattcaaacc tgacagagct |
| 601 | ctgcctattg accgtccgaa caccttggat aagtggtttc tgattttgag aggacagcag |
| 661 | agggctgtat cacacaagac atttggcatt agcctggaag aggtcctggt gaacgagttt |
| 721 | acccgccgca agcatcttga actgacagcc acgatgcagg ttgaagaagc caccggtcag |
| 781 | gctgcgggcc gtcgtcgggg aaacgtggtg cgaagggtgt ttggccgcat ccggcgcttt |
| 841 | ttcagtcgca ggcggaatga gcccaccttg ccccgggagt tcactcgccg tgggcgtcga |
| 901 | ggtgcagtgt ctgtggatag tctggctgag ctggaagacg gagccctgct gctgcagacc |
| 961 | ctgcagcttt caaaaatttc ctttccaatt ggccaacgac ttctgggatc caaaaggaag |
| 1021 | atgagtctca atccgattgc gaaacaaatc ccccaggttg ttgaggcttg ctgccaattc |
| 1081 | attgaaaaac atggcttaag cgcagtgggg atttttaccc ttgaatactc cgtgcagcga |
| 1141 | gtgcgtcagc tccgtgaaga atttgatcaa ggtctggatg tagtgctgga tgacaatcag |
| 1201 | aatgtgcatg atgtggctgc actcctcaag gagtttttcc gtgacatgaa ggattctctg |
| 1261 | ctgccagatg atctgtacat gtcattcctc ctgacagcaa ctttaaagcc ccaggatcag |
| 1321 | ctttctgccc tgcagttgct ggtctacctg atgccaccct gccacagtga taccctggag |
| 1381 | cgtctgctga aggccctgca taaaatcact gagaactgcg aggactcaat tggcattgat |
| 1441 | ggacagttgg tcccaggcaa ccgtatgact tccactaact tggccttggt gtttggatct |
| 1501 | gctctcctga aaaaaggaaa gtttggcaag agagagtcca ggaaaacaaa gctggggatt |
| 1561 | gatcactatg ttgcttctgt caatgtggtc cgtgccatga tagataactg ggatgtcctc |
| 1621 | ttccaggtgc ctccccatat tcagaggcag gttgctaagc gcgtgtggaa gtccagcccg |
| 1681 | gaagcacttg attttatcag acgcaggaac ttgaggaaga tccagagtgc acgcataaag |
| 1741 | atggaagagg atgcactact ttctgatcca gtggaaacct ctgctgaagc ccgggctgct |
| 1801 | gtccttgctc aaagcaagcc ttctgatgaa ggttcctctg aggagccagc tgtgccttcc |
| 1861 | ggcactgccc gttcccatga cgatgaggaa ggagcgggta accctcccat tccggagcaa |
| 1921 | gaccgcccat tgctccgtgt gccccgggag aaggaggcca aaactggcgt cagctacttc |
| 1981 | tttccttaga tgtttttcct tctataaggt gccagacagg ggaaaagggt gggggtacat |
| 2041 | ctgggatgtc acaggaaaca ttaaggagag agttgaaggt aaagatctga aggtaagaag |
| 2101 | gagttccacc tgatgctcgg gtcaggatga gaattccaaa cacactgcca gccccttcac |
| 2161 | tggggatgct tggtctcttc tgctggtaaa agcagagatg tttctgtgtc atgcccaagc |
| 2221 | tccccggtgc taccttgcct ttctctttta cccctgatct tggctttctc tctctctctg |
| 2281 | cagactttcc tttaattgat gtgacatttg tggtaaacac ctttcccagg gaacctcaca |
| 2341 | aatcttgaga tgctttccct tccccagatg ggattgcatg attccctgac tttcctaccc |
| 2401 | tcctccagag agctcagttg gaaaggccct caagaggcat gctagaacgt taggtcagcc |
| 2461 | tactgacagc tgacaaacaa ttaatgcgaa atcatgtcac accaacccat agccgtgtcc |
| 2521 | acgcagcaac tccaccacct taggatttcc ccctccaaat tattcagacc aatggcttgc |
| 2581 | caaatggcct ctcccaaaat tctgtacagt tttgctcagg tcacgccaac agggaaacct |
| 2641 | caagtgtagg tctaattagt gtttctggga tccaaagtta gaggaaaatt tagattttat |
| 2701 | tgcctggatc tgctttaaag acaattggtg tttacaccct cttgtcagca aaacagctag |
| 2761 | ttaggtaagg acatatagtt ccaagtaggt aaagtcactt gattacaaat gttcttaact |
| 2821 | atcgtctctg taattccttt atacaggaca gtacaaaatt gtgggacatg ctctggtaac |
| 2881 | acacagatat gggttgcata tgatccagaa ttacagctga tattatggat gacaactgct |
| 2941 | aaggtccata aaatgaagac tgtattgtat tgagggatag aaattgatca tttaatgggt |
| 3001 | aacaactgct gagctcaaag atttgtgatt gttaaaactt ctctggcatt taatcattaa |
| 3061 | taaacatctg tattgtgaca gc |

HOMO SAPIENS CBP/P300-INTERACTING TRANSACTIVATOR, WITH GLU/ASP-
RICH CARBOXY-TERMINAL DOMAIN, 1 (CITED1), MRNA (NM_004143.2)
SEQ ID NO: 5

| | |
|---|---|
| 1 | gtggaaattg aggggagaaa aaaaaaggga aaaaaagggt ctgtccttcc tgggattcct |
| 61 | agccgaggcc agtctgctgc cgtgtgcgtg tgcgtcaggg ctctccgggc ggcaatgggg |
| 121 | gcttgagagc cgggtcccca gcgccgggaa gggagcgcgg tggccgccac cgccaccgcc |
| 181 | ccggagtccg gcgccgaagc tgcgggcggg cgggcgggca ccagctcggt caggggctgc |
| 241 | ttggcgcggc actgtgcggt gcagcggcgg cgcggcgcgg tgcgggcttt tcccaggcgc |
| 301 | cccggggtcg ggtggccaac ggcgcggccg cgggcgctga gcgcgaccgg ttcgcggtag |
| 361 | cggtggcggc ggcgtgcgtg ccaggggctg ggggctccgc cgcctctctt gcggctcacc |
| 421 | gagctccgcg cttccctctc tccagggcag gcggcttctc agagcacaac agctccagct |
| 481 | ggcagcatca cttcccgcca atttatccaa cttctgccaa ggctctgaaa tgccaacaac |
| 541 | gtcgaggcct gcacttgatg tcaagggtgg cacctcacct gcgaaggagg atgccaacca |
| 601 | agagatgagc tccgtggcct actccaacct tgcggtgaaa gatcgcaaag cagtggccat |
| 661 | tctgcactac cctggggtag cctcaaatgg aaccaaggcc agtggggctc ccactagttc |
| 721 | ctcgggatct ccaataggct ctcctacaac cacccctccc actaaacccc catccttcaa |
| 781 | cctgcacccc gcccctcact tgctggctag tatgcagctg cagaaactta atagccagta |
| 841 | tcaggggatg gctgctgcca ctccaggcca acccggggag gcaggacccc tgcaaaactg |
| 901 | ggactttggg gcccaggcgg gaggggcaga atcactctct ccttctgctg gtgcccagag |
| 961 | ccctgctatc atcgattcgg acccagtgga tgaggaagtg ctgatgtcgc tggtggtgga |
| 1021 | actggggttg gaccgagcca atgagcttcc ggagctgtgg ctggggcaga atgagtttga |
| 1081 | cttcactgcg gactttccat ctagctgcta atgccaagtg tccctaaaga tggaggaata |
| 1141 | aagccaccaa ttctgttgta aataaaaata aagttactta caaaaaaaaa aaaaaaaaaa |
| 1201 | aaa |

HOMO SAPIENS ZINC FINGER, CCHC DOMAIN CONTAINING 12 (ZCCHC12),
MRNA (NM_173798.2)
SEQ ID NO: 6

| | |
|---|---|
| 1 | ggcgctgcct cgtctctgct acccctggtt gggcggccct gcgaagcagc tccttcgggc |
| 61 | agccccgggt cgcttagcgg ccaaggaggc ttcagttctt tgccgcctgc aaggcggaga |
| 121 | ccagaaggcg gaatccacag ctggcgacgc gggagcatct gctgtccacc agcggagcac |
| 181 | aggccatcaa agccgcatct gaacttgaat tctgtgcagc tgattgcaga gctggacccg |
| 241 | gatctgcgac cccctgtgga cagaggttga ccgtaccccg gagaggagct ttctcacgga |
| 301 | gggcactggt tgcagaggct ggaagtgaaa taaagacgcg ctcttgtttc agagttcgtc |
| 361 | ccctgctgag ataggaaggc agagccacct cctctcctct cccacctgca gattaagctt |
| 421 | ttctaaaaag cctaggcatc ttcttatatt cagataccct atcgtcgtca gtcatggcta |
| 481 | gcatcattgc acgtgtcggt aacagccggc ggctgaatgc acccttgccg ccttgggccc |
| 541 | attccatgct gaggtccctg gggagaagtc tcggtcctat aatggccagc atggcagaca |
| 601 | gaaacatgaa gttgttctcg gggagggtgg tgccagccca aggggaagaa acctttgaaa |
| 661 | actggctgac ccaagtcaat ggcgtcctgc cagattggaa tatgtctgag gaggaaaagc |
| 721 | tcaagcgctt gatgaaaacc cttaggggcc ctgcccgcga ggtcatgcgt gtgcttcagg |
| 781 | cgaccaaccc taacctaagt gtggcagatt tcttgcgagc catgaaattg gtgtttgggg |
| 841 | agtctgaaag cagtgtgact gcccatggta aattttttaa caccctacaa gctcaagggg |
| 901 | agaaagcctc cctttatgtg atccgtttag aggtgcagct ccagaacgct attcaggcag |
| 961 | gcattatagc tgagaaagat gcaaaccgga ctcgcttgca gcagctcctt ttaggcggtg |
| 1021 | agctgagtag ggacctccga ctcagactta aggattttct caggatgtat gcaaatgagc |
| 1081 | aggagcggct tcccaacttt ctggagttaa tcagaatggt aagggaggaa gaggattggg |
| 1141 | atgatgcttt tattaaacgg aagcgtccaa aaaggtctga gtcaatggtg gagagggcag |
| 1201 | tcagccctgt ggcatttcag ggctccccac cgatagtgat cggcagtgct gactgcaatg |
| 1261 | tgatagagat agatgatacc ctcgacgact ccgatgagga tgtgatcctg gtggagtctc |
| 1321 | aggaccctcc acttccatcc tggggtgccc ctcccctcag agacagggcc agacctcagg |
| 1381 | atgaagtgct ggtcattgat tccccccaca attccagggc tcagtttcct tccaccagtg |
| 1441 | gtggttctgg ctataagaat aacggtcctg gggagatgcg tagagccagg aagcgaaaac |
| 1501 | acacaatccg ctgttcgtat tgtggtgagg aaggccactc aaaagaaacc tgtgacaacg |
| 1561 | agagtgacaa ggcccaggtt tttgagaatt tgatcatcac tctccaggag ctgacccata |
| 1621 | ctgagatgga gaggtcaaga gtggcccctg gcgaatacaa tgacttctct gagccactgt |
| 1681 | aagggaccac ccccaggttt cagtgaaccc ttacctatat tcagcatcca gtagtgggaa |
| 1741 | aactggggtg ggggtggggg tgggacttct aactgcatga attaatccac aaagcggcta |
| 1801 | tcttttgggg tggagtagaa agggtcttgg ataccagcac attggaggga gatagcctga |
| 1861 | cctctgtcct tgctccttct ccctgcagcc tacgggtctg ttttctgtgt gtgcccattt |
| 1921 | ccttgacagc tttattcttt gtgaaagtgg tataatttat tgttaaatat ttgaacaata |
| 1981 | aaaaaggtac aaaaagtgaa gtacaaatta cccaaatctc tccaccctta tataatcatt |
| 2041 | gtcaaccctt tgatgagtga tatttcccta tacctatgta cccagataga tatatgcata |
| 2101 | gataaaagtg atgaaatata agtgctgttc tatctgtatt ttttcaccaa acaatatatg |
| 2161 | ttgtgagctt ctatgtcaat aaatatatat atcagcaaaa aaaaaaaaaa aa |

HOMO SAPIENS CLAUDIN 16 (CLDN16), MRNA (NM_006580.2)
SEQ ID NO: 7

| | |
|---|---|
| 1 | ccccacccga aacacactca gcccttgcac tgacctgcct tctgattgga ggctggttgc |
| 61 | ttcggataat gacctccagg accccactgt tggttacagc ctgtttgtat tattcttact |
| 121 | gcaactcaag acacctgcag cagggcgtga gaaaaagtaa aagaccagta ttttcacatt |
| 181 | gccaggtacc agaaacacag aagactgaca cccgccactt aagtggggcc agggctggtg |
| 241 | tctgcccatg ttgccatcct gatgggctgc ttgccacaat gagggatctt cttcaataca |
| 301 | tcgcttgctt ctttgccttt ttctctgctg ggtttttgat tgtggccacc tggactgact |
| 361 | gttggatggt gaatgctgat gactctctgg aggtgagcac aaaatgccga ggcctctggt |
| 421 | gggaatgcgt cacaaatgct tttgatggga ttcgcacctg tgatgagtac gattccatac |
| 481 | ttgcggagca tcccttgaag ctggtggtaa ctcgagcgtt gatgattact gcagatattc |
| 541 | tagctgggtt tggatttctc accctgctcc ttggtcttga ctgcgtgaaa ttcctccctg |
| 601 | atgagccgta cattaaagtc cgcatctgct ttgttgctgg agccacgtta ctaatagcag |
| 661 | gtaccccagg aatcattggc tctgtgtggt atgctgttga tgtgtatgtg gaacgttcta |
| 721 | ctttggtttt gcacaatata tttcttggta tccaatataa atttggttgg tcctgttggc |
| 781 | tcggaatggc tgggtctctg ggttgctttt tggctggagc tgttctcacc tgctgcttat |
| 841 | atctttttaa agatgttgga cctgagagaa actatcctta ttccttgagg aaagcctatt |
| 901 | cagccgcggg tgtttccatg gccaagtcat actcagcccc tcgcacagag acggccaaaa |
| 961 | tgtatgctgt agacacaagg gtgtaaaatg cacgtttcag ggtgtgtttg catatgattt |
| 1021 | aatcaatcag tatggttaca ttgataaaat agtaagtcaa tccaggaaca gttatttaga |
| 1081 | attcatattg aattaaatta attgctagct taatcaaaat gtttgattct cctatacttt |
| 1141 | ttctttctat tactcttata ttttcccgtc attctctctg ctaaccttcc accttatgca |
| 1201 | cacactttcc ctatatttta agataagtct gctaggatgt agaaatattt gtttgtgatt |
| 1261 | tctatatagc tattagagat tatgacatag taatattaaa atgaaatgat acttaaacag |
| 1321 | aaagcaattt ccaaagaggc cagggaccct aatctttgaa gagatgaaga aacttacttt |
| 1381 | tctccctggc ttttggttca ctttttgtac ttttaacaag tgggtgaatt atttgataat |
| 1441 | tttgaggaag attattcttt taaattcaaa ctagtatgtc aatgcctacc atta |

HOMO SAPIENS FIBRONECTIN 1 (FN1), TRANSCRIPT VARIANT 3, MRNA (NM_002026.2)
SEQ ID NO: 8

| | |
|---|---|
| 1 | gcccgcgccg gctgtgctgc acagggggag gagagggaac cccaggcgcg agcgggaaga |
| 61 | ggggacctgc agccacaact tctctggtcc tctgcatccc ttctgtccct ccacccgtcc |
| 121 | ccttccccac cctctggccc ccaccttctt ggaggcgaca acccccggga ggcattagaa |
| 181 | gggatttttc ccgcaggttg cgaagggaag caaacttggt ggcaacttgc ctcccggtgc |
| 241 | gggcgtctct cccccaccgt ctcaacatgc ttaggggtcc ggggcccggg ctgctgctgc |
| 301 | tggccgtcca gtgcctgggg acagcggtgc cctccacggg agcctcgaag agcaagaggc |
| 361 | aggctcagca aatggttcag ccccagtccc cggtggctgt cagtcaaagc aagcccggtt |
| 421 | gttatgacaa tggaaaacac tatcagataa atcaacagtg ggagcggacc tacctaggca |
| 481 | atgcgttggt ttgtacttgt tatggaggaa gccgaggttt taactgcgag agtaaacctg |
| 541 | aagctgaaga gacttgcttt gacaagtaca ctgggaacac ttaccgagtg ggtgacactt |
| 601 | atgagcgtcc taaagactcc atgatctggg actgtacctg catcggggct gggcgaggga |
| 661 | gaataagctg taccatcgca aaccgctgcc atgaaggggg tcagtcctac aagattggtg |
| 721 | acacctggag gagaccacat gagactggtg gttacatgtt agagtgtgtg tgtcttggta |
| 781 | atggaaaagg agaatggacc tgcaagccca tagctgagaa gtgttttgat catgctgctg |
| 841 | ggacttccta tgtggtcgga gaaacgtggg agaagcccta ccaaggctgg atgatggtag |
| 901 | attgtacttg cctgggagaa ggcagcggac gcatcacttg cacttctaga aatagatgca |
| 961 | acgatcagga cacaaggaca tcctatagaa ttggagacac ctggagcaag aaggataatc |
| 1021 | gaggaaacct gctccagtgc atctgcacag gcaacggccg aggagagtgg aagtgtgaga |
| 1081 | ggcacacctc tgtgcagacc acatcgagcg gatctggccc cttcaccgat gttcgtgcag |
| 1141 | ctgtttacca accgcagcct cacccccagc ctcctcccta tggccactgt gtcacagaca |
| 1201 | gtggtgtggt ctactctgtg gggatgcagt ggctgaagac acaaggaaat aagcaaatgc |
| 1261 | tttgcacgtg cctgggcaac ggagtcagct gccaagagac agctgtaacc cagacttacg |
| 1321 | gtggcaactc aaatggagag ccatgtgtct taccattcac ctacaatggc aggacgttct |
| 1381 | actcctgcac cacagaaggg cgacaggacg gacatctttg gtgcagcaca acttcgaatt |
| 1441 | atgagcagga ccagaaatac tctttctgca cagaccacac tgttttggtt cagactcgag |
| 1501 | gaggaaattc caatggtgcc ttgtgccact tccccttcct atacaacaac cacaattaca |
| 1561 | ctgattgcac ttctgagggc agaagagaca acatgaagtg gtgtgggacc acacagaact |
| 1621 | atgatgccga ccagaagttt gggttctgcc ccatggctgc ccacgaggaa atctgcacaa |
| 1681 | ccaatgaagg ggtcatgtac cgcattggag atcagtggga taagcagcat gacatgggtc |
| 1741 | acatgatgag gtgcacgtgt gttgggaatg gtcgtgggga atggacatgc attgcctact |
| 1801 | cgcagcttcg agatcagtgc attgttgatg acatcactta caatgtgaac gacacattcc |
| 1861 | acaagcgtca tgaagagggg cacatgctga actgtacatg cttcggtcag ggtcggggca |
| 1921 | ggtggaagtg tgatcccgtc gaccaatgcc aggattcaga gactgggacg ttttatcaaa |
| 1981 | ttggagattc atgggagaag tatgtgcatg gtgtcagata ccagtgctac tgctatggcc |
| 2041 | gtggcattgg ggagtggcat tgccaacctt tacagaccta tccaagctca agtggtcctg |
| 2101 | tcgaagtatt tatcactgag actccgagtc agcccaactc ccaccccatc cagtggaatg |
| 2161 | caccacagcc atctcacatt tccaagtaca ttctcaggtg gagacctaaa aattctgtag |
| 2221 | gccgttggaa ggaagctacc ataccaggcc acttaaactc ctacaccatc aaaggcctga |
| 2281 | agcctggtgt ggtatacgag ggccagctca tcagcatcca gcagtacggc caccaagaag |
| 2341 | tgactcgctt tgacttcacc accaccagca ccagcacacc tgtgaccagc aacaccgtga |
| 2401 | caggagagac gactcccttt tctcctcttg tggccacttc tgaatctgtg accgaaatca |
| 2461 | cagccagtag ctttgtggtc tcctgggtct cagcttccga caccgtgtcg ggattccggg |
| 2521 | tggaatatga gctgagtgag gagggagatg agccacagta cctggatctt ccaagcacag |
| 2581 | ccacttctgt gaacatccct gacctgcttc ctggccgaaa atacattgta aatgtctatc |
| 2641 | agatatctga ggatggggag cagagtttga tcctgtctac ttcacaaaca acagcgcctg |
| 2701 | atgcccctcc tgacccgact gtggaccaag ttgatgacac ctcaattgtt gttcgctgga |
| 2761 | gcagacccca ggctcccatc acagggtaca gaatagtcta ttcgccatca gtagaaggta |
| 2821 | gcagcacaga actcaacctt cctgaaactg caaactccgt caccctcagt gacttgcaac |
| 2881 | ctggtgttca gtataacatc actatctatg ctgtggaaga aaatcaagaa agtacacctg |
| 2941 | ttgtcattca acaagaaacc actggcaccc cacgctcaga tacagtgccc tctcccaggg |
| 3001 | acctgcagtt tgtggaagtg acagacgtga aggtcaccat catgtggaca ccgcctgaga |
| 3061 | gtgcagtgac cggctaccgt gtggatgtga tccccgtcaa cctgcctggc gagcacgggc |
| 3121 | agaggctgcc catcagcagg aacacctttg cagaagtcac cgggctgtcc cctggggtca |
| 3181 | cctattactt caaagtcttt gcagtgagcc atgggaggga gagcaagcct ctgactgctc |
| 3241 | aacagacaac caaactggat gctcccacta acctccagtt tgtcaatgaa actgattcta |
| 3301 | ctgtcctggt gagatggact ccacctcggg cccagataac aggataccga ctgaccgtgg |
| 3361 | gccttacccg aagaggacag cccaggcagt acaatgtggg tccctctgtc tccaagtacc |
| 3421 | cactgaggaa tctgcagcct gcatctgagt acaccgtatc cctcgtggcc ataaagggca |
| 3481 | accaagagag ccccaaagcc actggagtct ttaccacact gcagcctggg agctctattc |
| 3541 | caccttacaa caccgaggtg actgagacca ccattgtgat cacatggacg cctgctccaa |
| 3601 | gaattggttt taagctgggt gtacgaccaa gccagggagg agaggcacca cgagaagtga |
| 3661 | cttcagactc aggaagcatc gttgtgtccg gcttgactcc aggagtagaa tacgtctaca |
| 3721 | ccatccaagt cctgagagat ggacaggaaa gagatgcgcc aattgtaaac aaagtggtga |
| 3781 | caccattgtc tccaccaaca aacttgcatc tggaggcaaa ccctgacact ggagtgctca |
| 3841 | cagtctcctg ggagaggagc accaccccag acattactgg ttatagaatt accacaaccc |
| 3901 | ctacaaacgg ccagcaggga aattctttgg aagaagtggt ccatgctgat cagagctcct |
| 3961 | gcacttttga taacctgagt cccggcctgg agtacaatgt cagtgtttac actgtcaagg |
| 4021 | atgacaagga aagtgtccct atctctgata ccatcatccc agctgttcct cctcccactg |
| 4081 | acctgcgatt caccaacatt ggtccagaca ccatgcgtgt cacctgggct ccacccccat |
| 4141 | ccattgattt aaccaacttc ctggtgcgtt actcacctgt gaaaaatgag gaagatgttg |
| 4201 | cagagttgtc aatttctcct tcagacaatg cagtggtctt aacaaatctc ctgcctggta |
| 4261 | cagaatatgt agtgagtgtc tccagtgtct acgaacaaca tgagagcaca cctcttagag |
| 4321 | gaagacagaa aacaggtctt gattccccaa ctggcattga cttttctgat attactgcca |
| 4381 | actcttttac tgtgcactgg attgctcctc gagccaccat cactggctac aggatccgcc |
| 4441 | atcatcccga gcacttcagt gggagacctc gagaagatcg ggtgccccac tctcggaatt |
| 4501 | ccatcaccct caccaacctc actccaggca cagagtatgt ggtcagcatc gttgctctta |
| 4561 | atggcagaga ggaaagtccc ttattgattg gccaacaatc aacagtttct gatgttccga |
| 4621 | gggacctgga agttgttgct gcgaccccca ccagcctact gatcagctgg gatgctcctg |
| 4681 | ctgtcacagt gagatattac aggatcactt acggagagac aggaggaaat agccctgtcc |
| 4741 | aggagttcac tgtgcctggg agcaagtcta cagctaccat cagcggcctt aaacctggag |
| 4801 | ttgattatac catcactgtg tatgctgtca ctggccgtgg agacagcccc gcaagcagca |
| 4861 | agccaatttc cattaattac cgaacagaaa ttgacaaacc atcccagatg caagtgaccg |
| 4921 | atgttcagga caacagcatt agtgtcaagt ggctgccttc aagttcccct gttactggtt |
| 4981 | acagagtaac caccactccc aaaaatggac caggaccaac aaaaactaaa actgcaggtc |
| 5041 | cagatcaaac agaaatgact attgaaggct tgcagcccac agtggagtat gtggttagtg |
| 5101 | tctatgctca gaatccaagc ggagagagtc agcctctggt tcagactgca gtaaccaaca |
| 5161 | ttgatcgccc taaaggactg gcattcactg atgtggatgt cgattccatc aaaattgctt |
| 5221 | gggaaagccc acaggggcaa gtttccaggt acagggtgac ctactcgagc cctgaggatg |
| 5281 | gaatccatga gctattccct gcacctgatg gtgaagaaga cactgcagag ctgcaaggcc |
| 5341 | tcagaccggg ttctgagtac acagtcagtg tggttgcctt gcacgatgat atggagagcc |
| 5401 | agcccctgat tggaacccag tccacagcta ttcctgcacc aactgacctg aagttcactc |
| 5461 | aggtcacacc cacaagcctg agcgcccagt ggacaccacc caatgttcag ctcactggat |
| 5521 | atcgagtgcg ggtgaccccc aaggagaaga ccggaccaat gaaagaaatc aaccttgctc |
| 5581 | ctgacagctc atccgtggtt gtatcaggac ttatggtggc caccaaatat gaagtgagtg |
| 5641 | tctatgctct taaggacact ttgacaagca gaccagctca gggagttgtc accactctgg |
| 5701 | agaatgtcag cccaccaaga agggctcgtg tgacagatgc tactgagacc accatcacca |
| 5761 | ttagctggag aaccaagact gagacgatca ctggcttcca agttgatgcc gttccagcca |
| 5821 | atggccagac tccaatccag agaaccatca agccagatgt cagaagctac accatcacag |
| 5881 | gtttacaacc aggcactgac tacaagatct acctgtacac cttgaatgac aatgctcgga |
| 5941 | gctcccctgt ggtcatcgac gcctccactg ccattgatgc accatccaac ctgcgtttcc |
| 6001 | tggccaccac acccaattcc ttgctggtat catggcagcc gccacgtgcc aggattaccg |
| 6061 | gctacatcat caagtatgag aagcctgggt ctcctcccag agaagtggtc cctcggcccc |
| 6121 | gccctggtgt cacagaggct actattactg gcctggaacc gggaaccgaa tatacaattt |
| 6181 | atgtcattgc cctgaagaat aatcagaaga gcgagcccct gattggaagg aaaaagacag |
| 6241 | acgagcttcc ccaactggta acccttccac accccaatct tcatggacca gagatcttgg |
| 6301 | atgttccttc cacagttcaa aagacccctt tcgtcaccca ccctgggtat gacactggaa |
| 6361 | atggtattca gcttcctggc acttctggtc agcaacccag tgttgggcaa caaatgatct |
| 6421 | ttgaggaaca tggttttagg cggaccacac cgcccacaac ggccaccccc ataaggcata |
| 6481 | ggccaagacc atacccgccg aatgtaggac aagaagctct ctctcagaca accatctcat |
| 6541 | gggccccatt ccaggacact tctgagtaca tcatttcatg tcatcctgtt ggcactgatg |
| 6601 | aagaaccctt acagttcagg gttcctggaa cttctaccag tgccactctg acaggcctca |
| 6661 | ccagaggtgc cacctacaac atcatagtgg aggcactgaa agaccagcag aggcataagg |
| 6721 | ttcgggaaga ggttgttacc gtgggcaact ctgtcaacga aggcttgaac caacctacgg |
| 6781 | atgactcgtg ctttgacccc tacacagttt cccattatgc cgttggagat gagtgggaac |
| 6841 | gaatgtctga atcaggcttt aaactgttgt gccagtgctt aggctttgga agtggtcatt |
| 6901 | tcagatgtga ttcatctaga tggtgccatg acaatggtgt gaactacaag attggagaga |
| 6961 | agtgggaccg tcagggagaa aatggccaga tgatgagctg cacatgtctt gggaacggaa |
| 7021 | aaggagaatt caagtgtgac cctcatgagg caacgtgtta tgatgatggg aagacatacc |
| 7081 | acgtaggaga acagtggcag aaggaatatc tcggtgccat ttgctcctgc acatgctttg |
| 7141 | gaggccagcg gggctggcgc tgtgacaact gccgcagacc tgggggtgaa cccagtcccg |
| 7201 | aaggcactac tggccagtcc tacaaccagt attctcagag ataccatcag agaacaaaca |
| 7261 | ctaatgttaa ttgcccaatt gagtgcttca tgcctttaga tgtacaggct gacagagaag |
| 7321 | attcccgaga gtaaatcatc tttccaatcc agaggaacaa gcatgtctct ctgccaagat |
| 7381 | ccatctaaac tggagtgatg ttagcagacc cagcttagag ttcttctttc tttcttaagc |
| 7441 | cctttgctct ggaggaagtt ctccagcttc agctcaactc acagcttctc caagcatcac |
| 7501 | cctgggagtt tcctgagggt tttctcataa atgagggctg cacattgcct gttctgcttc |
| 7561 | gaagtattca ataccgctca gtattttaaa tgaagtgatt ctaagatttg gtttgggatc |
| 7621 | aataggaaag catatgcagc caaccaagat gcaaatgttt tgaaatgata tgaccaaaat |
| 7681 | tttaagtagg aaagtcaccc aaacacttct gctttcactt aagtgtctgg cccgcaatac |
| 7741 | tgtaggaaca agcatgatct tgttactgtg atattttaaa tatccacagt actcactttt |
| 7801 | tccaaatgat cctagtaatt gcctagaaat atctttctct tacctgttat ttatcaattt |
| 7861 | ttcccagtat ttttatacgg aaaaaattgt attgaaaaca cttagtatgc agttgataag |
| 7921 | aggaatttgg tataattatg gtgggtgatt attttttata ctgtatgtgc caaagcttta |
| 7981 | ctactgtgga aagacaactg ttttaataaa agatttacat tccacaactt gaagttcatc |
| 8041 | tatttgatat aagacacctt cgggggaaat aattcctgtg aatattcttt ttcaattcag |
| 8101 | caaacatttg aaaatctatg atgtgcaagt ctaattgttg atttcagtac aagattttct |
| 8161 | aaatcagttg ctacaaaaac tgattggttt ttgtcacttc atctcttcac taatggagat |
| 8221 | agctttacac tttctgcttt aatagattta agtggacccc aatatttatt aaaattgcta |
| 8281 | gtttaccgtt cagaagtata atagaaataa tctttagttg ctcttttcta accattgtaa |
| 8341 | ttcttccctt cttccctcca cctttccttc attgaataaa cctctgttca aagagattgc |
| 8401 | ctgcaaggga aataaaaatg actaagatat taaaaaaaaa aaaaaaaaa |

HOMO SAPIENS SERPIN PEPTIDASE INHIBITOR, CLADE A (ALPHA-1
ANTIPROTEINASE, ANTITRYPSIN), MEMBER 1 (SERPINA1), TRANSCRIPT
VARIANT 1, MRNA. (NM_000295.3)
SEQ ID NO: 9

| | |
|---|---|
| 1 | aatgactcct ttcggtaagt gcagtggaag ctgtacactg cccaggcaaa gcgtccgggc |
| 61 | agcgtaggcg ggcgactcag atcccagcca gtggacttag cccctgtttg ctcctccgat |
| 121 | aactggggtg accttggtta atattcacca gcagcctccc ccgttgcccc tctggatcca |
| 181 | ctgcttaaat acggacgagg acagggccct gtctcctcag cttcaggcac caccactgac |
| 241 | ctgggacagt gaatcgacaa tgccgtcttc tgtctcgtgg ggcatcctcc tgctggcagg |
| 301 | cctgtgctgc ctggtccctg tctccctggc tgaggatccc cagggagatg ctgcccagaa |
| 361 | gacagataca tcccaccatg atcaggatca cccaaccttc aacaagatca cccccaacct |
| 421 | ggctgagttc gccttcagcc tataccgcca gctggcacac cagtccaaca gcaccaatat |
| 481 | cttcttctcc ccagtgagca tcgctacagc ctttgcaatg ctctccctgg ggaccaaggc |
| 541 | tgacactcac gatgaaatcc tggagggcct gaatttcaac ctcacggaga ttccggaggc |
| 601 | tcagatccat gaaggcttcc aggaactcct ccgtaccctc aaccagccag acagccagct |
| 661 | ccagctgacc accggcaatg gcctgttcct cagcgagggc ctgaagctag tggataagtt |
| 721 | tttggaggat gttaaaaagt tgtaccactc agaagccttc actgtcaact tcggggacac |
| 781 | cgaagaggcc aagaaacaga tcaacgatta cgtggagaag ggtactcaag ggaaaattgt |
| 841 | ggatttggtc aaggagcttg acagagacac agtttttgct ctggtgaatt acatcttctt |
| 901 | taaaggcaaa tgggagagac cctttgaagt caaggacacc gaggaagagg acttccacgt |
| 961 | ggaccaggtg accaccgtga aggtgcctat gatgaagcgt ttaggcatgt ttaacatcca |
| 1021 | gcactgtaag aagctgtcca gctgggtgct gctgatgaaa tacctgggca atgccaccgc |
| 1081 | catcttcttc ctgcctgatg aggggaaact acagcacctg gaaaatgaac tcacccacga |
| 1141 | tatcatcacc aagttcctgg aaaatgaaga cagaaggtct gccagcttac atttacccaa |
| 1201 | actgtccatt actggaacct atgatctgaa gagcgtcctg ggtcaactgg gcatcactaa |
| 1261 | ggtcttcagc aatggggctg acctctccgg ggtcacagag gaggcacccc tgaagctctc |
| 1321 | caaggccgtg cataaggctg tgctgaccat cgacgagaaa gggactgaag ctgctggggc |
| 1381 | catgttttta gaggccatac ccatgtctat cccccccgag gtcaagttca acaaaccctt |
| 1441 | tgtcttctta atgattgaac aaaataccaa gtctcccctc ttcatgggaa aagtggtgaa |
| 1501 | tcccacccaa aaataactgc ctctcgctcc tcaacccctc ccctccatcc ctggccccct |
| 1561 | ccctggatga cattaaagaa gggttgagct ggtccctgcc tgcaaaa |

HOMO SAPIENS SERINE/THREONINE KINASE 32A (STK32A), MRNA. (NM_145001.2)
SEQ ID NO: 10

| | |
|---|---|
| 1 | ccttgctctt ggagttcttc tcttagtccc tgttccctgg atgaaagcat cgctccgagc |
| 61 | ctcatgggag gaatgaagga agaatcgaga ctagatatcc aactaaggct tcgggacatg |
| 121 | ttttgagcga agatgggtgt ttctgcccgg atagtataaa tcgaggatcc aggtctgggc |
| 181 | agattcaacc atgggagcca acacttcaag aaaaccacca gtgtttgatg aaaatgaaga |
| 241 | tgtcaacttt gaccactttg aaattttgcg agccattggg aaaggcagtt ttgggaaggt |
| 301 | ctgcattgta cagaagaatg ataccaagaa gatgtacgca atgaagtaca tgaataaaca |
| 361 | aaagtgcgtg gagcgcaatg aagtgagaaa tgtcttcaag gaactccaga tcatgcaggg |
| 421 | tctggagcac cctttcctgg ttaatttgtg gtattccttc caagatgagg aagacatgtt |
| 481 | catggtggtg gacctcctgc tgggtggaga cctgcgttat cacctgcaac agaacgtcca |
| 541 | cttcaaggaa gaaacagtga agctcttcat ctgtgagctg gtcatggccc tggactacct |
| 601 | gcagaaccag cgcatcattc acagggatat gaagcctgac aatattttac ttgacgaaca |
| 661 | tgatacctgg ctctcctaca agtcccactg aattggagtt tcaggagacc gaagcccagg |
| 721 | cacatgtatt ttgcaaaact acactgaagt ttctgataat gacggatatc aacaattaaa |
| 781 | cgcttacttc ttgtcaaaaa aaaaaaaaaa aaaaaaaaaa aaaa |

HOMO SAPIENS RPLK9433 (UNQ9433), MRNA. (NM_207413.1)
SEQ ID NO: 11

| | |
|---|---|
| 1 | attcggctcg agggcacact gaggaccgag tcccagtgca gctccagccg acccccggga |
| 61 | cgtagacaag ggcaggcgcg cggtgaagac tgcggccggc cgcgtagccg cgctgtggtc |
| 121 | ccgctggcct tctcttgggc cgcgcaccct ccggacctgc gtcgggatcc tcccggagtc |
| 181 | tcccgctctc tcctcccggc ttgcgaacat gcggcccctt aagcccggcg cccctttgcc |
| 241 | cgcactcttc ctgctggcgc tggctttgtc cccgcacgga gcccacggga ggccccgggg |
| 301 | gcgcagggga gcgcgcgtca cggataagga gcccaagccg ttgcttttcc tccccgcggc |
| 361 | cggggccggc cggactccca gcggctcccg gagcgcagaa atattcccaa gagactctaa |
| 421 | cttaaaagac aaattcataa agcatttcac agggccggtc acattttcac cagaatgcag |
| 481 | caaacatttc caccgactct attacaatac cagggagtgc tcaacgccag cttattacaa |
| 541 | aagatgtgct agattgttaa caagattagc agtgagtcca ctgtgctccc agacctagca |
| 601 | aaactaccct acatttccta agaatgtaca tctaatttga agaaaaagtg cctcaaatca |
| 661 | tgcaaaatgt aaaaaaagat gaaatttata tttttatgga tattaagatg agtaaaataa |
| 721 | gagacttccc agaaataact ggttagctgt ttcctgtcat agaatggagt ctttcttgct |
| 781 | ttatcttttt gtgtatacag taatttataa ttttgtaaaa cagagtttga atcgcatatt |
| 841 | gaaaattaga tattaaaaat tgtgtgattg tattttattt ttactagata tattattttc |
| 901 | tttatatggt taacattcta attaaacatt taattgtgta aattatatct gtgagtgcca |
| 961 | gtgagaaata atgatttttt tgtatatgac tgttagtata tatttgtcaa atcatcgtgt |
| 1021 | accaagttaa attttataat tcttatttaa tatttaaata catttattta gaaaaaaaaa |
| 1081 | aaaaaaaaaa aaaaaaaaaa aa |

HOMO SAPIENS CDNA CLONE IMAGE: 4811759 (BC030766)
SEQ ID NO: 12

| | |
|---|---|
| 1 | caaaagtaca gtaacgaagt attgaaaaga aaattttgga gacattggag catattatat |
| 61 | atagcttgtg gaaagacata aggctacaga tggaatggaa cattcctgtt ttcttgaaga |
| 121 | aattcacata cacatagctg acctgactag tacttcagct cttccacagc cttctataaa |
| 181 | ggttctttct tctgcaaaga aaacaaaaca aaacaaaaca aaacaaaaaa aaacaaaaaa |
| 241 | agcgcaaaaa acaaaaaaac aaaaaaaagc aaagtaaaat ttaaaaatac agaaaacaaa |
| 301 | caacaaaaaa gaattcaacc ataaatagtg actattattt tcagtgtgtc cttcatgtga |
| 361 | aagctattaa ggaccaaata tactactgtt cataagaaga aattactttc taaacagtaa |
| 421 | ctgaaaatac ttagagttaa acttgctgtg gattttgtct tggcagttgt catcttacat |
| 481 | tatttgtcaa aggaaatgtg tttggcagtt aaaaatcttt ccttagattt agtggtggac |
| 541 | tttaacctct taaataaatg ttagtatatc agattgtgtc cttgaaaaat attttacttg |
| 601 | tatgaatcat gacaacgtct aaatctttac tattcttctg gcaaaagcat cagtaagaaa |
| 661 | gaaggcgaaa aagagaagta tagcctttat gtcagaaaaa cattcttttt agctgcttac |
| 721 | tttctcatga aaagtaaaga tgtttacagt gtatgccaag ttttaagttt ctgtataaca |
| 781 | acaggtagag gttctaatca tattgaaaat tgtgttataa tggtctgagc catgttgcta |
| 841 | ggaaacaata ggttccaatt ttgtattcct gctctcctgt gctgaaaagt gactggatac |
| 901 | tgtacaggtt catgttctct ggctgcagtt aaatggtctt ttgcattttg ctctggcttt |
| 961 | caggccagaa gcatgcattt ttctacaaga gcatcacaac aacatgctgt aaatatttaa |
| 1021 | agttaaacat tatgtgttga tatttgaaag aaaagtactt tgaatatttc atttttaaaa |
| 1081 | aataaaattg ccaatgaaaa aaaaaaaaaa aa |

HOMO SAPIENS CDNA FLJ13457 FIS, CLONE PLACE1003343 (AK023519)
SEQ ID NO: 13

| | |
|---|---|
| 1 | agtctgtgag ctgggagcct gttggcaggt ccctcttttt attttcgctg agagctttct |
| 61 | tttactaaat gccaccatcc ttacctttca aggtgtctgc gtgcctaatt tttcctggtt |
| 121 | gttagacaag aacccggatt ttagttgaac tctggagcaa aaatcctgca tcatttgtag |
| 181 | gtaatgtttg ttttctttgg ttgcttttat gaccttctct ttattcctag tatttgggca |
| 241 | tttcacaaaa acatttataa atctgaatta aaaaacaaac tgaccttgcc aaggacccta |
| 301 | ctgatccctt ttcatataat tctatctgct aggtcttgtg ctgccttcta agttagttca |
| 361 | tcagttctat acttcagtat ttaattctat attcagccat gttatcaaat gtttatttca |
| 421 | acctttgagt ttctcatttg taatagtatt ttttcatttt tatgttttta aaagagttct |
| 481 | tttcatattt tactattttt gtttagtaac tttcagtact tgttgcgtag attttatttc |
| 541 | atctttatgt ttttgagatg tttgcataac acatttaaag acaattacat attgttgtat |
| 601 | gatttccatt tccttggtca agaattctcc catttgttgc atctctgaag agtttcccaa |
| 661 | ggtgggttaa tcttgaatat ggtaaacttg ctgccctcag ttttttagtt ttaatacttt |
| 721 | atcagttgtt tcttgttctt tttttttctg ggcatattat tacatcattt gaaaataatg |
| 781 | acaattttgg cctttctctt cgagattttg taactcattt tcttttcttt tcacatagaa |
| 841 | tttgataagg cctccagtac tttgtgatat agtagttgtg gggatggtcc ttcttgtctt |
| 901 | actgctgatc tcaagtttat gtttgtagca aatttttgct acatgaccat tacaagttta |
| 961 | agaggttacc aagagttttt attacaaata ccttagattt cagcaaagac tataaaaact |
| 1021 | gagataaaca tgtttttctt ctaaatagta atcatttaga caaatcacaa atgtgatatc |
| 1081 | agattagtgc atcctacact gtatggaaac tataaattca tgattttcaa aaattgtagg |
| 1141 | taaatattta tttttcaaaa taatttatcc atcttgcttt accaaaaaag gcaaggtctt |
| 1201 | gtttttcttt ccctatacag tagtaatggc tctttggtgt aaaaacttta taggacctaa |
| 1261 | agtaaaaaag aagataaaaa ccagtttgtc tttcaaagtc ttaaaatatt tcattaacct |
| 1321 | cgtagtgcct gatctctgtc accaagcaaa tagtctgaac tattatttcc aagctcaatt |
| 1381 | caaacaggta aagttttact acaattatat ctggccatca ctacagtgtt ttctccaatc |
| 1441 | acttccaaag cctacccctt ttctttatca tacttccacc tacattgcat aatacagagc |
| 1501 | ttagctagtg aggtacaagt gataaggatt atctccccag agtctccggg ccttggtaat |
| 1561 | tcctagacca cagcaccatg cattaggtta tagtcacagg cctctatata gtcttccaca |
| 1621 | tatcccatta cattttaaat cctatgcata ccctttatat cctgcactta tctgtcaaaa |
| 1681 | ttctaaggta taagattttt ttttacttat ccctgctgtt taattttgaa gaaagaaaaa |
| 1741 | tataaaagaa aattttttaa aaagaggcct tcatggatat caaagaatgc caacaataat |
| 1801 | gctgaagaaa gaagttattc tacagcaatg agacacaaag aaaagaattt tgtaaacatt |
| 1861 | agcatcttgg ttactggaga actataactt ttatgtagtc atgcttggaa aacactaaaa |
| 1921 | gggaaatcga gtctgtttga caatattctg tcttcactgt tgttcacttc ataatgtgtt |
| 1981 | ggaatataaa gttctataca gttaatatga agctctcttt agcatttaaa acatgatttg |
| 2041 | cattttcatg aggcattttg gctaatttta ttgatttcct tatatttcat agtccttagc |
| 2101 | cttatgagaa tcttatgttt ctgtgtgttt tctatcatgt agcacaattt ctgacacaca |
| 2161 | aaacatacaa taaacttgtg ttaatttttc tatcaaagtc agaatttatt cataaggaat |
| 2221 | ctgaagtaag gtgtactaag cttgtttatg ggttaagtga tatagccaaa ttcaaaactt |
| 2281 | tactttttat gtcagtctag aaatatctca gattaaaaca tatcacttct tagttccaat |
| 2341 | tagataaggg aaatctttta taataatgcc aggattgcta taatctgata ggagacaaca |
| 2401 | atttcatttt gctacaggaa aatttggagg ac |

HOMO SAPIENS SOLUTE CARRIER FAMILY 34 (SODIUM PHOSPHATE), MEMBER
2 (SLC34A2), MRNA (NM_006424.2)
SEQ ID NO: 14

| | |
|---|---|
| 1 | accttcgcca tatatacccg gggcgctgcg ctccacctgg ccgccgcctc cagcccagca |
| 61 | cctgcggagg gagcgctgac catggctccc tggcctgaat tgggagatgc ccagcccaac |
| 121 | cccgataagt acctcgaagg ggccgcaggt cagcagccca ctgcccctga taaaagcaaa |
| 181 | gagaccaaca aaacagataa cactgaggca cctgtaacca agattgaact tctgccgtcc |
| 241 | tactccacgg ctacactgat agatgagccc actgaggtgg atgacccctg gaacctaccc |
| 301 | actcttcagg actcggggat caagtggtca gagagagaca ccaaagggaa gattctctgt |
| 361 | ttcttccaag ggattgggag attgatttta cttctcggat ttctctactt tttcgtgtgc |
| 421 | tccctggata ttcttagtag cgccttccag ctggttggag gaaaaatggc aggacagttc |
| 481 | ttcagcaaca gctctattat gtccaaccct ttgttggggc tggtgatcgg ggtgctggtg |
| 541 | accgtcttgg tgcagagctc cagcacctca acgtccatcg ttgtcagcat ggtgtcctct |
| 601 | tcattgctca ctgttcgggc tgccatcccc attatcatgg gggccaacat tggaacgtca |
| 661 | atcaccaaca ctattgttgc gctcatgcag gtgggagatc ggagtgagtt cagaagagct |
| 721 | tttgcaggag ccactgtcca tgacttcttc aactggctgt ccgtgttggt gctcttgccc |
| 781 | gtggaggtgg ccacccatta cctcgagatc ataacccagc ttatagtgga gagcttccac |
| 841 | ttcaagaatg gagaagatgc cccagatctt ctgaaagtca tcactaagcc cttcacaaag |
| 901 | ctcattgtcc agctggataa aaaagttatc agccaaattg caatgaacga tgaaaaagcg |
| 961 | aaaaacaaga gtcttgtcaa gatttggtgc aaaactttta ccaacaagac ccagattaac |
| 1021 | gtcactgttc cctcgactgc taactgcacc tccccttccc tctgttggac ggatggcatc |
| 1081 | caaaactgga ccatgaagaa tgtgacctac aaggagaaca tcgccaaatg ccagcatatc |
| 1141 | tttgtgaatt tccacctccc ggatcttgct gtgggcacca tcttgctcat actctccctg |
| 1201 | ctggtcctct gtggttgcct gatcatgatt gtcaagatcc tgggctctgt gctcaagggg |
| 1261 | caggtcgcca ctgtcatcaa gaagaccatc aacactgatt tcccctttcc ctttgcatgg |
| 1321 | ttgactggct acctggccat cctcgtcggg gcaggcatga ccttcatcgt acagagcagc |
| 1381 | tctgtgttca cgtcggcctt gacccccctg attggaatcg gcgtgataac cattgagagg |
| 1441 | gcttatccac tcacgctggg ctccaacatc ggcaccacca ccaccgccat cctggccgcc |
| 1501 | ttagccagcc ctggcaatgc attgaggagt tcactccaga tcgccctgtg ccactttttc |
| 1561 | ttcaacatct ccggcatctt gctgtggtac ccgatcccgt tcactcgcct gcccatccgc |
| 1621 | atggccaagg ggctgggcaa catctctgcc aagtatcgct ggttcgccgt cttctacctg |
| 1681 | atcatcttct tcttcctgat cccgctgacg gtgtttggcc tctcgctggc cggctggcgg |
| 1741 | gtgctggttg gtgtcggggt tcccgtcgtc ttcatcatca tcctggtact gtgcctccga |
| 1801 | ctcctgcagt ctcgctgccc acgcgtcctg ccgaagaaac tccagaactg gaacttcctg |
| 1861 | ccgctgtgga tgcgctcgct gaagccctgg gatgccgtcg tctccaagtt caccggctgc |
| 1921 | ttccagatgc gctgctgctg ctgctgccgc gtgtgctgcc gcgcgtgctg cttgctgtgt |
| 1981 | ggctgcccca agtgctgccg ctgcagcaag tgctgcgagg acttggagga ggcgcaggag |
| 2041 | gggcaggatg tccctgtcaa ggctcctgag acctttgata acataaccat tagcagagag |
| 2101 | gctcagggtg aggtccctgc ctcggactca aagaccgaat gcacggcctt gtaggggacg |
| 2161 | ccccagattg tcagggatgg ggggatggtc cttgagtttt gcatgctctc ctccctccca |
| 2221 | cttctgcacc ctttcaccac ctcgaggaga tttgctcccc attagcgaat gaaattgatg |
| 2281 | cagtcctacc taactcgatt ccctttggct tggtggtagg cctgcagggc acttttattc |
| 2341 | caacccctgg tcactcagta atcttttact ccaggaaggc acaggatggt acctaaagag |
| 2401 | aattagagaa tgaacctggc gggacggatg tctaatcctg cgcctagctg ggttggtcag |
| 2461 | tagaacctat tttcagactc aaaaaccatc ttcagaaaga aaaggcccag ggaaggaatg |
| 2521 | tatgagaggc tctcccagat gaggaagtgt actctctatg actatcaagc tcaggcctct |
| 2581 | cccttttttt aaaccaaagt ctggcaacca agagcagcag ctccatggcc tccttgcccc |
| 2641 | agatcagcct gggtcagggg acatagtgtc attgtttgga aactgcagac cacaaggtgt |
| 2701 | gggtctatcc cacttcctag tgctccccac attccccatc agggcttcct cacgtggaca |
| 2761 | ggtgtgctag tccaggcagt tcacttgcag tttccttgtc ctcatgcttc ggggatggga |
| 2821 | gccacgcctg aactagagtt caggctggat acatgtgctc acctgctgct cttgtcttcc |
| 2881 | taagagacag agagtggggc agatggagga gaagaaagtg aggaatgagt agcatagcat |
| 2941 | tctgccaaaa gggccccaga ttcttaattt agcaaactaa gaagcccaat tcaaaagcat |
| 3001 | tgtggctaaa gtctaacgct cctctcttgg tcagataaca aaagccctcc ctgttggatc |
| 3061 | ttttgaaata aaacgtgcaa gttatccagg ctcgtagcct gcatgctgcc accttgaatc |
| 3121 | ccagggagta tctgcacctg gaatagctct ccacccctct ctgcctcctt actttctgtg |
| 3181 | caagatgact tcctgggtta acttccttct ttccatccac ccacccactg gaatctcttt |
| 3241 | ccaaacattt ttccattttc ccacagatgg gctttgatta gctgtcctct ctccatgcct |
| 3301 | gcaaagctcc agatttttgg ggaaagctgt acccaactgg actgcccagt gaactgggat |
| 3361 | cattaagtac agtcgagcac acgtgtgtgc atgggtcaaa ggggtgtgtt ccttctcatc |
| 3421 | ctagatgcct tctctgtgcc ttccacagcc tcctgcctga ttacaccact gcccccgccc |
| 3481 | caccctcagc catcccaatt cttcctggcc agtgcgctcc agccttatct aggaaaggag |
| 3541 | gagtgggtgt agccgtgcag caagattggg gcctccccca tcccagcttc tccaccatcc |
| 3601 | cagcaagtca ggatatcaga cagtcctccc ctgaccctcc cccttgtaga tatcaattcc |
| 3661 | caaacagagc caaatactct atatctatag tcacagccct gtacagcatt tttcataagt |
| 3721 | tatatagtaa atggtctgca tgatttgtgc ttctagtgct ctcatttgga aatgaggcag |
| 3781 | gcttcttcta tgaaatgtaa agaaagaaac cactttgtat attttgtaat accacctctg |
| 3841 | tggccatgcc tgccccgccc actctgtata tatgtaagtt aaacccgggc aggggctgtg |
| 3901 | gccgtctttg tactctggtg atttttaaaa attgaatctt tgtacttgca ttgattgtat |
| 3961 | aataattttg agaccaggtc tcgctgtgtt gctcaggctg gtctcaaact cctgagatca |
| 4021 | agcaatccgc ccacctcagc ctcccaaagt gctgagatca caggcgtgag ccaccaccag |
| 4081 | gcctgattgt aatttttttt tttttttttt tactggttat gggaagggag aaataaaatc |
| 4141 | atcaaaccca aaaaaaaaaa aaaaaaa |

HOMO SAPIENS MRNA; CDNA DKFZP686N1644 (FROM CLONE DKFZP686N1644)
(BX538295)
SEQ ID NO: 15

| | |
|---|---|
| 1 | gggatttcca cttttattgc aaaacatgct tgcttgatgc aatgcatttc agatctagaa |
| 61 | aaaaataatg caagcaagct acatttacag atgtgtaaac aggcaaaatt tccagaaccg |
| 121 | tccaatttta cttttccaaa tttatggaac caacccagat tttttaaatg aaaatactaa |
| 181 | aaccatatgt aaccgtaaga caagctaaaa tgtaagcaga cagcctggga agcactgccc |
| 241 | tactgtacag gtgtgatatt aagcacaagc catcctctgg cctcttatga gatgttgtta |
| 301 | ttctctgaaa aactttgatg gaaaaggttt ctgttaggct tagactgaat tagctaaatg |
| 361 | accatgccac agtattattg tatcactctt ctcacacacc taggcactgg ttgtggatag |
| 421 | ttgccatggt gacaatcaag ataatccaga tgacctaaac aaatgtctgt atgcagctga |
| 481 | cactgctatt ctccctcaca gtcttgtgaa gagccacagt agctatggca gacatttggt |
| 541 | tgatggtttc atcatttcct aatatgagag tctggcacta tgttggagag aggaatcact |
| 601 | tcctcagaga taggttcctt tcaactcaca tgtcaacagt tactgtacaa aaagctcaga |
| 661 | atacagtaat gattagagct tggcccctga ctttaggaac ttagaaccaa agcccaaagt |
| 721 | cccaggccaa gggcagacat tgtgagcagc agaagggtgt tttttatgga gagccccata |
| 781 | aatggatggg tgtagacctt gaagtctgag atgggaaata taccagaatg ctcaatgcat |
| 841 | cagacatgaa gccccacggg cttccatttg acatgctggc aatctgaagg acaaagggca |
| 901 | tgggttattt gggccttttc cccactcctt caatatggct gaaagtgcca gtggggatta |
| 961 | caacctaaga aaccctgtgc cccttcctga gggcttgatt attgaatcct gatcttcctg |
| 1021 | gaatctttca cttgagatga tcacctgcct aacatccaac atgttactat gctcttcttt |
| 1081 | atgtacatga gttactggaa accaacaata aaggagagtg agtcatactc tccacatatg |
| 1141 | cgccaattaa ttttttaact tacttgttag tattcttgaa gaacttacta tcttaagtat |
| 1201 | tttaccaata gatgatgcat tttcctgcaa ctcaatggag ttctacctta aagtatttac |
| 1261 | cattgatcca aaattggagg aactacctta gattctttta ataacctaga aaggattgga |
| 1321 | gcttgtatta tttcctaaac cataggcagg cagtctagct tccctgctgg atcataagac |
| 1381 | caggacagca gagattgggt ttgtattgat cagtagtaga gaagtattga ttgtctaaca |
| 1441 | tctagtaagt tgtctttatc agtatagaca tagacaaaat acatgtttta tgaatgaata |
| 1501 | aataataaga accttaagag ctgttttttt cccatttaag aaaacaaaaa ggaaaaggga |
| 1561 | aaaatgatct tctggaataa ctctgctttg atattctcct tttgttcccc aaaaaatata |
| 1621 | ttccagaaag gcaaaccaaa tctgctcaag ttgcctatgc acagacaatc tcaacaatat |
| 1681 | ggctcacagt gagcaagcag aaattcaggg tctggaattg aatgtgagtt atgggaagaa |
| 1741 | acagtgtgtg ctttcacaaa gatccttttg tcagacaagg gaacagcatg ataatccaat |
| 1801 | ggaactggtt aacatgaccc aagatgacat gaccacagat atgctggtcg ttctgaaact |
| 1861 | tgcccaagaa attgcaatcg tttcagcaga actccatgaa atattgacaa attaattcca |
| 1921 | tcatcaaact aggagtgata ttatcaggta aaactatgat ctcatgtatg tatttggtct |
| 1981 | ttctgggtca caatgtgaca tgaaaaattt taaaggtcat attttagcaa gatcaaaatg |
| 2041 | aattagataa atcaacttca caaatgttaa gcgaaacatg atcctattga gctgagaaag |
| 2101 | aagtacttga gatcgtttga gttttgacta tcaaatgtca gcacaaaagc aaatatcaaa |
| 2161 | agcttaagaa aaaaattatt tcaggaaaag aaaaacacat caaattgaga aagtgttgtt |
| 2221 | gaataacttt gtttttagaa ctggaaattt aggcttatta gacatgacat tgatattaca |
| 2281 | aagccctagt tttcatgaaa acataactag ataaccccaa ggtgtatagg ttaaaatgaa |
| 2341 | ctaattaatc aacaattatg catcctgtgt ctattttgtg caaggtactt cctagacact |
| 2401 | gtagtttaaa agaataattt cctgtagaaa aacctaatct aataggacct actcataatt |
| 2461 | tggcaaaatc atcctcataa ttccatgcta ctgaaatggg ggtgtaataa agaaaaaatc |
| 2521 | atttgtttat tcatttaaca gggtttatta agcaccaata tatgcccaga actttgcagt |
| 2581 | gcactggaag ttcagagata agaaaaccac agtttctgcc atcaaaatgc taggaatcta |
| 2641 | gaggtctcaa acatcagagg tcccttcaaa ctatgtctac caggaaaata aaatacctga |
| 2701 | tttcatccct gcaaacaact ctatacagac agaaatgctt cctagcttgg aaactataga |
| 2761 | aaattgaaca tgaaaatgag agcaaagatt ctacatcaaa atggcaatga tccaatcagc |
| 2821 | ccagtcaagg ctgagtgggt tgaatgggga cttcgagttt ggattcaatg ctttgaactt |
| 2881 | catagctcca gagaagctgt gcaaaaaggg gggattctgg ggaatctgag gaagattgtt |
| 2941 | ggagaaacca gctttcttct tgtctcctaa tccctgctaa gattgatacc agtcaaggag |
| 3001 | ctgagttact ttcatttcat tatgttgacc aagagaagct tatgatacag actttgacac |
| 3061 | agcacctatt tcaagagaaa gatgttactc tacttatggg aacctgcaga tcaaattctc |
| 3121 | ctggggcaag agaagaaatt tgttcagcag cctaagaagt caactgtact ttggacagtg |
| 3181 | ctgccaaaca caccccagga ttctcccacc cctgtgcttt cccagaagat gccacatagc |
| 3241 | tggtatttgg ttttcatgaa taaccaaaga ttcttctttg cttcctgaac cccaggcatc |
| 3301 | cttaggaatt tgcttcatgt cctcacttgt ccttgaaggc ttgtttttca ggatgaactt |
| 3361 | ttaaagtcta agttgtttat attcaaatca acccaatgct attgttgctg atattaaacc |
| 3421 | attccaaact gagccaactg gcccctaaac aagccagcag gcagcccctc actaatgccc |
| 3481 | agtttagaac atgttaggac caggctatct gcatagcttt agtggtttta gaagccagta |
| 3541 | atggcagtgt aaaagagaca ggaatcatac agagatgacc tggaagaaaa tttgagtttc |
| 3601 | agcacattca aaagcatttg actcttctgc cgtgttttga ttttcaagtg agacaatcaa |
| 3661 | tgtacacaca aagcctctag tacaatgact gatgcatact agggactgtt atttatgtta |
| 3721 | tttttttaca gtatgcatgg acttcattgt ctcagtcttt ggtgttactt atgaactcta |
| 3781 | aatttacacc aaaaaaattt tttttcagaa aaaggatcgt ggagaaacaa tcctaattat |
| 3841 | taaatcaaat gctttcacaa ttgcttttta aataagtaca tctctgcaaa taaaaatgct |
| 3901 | tgctcttgtc taccacaata aagaacagaa ggaatcttgg aatgaagaga ggcaggaatg |
| 3961 | ttggatctca gtggctgagt tgtggcaatt ccattggaca tatagtacca gatgttgcac |
| 4021 | catctggatg atgtgtagga gataaagcca aattgcaaaa ataaaagact catgatttcc |
| 4081 | caaggaaagt caactgtgca tggtggctat cagacaacac cctagcaatg aatgggggaa |
| 4141 | cttgcagaag aaagagttaa tgcttgctta aaccttccct ggggagattt gttcccaggc |
| 4201 | tttcacggct ttaacctcag caaccctgta agaaaattgt gattcatatt ttgatttgtt |
| 4261 | atcatagatg caaagagaca taatttcatg gattctaaga tacacagagc aagaagagat |
| 4321 | tttgatcacc cagaacaacc acctgtcttt ttagatcaag aagcagaagc tcagagcaaa |
| 4381 | gaacatcaca ccacgtccct cagtgattga agcagtgatt gagtcacaga ataaatctgg |
| 4441 | aactcaggtc ttctgatctt tgctccagat gttagagaca aaactaaaag taaaatacca |
| 4501 | agtgaaatca aagcatcacg attgagccca gaacatgaaa aagaacttcc tggcccactt |
| 4561 | gagaaactgt taaaccggac atacctttgg ggacttcttc ccttctctgg aataagattg |
| 4621 | atgtttccat gctgtgaaag acgatgatgt tccttctccc agattcctgc tgtcttcaaa |
| 4681 | aggcctagca aaaaccactg ctgctgggtg cagttgagaa agggaatgaa gaacaatccc |
| 4741 | atggccctgc aggcactcct cccctccacc tctctgccct ccacccctct gccctctcct |
| 4801 | accttgttcc ttcttggtcc tgccatgttt ggactggttc acctttttgg cccatcactt |
| 4861 | gggctcattc agcaacagtc atgccttagg ttgcatcctc atctataggc caattaaaca |
| 4921 | gggtttcttc ttgataaatc cctatggttt tgattttagg aagtcaggca tgatttcttt |
| 4981 | tgggatctat atgacccaaa gtggccagtt ttaccaaagg tgtccctttg cactgggatc |
| 5041 | tctgggtctc atttgaacac agcttgggtc ctcttgcctt gtcccagaga atccccatgt |
| 5101 | tgtcaggaca taagcagata ctctagggcc ttcaccaaaa gctacaaaga agcaaacagg |
| 5161 | actcaactta ggacatctta tggttatgta actaagagat tattttagga agtaaacagg |
| 5221 | cctctgttgg ctcaaatgaa catcttcatt gttagtttcc cttacattgt tctcgggatg |
| 5281 | aatcggccag aatttggcag ggctccccag cagtccttgt gaatccctgt ggtaggatgt |
| 5341 | ttcagttctc ctgagtcagg ctgaagtgaa agctcctaag agatattcag aaagaaggat |
| 5401 | ggtcaagttc tagaacaaca atagttggcc cagtaatttt aaagattgtc attctaccta |
| 5461 | agccttcatc ctccagatag cactgttatg ggcttaaaat aagcttcaag ggaaataatg |
| 5521 | aatttctttg acaccgccaa aaatcggctg tatataagaa taacttgaga atggcttaca |
| 5581 | atgaggtagt caaaagccca gctcagtgct caggaatgta tttgtaattc ctcttagact |
| 5641 | tgtcgaatgg actgcaattt gatacctaag agtaataaaa aatgagagca tattttatgt |
| 5701 | taatgttccc ttctcttcct ctgcccacaa aagtgaggtc agggaagggg agagaaactt |
| 5761 | atcctaagcg ctccttatga gttgcttgtg acatgatagc tctctctaaa gttcatgcaa |
| 5821 | tcctcatcgc aaccccatga gggagacgtt attgtctcca tcttatagat gaggaaactg |
| 5881 | aagacaaggc cttgtcaaag gtcacacatt gtaagaagta gagctgcata tttaaccaaa |
| 5941 | gcccgtgtac ctggaaggct cattctctct cccccttctt cctctcaaat caggccctag |
| 6001 | ctcctccaca atgcttcggg cttctacgtt cacttagctt tacagtaaaa tgatcacaga |
| 6061 | cccatgagtt ccctggagag tagagtgatg cctgggtcag ctgttcctct tgtctgttca |
| 6121 | gcaggtcctt tgagtggctt cacaatccag ctgtcagtgg cagaaaataa tgcctcataa |
| 6181 | attattcacc ttcttcacat tggaatccat tagctctcgg ggcttggtgt ggctgcagat |
| 6241 | catccacaag tgccccttgt tctctgcccc caaggagatc ctgtcattgt ttttaaccaa |
| 6301 | acaagttcaa atatctccac gtctctgctg actctggtct tttgtctcta gactgtacct |
| 6361 | ttatttctga gctctgaaat tctgcagaaa caggtgagtc tataaaaaca ttctgtggga |
| 6421 | ggccaaaagc tcagaacagt tgtgtctctc tttctcagta ttaaaagatc aaggtttccc |
| 6481 | atggaagcca accggatctg gtaattgggc ccttctatac ccctctgaat acttttccaa |
| 6541 | ggagggggca gttcccaccc ccatagttcc cctgagggtt aagagtccag gcggtggcca |
| 6601 | agttgcaaga tttggggcag ccattttgcc aaggatcaga gatttgctgt tggagaggtg |
| 6661 | aagtcggctc tgtgccatcc tgaaaaatca acttagaaag ataggtggac ttcatcagat |
| 6721 | ttgtcagtcc ttccttgttt taaagagaaa aatgatctct gccttgcttc agataggata |
| 6781 | tggctatttt ttttccttct ttttctaatg tagcagtgat ggaaaacacc agtctttgaa |
| 6841 | gaacaaatgg aggttcccag tgacatgaat ccattatatt tgattttgga ttgtattttt |
| 6901 | taaccccttt ttatggcata gaattctggt gccttgctcc ctgggtttgt ctctgtgcta |
| 6961 | caagtaggct ttcttcacat tctggcttac aggggaacac aactattcca caagtggcct |
| 7021 | ttagtgctct ttataatatg atttcctgta atttttaaac tgtatgtgta atttatattc |
| 7081 | tggctctgtc caatatttga acaactttaa taggttgatt tccatatata ttatgcaaat |
| 7141 | aattcttacc tgattttttt atgttctatc ttaaataaat actgcaccac ttattaataa |
| 7201 | atagacattt cagtgataaa aaaaaaaaaa aaaaa |

HOMO SAPIENS IGF-LIKE FAMILY MEMBER 2 (IGFL2), MRNA (NM_001002915.1)
SEQ ID NO: 16

| | |
|---|---|
| 1 | tttgggtttg gacgggtgcg tttgtttttc atagtgcagg tctggagccg gactgagatg |
| 61 | gggagtagag ggaccccttg aaacttcagg gtggatgaat atgaagtttc atcaccagct |
| 121 | gtgatgtttg atattggact gtggttgtgc cagttgacac tcaggagtgg agactagatc |
| 181 | tgcaatctgt tgggactgtg atgaggggat catcctgccc tcgaaccaga cccagccctg |
| 241 | tagcccaggc actattgcct ggagttcctg ggctctcagc gccaggaaat catgaggttc |
| 301 | agtgtctcag gcatgaggac cgactacccc aggagtgtgc tggctcctgc ttatgtgtca |
| 361 | gtctgtctcc tcctcttgtg tccaagggaa gtcatcgctc ccgctggctc agaaccatgg |
| 421 | ctgtgccagc cggcacccag gtgtggagac aagatctaca accccttgga gcagtgctgt |
| 481 | tacaatgacg ccatcgtgtc cctgagcgag acccgccaat gtggtccccc ctgcaccttc |
| 541 | tggccctgct ttgagctctg ctgtcttgat tcctttggcc tcacaaacga ttttgttgtg |
| 601 | aagctgaagg ttcagggtgt gaattcccag tgccactcat ctcccatctc cagtaaatgt |
| 661 | gaaagcagaa gacgttttcc ctgagaagac atagaaagaa aatcaacttt cactaaggca |
| 721 | tctcagaaac ataggctagg gtaatatgtg taccagtaga gaagcctgag gaatttacaa |
| 781 | aatgatgcag ctccaagcca ttgtatggcc catgtgggag actgatggga catggagaat |
| 841 | gacagtagat tatcaggaaa taaataaagt ggtttttcca atgtaaaaaa aaaa |

HOMO SAPIENS CHITINASE 3-LIKE 1 (CARTILAGE GLYCOPROTEIN-39) (CHI3L1),
MRNA. (NM_001276.2)
SEQ ID NO: 17

| | |
|---|---|
| 1 | cacatagctc agttcccata aaagggctgg tttgccgcgt cggggagtgg agtgggacag |
| 61 | gtatataaag gaagtacagg gcctggggaa gaggccctgt ctaggtagct ggcaccagga |
| 121 | gccgtgggca agggaagagg ccacaccctg ccctgctctg ctgcagccag aatgggtgtg |
| 181 | aaggcgtctc aaacaggctt tgtggtcctg gtgctgctcc agtgctgctc tgcatacaaa |
| 241 | ctggtctgct actacaccag ctggtcccag taccgggaag gcgatgggag ctgcttccca |
| 301 | gatgcccttg accgcttcct ctgtacccac atcatctaca gctttgccaa tataagcaac |
| 361 | gatcacatcg acacctggga gtggaatgat gtgacgctct acggcatgct caacacactc |
| 421 | aagaacagga accccaacct gaagactctc ttgtctgtcg gaggatggaa ctttgggtct |
| 481 | caaagatttt ccaagatagc ctccaacacc cagagtcgcc ggactttcat caagtcagta |
| 541 | ccgccatttc tgcgcaccca tggctttgat gggctggacc ttgcctggct ctaccctgga |
| 601 | cggagagaca aacagcattt taccacccta atcaaggaaa tgaaggccga atttataaag |
| 661 | gaagcccagc cagggaaaaa gcagctcctg ctcagcgcag cactgtctgc ggggaaggtc |
| 721 | accattgaca gcagctatga cattgccaag atatcccaac acctggattt cattagcatc |
| 781 | atgacctacg attttcatgg agcctggcgt gggaccacag gccatcacag tcccctgttc |
| 841 | cgaggtcagg aggatgcaag tcctgacaga ttcagcaaca ctgactatgc tgtggggtac |
| 901 | atgttgaggc tgggggctcc tgccagtaag ctggtgatgg gcatccccac cttcgggagg |
| 961 | agcttcactc tggcttcttc tgagactggt gttggagccc caatctcagg accgggaatt |
| 1021 | ccaggccggt tcaccaagga ggcagggacc cttgcctact atgagatctg tgacttcctc |
| 1081 | cgcggagcca cagtccatag aatcctcggc cagcaggtcc cctatgccac caagggcaac |
| 1141 | cagtgggtag gatacgacga ccaggaaagc gtcaaaagca aggtgcagta cctgaaggac |
| 1201 | aggcagctgg cgggcgccat ggtatqggcc ctggacctgg atgacttcca gggctccttc |
| 1261 | tgcggccagg atctgcgctt ccctctcacc aatgccatca aggatgcact cgctgcaacg |
| 1321 | tagccctctg ttctgcacac agcacggggg ccaaggatgc cccgtccccc tctggctcca |
| 1381 | gctggccggg agcctgatca cctgccctgc tgagtcccag gctgagcctc agtctccctc |
| 1441 | ccttggggcc tatgcagagg tccacaacac acagatttga gctcagccct ggtgggcaga |
| 1501 | gaggtaggga tggggctgtg gggatagtga ggcatcgcaa tgtaagactc gggattagta |
| 1561 | cacacttgtt gattaatgga aatgtttaca gatccccaag cctggcaagg gaatttcttc |
| 1621 | aactccctgc cccccagccc tccttatcaa aggacaccat tttggcaagc tctatcacca |
| 1681 | aggagccaaa catcctacaa gacacagtga ccatactaat tataccccct gcaaagccca |
| 1741 | gcttgaaacc ttcacttagg aacgtaatcg tgtcccctat cctacttccc cttcctaatt |
| 1801 | ccacagctgc tcaataaagt acaagagctt aacagtgaaa aaaaaaaaaa aaaaaaaaaa |
| 1861 | aaaaaaa |

HOMO SAPIENS CYTOCHROME P450, FAMILY 24, SUBFAMILY A, POLYPEPTIDE
1 (CYP24A1), NUCLEAR GENE ENCODING MITOCHONDRIAL PROTEIN, MRNA.
(NM_000782.3)
SEQ ID NO: 18

| | |
|---|---|
| 1 | tggagaggga caggaggaaa cgcagcgcca gcagcatctc atctaccctc cttgacacct |
| 61 | ccccgtggct ccagccagac cctagaggtc agccttgcgg accaacagga ggactcccag |
| 121 | ctttcccttt tcaagaggtc cccagacacc ggccaccctc ttccagcccc tgcggccagt |
| 181 | gcaaggaggc accaatgctc tgaggctgtc gcgtggtgca gcgtcgagca tcctcgccga |
| 241 | ggtcctttct gctgcctgtc ccgcctcacc ccgctccatc acaccagctg gccctctttg |
| 301 | cttccttttc ccagaatcgt taagccccga ctcccactag cacctcgtac caacctcgcc |
| 361 | ccaccccatc ctcctgcctt cccgcgctcc ggtgtccccc gctgccatga gctcccccat |
| 421 | cagcaagagc cgctcgcttg ccgccttcct gcagcagctg cgcagtccga ggcagccccc |
| 481 | gagactggtg acatctacgg cgtacacgtc ccctcagccg cgagaggtgc cagtctgccc |
| 541 | gctgacagct ggtggcgaga ctcagaacgc ggccgccctg ccgggcccca ccagctggcc |
| 601 | actgctgggc agcctgctgc agattctctg gaaagggggt ctcaagaaac agcacgacac |
| 661 | cctggtggag taccacaaga agtatggcaa gattttccgc atgaagttgg gttcctttga |
| 721 | gtcggtgcac ctgggctcgc catgcctgct ggaagcgctg taccgcaccg agagcgcgta |
| 781 | cccgcagcgg ctggagatca aaccgtggaa ggcctatcgc gactaccgca aagaaggcta |
| 841 | cgggctgctg atcctggaag gggaagactg gcagcgggtc cggagtgcct ttcaaaagaa |
| 901 | actaatgaaa ccaggggaag tgatgaagct ggacaacaaa atcaatgagg tcttggccga |
| 961 | ttttatgggc agaatagatg agctctgtga tgaaagaggc cacgtcgaag acttgtacag |
| 1021 | cgaactgaac aaatggtcgt ttgaaagtat ctgcctcgtg ttgtatgaga agagatttgg |
| 1081 | gcttctccag aagaatgcag gggatgaagc tgtgaacttc atcatggcca tcaaaacaat |
| 1141 | gatgagcacg tttgggagga tgatggtcac tccagtcgag ctgcacaaga gcctcaacac |
| 1201 | caaggtctgg caggaccaca ctctggcctg ggacaccatt ttcaaatcag tcaaagcttg |
| 1261 | tatcgacaac cggttagaga agtattctca gcagcctagt gcagatttcc tttgtgacat |
| 1321 | ttatcaccag aatcggcttt caaagaaaga attgtatgct gctgtcacag agctccagct |
| 1381 | ggctgcggtg gaaacgacag caaacagtct aatgtggatt ctctacaatt tatcccgtaa |
| 1441 | tccccaagtg caacaaaagc ttcttaagga aattcaaagt gtattacctg agaatcaggt |
| 1501 | gccacgggca gaagatttga ggaatatgcc gtatttaaaa gcctgtctga aagaatctat |
| 1561 | gaggcttacg ccgagtgtac catttacaac tcggactctt gacaaggcaa cagttctggg |
| 1621 | tgaatatgct ttacccaaag gaacagtgct catgctaaat acccaggtgt tgggatccag |
| 1681 | tgaagacaat tttgaagatt caagtcagtt tagacctgaa cgttggcttc aggagaagga |
| 1741 | aaaaattaat ccttttgcgc atcttccatt tggcgttgga aaaagaatgt gcattggtcg |
| 1801 | ccgattagca gagcttcaac tgcatttggc tctttgttgg attgtccgca aatacgacat |
| 1861 | ccaggccaca gacaatgagc ctgttgagat gctacactca ggcaccctgg tgcccagccg |
| 1921 | ggaactcccc atcgcgtttt gccagcgata atacgcctca gatggtggta tttgctaaca |
| 1981 | tcatatccaa ctcagggaag cggactgagt gctgggatcc aaggcattct acagggttca |
| 2041 | ctgctggttt acacttcacc tgtgtcagca ccatcttcag gtgcttagaa tggcctggga |
| 2101 | gcctgttctg tcttgcatct tccatgacat gaaagggagg ctggcacttg tcagtcaggt |
| 2161 | agaggttaca aaccgtttca ggccctgcct accacattca ctgtttgaat ctttaattcc |
| 2221 | caagaataag tttacatttc acaatgaatg acctacaaca gctaaatttt ctggggctgg |
| 2281 | gagtaatact gacaatccat ttactgtagc tctgcttaat gtactactta ggaaaatgtc |
| 2341 | cctgcttaat aatgtaagcc aagctaaatg atggttaaag ttatcaggcc tcccatgaaa |
| 2401 | ttgcgttctt cctgcattga aataaaaaca ttattgggaa actagagaac acctctattt |
| 2461 | ttaaaaggac tttaacgaag tcaaacaact tataagacta gtgattcact ggggcattat |
| 2521 | tttgttagag gaccttaaaa ttgtttattt tttaaatgtg attcctttat ggcattaggg |
| 2581 | taaagatgaa gcaataattt ttaaattgtg tatgtgcata tgaagcacag acatgcatgt |
| 2641 | gtgtgtgtgt ctgtgtgtgt gtgtccgtgt atgtgtgtgt gggttctaat ggtaatttgc |
| 2701 | ctcagtcatt tttttaatat ttgcagtact tgatttagga tctgtggtgc agggcaatgt |
| 2761 | ttcaaagttt agtcacagct taaaaacatt cagtgtgact ttaatattat aaaatgattt |
| 2821 | cccatgccat aatttttctg tctattaaat gggacaagtg taaagcatgc aaaagttaga |
| 2881 | gatctgttat ataacatttg ttttgtgatt tgaactccta ggaaaaatat gatttcataa |
| 2941 | atgtaaaatg cacagaaatg catgcaatac ttataagact taaaaattgt gtttacagat |
| 3001 | ggtttatttg tgcatatttt tactactgct tttcctaaat gcatactgta tataattctg |
| 3061 | tgtatttgat aaatatttct tcctacatta tatttttaga atatttcaga aatatacatt |
| 3121 | tatgtcttta tattgtaata aatatgtaca tatctaggta tatgctttct ctctgctgtg |
| 3181 | aaattatttt tagaattata aattcacgtc ttgtcagatt tcatctgtat accttcaaat |
| 3241 | tctctgaaag taaaaataaa agtttttaaa tattaaaaaa aaaaaaaaaa aaaaa |

HOMO SAPIENS HEDGEHOG ACYLTRANSFERASE-LIKE (HHATL), MRNA. (NM_020707.2)
SEQ ID NO: 19

| | |
|---|---|
| 1 | aggggctgaa tacacagagc gctgagagag tggggcagtg tggtcacgga cacaggtcat |
| 61 | gggggccttg gcaggagcgg ttttggcaga gggtggggcc ggtgcctctg gaaggtatga |
| 121 | agatgtaaat gaagccctgt ccaggctatg ggcatcaaga cagcattgcc ggcggctgag |
| 181 | ctgggcctct actctctggt gctgagtggg gccctggcct atgctggccg gggcctcctt |
| 241 | gagtacattg gccggaagat ggatgtggct gacttcgagt gggtgatgtg gttcacctcc |
| 361 | tttcgcaacg tcatcatctt tgccctctcc ggacatgtgc tgtttgctaa actctgcacg |
| 421 | atggttgccc caaagctccg ctcctggatg tatgctgtgt acggggcctt ggctgtgatg |
| 481 | ggcacaatgg gcccttggta cctgctgctg ctgcttggtc actgtgtggg cctctatgtg |
| 541 | gcctcgcttt tgggccagcc ctggctctgt cttggccttg gcttggccag cctggcctcc |
| 601 | ttcaagatgg accccctaat ctcttggcag agcgggtttg taacaggcac ttttgatctt |
| 661 | caagaggtgc tgtttcatgg gggcagcagc ttcacagtgc tgcgttgcgc cagctttgca |
| 721 | ctggagagct gtgcccaccc tgaccgccac tactccttag ctgacctgct caagtacaac |
| 781 | ttctacctgc ccttcttctt cttcgggcca atcatgacct ttgatcgctt ccatgctcag |
| 841 | gtgagccagg tggagccagt gagacgcgag ggtgagctgt ggcacatccg agcccaggca |
| 901 | ggcctaagcg tggtggccat catggccgtc gacatcttct ttcacttctt ctacatcctc |
| 961 | actatcccca gcgacctcaa gttcgccaac cgcctcccag acagtgccct cgctggccta |
| 1021 | gcctattcaa acctggtgta tgactgggtg aaggcggccg tcctctttgg tgttgtcaac |
| 1081 | actgtggcat gcctcgacca cctggaccca ccccagcctc ccaagtgcat caccgcactc |
| 1141 | tatgtcttta cggaaacgca ctttgaccgt ggcatcaacg actggctttg caaatatgtg |
| 1201 | tataaccaca ttggtgggga gcattccgct gtgatcccag agctggcagc cacagtggcc |
| 1261 | acatttgcca tcaccacact gtggcttggg ccttgtgaca ttgtctacct gtggtcattc |
| 1321 | cttaactgct ttggcctcaa ctttgagctc tggatgcaaa aactggcaga gtgggggccc |
| 1381 | ctagcacgaa ttgaggcctc tctgtcagtg cagatgtccc gtagggtccg ggccctgttt |
| 1441 | ggagccatga acttctgggc catcatcatg tacaaccttg tgagcctgaa cagcctcaaa |
| 1501 | ttcacagagc tggttgcccg gcgcctgcta ctcacagggt ttccccagac cacgctgtcc |
| 1561 | atcctgtttg tcacctactg tggcgtccag ctggtaaagg agcgtgagcg aaccttggca |
| 1621 | ctggaggagg agcagaagca ggacaaagag aagccggagt aggagggagc gggtagaggg |
| 1681 | atgggctctg ctcagctatt cttgggccag atggggcctg accgatagaa taaaagactt |
| 1741 | ttctacaaca aaaaaaaaaa aaaaaaaaaa aacaaaaaaa aaaaaaaaa |

HOMO SAPIENS INOSITOL-3-PHOSPHATE SYNTHASE 1 (ISYNA1), MRNA. (NM_016368.3)
SEQ ID NO: 20

| | |
|---|---|
| 1 | ccgcgctgtc cgccgccgct gcctgagtcg actctgcgcc gcccgccgcg atggaggccg |
| 61 | ccgcccagtt cttcgtcgag agcccggacg tggtctacgg ccccgaggcc atcgaggcgc |
| 121 | aatacgagta ccggacgacg cgcgtcagcc gcgagggtgg cgttctcaag gtgcacccca |
| 181 | cgtccacgcg cttcaccttc cggaccgccc ggcaggtgcc ccggctcggg gtcatgcttg |
| 241 | tcggctgggg cgggaacaac ggctccacac tcaccgccgc ggtgctggcc aatcgactgc |
| 301 | gtttgtcctg gcccacgcgc agcggccgca aggaggccaa ctactacggc tcgctgactc |
| 361 | aggcgggcac cgtgagcctg ggcctggacg ccgagggcca ggaggtgttc gtacccttca |
| 421 | gcgcggtgct gcccatggtg gcgcccaacg acctcgtgtt cgatggctgg gacatctcgt |
| 481 | cgctgaacct ggccgaggcg atgcggcgcg cgaaggtgct ggactggggg ctgcaggagc |
| 541 | aactgtggcc gcacatggag gccctgcggc cccggccttc tgtttacatc cccgaattca |
| 601 | tcgcggccaa ccagagcgcg cgcgcggaca acctcatccc aggctcgcgt gcgcagcagc |
| 661 | tggagcagat ccgcagggac atccgagact tccggtctag cgcggggctg gacaaagtca |
| 721 | tagtgctgtg gacggcgaac acggagcgct tctgtgaggt gattccaggc ctcaacgaca |
| 781 | cagccgagaa cctgctgcgc accattgagc tcggtctgga ggtgtcgccc tccacgctct |
| 841 | tcgccgtggc cagcatcctg gagggctgtg ccttcctcaa tgggtctccg cagaacaccc |
| 901 | tggtgcccgg agctcttgag ctcgcgtggc agcaccgggt ttttgtgggc ggagatgact |
| 961 | tcaagtcagg ccagaccaaa gtcaagtccg tgcttgtgga cttcctcatt ggctccggcc |
| 1021 | tcaagaccat gtccatcgtg agttacaacc acctgggcaa caacgatggg gagaacctat |
| 1081 | cggcgccatt gcagttccgc tctaaggagg tgtccaagag caacgtggtg gacgacatgg |
| 1141 | tgcagagcaa cccagtgctc tatacgcccg gcgaagagcc tgaccactgc gtggtcatca |
| 1201 | agtatgtgcc gtacgtgggt gacagcaagc gcgcgctgga tgagtatacc tcggagctga |
| 1261 | tgctgggcgg aaccaacaca ctggtgctgc acaacacgtg tgaggactcg ctgctggccg |
| 1321 | cacccatcat gctggaccta gcgctgctga ccgagctgtg ccagcgcgtg agcttctgca |
| 1381 | ctgacatgga ccccgagccg cagaccttcc accccgtgct gtccctgctc agcttcctct |
| 1441 | tcaaggcgcc actagtgccg cccggcagcc cggtggtcaa tgcgcttttc cgccagcgca |
| 1501 | gctgcatcga gaacatcctc agggcctgcg tggggctccc gccacagaac cacatgctcc |
| 1561 | tggaacacaa aatggagcgc ccagggccca gcctcaagcg agttggaccc gtggctgcca |
| 1621 | cctaccctat gttgaacaag aaaggaccgg tacccgctgc caccaatggc tgcaccggtg |
| 1681 | atgccaatgg gcatctgcaa gaggagcccc caatgcccac cacctgaggc cccggtcaca |
| 1741 | cagtttctcg gctcttcctc cccgctgccc cccacgaccc taccttgaag gcccccacaa |
| 1801 | ataaaggcgc tgccactcag ccctcaaaaa aaaaaaaaaa aaaaaaaaaa aa |

HOMO SAPIENS LIM DOMAIN ONLY 3 (RHOMBOTIN-LIKE 2) (LMO3),
TRANSCRIPT VARIANT 2, MRNA. (NM_001001395.1)
SEQ ID NO: 21

| | |
|---|---|
| 1 | gtagaacagt ggggagctgt gcggatgggt gaagtgcatg tatgcctgcc tagacgggcc |
| 61 | agaaaagcca aacttaagaa atctgcctat gtacagaaca agtcaactaa ggggttctca |
| 121 | ttaatctacc gtctgtgaat atcgttattt ttcaggtata caaatgctct cagtccagcc |
| 181 | agacaccaag ccgaaaggtt gtgctggctg caaccgaaag atcaaggacc ggtatcttct |
| 241 | aaaggcactg gacaaatact ggcatgaaga ctgcctgaag tgtgcctgct gtgactgtcg |
| 301 | cttgggagag gtgggctcca ccctgtacac taaagctaat cttatccttt gtcgcagaga |
| 361 | ctatctgagg ctctttggtg taacgggaaa ctgcgctgcc tgtagtaagc tcatccctgc |
| 421 | ctttgagatg gtgatgcgtg ccaaggacaa tgtttaccac ctggactgct ttgcatgtca |
| 481 | gctttgtaat cagagatttt gtgttggaga caaatttttc ctaaagaata acatgatcct |
| 541 | ttgccagacg gactacgagg aaggtttaat gaaagaaggt tatgcacccc aggttcgctg |
| 601 | atctatcaac atcaccccat taagaataca aagcactaca ttcttttatc ttttttgctc |
| 661 | cacatgtaca taagaattga cacaggaacc tactgaatag cgtagatata ggaaggcagg |
| 721 | atggttatat ggaataaaag gcggactgca tctgtatgta gtgaaattgc cccagttcag |
| 781 | agttgaatgt ttattattaa agaaaaaagt aatgtacata tggctggatt tttttgcttg |
| 841 | ctattcgttt ttgtgtcact tggcatgaga tgtttatttt ggactattgt atataatgta |
| 901 | ttgtaatatt tgaagcacaa atgtaataca gttttattgt gttaccattt gtgttccatt |
| 961 | tgcttctttg tattgttgca tttagtacaa tcagtgttta aacttactgt atatttatgc |
| 1021 | tttctgtatt taccagctat tttaaatgag ctgtaacttt ctagtaaaga attgaaaagc |
| 1081 | aaatctcact aatgatacac agatagataa agcaagtcta tcaacattaa aaatactaaa |
| 1141 | aaataaagac acacacagag cattttagtg acatccacta cttattgccg ctatgagtta |
| 1201 | gagtctatca gtgttcttgt tataaccccc tattttcagg gggttaaaaa tcagctttaa |
| 1261 | aaaaatacat aaaaatttca tcttaaagca ctttcatttt ataccaacgt gaaaagtgcc |
| 1321 | atttttagaa taactttaaa gcttaacagg tttcctttta atatcctttt tttgtgtgct |
| 1381 | ctttacttac acaatggctt tgttttgctt tttcagccac accccttatg tgaactagtg |
| 1441 | cctttgggta tcacgtaaaa ttttttccaa agggttactt taaaaatctg ttaccacaat |
| 1501 | tatgagatga tttttaagtg ataaattaaa cttcttcttg tataaattct gcccagatct |
| 1561 | ctccacaaga gctgagggtt tcataacttt atggcttaat aaatgtatga cactgaaaag |
| 1621 | atttgagtgt gaatctactg aaatcactat aatgcacatt gaagctatga tggtatttga |
| 1681 | gtagtgaggt tacttttgat cggagcaaca taatgctcat agaatcttct agaagaagag |
| 1741 | aaacaaaggg attgataaaa tgctgagaac tagtgattat atatttttct gtatttacct |
| 1801 | gacatttatt ttaatgttca aaaagtaaac actttaagtt tgatgtgttt tactctctca |
| 1861 | ttgttttaag taattgccaa ctcagaatac atcattctta ggctgaaatt tgtctttcca |
| 1921 | ttttttaagg tgaaatagta ctaccttacg tgatagcata caaagaagaa agctctagaa |
| 1981 | agagaaatta tggagaatga ttatttaaat tacaattaag gaaatgagaa tatgatcccc |
| 2041 | tcttccgagt tgcccacaaa cttgcttctt tgcttttgct ccctgtaata gaactacttt |
| 2101 | tcaacaaatc taattttgca cggcaccgtt aaccatattt tcactacagc aaacttagtg |
| 2161 | ctattcgttt tctttttctt tgtttttttc ttgatcactt gtataggaaa caatattttc |
| 2221 | cagtgttatt tgcatatata ttttgtcctt ccaatatatg cattacagat gaaaattaaa |
| 2281 | tgttatacct gaattcttgg gttggggcca aaatattaag ctgaaaataa tgctggtgtg |
| 2341 | gatttgtttt aaaacaaagc tttattatga acatgcatgt gaatctggat attgcctctt |
| 2401 | atttttaaga aaatggttct gtgaaaagtg aatgatatgt atttttccaa atgcttcatg |
| 2461 | gttaggagtc ttcaagttcc atgttcccca gatttgagat atactaaaga aagaaattca |
| 2521 | aaagtagcta tttggggccc acaaaaataa ctattatttt agccttagag ccttacactt |
| 2581 | gtttcatgaa gagaaaggac ttgcataacc aaaataaaca aagcaagaca aattaaaaat |
| 2641 | atgtggggga gagatcagtg aaaagtggtt ttcttaatgc agccctgctg gtccccatta |
| 2701 | acaattgctt gaaattcaca tggatgtaaa attataattg tcaggatctt attcagatga |
| 2761 | tcttttaagg tttaactggt tttgcttttg tttatctata tgtcaaaata cttgtaaatt |
| 2821 | gggaacaaac ttctctcagc ttcttgaagt tgttcaacta tccttgccac tggaagacca |
| 2881 | aacaaggttt tcactgcttt ttcttttaca taatatgctg agaattattt cttatgcttt |
| 2941 | ttactacaaa caaaattact cacctggatt aaagattaag gccttaatct gtttagatta |
| 3001 | tctttaatct ccatgaaatc gtgaaataag acaagaatag tgtttcagct gtaggccatt |
| 3061 | ttacagctaa ttgcccataa attgtagcat ttattgacct gaagtactaa gctaattgtc |
| 3121 | ttgactactc aaagcccctg aattgttgtc aactttcccc tttgtgttgt gtagccctaa |
| 3181 | cgtcatttag cttgttgtct gatgcctcca gtaggacacc tccgatggag ctttgatttc |
| 3241 | tgagcagcga aagctccctt cctaagatgc atctcgcata ggctgcctat gatgaaggac |
| 3301 | cgtgcacctc cactccaaca gagtgctgag tttaaaagtt gacctgtgtt tgtaatttca |
| 3361 | ctttcatctt gcttaataaa tatctgctgg attctttcat tcactttttt acatttggat |
| 3421 | ttatgttttt aataaaaggg gtgttacact |

HOMO SAPIENS MICRORNA 221 (MIR221), MICRORNA. (NR_029635.1)
SEQ ID NO: 22

| | |
|---|---|
| 1 | tgaacatcca ggtctggggc atgaacctgg catacaatgt agatttctgt gttcgttagg |
| 61 | caacagctac attgtctgct gggtttcagg ctacctggaa acatgttctc |

HOMO SAPIENS PROPROTEIN CONVERTASE SUBTILISIN/KEXIN TYPE 1
INHIBITOR (PCSK1N), MRNA. (NM_013271.2)
SEQ ID NO: 23

| | |
|---|---|
| 1 | tccggagccc ggctcgctgg ggcagcatgg cggggtcgcc gctgctctgg gggccgcggg |
| 61 | ccgggggcgt cggccttttg gtgctgctgc tgctcggcct gtttcggccg ccccccgcgc |
| 121 | tctgcgcgcg gccggtaaag gagccccgcg gcctaagcgc agcgtctccg cccttggctg |
| 181 | agactggcgc tcctcgccgc ttccggcggt cagtgccccg aggtgaggcg gcgggggcgg |
| 241 | tgcaggagct ggcgcgggcg ctggcgcatc tgctggaggc cgaacgtcag gagcgggcgc |
| 301 | gggccgaggc gcaggaggct gaggatcagc aggcgcgcgt cctggcgcag ctgctgcgcg |
| 361 | tctggggcgc cccccgcaac tctgatccgg ctctgggcct ggacgacgac cccgacgcgc |
| 421 | ctgcagcgca gctcgctcgc gctctgctcc gcgcccgcct tgaccctgcc gccctcgcag |
| 481 | cccagcttgt ccccgcgccc gtccccgccg cggcgctccg accccggccc ccggtctacg |
| 541 | acgacggccc cgcgggcccg gatgctgagg aggcaggcga cgagacaccc gacgtggacc |
| 601 | ccgagctgtt gaggtacttg ctgggacgga ttcttgcggg aagcgcggac tccgaggggg |
| 661 | tggcagcccc gcgccgcctc cgccgtgccg ccgaccacga tgtgggctct gagctgcccc |
| 721 | ctgagggcgt gctgggggcg ctgctgcgtg tgaaacgcct agagaccccg gcgccccagg |
| 781 | tgcctgcacg ccgcctcttg ccaccctgag cactgcccgg atcccgtgca ccctgggacc |
| 841 | cagaagtgcc cccgccatcc cgccaccagg actgctcccc gccagcacgt ccagagcaac |
| 901 | ttaccccggc cagccagccc tctcacccga ggatccctac cccctggccc cacaataaac |
| 961 | atgatctgaa gcaaaaaaaa aaaaaaaaaa |

HOMO SAPIENS SECRETOGRANIN V (7B2 PROTEIN) (SCG5), MRNA. (NM_003020A)
SEQ ID NO: 24

| | |
|---|---|
| 1 | cgctcctcgg gctgcccctc ggttgacaat ggtctccagg atggtctcta ccatgctatc |
| 61 | tggcctactg ttttggctgg catctggatg gactccagca tttgcttaca gcccccggac |
| 121 | ccctgaccgg gtctcagaag cagatatcca gaggctgctt catggtgtta tggagcaatt |
| 181 | gggcattgcc aggccccgag tggaatatcc agctcaccag gccatgaatc ttgtgggccc |
| 241 | ccagagcatt gaaggtggag ctcatgaagg acttcagcat ttgggtcctt ttggcaacat |
| 301 | ccccaacatc gtggcagagt tgactggaga caacattcct aaggacttta gtgaggatca |
| 361 | ggggtaccca gaccctccaa atccctgtcc tgttggaaaa acagatgatg gatgtctaga |
| 421 | aaacacccct gacactgcag agttcagtcg agagttccag ttgcaccagc atctctttga |
| 481 | tccggaacat gactatccag gcttgggcaa gtggaacaag aaactccttt acgagaagat |
| 541 | gaagggagga gagagacgaa agcggaggag tgtcaatcca tatctacaag gacagagact |
| 601 | ggataatgtt gttgcaaaga agtctgtccc ccatttttca gatgaggata aggatccaga |
| 661 | gtaaagagaa gatgctagac gaaaacccac attacctgtt aggcctcagc atggcttatg |
| 721 | tgcacgtgta aatggagtcc ctgtgaatga cagcatgttt cttacataga taattatgga |
| 781 | tacaaagcag ctgtatgtag atagtgtatt gtcttcacac cgatgattct gctttttgct |
| 841 | aaattagaat aagagctttt ttgtttcttg ggtttttaaa atgtgaatct gcaatgatca |
| 901 | taaaaattaa aatgtgaatg tcaacaataa aaagcaagac tatgaaaggc tcagatttct |
| 961 | tgcagtttaa aatggtgtct gaggttgtac tattttggcc aagtctgtag aaagctgtca |
| 1021 | tttgattttg attatgtagt tcatccagcc cttgggcatt gttatacacc agtaaagaag |
| 1081 | gctgtactca agaggaggag ctgacacatt tcacttggct gcgtcttaat aaacatgaat |
| 1141 | gcaagcattg gc |

DKFZP781A1072_S1 781 (SYNONYM: HLCC4) HOMO SAPIENS CDNA CLONE
DKFZP781A1072 3, MRNA SEQUENCE (BX955517)
SEQ ID NO: 25
TTTTTTTTTTTTTTTTTTTCTTTTAACGGGCTTCAATTGCAGAGTTGAATCATTCATTAT
TTTCAGTTCATAATACATTGTCATCATTGGTCCAGATAGGTTGAATTCTTATTTCATTCA
TTCATTACTCATTCATTTATTCATTCAGTTACAGTGCCATCAATGTCCATACACCCACCG
CTCTACTCAAGAACTTGAACTGCTATTTACCTATATGTTCCTCTATTATCCTACCTCCTA
CCTCCCCACACAGCAGCAATCAGTATCCAGAAGTTTATGATTATCACTCCTTTGCTTTAA
ACAGTTGGATTACATGTATATGTATGCCTAAATTATAGTTTTGTGGGGTTTTTGTGAACC
CCACAAGAGTGAAATGTTCTAAGTCTTGTGGGACTTGTTTTCATTTAATATCTTATTGAA
TGACACTCATATTGTTGTGGGTAGCTGTAGGTCACTCCACTTCACAGTTGTGTAATATTC
TGTCATGTGATTTTACCATAAGCTCTCCACCCATTTTCCTGAGGATAGCTATTTGGATTA
TTTCCAGTTCTTTATTAAGAACAATACTATCTTGAACATCCTTATATGTGTATTTCCTAG
TACTCCTTTATAAAAATTTCCTTTGGAAATATACCAAGAATTGGAAATTTGGGGTTGTAA
GG
PREDICTED: HOMO SAPIENS CYSTATIN E/M (CST6), MRNA. (XM_001129442.1)
SEQ ID NO: 26

| | |
|---|---|
| 1 | atgtaccttg tgacagtgtg gcctctcaga tccagcccga ctcagccagc aggacaaagg |
| 61 | gaccacggct tggaatgctg gcgcagaaag cagacctggc tgctggtggc cggcatcaag |
| 121 | tacttcctga cgatggagat ggggagcaca gactgccgca agaccagggt cactggagac |
| 181 | cacgtcgacc tcaccacttg ccccctggca gcaggggcgc agcaggagaa gctgcgctgt |
| 241 | gactttgagg tccttgtggt tccctggcag aactcctctc agctcctaaa gcacaactgt |
| 301 | gtgcagatgt gataagtccc cgagggcgaa ggccattggg tttggggcca tggtggaggg |
| 361 | cacttcaggt ccgtgggccg tatctgtcac aataaatggc cagtgctgct tcttgcattg |
| 421 | gtttcttcca agtg |

HOMO SAPIENS SURFACTANT PROTEIN B (SFTPB), TRANSCRIPT VARIANT 1,
MRNA. (NM_000542.2)
SEQ ID NO: 27

| | |
|---|---|
| 1 | gccatggctg agtcacacct gctgcagtgg ctgctgctgc tgctgcccac gctctgtggc |
| 61 | ccaggcactg ctgcctggac cacctcatcc ttggcctgtg cccagggccc tgagttctgg |
| 121 | tgccaaagcc tggagcaagc attgcagtgc agagccctag ggcattgcct acaggaagtc |
| 181 | tggggacatg tgggagccga tgacctatgc caagagtgtg aggacatcgt ccacatcctt |
| 241 | aacaagatgg ccaaggaggc cattttccag gacacgatga ggaagttcct ggagcaggag |
| 301 | tgcaacgtcc tccccttgaa gctgctcatg ccccagtgca accaagtgct tgacgactac |
| 361 | ttccccctgg tcatcgacta cttccagaac cagactgact caaacggcat ctgtatgcac |
| 421 | ctgggcctgt gcaaatcccg gcagccagag ccagagcagg agccagggat gtcagacccc |
| 481 | ctgcccaaac ctctgcggga ccctctgcca gaccctctgc tggacaagct cgtcctccct |
| 541 | gtgctgcccg gggccctcca ggcgaggcct gggcctcaca cacaggatct ctccgagcag |
| 601 | caattcccca ttcctctccc ctattgctgg ctctgcaggg ctctgatcaa gcggatccaa |
| 661 | gccatgattc ccaagggtgc gctagctgtg gcagtggccc aggtgtgccg cgtggtacct |
| 721 | ctggtggcgg gcggcatctg ccagtgcctg gctgagcgct actccgtcat cctgctcgac |
| 781 | acgctgctgg gccgcatgct gccccagctg gtctgccgcc tcgtcctccg gtgctccatg |
| 841 | gatgacagcg ctggcccaag gtcgccgaca ggagaatggc tgccgcgaga ctctgagtgc |
| 901 | cacctctgca tgtccgtgac cacccaggcc gggaacagca gcgagcaggc cataccacag |
| 961 | gcaatgctcc aggcctgtgt tggctcctgg ctggacaggg aaaagtgcaa gcaatttgtg |
| 1021 | gagcagcaca cgccccagct gctgaccctg gtgcccaggg gctgggatgc ccacaccacc |
| 1081 | tgccaggccc tcggggtgtg tgggaccatg tccagccctc tccagtgtat ccacagcccc |
| 1141 | gacctttgat gagaactcag ctgtccagct gcaaaggaaa agccaagtga gacgggctct |
| 1201 | gggaccatgg tgaccaggct cttcccctgc tccctggccc tcgccagctg ccaggctgaa |
| 1261 | aagaagcctc agctcccaca ccgccctcct caccgccctt cctcggcagt cacttccact |
| 1321 | ggtggaccac gggcccccag ccctgtgtcg gccttgtctg tctcagctca accacagtct |
| 1381 | gacaccagag cccacttcca tcctctctgg tgtgaggcac agcgagggca gcatctggag |
| 1441 | gagctctgca gcctccacac ctaccacgac ctcccagggc tgggctcagg aaaaaccagc |
| 1501 | cactgcttta caggacaggg ggttgaagct gagccccgcc tcacacccac ccccatgcac |
| 1561 | tcaaagattg gattttacag ctacttgcaa ttcaaaattc agaagaataa aaaatgggaa |
| 1621 | catacagaac tctaaaagat agacatcaga aattgttaag ttaagctttt tcaaaaaatc |
| 1681 | agcaattccc cagcgtagtc aagggtggac actgcacgct ctggcatgat gggatggcga |
| 1741 | ccgggcaagc tttcttcctc gagatgctct gctgcttgag agctattgct ttgttaagat |
| 1801 | ataaaaaggg gtttcttttt gtctttctgt aaggtggact tccagctttt gattgaaagt |
| 1861 | cctagggtga ttctatttct gctgtgattt atctgctgaa agctcagctg gggttgtgca |
| 1921 | agctagggac ccattcctgt gtaatacaat gtctgcacca atgctaataa agtcctattc |
| 1981 | tcttttatga aaaaaaaaaa aaaaaaaaaa aaaaaaaaaa aaaaaa |

HOMO SAPIENS SOLUTE CARRIER FAMILY 27 (FATTY ACID TRANSPORTER),
MEMBER 6 (SLC27A6), TRANSCRIPT VARIANT 1, MRNA (NM_014031.3)
SEQ ID NO: 28

| | |
|---|---|
| 1 | gacgtggtgc tgagcccctg cgcggtttct ggtgcgtaga gactgtaaat cgctgcgctt |
| 61 | ctcagtcatc atcatcccag cttttcccgg ctcgaattca gcctccaact caagctcgcg |
| 121 | ggaaagacta cctgagagga gaaaagcttc tgtccctgga ccttcttctg agggtggtga |
| 181 | ggtttgttta gggtcgcaga agcagggagg actgactcag ccctcacaga gaagaggatt |
| 241 | cctccccatc ccgcttcgcc ccggaaaagc tgacaagaac ttcaggtgta agccctgagt |
| 301 | agtgaggatc tgcggtctcc gtggagagct gtgcctggaa gagaaggacg ctggtggggg |
| 361 | ctgagatcag agctgtcttc tggcccagtt gcccccatgc ttctgtcatg gctaacagtt |
| 421 | ctaggggctg gaatggtcgt cctgcacttc ttgcagaaac tcctgttccc ttacttttgg |
| 481 | gatgacttct ggttcgtgtt gaaggtggtg ctcattataa ttcggctgaa gaagtatgaa |
| 541 | aagagagggg agctggtgac tgtgctggat aaattcttga gtcatgccaa aagacaacct |
| 601 | cggaaacctt tcatcatcta tgagggagac atctacacct atcaggatgt agacaaaagg |
| 661 | agcagcagag tggcccatgt cttcctgaac cattcctctc tgaaaaaggg ggacacggtg |
| 721 | gctctgctga tgagcaatga gccggacttc gttcacgtgt ggttcggcct cgccaagctg |
| 781 | ggctgcgtgg tggcctttct caacaccaac attcgctcca actccctcct gaattgcatc |
| 841 | cgcgcctgtg ggcccagagc cctagtggtg ggcgcagatt tgcttggaac ggtagaagaa |
| 901 | atccttccaa gcctctcaga aaatatcagt gtttggggga tgaaagattc tgttccacaa |
| 961 | ggtgtaattt cactcaaaga aaaactgagc acctcacctg atgagcccgt gccacgcagc |
| 1021 | caccatgttg tctcactcct caagtctact tgtctttaca tttttacctc tggaacaaca |
| 1081 | ggtctaccaa aagcagctgt gattagtcag ctgcaggttt taaggggttc tgctgtcctg |
| 1141 | tgggcttttg gttgtactgc tcatgacatt gtttatataa cccttcctct gtatcatagt |
| 1201 | tcagcagcta tcctgggaat ttctggatgt gttgagttgg gtgccacttg tgtgttaaag |
| 1261 | aagaaatttt cagcaagcca gttttggagt gactgcaaga agtatgatgt gactgtgttt |
| 1321 | cagtatattg gagaactttg tcgctacctt tgcaaacaat ctaagagaga aggagaaaag |
| 1381 | gatcataagg tgcgtttggc aattggaaat ggcatacgga gtgatgtatg gagagaattt |
| 1441 | ttagacagat ttggaaatat aaaggtgtgt gaactttatg cagctaccga atcaagcata |
| 1501 | tctttcatga actacactgg gagaattgga gcaattggga gaacaaattt gttttacaaa |
| 1561 | cttctttcca cttttgactt aataaagtat gactttcaga aagatgaacc catgagaaat |
| 1621 | gagcagggtt ggtgtattca tgtgaaaaaa ggagaacctg gacttctcat ttctcgagtg |
| 1681 | aatgcaaaaa atcccttctt tggctatgct gggccttata agcacacaaa agacaaattg |
| 1741 | ctttgtgatg tttttaagaa gggagatgtt taccttaata ctggagactt aatagtccag |
| 1801 | gatcaggaca atttccttta tttttgggac cgtactggag acactttcag atggaaagga |
| 1861 | gaaaatgtcg caaccactga ggttgctgat gttattggaa tgttggattt catacaggaa |
| 1921 | gcaaacgtct atggtgtggc tatatcaggt tatgaaggaa gagcaggaat ggcttctatt |
| 1981 | attttaaaac caaatacatc tttagatttg gaaaaagttt atgaacaagt tgtaacattt |
| 2041 | ctaccagctt atgcttgtcc acgattttta agaattcagg aaaaaatgga agcaacagga |
| 2101 | acattcaaac tattgaagca tcagttggtg gaagatggat ttaatccact gaaaatttct |
| 2161 | gaaccacttt acttcatgga taacttgaaa aagtcttatg ttctactgac cagggaactt |
| 2221 | tatgatcaaa taatgttagg ggaaataaaa ctttaagatt tttatatcta gaactttcat |
| 2281 | atgctttctt aggaagagtg agaggggggt atatgattct ttatgaaatg gggaaaggga |
| 2341 | gctaacatta attatgcatg tactatattt ccttaatatg agagataatt ttttaattgc |
| 2401 | ataagaattt taatttcttt taattgatat aaacagtagt tgattattct ttttatctat |
| 2461 | ttggagattc agtgcataac taagtatttt ccttaatact aaagatttta aataataaat |
| 2521 | agtggctagc ggtttggaca atcactaaaa atgtactttc taataagtaa aatttctaat |
| 2581 | tttgaataaa agattaaatt ttactgaaat attttaa |

HOMO SAPIENS TRANSMEMBRANE PROTEIN 233 (TMEM233), MRNA. (NM_001136534A)
SEQ ID NO: 29

| | |
|---|---|
| 1 | aatttggaac ttaatgggcc tttgcgtcct ccttccctga gcctcctttt attccagact |
| 61 | tctcagtgtg agtctgtgcg tccctccgac gatctcaggg agtggggtgc cttcatctgc |
| 121 | ctgttccctg ttcctcaggc tgacgctccc gctgtcctcc ccgcctcccc tcactccttt |
| 181 | tctccctccc ttcctccttg tggggaggct cttggccagg gtccctgagc ccgggcgggt |
| 241 | gctggcagag gacgcagaag gggtgaggtc acgtctccct tgagccccga gccgctggct |
| 301 | tttcagagcc tcgccacaag ccggcggcca gagccccaga ccacacagac cgtgcgctcc |
| 361 | tccgccctcc cggcgccgcc ggcctcgccc atgtctcagt acgcccctag cccggacttc |
| 421 | aagagggctt tggacagcag tcccgaggcc aacactgaag atgacaagac cgaggaggac |
| 481 | gtgcccatgc ccaagaacta cctgtggctc accatcgtct cgtgtttttg ccctgcgtac |
| 541 | cccatcaaca tcgtggcttt ggtcttttcc atcatgtctc tgaacagcta caacgatgga |
| 601 | gactacgaag gagccaggcg gcttgggcgg aatgctaagt gggtagccat cgcctccatc |
| 661 | atcattggcc ttctcatcat cggcatttct tgtgcagttc acttcacaag gaatgcctga |
| 721 | ggaaccagcg gtcagtgggc tgtgagcgtg gaggatggac ctcatccaca cacaccccaa |
| 781 | aggagtttct aaggaatgga tccttgactt cagactgtga gatcttttcc tccaggactc |
| 841 | tccagaggca ggtccctggc aaatgaacaa gaaaaaaaaa aaaaaaaagt ccaaaattta |
| 901 | ggcaatccaa gctgcacagc cggatcagcc aaagtcattg atttgtaaaa atgaaaagaa |
| 961 | aacagaaaaa agaaaaatga agtctcactg tctcagttta gcgaatcccg ttgtgtccac |
| 1021 | tcctgtcctc cagaggcgag cctcaggaaa tcacataact tttcactgag gggatccagg |
| 1081 | gggtctccat atagggggag atggaggttt ctaggaagag cagcaggtgc tggtatttac |
| 1141 | aatgttgagc acaaacattt gcagcatgtt taaaattgtc tagtagagtt caagttgtgg |
| 1201 | atttgctttt ccttttattc ttataacctt cagtaactcc tcctctggga gtcagcactc |
| 1261 | ccatgcccag agttcaccca tctggtcatc aaacactcaa agaaggggct tttctggcct |
| 1321 | tttgtcttga tgcttatatt tccaaatagg ccccctccct tgcttgcatc cacgttggtc |
| 1381 | aacttgacca aaacctcact cttcactcaa acaggctctg agaatggact tagtggccaa |
| 1441 | ttctaggtac atgagcactt cctgtatccc agttttggga ataaactggc tgtatttata |
| 1501 | gaatgtgctt tttttttttc aatttctcac tctctctcct atctctagca agtctcaggc |
| 1561 | aagatctttg att |

IMMUNOGLOBULIN SUPERFAMILY MEMBER 1 ISOFORM 1 PRECURSOR [HOMO SAPIENS]
(NP_001546.2)
SEQ ID NO: 30

| | |
|---|---|
| 1 | mtldrpgega tmlktftvll fcirmslgmt sivmdpqpel wiesnypqap wenitlwcrs |
| 61 | psrisskfll lkdktqmtwi rpshktfqvs fligaltesn aglyrccywk etgwskpskv |
| 121 | leleapgqlp kpifwiqaet palpgcnvni lchgwlqdlv fmlfkegyae pvdyqvptgt |
| 181 | maifsidnlt pedegvyicr thiqmlptlw sepsnplklv vaglypkptl tahpgpimap |
| 241 | geslnlrcqg piygmtfalm rvedleksfy hkktiknean fffqslkiqd tghylcfyyd |
| 301 | asyrgsllsd vlkiwvtdtf pktwllarps avvqmgqnvs lrcrgpvdgv glalykkged |
| 361 | kplqfldats iddntsffln nvtysdtgiy schylltwkt sirmpshntv elmvvdkppk |
| 421 | pslsawpstv fklgkaitlq crvshpvlef sleweeretf qkfsvngdfi isnvdgkgtg |
| 481 | tyscsyrvet hpniwshrse plklmgpagy ltwnyvlnea irlslimqlv alllvvlwir |
| 541 | wkcrrlrire awllgtaqgv tmlfivtall ccglcngvli eeteivmptp kpelwaetnf |
| 601 | plapwknltl wcrspsgstk efvllkdgtg wiatrpaseq vraafplgal tqshtgsyhc |
| 661 | hsweemavse psealelvgt dilpkpvisa sptirgqelq lrckgwlagm gfalykegeq |
| 721 | epvqqlgavg reafftiqrm edkdegnysc rthtekrpfk wsepseplel vikemypkpf |
| 781 | fktwaspvvt pgarvtfncs tphqhmsfil ykdgseiass drswaspgas aahfliisvg |
| 841 | igdggnyscr yydfsiwsep sdpvelvvte fypkptllaq pgpvvfpgks vilrcqgtfq |
| 901 | gmrfallqeg ahvplqfrsv sgnsadfllh tvgaedsgny sciyyettms nrgsylsmpl |
| 961 | miwvtdtfpk pwlfaepssv vpmgqnvtlw crgpvhgvgy ilhkegeats mqlwgstsnd |
| 1021 | gafpitnisg tsmgrysccy hpdwtssiki qpsntlellv tgllpkpsll aqpgpmvapg |
| 1081 | enmtlqcqge lpdstfvllk egaqepleqq rpsgyradfw mpavrgedsg iyscvyylds |
| 1141 | tpfaasnhsd sleiwvtdkp pkpslsawps tmfklgkdit lqcrgplpgv efvlehdgee |
| 1201 | apqqfsedgd fvinnvegkg ignyscsyrl qaypdiwsep sdplelvgaa gpvagectvg |
| 1261 | nivrsslivv vvvalgvvla iewkkwprlr trgsetdgrd qtialeecnq egepgtpans |
| 1321 | psstsqrisv elpvpi |

HOMO SAPIENS POLY (ADP-RIBOSE) POLYMERASE FAMILY, MEMBER 1 (PARD1),
MRNA (NM_00161 8.2)
SEQ ID NO: 31

| | |
|---|---|
| 1 | aatctatcag ggaacggcgg tggccggtgc ggcgtgttcg gtgcgctctg gccgctcagg |
| 61 | ccgtgcggct gggtgagcgc acgcgaggcg gcgaggcggc aagcgtgttt ctaggtcgtg |
| 121 | gcgtcgggct tccggagctt tggcggcagc taggggagga tggcggagtc ttcggataag |
| 181 | ctctatcgag tcgagtacgc caagagcggg cgcgcctctt gcaagaaatg cagcgagagc |
| 241 | atccccaagg actcgctccg gatggccatc atggtgcagt cgcccatgtt tgatggaaaa |
| 301 | gtcccacact ggtaccactt ctcctgcttc tggaaggtgg gccactccat ccggcaccct |
| 361 | gacgttgagg tggatgggtt ctctgagctt cggtgggatg accagcagaa agtcaagaag |
| 421 | acagcggaag ctggaggagt gacaggcaaa ggccaggatg gaattggtag caaggcagag |
| 481 | aagactctgg gtgactttgc agcagagtat gccaagtcca acagaagtac gtgcaagggg |
| 541 | tgtatggaga agatagaaaa gggccaggtg cgcctgtcca agaagatggt ggacccggag |
| 601 | aagccacagc taggcatgat tgaccgctgg taccatccag gctgctttgt caagaacagg |
| 661 | gaggagctgg gtttccggcc cgagtacagt gcgagtcagc tcaagggctt cagcctcctt |
| 721 | gctacagagg ataaagaagc cctgaagaag cagctcccag gagtcaagag tgaaggaaag |
| 781 | agaaaaggcg atgaggtgga tggagtggat gaagtggcga agaagaaatc taaaaaagaa |
| 841 | aaagacaagg atagtaagct tgaaaaagcc ctaaaggctc agaacgacct gatctggaac |
| 901 | atcaaggacg agctaaagaa agtgtgttca actaatgacc tgaaggagct actcatcttc |
| 961 | aacaagcagc aagtgccttc tggggagtcg gcgatcttgg accgagtagc tgatggcatg |
| 1021 | gtgttcggtg ccctccttcc ctgcgaggaa tgctcgggtc agctggtctt caagagcgat |
| 1081 | gcctattact gcactgggga cgtcactgcc tggaccaagt gtatggtcaa gacacagaca |
| 1141 | cccaaccgga aggagtgggt aaccccaaag gaattccgag aaatctctta cctcaagaaa |
| 1201 | ttgaaggtta aaaagcagga ccgtatattc cccccagaaa ccagcgcctc cgtggcggcc |
| 1261 | acgcctccgc cctccacagc ctcggctcct gctgctgtga actcctctgc ttcagcagat |
| 1321 | aagccattat ccaacatgaa gatcctgact ctcgggaagc tgtcccggaa caaggatgaa |
| 1381 | gtgaaggcca tgattgagaa actcgggggg aagttgacgg ggacggccaa caaggcttcc |
| 1441 | ctgtgcatca gcaccaaaaa ggaggtggaa aagatgaata agaagatgga ggaagtaaag |
| 1501 | gaagccaaca tccgagttgt gtctgaggac ttcctccagg acgtctccgc ctccaccaag |
| 1561 | agccttcagg agttgttctt agcgcacatc ttgtcccctt ggggggcaga ggtgaaggca |
| 1621 | gagcctgttg aagttgtggc cccaagaggg aagtcagggg ctgcgctctc caaaaaaagc |
| 1681 | aagggccagg tcaaggagga aggtatcaac aaatctgaaa agagaatgaa attaactctt |
| 1741 | aaaggaggag cagctgtgga tcctgattct ggactggaac actctgcgca tgtcctggag |
| 1801 | aaaggtggga aggtcttcag tgccaccctt ggcctggtgg acatcgttaa aggaaccaac |
| 1861 | tcctactaca agctgcagct tctggaggac gacaaggaaa acaggtattg gatattcagg |
| 1921 | tcctggggcc gtgtgggtac ggtgatcggt agcaacaaac tggaacagat gccgtccaag |
| 1981 | gaggatgcca ttgagcagtt catgaaatta tatgaagaaa aaaccgggaa cgcttggcac |
| 2041 | tccaaaaatt tcacgaagta tcccaaaaag ttttaccccc tggagattga ctatggccag |
| 2101 | gatgaagagg cagtgaagaa gctcacagta aatcctggca ccaagtccaa gctccccaag |
| 2161 | ccagttcagg acctcatcaa gatgatcttt gatgtggaaa gtatgaagaa agccatggtg |
| 2221 | gagtatgaga tcgaccttca gaagatgccc ttggggaagc tgagcaaaag gcagatccag |
| 2282 | gccgcatact ccatcctcag tgaggtccag caggcggtgt ctcagggcag cagcgactct |
| 2341 | cagatcctgg atctctcaaa tcgcttttac accctgatcc cccacgactt tgggatgaag |
| 2401 | aagcctccgc tcctgaacaa tgcagacagt gtgcaggcca aggtggaaat gcttgacaac |
| 2461 | ctgctggaca tcgaggtggc ctacagtctg ctcaggggag ggtctgatga tagcagcaag |
| 2521 | gatcccatcg atgtcaacta tgagaagctc aaaactgaca ttaaggtggt tgacagagat |
| 2581 | tctgaagaag ccgagatcat caggaagtat gttaagaaca ctcatgcaac cacacacagt |
| 2641 | gcgtatgact tggaagtcat cgatatcttt aagatagagc gtgaaggcga atgccagcgt |
| 2701 | tacaagccct ttaagcagct tcataaccga agattgctgt ggcacgggtc caggaccacc |
| 2761 | aactttgctg ggatcctgtc ccagggtctt cggatagccc cgcctgaagc gcccgtgaca |
| 2821 | ggctacatgt ttggtaaagg gatctatttc gctgacatgg tctccaagag tgccaactac |
| 2881 | taccatacgt ctcagggaga cccaataggc ttaatcctgt tgggagaagt tgcccttgga |
| 2941 | aacatgtatg aactgaagca cgcttcacat atcagcaggt tacccaaggg caagcacagt |
| 3001 | gtcaaaggtt tgggcaaaac tacccctgat ccttcagcta acattagtct ggatggtgta |
| 3061 | gacgttcctc ttgggaccgg gatttcatct ggtgtgatag acacctctct actatataac |
| 3121 | gagtacattg tctatgatat tgctcaggta aatctgaagt atctgctgaa actgaaattc |
| 3181 | aattttaaga cctccctgtg gtaattggga gaggtagccg agtcacaccc ggtggctgtg |
| 3241 | gtatgaattc acccgaagcg cttctgcacc aactcacctg gccgctaagt tgctgatggg |
| 3301 | tagtacctgt actaaaccac ctcagaaagg attttacaga aacgtgttaa aggttttctc |
| 3361 | taacttctca agtcccttgt tttgtgttgt gtctgtgggg aggggttgtt ttggggttgt |
| 3421 | ttttgttttt tcttgccagg tagataaaac tgacatagag aaaaggctgg agagagattc |
| 3481 | tgttgcatag actagtccta tggaaaaaac caaagcttcg ttagaatgtc tgccttactg |
| 3541 | gtttccccag ggaaggaaaa atacacttcc accctttttt ctaagtgttc gtctttagtt |
| 3601 | ttgattttgg aaagatgtta agcatttatt tttagttaaa ataaaaacta atttcatact |
| 3661 | atttagattt tcttttttat cttgcactta ttgtcccctt tttagttttt tttgtttgcc |
| 3721 | tcttgtggtg aggggtgtgg gaagaccaaa ggaaggaacg ctaacaattt ctcatactta |
| 3781 | gaaacaaaaa gagctttcct tctccaggaa tactgaacat gggagctctt gaaatatgta |
| 3841 | gtattaaaag ttgcatttg |

HOMO SAPIENS GLYCERALDEHYDE-3-PHOSPHATE DEHYDROGENASE (GAPDH), MRNA
(NM_002046.2)
SEQ ID NO: 96

| | |
|---|---|
| 1 | ctctctgctc ctcctgttcg acagtcagcc gcatcttctt ttgcgtcgcc agccgagcca |
| 61 | catcgctcag acaccatggg gaaggtgaag gtcggagtca acggatttgg tcgtattggg |
| 121 | cgcctggtca ccagggctgc ttttaactct ggtaaagtgg atattgttgc catcaatgac |
| 181 | cccttcattg acctcaacta catggtttac atgttccaat atgattccac ccatggcaaa |
| 241 | ttccatggca ccgtcaaggc tgagaacggg aagcttgtca tcaatggaaa tcccatcacc |
| 301 | atcttccagg agcgagatcc ctccaaaatc aagtggggcg atgctggcgc tgagtacgtc |
| 361 | gtggagtcca ctggcgtctt caccaccatg gagaaggctg gggctcattt gcagggggga |
| 421 | gccaaaaggg tcatcatctc tgccccctct gctgatgccc ccatgttcgt catgggtgtg |
| 481 | aaccatgaga agtatgacaa cagcctcaag atcatcagca atgcctcctg caccaccaac |
| 541 | tgcttagcac ccctggccaa ggtcatccat gacaactttg gtatcgtgga aggactcatg |
| 601 | accacagtcc atgccatcac tgccacccag aagactgtgg atggcccctc cgggaaactg |
| 661 | tggcgtgatg gccgcggggc tctccagaac atcatccctg cctctactgg cgctgccaag |
| 721 | gctgtgggca aggtcatccc tgagctgaac gggaagctca ctggcatggc cttccgtgtc |
| 781 | cccactgcca acgtgtcagt ggtggacctg acctgccgtc tagaaaaacc tgccaaatat |
| 841 | gatgacatca agaaggtggt gaagcaggcg tcggagggcc ccctcaaggg catcctgggc |
| 901 | tacactgagc accaggtggt ctcctctgac ttcaacagcg acacccactc ctccaccttt |
| 961 | gacgctgggg ctggcattgc cctcaacgac cactttgtca agctcatttc ctggtatgac |
| 1021 | aacgaatttg gctacagcaa cagggtggtg gacctcatgg cccacatggc ctccaaggag |
| 1081 | taagacccct ggaccaccag ccccagcaag agcacaagag gaagagagag accctcactg |
| 1141 | ctggggagtc cctgccacac tcagtccccc accacactga atctcccctc ctcacagttg |
| 1201 | ccatgtagac cccttgaaga ggggaggggc ctagggagcc gcaccttgtc atgtaccatc |
| 1261 | aataaagtac cctgtgctca acc |

TABLE 1

| | | | SEQ | | |
|---|---|---|---|---|---|
| Symbol | Definition | Accession | ID NO. | Probe_Id | Probe_Sequence |
| IGSF1 | Homo sapi [symbol] | NM_00155 [symbol] | 96 | ILMN_1679299 | CCCTGCAAGTCAGCCC |
| | | | | | CATCTGCTGTTCCTTG |
| | | | | | GTCTCTAATCACCTGA |
| | | | | | GC |
| IGSF21 | Homo sapi [symbol] | NM_03288 [symbol] | 97 | ILMN_1730039 | ACCTTGGTGCTCGCCC |
| | | | | | TGACAGTGATTCTGGA |
| | | | | | GCTGACGTGAAGGCAC |
| | | | | | CC |
| TM7SF4 | Homo sapi [symbol] | NM_03078 [symbol] | 98 | ILMN_1793730 | GCAGCACCTGGTTATG |
| | | | | | CCTCCTTTCATCTCAA |
| | | | | | AGCCAAAGAGCTGCCA |
| | | | | | GG |
| FLJ30058 | Homo sapi [symbol] | NM_14496 [symbol] | 99 | ILMN_1705466 | GTACAGTTTTGCTCAG |
| | | | | | GTCACGCCAACAGGGA |
| | | | | | AACCTCAAGTGTAGGT |
| | | | | | CT |
| CITED1 | Homo sapi [symbol] | NM_00414 [symbol] | 100 | ILMN_1691641 | GCTCCCACTAGTTCCT |
| | | | | | CGGGATCTCCAATAGG |
| | | | | | CTCTCCTACAACCACC |
| | | | | | CC |
| ZCCHC12 | Homo sapi [symbol] | NM_17379 [symbol] | 101 | ILMN_1679984 | CCCTGCAGCCTACGGG |
| | | | | | TCTGTTTTCTGTGTGT |
| | | | | | GCCCATTTCCTTGACA |
| | | | | | GC |
| CLDN16 | Homo sapi [symbol] | NM_00658 [symbol] | 102 | ILMN_1707670 | CAGCCCCTCGCACAGA |
| | | | | | GACGGCCAAAATGTAT |
| | | | | | GCTGTAGACACAAGGG |
| | | | | | TG |
| FN1 | Homo sapi [symbol] | NM_00202 [symbol] | 103 | ILMN_1778237 | GCAGGTGGAAGTGTGA |
| | | | | | TCCCGTCGACCAATGC |
| | | | | | CAGGATTCAGAGACTG |
| | | | | | GG |
| SERPINA1 | Homo sapi [symbol] | NM_00029 [symbol] | 104 | ILMN_1764980 | AGTGGACTTAGCCCCT |
| | | | | | GTTTGCTCCTCCGATA |
| | | | | | ACTGGGGTGACCTTGG |
| | | | | | TT |
| STK32A | Homo sapi [symbol] | NM_14500 [symbol] | 105 | ILMN_1756612 | GGTCATGGCCCTGGAC |
| | | | | | TACCTGCAGAACCAGC |
| | | | | | GCATCATTCACAGGGA |
| | | | | | TA |
| UNQ9433 | Homo sapi [symbol] | NM_20741 [symbol] | 106 | ILMN_2091217 | AGACTTCCCAGAAATA |
| | | | | | ACTGGTTAGCTGTTTC |
| | | | | | CTGTCATAGAATGGAG |
| | | | | | TC |
| BC030766 | Homo sapi [symbol] | BC030766 | 107 | ILMN_1904578 | CTCTGGCTGCAGTTAA |
| | | | | | ATGGTCTTTTGCATTT |
| | | | | | TGCTCTGGCTTTCAGG |
| | | | | | CC |
| AK023519 | Homo sapi [symbol] | AK023519 | 108 | ILMN_1913510 | CAGAGTCTCCGGGCCT |
| | | | | | TGGTAATTCCTAGACC |
| | | | | | ACAGCACCATGCATTA |
| | | | | | GG |
| SLC34A2 | Homo sapi [symbol] | NM_00642 [symbol] | 109 | ILMN_2184109 | ATCTAGGAAAGGAGGA |
| | | | | | GTGGGTGTAGCCGTGC |
| | | | | | AGCAAGATTGGGGCCT |
| | | | | | CC |
| BX538295 | Homo sapi [symbol] | BX538295 | 110 | ILMN_1861270 | TCTGGCTTACAGGGGA |
| | | | | | ACACAACTATTCCACA |
| | | | | | AGTGGCCTTTAGTGCT |
| | | | | | CT |
| IGFL2 | Homo sapi [symbol] | NM_00100 [symbol] | 111 | ILMN_1790227 | GCTGGCTCCTGCTTAT |
| | | | | | GTGTCAGTCTGTCTCC |
| | | | | | TCCTCTTGTGTCCAAG |
| | | | | | GG |
| CHI3L1 | Homo sapi [symbol] | NM_00127 [symbol] | 112 | ILMN_3307868 | GGGATGGGGCTGTGGG |
| | | | | | GATAGTGAGGCATCGC |
| | | | | | AATGTAAGACTCGGGA |
| | | | | | TT |
| CYP24A1 | Home sapi [symbol] | NM_00078 [symbol] | 113 | ILMN_1685663 | GATTTAGGATCTGTGG |
| | | | | | TGCAGGGCAATGTTTC |
| | | | | | AAAGTTTAGTCACAGC |
| | | | | | TT |
| HHATL | Homo sapi [symbol] | NM_02070 [symbol] | 114 | ILMN_1691355 | AGGAGCAGAAGCAGGA |
| | | | | | CAAAGAGAAGCCGGAG |
| | | | | | TAGGAGGGAGCGGGTA |
| | | | | | GA |
| ISYNA1 | Homo sapi [symbol] | NM_01636 [symbol] | 115 | ILMN_1747934 | TTCCTCCCCGCTGCCC |
| | | | | | CCCACGACCCTACCTT |
| | | | | | GAAGGCCCCCACAAAT |
| | | | | | AA |
| LM03 | Homo sapi [symbol] | NM_00100 [symbol] | 116 | ILMN_1694913 | ACAGTGGGGAGCTGTG |
| | | | | | CGGATGGGTGAAGTGC |
| | | | | | ATGTATGCCTGCCTAG |
| | | | | | AC |
| MIR221 | Homo sapi [symbol] | NR_02963 [symbol] | 117 | ILMN_3310326 | TGTTCGTTAGGCAACA |
| | | | | | GCTACATTGTCTGCTG |
| | | | | | GGTTTCAGGCTACCTG |
| | | | | | GA |
| PCSK1N | Homo sapi [symbol] | NM_01327 [symbol] | 118 | ILMN_1755582 | AGCTGTTGAGGTACTT |
| | | | | | GCTGGGACGGATTCTT |
| | | | | | GCGGGAAGCGCGGACT |
| | | | | | CC |
| SCG5 | Homo sapi [symbol] | NM_00302 [symbol] | 119 | ILMN_2065773 | AGAAGGCTGTACTCAA |
| | | | | | GAGGAGGAGCTGACAC |
| | | | | | ATTTCACTTGGCTGCG |
| | | | | | TC |
| BX955517 | DKFZp781 [symbol] | BX955517 | 120 | ILMN_1880849 | CCTACCTCCTACCTCC |
| | | | | | CCACACAGCAGCAATC |
| | | | | | AGTATCCAGAAGTTTA |
| | | | | | TG |
| CST6 | PREDICTED [symbol] | XM_00112 [symbol] | 121 | ILMN_1697733 | CAGCAGGAGAAGCTGC |
| | | | | | GCTGTGACTTTGAGGT |
| | | | | | CCTTGTGGTTCCCTGG |
| | | | | | CA |
| SFTPB | Homo sapi [symbol] | NM_00054 [symbol] | 122 | ILMN_2359835 | CCTCTCCAGTGTATCC |
| | | | | | ACAGCCCCGACCTTTG |
| | | | | | ATGAGAACTCAGCTGT |
| | | | | | CC |
| SLC27A6 | Homo sapi [symbol] | NM_01403 [symbol] | 123 | ILMN_2377199 | CTCATTTCTCGAGTGA |
| | | | | | ATGCAAAAAATCCCTT |
| | | | | | CTTTGGCTATGCTGGG |
| | | | | | CC |
| TMEM233 | Homo sapi [symbol] | NM_00113 [symbol] | 124 | ILMN_3242676 | TTTCCAAATAGGCCCC |
| | | | | | CTCCCTTGCTTGCATC |
| | | | | | CACGTTGGTCAACTTG |
| | | | | | AC |

[symbol]

indicates data missing or illegible when filed

1. A method of detecting thyroid cancer in a subject comprising a) obtaining a sample from a subject b) contacting the sample obtained from the subject with one or more agents that detect expression of a panel of markers encoded by the genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19, or a complement thereof; c) contacting a non-cancerous cell, with the one or more agents from b); and d) comparing the expression level of the panel of markers encoded for by the genes IGSF1, IGSF21, TM7SF4, FLJ30058, CITED1, ZCCHC12, CLDN6, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19, or a complement thereof in the sample obtained from the subject with the expression level of the panel of markers encoded for by the genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a complement thereof, in the non-cancerous cell, wherein a higher level of expression of the panel of markers encoded for by genes IGSF1, IGSF21, TM7SF4, FLJ30058, CITED1, ZCCHC12, CLDN16, FN1, SERPINA1, STK32A, UNQ9433, BC030766, AK023519, SLC34A2, BX538295, IGFL2, CHI3L1, CYP24A1, HHATL, ISYNA1, LMO3, MIR221, PCSK1N, SCG5, BX955517, CST6, SFTPB, SLC27A6, TMEM233, NMU KIAA1324, CCDC85A, CRABP2, C14orf78, TNFRSF11B, AHNAK2, CYTOKERATINE19 or a complement thereof in the sample compared to the non-cancerous cell indicates that the subject has thyroid cancer.

2. The method of claim 1, wherein the subject is a human.

3. The method of claim 1, wherein the sample is a bodily fluid.

4. The method of claim 3, wherein the bodily fluid is blood.

5. The method of claim 3, wherein the bodily fluid is serum.

6. The method of claim 3, wherein the bodily fluid is urine.

7. The method of claim 1, wherein the sample is a tissue sample.

8. The method of claim 1, wherein the sample is compised of cells.

9. The method of claim 1, wherein the one or more agents is a nucleic acid.

10. The method of claim 1, wherein the one or more agents is a protein.

11. The method of claim 10, wherein the protein is an antibody.

12. A kit for detecting thyroid cancer in sample comprising one or more agents of claim 1.

13. The kit of claim 12, wherein the one or more agents is a nucleic acid.

14. The kit of claim 12, wherein the one or more agents is a protein.

15. The kit of claim 12, wherein the protein is an antibody.

16. The kit of claim 12, further comprising one or more controls.

17. The kit of claim 16 wherein the control is a positive control.

18. The kit of claim 17 wherein the positive control comprises thyroid cancer cells.

19. The kit of claim 16, wherein the one or more controls is a negative control.

20. The kit of claim 19, wherein the negative control comprises non-cancerous thyroid cells.


SEQUENCE LISTING (96 sequences)

SEQ ID NO: 1 - DNA, 4492 bp, Homo sapiens
gggcagtttgctgcatctggaggagctcactggagaatctccaacatcggagcgggccttcaactaccatcccaccacctgctgaggagaaaaattcttcaagactcagagcacacagccagcaccagaggccccatgaccctggacagaccaggggagggggccaccatgctgaagacattcactgttttgctcttttgcattcggatgagtctgggtatgacatcgatagtgatggaccctcaaccggagttgtggatagagtccaactacccccaggccccttgggagaacatcacgctttggtgccgaagcccctctcggatatcaagcaagttcctgctgctgaaggataagacacagatgacctggatccgcccttcccacaagaccttccaagtttcattccttataggtgcccttactgagtccaatgcaggtctttaccggtgctgctactggaaggagacaggctggtcaaagcccagtaaagttctagagttggaggcaccaggccaactgcccaagcccatcttctggattcaggctgagacccccgctcttcctgggtgtaatgttaacatcctctgccatggctggctgcaggatttggtattcatgctgtttaaagagggatatgcagagcctgtggattaccaagtcccaactgggacaatggccatattctccattgacaacctgacacctgaggatgaaggggtttacatctgccgcactcatatccagatgctccccaccctgtggtcagagcccagcaaccccctgaagctggttgtagcaggactctaccccaaaccaactttgacagcccatcctgggcccatcatggcacctggagaaagcctgaatctcaggtgccaagggccaatctatggaatgacctttgctctaatgagggttgaagacttggagaagtccttttaccacaagaagacaataaaaaatgaggcaaatttcttcttccagtctttgaagatccaagatactggacattacctctgtttttactatgacgcatcatatagaggttcactccttagtgatgtcctgaaaatctgggtaactgacactttccccaagacctggctacttgctcggcccagtgctgtggtccaaatgggtcagaatgtgagcctacggtgtcgaggaccagtggatggagtgggtcttgcactctataagaaaggagaagacaaaccacttcaatttttggatgccaccagcatcgatgacaacacatcattcttcctcaacaatgtaacctacagtgatactggcatctatagctgccactatcttctcacctggaagacctccattaggatgccatcacacaacactgtggagcttatggttgtagataagccccccaaaccctccctgtcagcttggccaagcactgtgttcaagctaggaaaggccatcacccttcagtgccgagtatctcatccagtactggaattttctctggaatgggaagaaagagaaacattccaaaaattctcagtaaacggagacttcatcatcagtaatgttgacgggaaaggcacagggacctacagttgcagctatcgcgtagagacacatcctaacatctggtcacatcgcagtgagcccctgaagctgatggggccagcaggctatctcacctggaattacgttctgaatgaagctatcaggttgtctctaatcatgcagcttgttgccttgctgttggtagtgctgtggataaggtggaagtgtcggagactcagaatcagagaagcctggttgctgggaacagctcaaggggtcaccatgctcttcatagtcacggcccttctctgctgtggactgtgcaatggggtattgatagaagagactgaaatagtcatgccaacccctaagcctgagctgtgggcagagaccaactttcctctggccccgtggaagaacttaaccctctggtgcagaagcccttctggctcaactaaggagtttgtgttgctgaaggatgggaccgggtggatcgccactcgcccggcctcagagcaggtccgggctgccttcccccttggcgccctgacccagagccacaccgggagctaccactgccattcatgggaggagatggctgtatcggagcccagtgaggcacttgagctggtggggacagacatcctccccaaacctgtcatttctgcttcccccacaatccggggccaggaactacaactccggtgcaaaggatggctggcaggcatggggtttgctctgtataaggagggagagcaagaacctgtccagcaacttggtgctgttggaagagaagccttctttacaatccagagaatggaggataaagacgaaggcaattacagctgccgcactcacactgaaaaacgccccttcaagtggtctgagcccagtgagccgctggagcttgtcataaaagaaatgtaccctaagcccttcttcaagacatgggccagccctgtggtcacccctggtgcccgagtgactttcaattgctccaccccccaccagcatatgagctttattctttacaaagatggaagtgaaatagcatccagtgacaggtcctgggcaagtccgggggccagtgcagctcactttctaatcatttcggtgggcattggtgatggagggaattacagctgccgatattatgacttttctatctggtctgagcccagcgaccctgtggagctcgtggtgacagaattctaccccaaacccactctcctggcacagccaggtcctgtggtgtttcctgggaagagtgtgatcctgcgctgccaagggactttccagggcatgaggttcgccctcttgcaggagggagcccatgttcccttacagtttcggagtgtctcagggaactcagctgacttccttctccacactgttggagcagaggactctgggaactatagctgtatctactatgagacaaccatgtcaaacagggggtcatatctcagtatgccccttatgatctgggtgactgacacattccctaagccatggttgtttgctgagcccagttctgtggttcccatggggcagaatgttactctctggtgccgagggccggtccatggagtaggatacattctgcacaaagaaggagaagccacttcaatgcagctctggggatccaccagtaatgacggggcattccccatcaccaatatatctggtactagcatggggcgttacagctgctgctaccaccctgactggaccagttctatcaagatacaacctagcaacaccctggaactcctagtcacaggcttactccccaaacccagcctattagcccagcctggtcccatggtggcccctggcgaaaatatgactcttcagtgtcaaggggaactgccagactcaacatttgtcctgttgaaggagggggctcaggagcctttagagcaacagaggccaagtgggtacagggctgacttctggatgccagcagtgagaggtgaagactctgggatctatagctgtgtttattatttggactctactccctttgcagcttcaaatcacagtgactccctggagatctgggtgactgataagccccctaaaccctctctgtcagcctggcccagcaccatgttcaagttagggaaggacatcacccttcagtgccgaggacccctgccaggtgttgaatttgtcctagaacatgatggagaagaagcacctcagcagttttcagaggatggagactttgtcatcaacaacgtagaaggaaaaggcattggaaactacagctgcagctaccgcctccaggcctaccctgatatctggtcagagcctagtgatcccctggagctggtgggggcagcagggcctgttgctcaggagtgcactgtagggaacattgtccgaagtagcctaatcgtggtggttgttgtagccttgggggtagtgctagccatagagtggaagaagtggcctcgactgcgaaccagaggctcagagacagacggaagagaccagaccattgcccttgaagagtgtaaccaagaaggagaaccaggcacccctgccaattctccttcatcaacctctcagagaatctctgtggaactgcccgttccaatataataatctcctcctttacaagagctttcctctcctctctcttgctctcagagacctataaatccaaccagttaccctgcaagtcagccccatctgctgttccttggtctctaatcacctgagctgggtaaaggggattctgggagttgagagctctgccagggtgagatgtttcctgaagagaggttccccacccctgtaactcctcactgtactgatttactggcgcatgaaattctattaaaaatgcattcttctgaataaaaagagtattcactatttaacttcaaaaaaaaaaaaaaaaaaaaaaaaaaaaaaaaaaaaaaaaaaaaaaaaaaaaaaaaaaaaa

SEQ ID NO: 2 - DNA, 1943 bp, Homo sapiens
aggaggggggtgctcgcgccgccgggagaggcgagcgcgaggcagagagcgcgattcggctccaaactccggcgctgcagccgatcggactctgggccgcggtgggcaccgcgcgcagctagggagccgagaaccgcggcgagccccgaggacgcccagagcgcgagggtcgctgcgcctcgcagagccggagccgagtcgagccgggcgcccgggctgcctggccgcggcggcatgggggcgcccccgcggctctccgcgctgcccgccaccgcctcggccagtggccggaggcaggagcgcgtctgagcccatggcgaggggacccgccgccaccgcctccacccccgccgccccgccaccgccgccagctcccgggcaccatgcgaaccgccccgagcctccgccgctgcgtctgcctgctgctcgccgcgatcctggacctggcgcgcggctacctgacagtcaacattgagcctctcccccctgtggtggctggagacgccgtgactttgaagtgtaacttcaagacagatgggcgcatgcgggagatcgtgtggtaccgggtgacggatggtggcaccatcaagcaaaagatcttcaccttcgacgccatgttctccaccaactactcacacatggagaactaccgcaagcgagaggacctggtgtaccagtccactgtgaggctgcccgaggtccggatctcagacaatggtccctatgagtgccatgtgggcatctacgaccgcgccaccagggagaaggtggtcctggcatcaggcaacatcttcctcaacgtcatggctcctcccacctccattgaagtggtggctgctgacacaccagcccccttcagccgctaccaagcccagaacttcacgctggtctgcatcgtgtctggaggaaaaccagcacccatggtttatttcaaacgagatggggaaccaatcgacgcagtgcccctatcagagccaccagctgcgagctccggccccctacaggacagcaggcccttccgcagccttctgcaccgtgacctggatgacaccaagatgcagaagtcactgtccctcctggacgccgagaaccggggtgggcgaccctacacggagcgcccctcccgtggcctgaccccagatcccaacatcctcctccagccaaccacagagaacataccagagacggtcgtgagccgtgagtttccccgctgggtccacagcgccgagcccacctacttcctgcgccacagccgcaccccgagcagtgacggcactgtggaagtacgtgccctgctcacctggaccctcaacccacagatcgacaacgaggccctcttcagctgcgaggtcaagcacccagctctgtcgatgcccatgcaggcagaggtcacgctggttgcccccaaaggacccaaaattgtgatgacgcccagcagagcccgggtaggggacacagtgaggattctggtccatgggtttcagaacggagtcttcccggagcccatgttcacgtggacgcgggttgggagccgcctcctggacggcagcgctgagttcgacgggaaggagctggtgctggagcgggttcccgccgagctcaatggctccatgtatcgctgcaccgcccagaacccactgggctccaccgacacgcacacccggctcatcgtgtttgaaaacccaaatatcccaagaggaacggaggactctaatggttccattggccccactggtgcccggctcaccttggtgctcgccctgacagtgattctggagctgacgtgaaggcacccgccccggccactccatcaggcactgacatctccgcgaccggttttcatttcttttctaaactatttccagtcttgttcttagtctctttccatctgtgtcttggcttcttcagtcggtttaattaaaacaaacagaacaattttcccc

SEQ ID NO: 3 - DNA, 1952 bp, Homo sapiens
gcatttctgcattcgaagaagaatctgagagaaacctgacgcagggagcatgggtatctggacctcaggcactgatatcttcctaagtctttgggagatttacgtgtctccaagaagccccggatggatggactttatccagcatttgggagtttgctgtttggttgctcttatttcagtgggcctcctgtctgtggccgcctgctggtttctgccatcaatcatagcggccgctgcctcctggattatcacgtgtgttctgctgtgttgctccaagcatgcacgatgttttattcttcttgtctttctctcttgtggcctgcgtgaaggcaggaatgctttgattgcagctggcacagggatcgtcatcttgggacacgtagaaaatatttttcacaactttaaaggtctcctagatggtatgacttgcaacctaagggcaaagagcttttccatacattttccacttttgaaaaaatatattgaggcaattcagtggatttatggccttgccactccactaagtgtatttgatgaccttgtttcttggaaccagaccctggcagtctctcttttcagtcccagccatgtcctggaggcacagctaaatgacagcaaaggggaagtcctgagcgtcttgtaccagatggcaacaaccacagaggtgttgtcctccctgggtcagaagctacttgcctttgcagggctttcgctcgtcctgcttggcactggcctcttcatgaagcgatttttgggcccttgtggttggaagtatgaaaacatctacatcaccagacaatttgttcagtttgatgaaagggagagacatcaacagaggccctgtgtgctcccgctgaataaggaggaaaggaggaagtatgtcatcatcccgactttctggccgactcctaaagaaaggaaaaacctggggctgtttttcctccccatacttatccatctctgcatctgggtgctgtttgcagctgtagattatctgctgtatcggctcattttctcagtgagcaagcagtttcaaagcttgccagggtttgaggttcacttgaaactgcacggagagaaacaaggaactcaagatattatccatgattcttcctttaatatatctgtgtttgaacccaactgtatcccaaaaccaaaattccttctatctgagacctgggttcctctcagtgttattcttttgatattagtgatgctgggactgttgtcctctatccttatgcaacttaaaatcctggtgtcagcatctttctaccccagcgtggagaggaagcgcatccaatatctgcatgcaaagctgcttaaaaaaagatcaaagcagccgctgggagaagtcaaaagacggctgagtctctatcttacaaagattcatttctggcttccagtcctgaaaatgattaggaagaagcaaatggacatggcaagtgcagacaagtcatgagagaccccgactactcctcagccacatcgcaccaacaattctcttcaggtctaggatggcagtcactattcatgccggataatagagaactatgtgacgcagtcctctcaggagtctgagtttacagagccaacttgcagcacctggttatgcctcctttcatctcaaagccaaagagctgccaggtaaatggttatgtggtctatgttccaaacaaaccacatgatcttgcctgtgtcacaatgtaacaagactctagctgggtcccctggtgatgagtttcagcatagaataatgttcaaggaaaagaaaacgaaaacagtttaaatctctaccacagcctcacaagcaaatgctaaggggaacatacatgtaaaaagccagcaaactatcttcaaactcttccgtccttaatgtcttccatggctattgcccccacaatggtctcttttctccctgctcccttattaaagaactctttctgaaaccc

SEQ ID NO: 4 - DNA, 3082 bp, Homo sapiens
atgctcgaggtgtccggtgaccgaaacactcttagctgggtgcagggtgcggctcagggcgtggtcaccgggggctacttagggccgccggtgcgggggacgacgcaaaggttaactgcgagctgccgggcactcagcgcgggtcatggcgtggatactggactgccttttcgcctcggcctttgagccccgcccccgccgtgcaaaaacaaccagaggctgctctgcttgagggtgaagccgcctcccagttttccctccccctctacccccacccccatagttctctccaccaggtccagtgacaattggatgatgcagccttgataatcatccgattccagaatgggtggctgcattccttttctgaaggcagcaagggcactgtgccccagaatcatgccccctttgctgttgttgtccgccttcatttttttagtgagtgtcttgggaggagccccaggacacaaccccgaccgcaggacgaagatggtatcgatacacagcctctctgagctggagcgtctgaagctgcaagagactgcttaccacgaactcgtggccagacatttcctgtccgaattcaaacctgacagagctctgcctattgaccgtccgaacaccttggataagtggtttctgattttgagaggacagcagagggctgtatcacacaagacatttggcattagcctggaagaggtcctggtgaacgagtttacccgccgcaagcatcttgaactgacagccacgatgcaggttgaagaagccaccggtcaggctgcgggccgtcgtcggggaaacgtggtgcgaagggtgtttggccgcatccggcgctttttcagtcgcaggcggaatgagcccaccttgccccgggagttcactcgccgtgggcgtcgaggtgcagtgtctgtggatagtctggctgagctggaagacggagccctgctgctgcagaccctgcagctttcaaaaatttcctttccaattggccaacgacttctgggatccaaaaggaagatgagtctcaatccgattgcgaaacaaatcccccaggttgttgaggcttgctgccaattcattgaaaaacatggcttaagcgcagtggggatttttacccttgaatactccgtgcagcgagtgcgtcagctccgtgaagaatttgatcaaggtctggatgtagtgctggatgacaatcagaatgtgcatgatgtggctgcactcctcaaggagtttttccgtgacatgaaggattctctgctgccagatgatctgtacatgtcattcctcctgacagcaactttaaagccccaggatcagctttctgccctgcagttgctggtctacctgatgccaccctgccacagtgataccctggagcgtctgctgaaggccctgcataaaatcactgagaactgcgaggactcaattggcattgatggacagttggtcccaggcaaccgtatgacttccactaacttggccttggtgtttggatctgctctcctgaaaaaaggaaagtttggcaagagagagtccaggaaaacaaagctggggattgatcactatgttgcttctgtcaatgtggtccgtgccatgatagataactgggatgtcctcttccaggtgcctccccatattcagaggcaggttgctaagcgcgtgtggaagtccagcccggaagcacttgattttatcagacgcaggaacttgaggaagatccagagtgcacgcataaagatggaagaggatgcactactttctgatccagtggaaacctctgctgaagcccgggctgctgtccttgctcaaagcaagccttctgatgaaggttcctctgaggagccagctgtgccttccggcactgcccgttcccatgacgatgaggaaggagcgggtaaccctcccattccggagcaagaccgcccattgctccgtgtgccccgggagaaggaggccaaaactggcgtcagctacttctttccttagatgtttttccttctataaggtgccagacaggggaaaagggtgggggtacatctgggatgtcacaggaaacattaaggagagagttgaaggtaaagatctgaaggtaagaaggagttccacctgatgctcgggtcaggatgagaattccaaacacactgccagccccttcactggggatgcttggtctcttctgctggtaaaagcagagatgtttctgtgtcatgcccaagctccccggtgctaccttgcctttctcttttacccctgatcttggctttctctctctctctgcagactttcctttaattgatgtgacatttgtggtaaacacctttcccagggaacctcacaaatcttgagatgctttcccttccccagatgggattgcatgattccctgactttcctaccctcctccagagagctcagttggaaaggccctcaagaggcatgctagaacgttaggtcagcctactgacagctgacaaacaattaatgcgaaatcatgtcacaccaacccatagccgtgtccacgcagcaactccaccaccttaggatttccccctccaaattattcagaccaatggcttgccaaatggcctctcccaaaattctgtacagttttgctcaggtcacgccaacagggaaacctcaagtgtaggtctaattagtgtttctgggatccaaagttagaggaaaatttagattttattgcctggatctgctttaaagacaattggtgtttacaccctcttgtcagcaaaacagctagttaggtaaggacatatagttccaagtaggtaaagtcacttgattacaaatgttcttaactatcgtctctgtaattcctttatacaggacagtacaaaattgtgggacatgctctggtaacacacagatatgggttgcatatgatccagaattacagctgatattatggatgacaactgctaaggtccataaaatgaagactgtattgtattgagggatagaaattgatcatttaatgggtaacaactgctgagctcaaagatttgtgattgttaaaacttctctggcatttaatcattaataaacatctgtattgtgacagc

SEQ ID NO: 5 - DNA, 1203 bp, Homo sapiens
gtggaaattgaggggagaaaaaaaaagggaaaaaaagggtctgtccttcctgggattcctagccgaggccagtctgctgccgtgtgcgtgtgcgtcagggctctccgggcggcaatgggggcttgagagccgggtccccagcgccgggaagggagcgcggtggccgccaccgccaccgccccggagtccggcgccgaagctgcgggcgggcgggcgggcaccagctcggtcaggggctgcttggcgcggcactgtgcggtgcagcggcggcgcggcgcggtgcgggcttttcccaggcgccccggggtcgggtggccaacggcgcggccgcgggcgctgagcgcgaccggttcgcggtagcggtggcggcggcgtgcgtgccaggggctgggggctccgccgcctctcttgcggctcaccgagctccgcgcttccctctctccagggcaggcggcttctcagagcacaacagctccagctggcagcatcacttcccgccaatttatccaacttctgccaaggctctgaaatgccaacaacgtcgaggcctgcacttgatgtcaagggtggcacctcacctgcgaaggaggatgccaaccaagagatgagctccgtggcctactccaaccttgcggtgaaagatcgcaaagcagtggccattctgcactaccctggggtagcctcaaatggaaccaaggccagtggggctcccactagttcctcgggatctccaataggctctcctacaaccacccctcccactaaacccccatccttcaacctgcaccccgcccctcacttgctggctagtatgcagctgcagaaacttaatagccagtatcaggggatggctgctgccactccaggccaacccggggaggcaggacccctgcaaaactgggactttggggcccaggcgggaggggcagaatcactctctccttctgctggtgcccagagccctgctatcatcgattcggacccagtggatgaggaagtgctgatgtcgctggtggtggaactggggttggaccgagccaatgagcttccggagctgtggctggggcagaatgagtttgacttcactgcggactttccatctagctgctaatgccaagtgtccctaaagatggaggaataaagccaccaattctgttgtaaataaaaataaagttacttacaaaaaaaaaaaaaaaaaaaaaa

SEQ ID NO: 6 - DNA, 2212 bp, Homo sapiens
ggcgctgcctcgtctctgctacccctggttgggcggccctgcgaagcagctccttcgggcagccccgggtcgcttagcggccaaggaggcttcagttctttgccgcctgcaaggcggagaccagaaggcggaatccacagctggcgacgcgggagcatctgctgtccaccagcggagcacaggccatcaaagccgcatctgaacttgaattctgtgcagctgattgcagagctggacccggatctgcgaccccctgtggacagaggttgaccgtaccccggagaggagctttctcacggagggcactggttgcagaggctggaagtgaaataaagacgcgctcttgtttcagagttcgtcccctgctgagataggaaggcagagccacctcctctcctctcccacctgcagattaagcttttctaaaaagcctaggcatcttcttatattcagataccctatcgtcgtcagtcatggctagcatcattgcacgtgtcggtaacagccggcggctgaatgcacccttgccgccttgggcccattccatgctgaggtccctggggagaagtctcggtcctataatggccagcatggcagacagaaacatgaagttgttctcggggagggtggtgccagcccaaggggaagaaacctttgaaaactggctgacccaagtcaatggcgtcctgccagattggaatatgtctgaggaggaaaagctcaagcgcttgatgaaaacccttaggggccctgcccgcgaggtcatgcgtgtgcttcaggcgaccaaccctaacctaagtgtggcagatttcttgcgagccatgaaattggtgtttggggagtctgaaagcagtgtgactgcccatggtaaattttttaacaccctacaagctcaaggggagaaagcctccctttatgtgatccgtttagaggtgcagctccagaacgctattcaggcaggcattatagctgagaaagatgcaaaccggactcgcttgcagcagctccttttaggcggtgagctgagtagggacctccgactcagacttaaggattttctcaggatgtatgcaaatgagcaggagcggcttcccaactttctggagttaatcagaatggtaagggaggaagaggattgggatgatgcttttattaaacggaagcgtccaaaaaggtctgagtcaatggtggagagggcagtcagccctgtggcatttcagggctccccaccgatagtgatcggcagtgctgactgcaatgtgatagagatagatgataccctcgacgactccgatgaggatgtgatcctggtggagtctcaggaccctccacttccatcctggggtgcccctcccctcagagacagggccagacctcaggatgaagtgctggtcattgattccccccacaattccagggctcagtttccttccaccagtggtggttctggctataagaataacggtcctggggagatgcgtagagccaggaagcgaaaacacacaatccgctgttcgtattgtggtgaggaaggccactcaaaagaaacctgtgacaacgagagtgacaaggcccaggtttttgagaatttgatcatcactctccaggagctgacccatactgagatggagaggtcaagagtggcccctggcgaatacaatgacttctctgagccactgtaagggaccacccccaggtttcagtgaacccttacctatattcagcatccagtagtgggaaaactggggtgggggtgggggtgggacttctaactgcatgaattaatccacaaagcggctatcttttggggtggagtagaaagggtcttggataccagcacattggagggagatagcctgacctctgtccttgctccttctccctgcagcctacgggtctgttttctgtgtgtgcccatttccttgacagctttattctttgtgaaagtggtataatttattgttaaatatttgaacaataaaaaaggtacaaaaagtgaagtacaaattacccaaatctctccacccttatataatcattgtcaaccctttgatgagtgatatttccctatacctatgtacccagatagatatatgcatagataaaagtgatgaaatataagtgctgttctatctgtattttttcaccaaacaatatatgttgtgagcttctatgtcaataaatatatatatcagcaaaaaaaaaaaaaaaa

SEQ ID NO: 7 - DNA, 1494 bp, Homo sapiens
ccccacccgaaacacactcagcccttgcactgacctgccttctgattggaggctggttgcttcggataatgacctccaggaccccactgttggttacagcctgtttgtattattcttactgcaactcaagacacctgcagcagggcgtgagaaaaagtaaaagaccagtattttcacattgccaggtaccagaaacacagaagactgacacccgccacttaagtggggccagggctggtgtctgcccatgttgccatcctgatgggctgcttgccacaatgagggatcttcttcaatacatcgcttgcttctttgcctttttctctgctgggtttttgattgtggccacctggactgactgttggatggtgaatgctgatgactctctggaggtgagcacaaaatgccgaggcctctggtgggaatgcgtcacaaatgcttttgatgggattcgcacctgtgatgagtacgattccatacttgcggagcatcccttgaagctggtggtaactcgagcgttgatgattactgcagatattctagctgggtttggatttctcaccctgctccttggtcttgactgcgtgaaattcctccctgatgagccgtacattaaagtccgcatctgctttgttgctggagccacgttactaatagcaggtaccccaggaatcattggctctgtgtggtatgctgttgatgtgtatgtggaacgttctactttggttttgcacaatatatttcttggtatccaatataaatttggttggtcctgttggctcggaatggctgggtctctgggttgctttttggctggagctgttctcacctgctgcttatatctttttaaagatgttggacctgagagaaactatccttattccttgaggaaagcctattcagccgcgggtgtttccatggccaagtcatactcagcccctcgcacagagacggccaaaatgtatgctgtagacacaagggtgtaaaatgcacgtttcagggtgtgtttgcatatgatttaatcaatcagtatggttacattgataaaatagtaagtcaatccaggaacagttatttagaattcatattgaattaaattaattgctagcttaatcaaaatgtttgattctcctatactttttctttctattactcttatattttcccgtcattctctctgctaaccttccaccttatgcacacactttccctatattttaagataagtctgctaggatgtagaaatatttgtttgtgatttctatatagctattagagattatgacatagtaatattaaaatgaaatgatacttaaacagaaagcaatttccaaagaggccagggaccctaatctttgaagagatgaagaaacttacttttctccctggcttttggttcactttttgtacttttaacaagtgggtgaattatttgataattttgaggaagattattcttttaaattcaaactagtatgtcaatgcctaccatta

SEQ ID NO: 8 - DNA, 8449 bp, Homo sapiens
gcccgcgccggctgtgctgcacagggggaggagagggaaccccaggcgcgagcgggaagaggggacctgcagccacaacttctctggtcctctgcatcccttctgtccctccacccgtccccttccccaccctctggcccccaccttcttggaggcgacaacccccgggaggcattagaagggatttttcccgcaggttgcgaagggaagcaaacttggtggcaacttgcctcccggtgcgggcgtctctcccccaccgtctcaacatgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacccgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggacagcccaggcagtacaatgtgggtccctctgtctccaagtacccactgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccattgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagagacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggagttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttatgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattccacaacttgaagttcatctatttgatataagacaccttcgggggaaataattcctgtgaatattctttttcaattcagcaaacatttgaaaatctatgatgtgcaagtctaattgttgatttcagtacaagattttctaaatcagttgctacaaaaactgattggtttttgtcacttcatctcttcactaatggagatagctttacactttctgctttaatagatttaagtggaccccaatatttattaaaattgctagtttaccgttcagaagtataatagaaataatctttagttgctcttttctaaccattgtaattcttcccttcttccctccacctttccttcattgaataaacctctgttcaaagagattgcctgcaagggaaataaaaatgactaagatattaaaaaaaaaaaaaaaaaa

SEQ ID NO: 9 - DNA, 1607 bp, Homo sapiens
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcaaaa

SEQ ID NO: 10 - DNA, 824 bp, Homo sapiens
ccttgctcttggagttcttctcttagtccctgttccctggatgaaagcatcgctccgagcctcatgggaggaatgaaggaagaatcgagactagatatccaactaaggcttcgggacatgttttgagcgaagatgggtgtttctgcccggatagtataaatcgaggatccaggtctgggcagattcaaccatgggagccaacacttcaagaaaaccaccagtgtttgatgaaaatgaagatgtcaactttgaccactttgaaattttgcgagccattgggaaaggcagttttgggaaggtctgcattgtacagaagaatgataccaagaagatgtacgcaatgaagtacatgaataaacaaaagtgcgtggagcgcaatgaagtgagaaatgtcttcaaggaactccagatcatgcagggtctggagcaccctttcctggttaatttgtggtattccttccaagatgaggaagacatgttcatggtggtggacctcctgctgggtggagacctgcgttatcacctgcaacagaacgtccacttcaaggaagaaacagtgaagctcttcatctgtgagctggtcatggccctggactacctgcagaaccagcgcatcattcacagggatatgaagcctgacaatattttacttgacgaacatgatacctggctctcctacaagtcccactgaattggagtttcaggagaccgaagcccaggcacatgtattttgcaaaactacactgaagtttctgataatgacggatatcaacaattaaacgcttacttcttgtcaaaaaaaaaaaaaaaaaaaaaaaaaaaaa

SEQ ID NO: 11 - DNA, 1102 bp, Homo sapiens
attcggctcgagggcacactgaggaccgagtcccagtgcagctccagccgacccccgggacgtagacaagggcaggcgcgcggtgaagactgcggccggccgcgtagccgcgctgtggtcccgctggccttctcttgggccgcgcaccctccggacctgcgtcgggatcctcccggagtctcccgctctctcctcccggcttgcgaacatgcggccccttaagcccggcgcccctttgcccgcactcttcctgctggcgctggctttgtccccgcacggagcccacgggaggccccgggggcgcaggggagcgcgcgtcacggataaggagcccaagccgttgcttttcctccccgcggccggggccggccggactcccagcggctcccggagcgcagaaatattcccaagagactctaacttaaaagacaaattcataaagcatttcacagggccggtcacattttcaccagaatgcagcaaacatttccaccgactctattacaataccagggagtgctcaacgccagcttattacaaaagatgtgctagattgttaacaagattagcagtgagtccactgtgctcccagacctagcaaaactaccctacatttcctaagaatgtacatctaatttgaagaaaaagtgcctcaaatcatgcaaaatgtaaaaaaagatgaaatttatatttttatggatattaagatgagtaaaataagagacttcccagaaataactggttagctgtttcctgtcatagaatggagtctttcttgctttatctttttgtgtatacagtaatttataattttgtaaaacagagtttgaatcgcatattgaaaattagatattaaaaattgtgtgattgtattttatttttactagatatattattttctttatatggttaacattctaattaaacatttaattgtgtaaattatatctgtgagtgccagtgagaaataatgatttttttgtatatgactgttagtatatatttgtcaaatcatcgtgtaccaagttaaattttataattcttatttaatatttaaatacatttatttagaaaaaaaaaaaaaaaaaaaaaaaaaaaaaaa

SEQ ID NO: 12 - DNA, 1112 bp, Homo sapiens
caaaagtacagtaacgaagtattgaaaagaaaattttggagacattggagcatattatatatagcttgtggaaagacataaggctacagatggaatggaacattcctgttttcttgaagaaattcacatacacatagctgacctgactagtacttcagctcttccacagccttctataaaggttctttcttctgcaaagaaaacaaaacaaaacaaaacaaaacaaaaaaaaacaaaaaaagcgcaaaaaacaaaaaaacaaaaaaaagcaaagtaaaatttaaaaatacagaaaacaaacaacaaaaaagaattcaaccataaatagtgactattattttcagtgtgtccttcatgtgaaagctattaaggaccaaatatactactgttcataagaagaaattactttctaaacagtaactgaaaatacttagagttaaacttgctgtggattttgtcttggcagttgtcatcttacattatttgtcaaaggaaatgtgtttggcagttaaaaatctttccttagatttagtggtggactttaacctcttaaataaatgttagtatatcagattgtgtccttgaaaaatattttacttgtatgaatcatgacaacgtctaaatctttactattcttctggcaaaagcatcagtaagaaagaaggcgaaaaagagaagtatagcctttatgtcagaaaaacattctttttagctgcttactttctcatgaaaagtaaagatgtttacagtgtatgccaagttttaagtttctgtataacaacaggtagaggttctaatcatattgaaaattgtgttataatggtctgagccatgttgctaggaaacaataggttccaattttgtattcctgctctcctgtgctgaaaagtgactggatactgtacaggttcatgttctctggctgcagttaaatggtcttttgcattttgctctggctttcaggccagaagcatgcatttttctacaagagcatcacaacaacatgctgtaaatatttaaagttaaacattatgtgttgatatttgaaagaaaagtactttgaatatttcatttttaaaaaataaaattgccaatgaaaaaaaaaaaaaaaa

SEQ ID NO: 13 - DNA, 2432 bp, Homo sapiens
agtctgtgagctgggagcctgttggcaggtccctctttttattttcgctgagagctttcttttactaaatgccaccatccttacctttcaaggtgtctgcgtgcctaatttttcctggttgttagacaagaacccggattttagttgaactctggagcaaaaatcctgcatcatttgtaggtaatgtttgttttctttggttgcttttatgaccttctctttattcctagtatttgggcatttcacaaaaacatttataaatctgaattaaaaaacaaactgaccttgccaaggaccctactgatcccttttcatataattctatctgctaggtcttgtgctgccttctaagttagttcatcagttctatacttcagtatttaattctatattcagccatgttatcaaatgtttatttcaacctttgagtttctcatttgtaatagtattttttcatttttatgtttttaaaagagttcttttcatattttactatttttgtttagtaactttcagtacttgttgcgtagattttatttcatctttatgtttttgagatgtttgcataacacatttaaagacaattacatattgttgtatgatttccatttccttggtcaagaattctcccatttgttgcatctctgaagagtttcccaaggtgggttaatcttgaatatggtaaacttgctgccctcagttttttagttttaatactttatcagttgtttcttgttctttttttttctgggcatattattacatcatttgaaaataatgacaattttggcctttctcttcgagattttgtaactcattttcttttcttttcacatagaatttgataaggcctccagtactttgtgatatagtagttgtggggatggtccttcttgtcttactgctgatctcaagtttatgtttgtagcaaatttttgctacatgaccattacaagtttaagaggttaccaagagtttttattacaaataccttaaatctcagcaaagactataaaaactgagataaacatgtttttcttctaaatagtaatcatttagacaaatcacaaatgtgatatcagattagtgcatcctacactgtatggaaactataaattcatgattttcaaaaattgtaggtaaatatttatttttcaaaataatttatccatcttgctttaccaaaaaaggcaaggtcttgtttttctttccctatacagtagtaatggctctttggtgtaaaaactttataggacctaaagtaaaaaagaagataaaaaccagtttgtctttcaaagtcttaaaatatttcattaacctcgtagtgcctgatctctgtcaccaagcaaatagtctgaactattatttccaagctcaattcaaacaggtaaagttttactacaattatatctggccatcactacagtgttttctccaatcacttccaaagcctaccccttttctttatcatacttccacctacattgcataatacagagcttagctagtgaggtacaagtgataaggattatctccccagagtctccgggccttggtaattcctagaccacagcaccatgcattaggttatagtcacaggcctctatatagtcttccacatatcccattacattttaaatcctatgcataccctttatatcctgcacttatctgtcaaaattctaaggtataagatttttttttacttatccctgctgtttaattttgaagaaagaaaaatataaaagaaaattttttaaaaagaggccttcatggatatcaaagaatgccaacaataatgctgaagaaagaagttattctacagcaatgagacacaaagaaaagaattttgtaaacattagcatcttggttactggagaactataacttttatgtagtcatgcttggaaaacactaaaagggaaatcgagtctgtttgacaatattctgtcttcactgttgttcacttcataatgtgttggaatataaagttctatacagttaatatgaagctctctttagcatttaaaacatgatttgcattttcatgaggcattttggctaattttattgatttccttatatttcatagtccttagccttatgagaatcttatgtttctgtgtgttttctatcatgtagcacaatttctgacacacaaaacatacaataaacttgtgttaatttttctatcaaagtcagaatttattcataaggaatctgaagtaaggtgtactaagcttgtttatgggttaagtgatatagccaaattcaaaactttactttttatgtcagtctagaaatatctcagattaaaacatatcacttcttagttccaattagataagggaaatcttttataataatgccaggattgctataatctgataggagacaacaatttcattttgctacaggaaaatttggaggac

SEQ ID NO: 14 - DNA, 4167 bp, Homo sapiens
accttcgccatatatacccggggcgctgcgctccacctggccgccgcctccagcccagcacctgcggagggagcgctgaccatggctccctggcctgaattgggagatgcccagcccaaccccgataagtacctcgaaggggccgcaggtcagcagcccactgcccctgataaaagcaaagagaccaacaaaacagataacactgaggcacctgtaaccaagattgaacttctgccgtcctactccacggctacactgatagatgagcccactgaggtggatgacccctggaacctacccactcttcaggactcggggatcaagtggtcagagagagacaccaaagggaagattctctgtttcttccaagggattgggagattgattttacttctcggatttctctactttttcgtgtgctccctggatattcttagtagcgccttccagctggttggaggaaaaatggcaggacagttcttcagcaacagctctattatgtccaaccctttgttggggctggtgatcggggtgctggtgaccgtcttggtgcagagctccagcacctcaacgtccatcgttgtcagcatggtgtcctcttcattgctcactgttcgggctgccatccccattatcatgggggccaacattggaacgtcaatcaccaacactattgttgcgctcatgcaggtgggagatcggagtgagttcagaagagcttttgcaggagccactgtccatgacttcttcaactggctgtccgtgttggtgctcttgcccgtggaggtggccacccattacctcgagatcataacccagcttatagtggagagcttccacttcaagaatggagaagatgccccagatcttctgaaagtcatcactaagcccttcacaaagctcattgtccagctggataaaaaagttatcagccaaattgcaatgaacgatgaaaaagcgaaaaacaagagtcttgtcaagatttggtgcaaaacttttaccaacaagacccagattaacgtcactgttccctcgactgctaactgcacctccccttccctctgttggacggatggcatccaaaactggaccatgaagaatgtgacctacaaggagaacatcgccaaatgccagcatatctttgtgaatttccacctcccggatcttgctgtgggcaccatcttgctcatactctccctgctggtcctctgtggttgcctgatcatgattgtcaagatcctgggctctgtgctcaaggggcaggtcgccactgtcatcaagaagaccatcaacactgatttcccctttccctttgcatggttgactggctacctggccatcctcgtcggggcaggcatgaccttcatcgtacagagcagctctgtgttcacgtcggccttgacccccctgattggaatcggcgtgataaccattgagagggcttatccactcacgctgggctccaacatcggcaccaccaccaccgccatcctggccgccttagccagccctggcaatgcattgaggagttcactccagatcgccctgtgccactttttcttcaacatctccggcatcttgctgtggtacccgatcccgttcactcgcctgcccatccgcatggccaaggggctgggcaacatctctgccaagtatcgctggttcgccgtcttctacctgatcatcttcttcttcctgatcccgctgacggtgtttggcctctcgctggccggctggcgggtgctggttggtgtcggggttcccgtcgtcttcatcatcatcctggtactgtgcctccgactcctgcagtctcgctgcccacgcgtcctgccgaagaaactccagaactggaacttcctgccgctgtggatgcgctcgctgaagccctgggatgccgtcgtctccaagttcaccggctgcttccagatgcgctgctgctgctgctgccgcgtgtgctgccgcgcgtgctgcttgctgtgtggctgccccaagtgctgccgctgcagcaagtgctgcgaggacttggaggaggcgcaggaggggcaggatgtccctgtcaaggctcctgagacctttgataacataaccattagcagagaggctcagggtgaggtccctgcctcggactcaaagaccgaatgcacggccttgtaggggacgccccagattgtcagggatggggggatggtccttgagttttgcatgctctcctccctcccacttctgcaccctttcaccacctcgaggagatttgctccccattagcgaatgaaattgatgcagtcctacctaactcgattccctttggcttggtggtaggcctgcagggcacttttattccaacccctggtcactcagtaatcttttactccaggaaggcacaggatggtacctaaagagaattagagaatgaacctggcgggacggatgtctaatcctgcgcctagctgggttggtcagtagaacctattttcagactcaaaaaccatcttcagaaagaaaaggcccagggaaggaatgtatgagaggctctcccagatgaggaagtgtactctctatgactatcaagctcaggcctctccctttttttaaaccaaagtctggcaaccaagagcagcagctccatggcctccttgccccagatcagcctgggtcaggggacatagtgtcattgtttggaaactgcagaccacaaggtgtgggtctatcccacttcctagtgctccccacattccccatcagggcttcctcacgtggacaggtgtgctagtccaggcagttcacttgcagtttccttgtcctcatgcttcggggatgggagccacgcctgaactagagttcaggctggatacatgtgctcacctgctgctcttgtcttcctaagagacagagagtggggcagatggaggagaagaaagtgaggaatgagtagcatagcattctgccaaaagggccccagattcttaatttagcaaactaagaagcccaattcaaaagcattgtggctaaagtctaacgctcctctcttggtcagataacaaaagccctccctgttggatcttttgaaataaaacgtgcaagttatccaggctcgtagcctgcatgctgccaccttgaatcccagggagtatctgcacctggaatagctctccacccctctctgcctccttactttctgtgcaagatgacttcctgggttaacttccttctttccatccacccacccactggaatctctttccaaacatttttccattttcccacagatgggctttgattagctgtcctctctccatgcctgcaaagctccagatttttggggaaagctgtacccaactggactgcccagtgaactgggatcattaagtacagtcgagcacacgtgtgtgcatgggtcaaaggggtgtgttccttctcatcctagatgccttctctgtgccttccacagcctcctgcctgattacaccactgcccccgccccaccctcagccatcccaattcttcctggccagtgcgctccagccttatctaggaaaggaggagtgggtgtagccgtgcagcaagattggggcctcccccatcccagcttctccaccatcccagcaagtcaggatatcagacagtcctcccctgaccctcccccttgtagatatcaattcccaaacagagccaaatactctatatctatagtcacagccctgtacagcatttttcataagttatatagtaaatggtctgcatgatttgtgcttctagtgctctcatttggaaatgaggcaggcttcttctatgaaatgtaaagaaagaaaccactttgtatattttgtaataccacctctgtggccatgcctgccccgcccactctgtatatatgtaagttaaacccgggcaggggctgtggccgtctttgtactctggtgatttttaaaaattgaatctttgtacttgcattgattgtataataattttgagaccaggtctcgctgtgttgctcaggctggtctcaaactcctgagatcaagcaatccgcccacctcagcctcccaaagtgctgagatcacaggcgtgagccaccaccaggcctgattgtaatttttttttttttttttttactggttatgggaagggagaaataaaatcatcaaacccaaaaaaaaaaaaaaaaaa

SEQ ID NO: 15 - DNA, 7235 bp, Homo sapiens
gggatttccacttttattgcaaaacatgcttgcttgatgcaatgcatttcagatctagaaaaaaataatgcaagcaagctacatttacagatgtgtaaacaggcaaaatttccagaaccgtccaattttacttttccaaatttatggaaccaacccagattttttaaatgaaaatactaaaaccatatgtaaccgtaagacaagctaaaatgtaagcagacagcctgggaagcactgccctactgtacaggtgtgatattaagcacaagccatcctctggcctcttatgagatgttgttattctctgaaaaactttgatggaaaaggtttctgttaggcttagactgaattagctaaatgaccatgccacagtattattgtatcactcttctcacacacctaggcactggttgtggatagttgccatggtgacaatcaagataatccagatgacctaaacaaatgtctgtatgcagctgacactgctattctccctcacagtcttgtgaagagccacagtagctatggcagacatttggttgatggtttcatcatttcctaatatgagagtctggcactatgttggagagaggaatcacttcctcagagataggttcctttcaactcacatgtcaacagttactgtacaaaaagctcagaatacagtaatgattagagcttggcccctgactttaggaacttagaaccaaagcccaaagtcccaggccaagggcagacattgtgagcagcagaagggtgttttttatggagagccccataaatggatgggtgtagaccttgaagtctgagatgggaaatataccagaatgctcaatgcatcagacatgaagccccacgggcttccatttgacatgctggcaatctgaaggacaaagggcatgggttatttgggccttttccccactccttcaatatggctgaaagtgccagtggggattacaacctaagaaaccctgtgccccttcctgagggcttgattattgaatcctgatcttcctggaatctttcacttgagatgatcacctgcctaacatccaacatgttactatgctcttctttatgtacatgagttactggaaaccaacaataaaggagagtgagtcatactctccacatatgcgccaattaattttttaacttacttgttagtattcttgaagaacttactatcttaagtattttaccaatagatgatgcattttcctgcaactcaatggagttctaccttaaagtatttaccattgatccaaaattggaggaactaccttagattcttttaataacctagaaaggattggagcttgtattatttcctaaaccataggcaggcagtctagcttccctgctggatcataagaccaggacagcagagattgggtttgtattgatcagtagtagagaagtattgattgtctaacatctagtaagttgtctttatcagtatagacatagacaaaatacatgttttatgaatgaataaataataagaaccttaagagctgtttttttcccatttaagaaaacaaaaaggaaaagggaaaaatgatcttctggaataactctgctttgatattctccttttgttccccaaaaaatatattccagaaaggcaaaccaaatctgctcaagttgcctatgcacagacaatctcaacaatatggctcacagtgagcaagcagaaattcagggtctggaattgaatgtgagttatgggaagaaacagtgtgtgctttcacaaagatccttttgtcagacaagggaacagcatgataatccaatggaactggttaacatgacccaagatgacatgaccacagatatgctggtcgttctgaaacttgcccaagaaattgcaatcgtttcagcagaactccatgaaatattgacaaattaattccatcatcaaactaggagtgatattatcaggtaaaactatgatctcatgtatgtatttggtctttctgggtcacaatgtgacatgaaaaattttaaaggtcatattttagcaagatcaaaatgaattagataaatcaacttcacaaatgttaagcgaaacatgatcctattgagctgagaaagaagtacttgagatcgtttgagttttgactatcaaatgtcagcacaaaagcaaatatcaaaagcttaagaaaaaaattatttcaggaaaagaaaaacacatcaaattgagaaagtgttgttgaataactttgtttttagaactggaaatttaggcttattagacatgacattgatattacaaagccctagttttcatgaaaacataactagataaccccaaggtgtataggttaaaatgaactaattaatcaacaattatgcatcctgtgtctattttgtgcaaggtacttcctagacactgtagtttaaaagaataatttcctgtagaaaaacctaatctaataggacctactcataatttggcaaaatcatcctcataattccatgctactgaaatgggggtgtaataaagaaaaaatcatttgtttattcatttaacagggtttattaagcaccaatatatgcccagaactttgcagtgcactggaagttcagagataagaaaaccacagtttctgccatcaaaatgctaggaatctagaggtctcaaacatcagaggtcccttcaaactatgtctaccaggaaaataaaatacctgatttcatccctgcaaacaactctatacagacagaaatgcttcctagcttggaaactatagaaaattgaacatgaaaatgagagcaaagattctacatcaaaatggcaatgatccaatcagcccagtcaaggctgagtgggttgaatggggacttcgagtttggattcaatgctttgaacttcatagctccagagaagctgtgcaaaaaggggggattctggggaatctgaggaagattgttggagaaaccagctttcttcttgtctcctaatccctgctaagattgataccagtcaaggagctgagttactttcatttcattatgttgaccaagagaagcttatgatacagactttgacacagcacctatttcaagagaaagatgttactctacttatgggaacctgcagatcaaattctcctggggcaagagaagaaatttgttcagcagcctaagaagtcaactgtactttggacagtgctgccaaacacaccccaggattctcccacccctgtgctttcccagaagatgccacatagctggtatttggttttcatgaataaccaaagattcttctttgcttcctgaaccccaggcatccttaggaatttgcttcatgtcctcacttgtccttgaaggcttgtttttcaggatgaacttttaaagtctaagttgtttatattcaaatcaacccaatgctattgttgctgatattaaaccattccaaactgagccaactggcccctaaacaagccagcaggcagcccctcactaatgcccagtttagaacatgttaggaccaggctatctgcatagctttagtggttttagaagccagtaatggcagtgtaaaagagacaggaatcatacagagatgacctggaagaaaatttgagtttcagcacattcaaaagcatttgactcttctgccgtgttttgattttcaagtgagacaatcaatgtacacacaaagcctctagtacaatgactgatgcatactagggactgttatttatgttatttttttacagtatgcatggacttcattgtctcagtctttggtgttacttatgaactctaaatttacaccaaaaaaattttttttcagaaaaaggatcgtggagaaacaatcctaattattaaatcaaatgctttcacaattgctttttaaataagtacatctctgcaaataaaaatgcttgctcttgtctaccacaataaagaacagaaggaatcttggaatgaagagaggcaggaatgttggatctcagtggctgagttgtggcaattccattggacatatagtaccagatgttgcaccatctggatgatgtgtaggagataaagccaaattgcaaaaataaaagactcatgatttcccaaggaaagtcaactgtgcatggtggctatcagacaacaccctagcaatgaatgggggaacttgcagaagaaagagttaatgcttgcttaaaccttccctggggagatttgttcccaggctttcacggctttaacctcagcaaccctgtaagaaaattgtgattcatattttgatttgttatcatagatgcaaagagacataatttcatggattctaagatacacagagcaagaagagattttgatcacccagaacaaccacctgtctttttagatcaagaagcagaagctcagagcaaagaacatcacaccacgtccctcagtgattgaagcagtgattgagtcacagaataaatctggaactcaggtcttctgatctttgctccagatgttagagacaaaactaaaagtaaaataccaagtgaaatcaaagcatcacgattgagcccagaacatgaaaaagaacttcctggcccacttgagaaactgttaaaccggacatacctttggggacttcttcccttctctggaataagattgatgtttccatgctgtgaaagacgatgatgttccttctcccagattcctgctgtcttcaaaaggcctagcaaaaaccactgctgctgggtgcagttgagaaagggaatgaagaacaatcccatggccctgcaggcactcctcccctccacctctctgccctccacccctctgccctctcctaccttgttccttcttggtcctgccatgtttggactggttcacctttttggcccatcacttgggctcattcagcaacagtcatgccttaggttgcatcctcatctataggccaattaaacagggtttcttcttgataaatccctatggttttgattttaggaagtcaggcatgatttcttttgggatctatatgacccaaagtggccagttttaccaaaggtgtccctttgcactgggatctctgggtctcatttgaacacagcttgggtcctcttgccttgtcccagagaatccccatgttgtcaggacataagcagatactctagggccttcaccaaaagctacaaagaagcaaacaggactcaacttaggacatcttatggttatgtaactaagagattattttaggaagtaaacaggcctctgttggctcaaatgaacatcttcattgttagtttcccttacattgttctcgggatgaatcggccagaatttggcagggctccccagcagtccttgtgaatccctgtggtaggatgtttcagttctcctgagtcaggctgaagtgaaagctcctaagagatattcagaaagaaggatggtcaagttctagaacaacaatagttggcccagtaattttaaagattgtcattctacctaagccttcatcctccagatagcactgttatgggcttaaaataagcttcaagggaaataatgaatttctttgacaccgccaaaaatcggctgtatataagaataacttgagaatggcttacaatgaggtagtcaaaagcccagctcagtgctcaggaatgtatttgtaattcctcttagacttgtcgaatggactgcaatttgatacctaagagtaataaaaaatgagagcatattttatgttaatgttcccttctcttcctctgcccacaaaagtgaggtcagggaaggggagagaaacttatcctaagcgctccttatgagttgcttgtgacatgatagctctctctaaagttcatgcaatcctcatcgcaaccccatgagggagacgttattgtctccatcttatagatgaggaaactgaagacaaggccttgtcaaaggtcacacattgtaagaagtagagctgcatatttaaccaaagcccgtgtacctggaaggctcattctctctcccccttcttcctctcaaatcaggccctagctcctccacaatgcttcgggcttctacgttcacttagctttacagtaaaatgatcacagacccatgagttccctggagagtagagtgatgcctgggtcagctgttcctcttgtctgttcagcaggtcctttgagtggcttcacaatccagctgtcagtggcagaaaataatgcctcataaattattcacctggttcacattggaatccattagctctcggggcttggtgtggctgcagatcatccacaagtgccccttgttctctgcccccaaggagatcctgtcattgtttttaaccaaacaagttcaaatatctccacgtctctgctgactctggtcttttgtctctagactgtacctttatttctgagctctgaaattctgcagaaacaggtgagtctataaaaacattctgtgggaggccaaaagctcagaacagttgtgtctctctttctcagtattaaaagatcaaggtttcccatggaagccaaccggatctggtaattgggcccttctatacccctctgaatacttttccaaggagggggcagttcccacccccatagttcccctgagggttaagagtccaggcggtggccaagttgcaagatttggggcagccattttgccaaggatcagagatttgctgttggagaggtgaagtcggctctgtgccatcctgaaaaatcaacttagaaagataggtggacttcatcagatttgtcagtccttccttgttttaaagagaaaaatgatctctgccttgcttcagataggatatggctattttttttccttctttttctaatgtagcagtgatggaaaacaccagtctttgaagaacaaatggaggttcccagtgacatgaatccattatatttgattttggattgtattttttaacccctttttatggcatagaattctggtgccttgctccctgggtttgtctctgtgctacaagtaggctttcttcacattctggcttacaggggaacacaactattccacaagtggcctttagtgctctttataatatgatttcctgtaatttttaaactgtatgtgtaatttatattctggctctgtccaatatttgaacaactttaataggttgatttccatatatattatgcaaataattcttacctgatttttttatgttctatcttaaataaatactgcaccacttattaataaatagacatttcagtgataaaaaaaaaaaaaaaaaa

SEQ ID NO: 16 - DNA, 894 bp, Homo sapiens
tttgggtttggacgggtgcgtttgtttttcatagtgcaggtctggagccggactgagatggggagtagagggaccccttgaaacttcagggtggatgaatatgaagtttcatcaccagctgtgatgtttgatattggactgtggttgtgccagttgacactcaggagtggagactagatctgcaatctgttgggactgtgatgaggggatcatcctgccctcgaaccagacccagccctgtagcccaggcactattgcctggagttcctgggctctcagcgccaggaaatcatgaggttcagtgtctcaggcatgaggaccgactaccccaggagtgtgctggctcctgcttatgtgtcagtctgtctcctcctcttgtgtccaagggaagtcatcgctcccgctggctcagaaccatggctgtgccagccggcacccaggtgtggagacaagatctacaaccccttggagcagtgctgttacaatgacgccatcgtgtccctgagcgagacccgccaatgtggtcccccctgcaccttctggccctgctttgagctctgctgtcttgattcctttggcctcacaaacgattttgttgtgaagctgaaggttcagggtgtgaattcccagtgccactcatctcccatctccagtaaatgtgaaagcagaagacgttttccctgagaagacatagaaagaaaatcaactttcactaaggcatctcagaaacataggctagggtaatatgtgtaccagtagagaagcctgaggaatttacaaaatgatgcagctccaagccattgtatggcccatgtgggagactgatgggacatggagaatgacagtagattatcaggaaataaataaagtggtttttccaatgtaaaaaaaaaa

SEQ ID NO: 17 - DNA, 1867 bp, Homo sapiens
cacatagctcagttcccataaaagggctggtttgccgcgtcggggagtggagtgggacaggtatataaaggaagtacagggcctggggaagaggccctgtctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccatttctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaatcctcggccagcaggtcccctatgccaccaagggcaaccagtgggtaggatacgacgaccaggaaagcgtcaaaagcaaggtgcagtacctgaaggacaggcagctggcgggcgccatggtatgggccctggacctggatgacttccagggctccttctgcggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgattaatggaaatgtttacagatccccaagcctggcaagggaatttcttcaactccctgccccccagccctccttatcaaaggacaccattttggcaagctctatcaccaaggagccaaacatcctacaagacacagtgaccatactaattataccccctgcaaagcccagcttgaaaccttcacttaggaacgtaatcgtgtcccctatcctacttccccttcctaattccacagctgctcaataaagtacaagagcttaacagtgaaaaaaaaaaaaaaaaaaaaaaaaaaaaaa

SEQ ID NO: 18 - DNA, 3295 bp, Homo sapiens
tggagagggacaggaggaaacgcagcgccagcagcatctcatctaccctccttgacacctccccgtggctccagccagaccctagaggtcagccttgcggaccaacaggaggactcccagctttcccttttcaagaggtccccagacaccggccaccctcttccagcccctgcggccagtgcaaggaggcaccaatgctctgaggctgtcgcgtggtgcagcgtcgagcatcctcgccgaggtcctttctgctgcctgtcccgcctcaccccgctccatcacaccagctggccctctttgcttccttttcccagaatcgttaagccccgactcccactagcacctcgtaccaacctcgccccaccccatcctcctgccttcccgcgctccggtgtcccccgctgccatgagctcccccatcagcaagagccgctcgcttgccgccttcctgcagcagctgcgcagtccgaggcagcccccgagactggtgacatctacggcgtacacgtcccctcagccgcgagaggtgccagtctgcccgctgacagctggtggcgagactcagaacgcggccgccctgccgggccccaccagctggccactgctgggcagcctgctgcagattctctggaaagggggtctcaagaaacagcacgacaccctggtggagtaccacaagaagtatggcaagattttccgcatgaagttgggttcctttgagtcggtgcacctgggctcgccatgcctgctggaagcgctgtaccgcaccgagagcgcgtacccgcagcggctggagatcaaaccgtggaaggcctatcgcgactaccgcaaagaaggctacgggctgctgatcctggaaggggaagactggcagcgggtccggagtgcctttcaaaagaaactaatgaaaccaggggaagtgatgaagctggacaacaaaatcaatgaggtcttggccgattttatgggcagaatagatgagctctgtgatgaaagaggccacgtcgaagacttgtacagcgaactgaacaaatggtcgtttgaaagtatctgcctcgtgttgtatgagaagagatttgggcttctccagaagaatgcaggggatgaagctgtgaacttcatcatggccatcaaaacaatgatgagcacgtttgggaggatgatggtcactccagtcgagctgcacaagagcctcaacaccaaggtctggcaggaccacactctggcctgggacaccattttcaaatcagtcaaagcttgtatcgacaaccggttagagaagtattctcagcagcctagtgcagatttcctttgtgacatttatcaccagaatcggctttcaaagaaagaattgtatgctgctgtcacagagctccagctggctgcggtggaaacgacagcaaacagtctaatgtggattctctacaatttatcccgtaatccccaagtgcaacaaaagcttcttaaggaaattcaaagtgtattacctgagaatcaggtgccacgggcagaagatttgaggaatatgccgtatttaaaagcctgtctgaaagaatctatgaggcttacgccgagtgtaccatttacaactcggactcttgacaaggcaacagttctgggtgaatatgctttacccaaaggaacagtgctcatgctaaatacccaggtgttgggatccagtgaagacaattttgaagattcaagtcagtttagacctgaacgttggcttcaggagaaggaaaaaattaatccttttgcgcatcttccatttggcgttggaaaaagaatgtgcattggtcgccgattagcagagcttcaactgcatttggctctttgttggattgtccgcaaatacgacatccaggccacagacaatgagcctgttgagatgctacactcaggcaccctggtgcccagccgggaactccccatcgcgttttgccagcgataatacgcctcagatggtggtatttgctaacatcatatccaactcagggaagcggactgagtgctgggatccaaggcattctacagggttcactgctggtttacacttcacctgtgtcagcaccatcttcaggtgcttagaatggcctgggagcctgttctgtcttgcatcttccatgacatgaaagggaggctggcacttgtcagtcaggtagaggttacaaaccgtttcaggccctgcctaccacattcactgtttgaatctttaattcccaagaataagtttacatttcacaatgaatgacctacaacagctaaattttctggggctgggagtaatactgacaatccatttactgtagctctgcttaatgtactacttaggaaaatgtccctgcttaataatgtaagccaagctaaatgatggttaaagttatcaggcctcccatgaaattgcgttcttcctgcattgaaataaaaacattattgggaaactagagaacacctctatttttaaaaggactttaacgaagtcaaacaacttataagactagtgattcactggggcattattttgttagaggaccttaaaattgtttattttttaaatgtgattcctttatggcattagggtaaagatgaagcaataatttttaaattgtgtatgtgcatatgaagcacagacatgcatgtgtgtgtgtgtctgtgtgtgtgtgtccgtgtatgtgtgtgtgggttctaatggtaatttgcctcagtcatttttttaatatttgcagtacttgatttaggatctgtggtgcagggcaatgtttcaaagtttagtcacagcttaaaaacattcagtgtgactttaatattataaaatgatttcccatgccataatttttctgtctattaaatgggacaagtgtaaagcatgcaaaagttagagatctgttatataacatttgttttgtgatttgaactcctaggaaaaatatgatttcataaatgtaaaatgcacagaaatgcatgcaatacttataagacttaaaaattgtgtttacagatggtttatttgtgcatatttttactactgcttttcctaaatgcatactgtatataattctgtgtatttgataaatatttcttcctacattatatttttagaatatttcagaaatatacatttatgtctttatattgtaataaatatgtacatatctaggtatatgctttctctctgctgtgaaattatttttagaattataaattcacgtcttgtcagatttcatctgtataccttcaaattctctgaaagtaaaaataaaagtttttaaatattaaaaaaaaaaaaaaaaaaaaa

SEQ ID NO: 19 - DNA, 1789 bp, Homo sapiens
aggggctgaatacacagagcgctgagagagtggggcagtgtggtcacggacacaggtcatgggggccttggcaggagcggttttggcagagggtggggccggtgcctctggaaggtatgaagatgtaaatgaagccctgtccaggctatgggcatcaagacagcattgccggcggctgagctgggcctctactctctggtgctgagtggggccctggcctatgctggccggggcctccttgaggcttcacaagatggggcccacaggaaggccttccgggagtctgtgcgacctggctgggagtacattggccggaagatggatgtggctgacttcgagtgggtgatgtggttcacctcctttcgcaacgtcatcatctttgccctctccggacatgtgctgtttgctaaactctgcacgatggttgccccaaagctccgctcctggatgtatgctgtgtacggggccttggctgtgatgggcacaatgggcccttggtacctgctgctgctgcttggtcactgtgtgggcctctatgtggcctcgcttttgggccagccctggctctgtcttggccttggcttggccagcctggcctccttcaagatggaccccctaatctcttggcagagcgggtttgtaacaggcacttttgatcttcaagaggtgctgtttcatgggggcagcagcttcacagtgctgcgttgcgccagctttgcactggagagctgtgcccaccctgaccgccactactccttagctgacctgctcaagtacaacttctacctgcccttcttcttcttcgggccaatcatgacctttgatcgcttccatgctcaggtgagccaggtggagccagtgagacgcgagggtgagctgtggcacatccgagcccaggcaggcctaagcgtggtggccatcatggccgtcgacatcttctttcacttcttctacatcctcactatccccagcgacctcaagttcgccaaccgcctcccagacagtgccctcgctggcctagcctattcaaacctggtgtatgactgggtgaaggcggccgtcctctttggtgttgtcaacactgtggcatgcctcgaccacctggacccaccccagcctcccaagtgcatcaccgcactctacgtctttacggaaacgcactttgaccgtggcatcaacgactggctttgcaaatatgtgtataaccacattggtggggagcattccgctgtgatcccagagctggcagccacagtggccacatttgccatcaccacactgtggcttgggccttgtgacattgtctacctgtggtcattccttaactgctttggcctcaactttgagctctggatgcaaaaactggcagagtgggggcccctagcacgaattgaggcctctctgtcagtgcagatgtcccgtagggtccgggccctgtttggagccatgaacttctgggccatcatcatgtacaaccttgtgagcctgaacagcctcaaattcacagagctggttgcccggcgcctgctactcacagggtttccccagaccacgctgtccatcctgtttgtcacctactgtggcgtccagctggtaaaggagcgtgagcgaaccttggcactggaggaggagcagaagcaggacaaagagaagccggagtaggagggagcgggtagagggatgggctctgctcagctattcttgggccagatggggcctgaccgatagaataaaagacttttctacaacaaaaaaaaaaaaaaaaaaaaaaacaaaaaaaaaaaaaaaa

SEQ ID NO: 20 - DNA, 1852 bp, Homo sapiens
ccgcgctgtccgccgccgctgcctgagtcgactctgcgccgcccgccgcgatggaggccgccgcccagttcttcgtcgagagcccggacgtggtctacggccccgaggccatcgaggcgcaatacgagtaccggacgacgcgcgtcagccgcgagggtggcgttctcaaggtgcaccccacgtccacgcgcttcaccttccggaccgcccggcaggtgccccggctcggggtcatgcttgtcggctggggcgggaacaacggctccacactcaccgccgcggtgctggccaatcgactgcgtttgtcctggcccacgcgcagcggccgcaaggaggccaactactacggctcgctgactcaggcgggcaccgtgagcctgggcctggacgccgagggccaggaggtgttcgtacccttcagcgcggtgctgcccatggtggcgcccaacgacctcgtgttcgatggctgggacatctcgtcgctgaacctggccgaggcgatgcggcgcgcgaaggtgctggactgggggctgcaggagcaactgtggccgcacatggaggccctgcggccccggccttctgtttacatccccgaattcatcgcggccaaccagagcgcgcgcgcggacaacctcatcccaggctcgcgtgcgcagcagctggagcagatccgcagggacatccgagacttccggtctagcgcggggctggacaaagtcatagtgctgtggacggcgaacacggagcgcttctgtgaggtgattccaggcctcaacgacacagccgagaacctgctgcgcaccattgagctcggtctggaggtgtcgccctccacgctcttcgccgtggccagcatcctggagggctgtgccttcctcaatgggtctccgcagaacaccctggtgcccggagctcttgagctcgcgtggcagcaccgggtttttgtgggcggagatgacttcaagtcaggccagaccaaagtcaagtccgtgcttgtggacttcctcattggctccggcctcaagaccatgtccatcgtgagttacaaccacctgggcaacaacgatggggagaacctatcggcgccattgcagttccgctctaaggaggtgtccaagagcaacgtggtggacgacatggtgcagagcaacccagtgctctatacgcccggcgaagagcctgaccactgcgtggtcatcaagtatgtgccgtacgtgggtgacagcaagcgcgcgctggatgagtatacctcggagctgatgctgggcggaaccaacacactggtgctgcacaacacgtgtgaggactcgctgctggccgcacccatcatgctggacctagcgctgctgaccgagctgtgccagcgcgtgagcttctgcactgacatggaccccgagccgcagaccttccaccccgtgctgtccctgctcagcttcctcttcaaggcgccactagtgccgcccggcagcccggtggtcaatgcgcttttccgccagcgcagctgcatcgagaacatcctcagggcctgcgtggggctcccgccacagaaccacatgctcctggaacacaaaatggagcgcccagggcccagcctcaagcgagttggacccgtggctgccacctaccctatgttgaacaagaaaggaccggtacccgctgccaccaatggctgcaccggtgatgccaatgggcatctgcaagaggagcccccaatgcccaccacctgaggccccggtcacacagtttctcggctcttcctccccgctgccccccacgaccctaccttgaaggcccccacaaataaaggcgctgccactcagccctcaaaaaaaaaaaaaaaaaaaaaaaaaaa

SEQ ID NO: 21 - DNA, 3450 bp, Homo sapiens
gtagaacagtggggagctgtgcggatgggtgaagtgcatgtatgcctgcctagacgggccagaaaagccaaacttaagaaatctgcctatgtacagaacaagtcaactaaggggttctcattaatctaccgtctgtgaatatcgttatttttcaggtatacaaatgctctcagtccagccagacaccaagccgaaaggttgtgctggctgcaaccgaaagatcaaggaccggtatcttctaaaggcactggacaaatactggcatgaagactgcctgaagtgtgcctgctgtgactgtcgcttgggagaggtgggctccaccctgtacactaaagctaatcttatcctttgtcgcagagactatctgaggctctttggtgtaacgggaaactgcgctgcctgtagtaagctcatccctgcctttgagatggtgatgcgtgccaaggacaatgtttaccacctggactgctttgcatgtcagctttgtaatcagagattttgtgttggagacaaatttttcctaaagaataacatgatcctttgccagacggactacgaggaaggtttaatgaaagaaggttatgcaccccaggttcgctgatctatcaacatcaccccattaagaatacaaagcactacattcttttatcttttttgctccacatgtacataagaattgacacaggaacctactgaatagcgtagatataggaaggcaggatggttatatggaataaaaggcggactgcatctgtatgtagtgaaattgccccagttcagagttgaatgtttattattaaagaaaaaagtaatgtacatatggctggatttttttgcttgctattcgtttttgtgtcacttggcatgagatgtttattttggactattgtatataatgtattgtaatatttgaagcacaaatgtaatacagttttattgtgttaccatttgtgttccatttgcttctttgtattgttgcatttagtacaatcagtgtttaaacttactgtatatttatgctttctgtatttaccagctattttaaatgagctgtaactttctagtaaagaattgaaaagcaaatctcactaatgatacacagatagataaagcaagtctatcaacattaaaaatactaaaaaataaagacacacacagagcattttagtgacatccactacttattgccgctatgagttagagtctatcagtgttcttgttataaccccctattttcagggggttaaaaatcagctttaaaaaaatacataaaaatttcatcttaaagcactttcattttataccaacgtgaaaagtgccatttttagaataactttaaagcttaacaggtttccttttaatatcctttttttgtgtgctctttacttacacaatggctttgttttgctttttcagccacaccccttatgtgaactagtgcctttgggtatcacgtaaaattttttccaaagggttactttaaaaatctgttaccacaattatgagatgatttttaagtgataaattaaacttcttcttgtataaattctgcccagatctctccacaagagctgagggtttcataactttatggcttaataaatgtatgacactgaaaagatttgagtgtgaatctactgaaatcactataatgcacattgaagctatgatggtatttgagtagtgaggttacttttgatcggagcaacataatgctcatagaatcttctagaagaagagaaacaaagggattgataaaatgctgagaactagtgattatatatttttctgtatttacctgacatttattttaatgttcaaaaagtaaacactttaagtttgatgtgttttactctctcattgttttaagtaattgccaactcagaatacatcattcttaggctgaaatttgtctttccattttttaaggtgaaatagtactaccttacgtgatagcatacaaagaagaaagctctagaaagagaaattatggagaatgattatttaaattacaattaaggaaatgagaatatgatcccctcttccgagttgcccacaaacttgcttctttgcttttgctccctgtaatagaactacttttcaacaaatctaattttgcacggcaccgttaaccatattttcactacagcaaacttagtgctatgggttttctttttctttgtttttttcttgatcacttgtataggaaacaatattttccagtgttatttgcatatatattttgtccttccaatatatgcattacagatgaaaattaaatgttatacctgaattcttgggttggggccaaaatattaagctgaaaataatgctggtgtggatttgttttaaaacaaagctttattatgaacatgcatgtgaatctggatattgcctcttatttttaagaaaatggttctgtgaaaagtgaatgatatgtatttttccaaatgcttcatggttaggagtcttcaagttccatgttccccagatttgagatatactaaagaaagaaattcaaaagtagctatttggggcccacaaaaataactattattttagccttagagccttacacttgtttcatgaagagaaaggacttgcataaccaaaataaacaaagcaagacaaattaaaaatatgtgggggagagatcagtgaaaagtggttttcttaatgcagccctgctggtccccattaacaattgcttgaaattcacatggatgtaaaattataattgtcaggatcttattcagatgatcttttaaggtttaactggttttgcttttgtttatctatatgtcaaaatacttgtaaattgggaacaaacttctctcagcttcttgaagttgttcaactatccttgccactggaagaccaaacaaggttttcactgctttttcttttacataatatgctgagaattatttcttatgctttttactacaaacaaaattactcacctggattaaagattaaggccttaatctgtttagattatctttaatctccatgaaatcgtgaaataagacaagaatagtgtttcagctgtaggccattttacagctaattgcccataaattgtagcatttattgacctgaagtactaagctaattgtcttgactactcaaagcccctgaattgttgtcaactttcccctttgtgttgtgtagccctaacgtcatttagcttgttgtctgatgcctccagtaggacacctccgatggagctttgatttctgagcagcgaaagctcccttcctaagatgcatctcgcataggctgcctatgatgaaggaccgtgcacctccactccaacagagtgctgagtttaaaagttgacctgtgtttgtaatttcactttcatcttgcttaataaatatctgctggattctttcattcacttttttacatttggatttatgtttttaataaaaggggtgttacact

SEQ ID NO: 22 - DNA, 110 bp, Homo sapiens
tgaacatccaggtctggggcatgaacctggcatacaatgtagatttctgtgttcgttaggcaacagctacattgtctgctgggtttcaggctacctggaaacatgttctc

SEQ ID NO: 23 - DNA, 990 bp, Homo sapiens
tccggagcccggctcgctggggcagcatggcggggtcgccgctgctctgggggccgcgggccgggggcgtcggccttttggtgctgctgctgctcggcctgtttcggccgccccccgcgctctgcgcgcggccggtaaaggagccccgcggcctaagcgcagcgtctccgcccttggctgagactggcgctcctcgccgcttccggcggtcagtgccccgaggtgaggcggcgggggcggtgcaggagctggcgcgggcgctggcgcatctgctggaggccgaacgtcaggagcgggcgcgggccgaggcgcaggaggctgaggatcagcaggcgcgcgtcctggcgcagctgctgcgcgtctggggcgccccccgcaactctgatccggctctgggcctggacgacgaccccgacgcgcctgcagcgcagctcgctcgcgctctgctccgcgcccgccttgaccctgccgccctcgcagcccagcttgtccccgcgcccgtccccgccgcggcgctccgaccccggcccccggtctacgacgacggccccgcgggcccggatgctgaggaggcaggcgacgagacacccgacgtggaccccgagctgttgaggtacttgctgggacggattcttgcgggaagcgcggactccgagggggtggcagccccgcgccgcctccgccgtgccgccgaccacgatgtgggctctgagctgccccctgagggcgtgctgggggcgctgctgcgtgtgaaacgcctagagaccccggcgccccaggtgcctgcacgccgcctcttgccaccctgagcactgcccggatcccgtgcaccctgggacccagaagtgcccccgccatcccgccaccaggactgctccccgccagcacgtccagagcaacttaccccggccagccagccctctcacccgaggatccctaccccctggccccacaataaacatgatctgaagcaaaaaaaaaaaaaaaaaa

SEQ ID NO: 24 - DNA, 1152 bp, Homo sapiens
cgctcctcgggctgcccctcggttgacaatggtctccaggatggtctctaccatgctatctggcctactgttttggctggcatctggatggactccagcatttgcttacagcccccggacccctgaccgggtctcagaagcagatatccagaggctgcttcatggtgttatggagcaattgggcattgccaggccccgagtggaatatccagctcaccaggccatgaatcttgtgggcccccagagcattgaaggtggagctcatgaaggacttcagcatttgggtccttttggcaacatccccaacatcgtggcagagttgactggagacaacattcctaaggactttagtgaggatcaggggtacccagaccctccaaatccctgtcctgttggaaaaacagatgatggatgtctagaaaacacccctgacactgcagagttcagtcgagagttccagttgcaccagcatctctttgatccggaacatgactatccaggcttgggcaagtggaacaagaaactcctttacgagaagatgaagggaggagagagacgaaagcggaggagtgtcaatccatatctacaaggacagagactggataatgttgttgcaaagaagtctgtcccccatttttcagatgaggataaggatccagagtaaagagaagatgctagacgaaaacccacattacctgttaggcctcagcatggcttatgtgcacgtgtaaatggagtccctgtgaatgacagcatgtttcttacatagataattatggatacaaagcagctgtatgtagatagtgtattgtcttcacaccgatgattctgctttttgctaaattagaataagagcttttttgtttcttgggtttttaaaatgtgaatctgcaatgatcataaaaattaaaatgtgaatgtcaacaataaaaagcaagactatgaaaggctcagatttcttgcagtttaaaatggtgtctgaggttgtactattttggccaagtctgtagaaagctgtcatttgattttgattatgtagttcatccagcccttgggcattgttatacaccagtaaagaaggctgtactcaagaggaggagctgacacatttcacttggctgcgtcttaataaacatgaatgcaagcattggc

SEQ ID NO: 25 - DNA, 662 bp, Homo sapiens
tttttttttttttttttttcttttaacgggcttcaattgcagagttgaatcattcattattttcagttcataatacattgtcatcattggtccagataggttgaattcttatttcattcattcattactcattcatttattcattcagttacagtgccatcaatgtccatacacccaccgctctactcaagaacttgaactgctatttacctatatgttcctctattatcctacctcctacctccccacacagcagcaatcagtatccagaagtttatgattatcactcctttgctttaaacagttggattacatgtatatgtatgcctaaattatagttttgtggggtttttgtgaaccccacaagagtgaaatgttctaagtcttgtgggacttgttttcatttaatatcttattgaatgacactcatattgttgtgggtagctgtaggtcactccacttcacagttgtgtaatattctgtcatgtgattttaccataagctctccacccattttcctgaggatagctatttggattatttccagttctttattaagaacaatactatcttgaacatccttatatgtgtatttcctagtactcctttataaaaatttcctttggaaatataccaagaattggaaatttggggttgtaagg

SEQ ID NO: 26 - DNA, 434 bp, Homo sapiens
atgtaccttgtgacagtgtggcctctcagatccagcccgactcagccagcaggacaaagggaccacggcttggaatgctggcgcagaaagcagacctggctgctggtggccggcatcaagtacttcctgacgatggagatggggagcacagactgccgcaagaccagggtcactggagaccacgtcgacctcaccacttgccccctggcagcaggggcgcagcaggagaagctgcgctgtgactttgaggtccttgtggttccctggcagaactcctctcagctcctaaagcacaactgtgtgcagatgtgataagtccccgagggcgaaggccattgggtttggggccatggtggagggcacttcaggtccgtgggccgtatctgtcacaataaatggccagtgctgcttcttgcattggtttcttccaagtg

SEQ ID NO: 27 - DNA, 2026 bp, Homo sapiens
gccatggctgagtcacacctgctgcagtggctgctgctgctgctgcccacgctctgtggcccaggcactgctgcctggaccacctcatccttggcctgtgcccagggccctgagttctggtgccaaagcctggagcaagcattgcagtgcagagccctagggcattgcctacaggaagtctggggacatgtgggagccgatgacctatgccaagagtgtgaggacatcgtccacatccttaacaagatggccaaggaggccattttccaggacacgatgaggaagttcctggagcaggagtgcaacgtcctccccttgaagctgctcatgccccagtgcaaccaagtgcttgacgactacttccccctggtcatcgactacttccagaaccagactgactcaaacggcatctgtatgcacctgggcctgtgcaaatcccggcagccagagccagagcaggagccagggatgtcagaccccctgcccaaacctctgcgggaccctctgccagaccctctgctggacaagctcgtcctccctgtgctgcccggggccctccaggcgaggcctgggcctcacacacaggatctctccgagcagcaattccccattcctctcccctattgctggctctgcagggctctgatcaagcggatccaagccatgattcccaagggtgcgctagctgtggcagtggcccaggtgtgccgcgtggtacctctggtggcgggcggcatctgccagtgcctggctgagcgctactccgtcatcctgctcgacacgctgctgggccgcatgctgccccagctggtctgccgcctcgtcctccggtgctccatggatgacagcgctggcccaaggtcgccgacaggagaatggctgccgcgagactctgagtgccacctctgcatgtccgtgaccacccaggccgggaacagcagcgagcaggccataccacaggcaatgctccaggcctgtgttggctcctggctggacagggaaaagtgcaagcaatttgtggagcagcacacgccccagctgctgaccctggtgcccaggggctgggatgcccacaccacctgccaggccctcggggtgtgtgggaccatgtccagccctctccagtgtatccacagccccgacctttgatgagaactcagctgtccagctgcaaaggaaaagccaagtgagacgggctctgggaccatggtgaccaggctcttcccctgctccctggccctcgccagctgccaggctgaaaagaagcctcagctcccacaccgccctcctcaccgcccttcctcggcagtcacttccactggtggaccacgggcccccagccctgtgtcggccttgtctgtctcagctcaaccacagtctgacaccagagcccacttccatcctctctggtgtgaggcacagcgagggcagcatctggaggagctctgcagcctccacacctaccacgacctcccagggctgggctcaggaaaaaccagccactgctttacaggacagggggttgaagctgagccccgcctcacacccacccccatgcactcaaagattggattttacagctacttgcaattcaaaattcagaagaataaaaaatgggaacatacagaactctaaaagatagacatcagaaattgttaagttaagctttttcaaaaaatcagcaattccccagcgtagtcaagggtggacactgcacgctctggcatgatgggatggcgaccgggcaagctttcttcctcgagatgctctgctgcttgagagctattgctttgttaagatataaaaaggggtttctttttgtctttctgtaaggtggacttccagcttttgattgaaagtcctagggtgattctatttctgctgtgatttatctgctgaaagctcagctggggttgtgcaagctagggacccattcctgtgtaatacaatgtctgcaccaatgctaataaagtcctattctcttttatgaaaaaaaaaaaaaaaaaaaaaaaaaaaaaaaaaaaaa

SEQ ID NO: 28 - DNA, 2617 bp, Homo sapiens
gacgtggtgctgagcccctgcgcggtttctggtgcgtagagactgtaaatcgctgcgcttctcagtcatcatcatcccagcttttcccggctcgaattcagcctccaactcaagctcgcgggaaagactacctgagaggagaaaagcttctgtccctggaccttcttctgagggtggtgaggtttgtttagggtcgcagaagcagggaggactgactcagccctcacagagaagaggattcctccccatcccgcttcgccccggaaaagctgacaagaacttcaggtgtaagccctgagtagtgaggatctgcggtctccgtggagagctgtgcctggaagagaaggacgctggtgggggctgagatcagagctgtcttctggcccagttgcccccatgcttctgtcatggctaacagttctaggggctggaatggtcgtcctgcacttcttgcagaaactcctgttcccttacttttgggatgacttctggttcgtgttgaaggtggtgctcattataattcggctgaagaagtatgaaaagagaggggagctggtgactgtgctggataaattcttgagtcatgccaaaagacaacctcggaaacctttcatcatctatgagggagacatctacacctatcaggatgtagacaaaaggagcagcagagtggcccatgtcttcctgaaccattcctctctgaaaaagggggacacggtggctctgctgatgagcaatgagccggacttcgttcacgtgtggttcggcctcgccaagctgggctgcgtggtggcctttctcaacaccaacattcgctccaactccctcctgaattgcatccgcgcctgtgggcccagagccctagtggtgggcgcagatttgcttggaacggtagaagaaatccttccaagcctctcagaaaatatcagtgtttgggggatgaaagattctgttccacaaggtgtaatttcactcaaagaaaaactgagcacctcacctgatgagcccgtgccacgcagccaccatgttgtctcactcctcaagtctacttgtctttacatttttacctctggaacaacaggtctaccaaaagcagctgtgattagtcagctgcaggttttaaggggttctgctgtcctgtgggcttttggttgtactgctcatgacattgtttatataacccttcctctgtatcatagttcagcagctatcctgggaatttctggatgtgttgagttgggtgccacttgtgtgttaaagaagaaattttcagcaagccagttttggagtgactgcaagaagtatgatgtgactgtgtttcagtatattggagaactttgtcgctacctttgcaaacaatctaagagagaaggagaaaaggatcataaggtgcgtttggcaattggaaatggcatacggagtgatgtatggagagaatttttagacagatttggaaatataaaggtgtgtgaactttatgcagctaccgaatcaagcatatctttcatgaactacactgggagaattggagcaattgggagaacaaatttgttttacaaacttctttccacttttgacttaataaagtatgactttcagaaagatgaacccatgagaaatgagcagggttggtgtattcatgtgaaaaaaggagaacctggacttctcatttctcgagtgaatgcaaaaaatcccttctttggctatgctgggccttataagcacacaaaagacaaattgctttgtgatgtttttaagaagggagatgtttaccttaatactggagacttaatagtccaggatcaggacaatttcctttatttttgggaccgtactggagacactttcagatggaaaggagaaaatgtcgcaaccactgaggttgctgatgttattggaatgttggatttcatacaggaagcaaacgtctatggtgtggctatatcaggttatgaaggaagagcaggaatggcttctattattttaaaaccaaatacatctttagatttggaaaaagtttatgaacaagttgtaacatttctaccagcttatgcttgtccacgatttttaagaattcaggaaaaaatggaagcaacaggaacattcaaactattgaagcatcagttggtggaagatggatttaatccactgaaaatttctgaaccactttacttcatggataacttgaaaaagtcttatgttctactgaccagggaactttatgatcaaataatgttaggggaaataaaactttaagatttttatatctagaactttcatatgctttcttaggaagagtgagaggggggtatatgattctttatgaaatggggaaagggagctaacattaattatgcatgtactatatttccttaatatgagagataattttttaattgcataagaattttaatttcttttaattgatataaacagtagttgattattctttttatctatttggagattcagtgcataactaagtattttccttaatactaaagattttaaataataaatagtggctagcggtttggacaatcactaaaaatgtactttctaataagtaaaatttctaattttgaataaaagattaaattttactgaaatattttaa

SEQ ID NO: 29 - DNA, 1573 bp, Homo sapiens
aatttggaacttaatgggcctttgcgtcctccttccctgagcctccttttattccagacttctcagtgtgagtctgtgcgtccctccgacgatctcagggagtggggtgccttcatctgcctgttccctgttcctcaggctgacgctcccgctgtcctccccgcctcccctcactccttttctccctcccttcctccttgtggggaggctcttggccagggtccctgagcccgggcgggtgctggcagaggacgcagaaggggtgaggtcacgtctcccttgagccccgagccgctggcttttcagagcctcgccacaagccggcggccagagccccagaccacacagaccgtgcgctcctccgccctcccggcgccgccggcctcgcccatgtctcagtacgcccctagcccggacttcaagagggctttggacagcagtcccgaggccaacactgaagatgacaagaccgaggaggacgtgcccatgcccaagaactacctgtggctcaccatcgtctcgtgtttttgccctgcgtaccccatcaacatcgtggctttggtcttttccatcatgtctctgaacagctacaacgatggagactacgaaggagccaggcggcttgggcggaatgctaagtgggtagccatcgcctccatcatcattggccttctcatcatcggcatttcttgtgcagttcacttcacaaggaatgcctgaggaaccagcggtcagtgggctgtgagcgtggaggatggacctcatccacacacaccccaaaggagtttctaaggaatggatccttgacttcagactgtgagatcttttcctccaggactctccagaggcaggtccctggcaaatgaacaagaaaaaaaaaaaaaaaaagtccaaaatttaggcaatccaagctgcacagccggatcagccaaagtcattgatttgtaaaaatgaaaagaaaacagaaaaaagaaaaatgaagtctcactgtctcagtttagcgaatcccgttgtgtccactcctgtcctccagaggcgagcctcaggaaatcacataacttttcactgaggggatccagggggtctccatatagggggagatggaggtttctaggaagagcagcaggtgctggtatttacaatgttgagcacaaacatttgcagcatgtttaaaattgtctagtagagttcaagttgtggatttgcttttccttttattcttataaccttcagtaactcctcctctgggagtcagcactcccatgcccagagttcacccatctggtcatcaaacactcaaagaaggggcttttctggccttttgtcttgatgcttatatttccaaataggccccctcccttgcttgcatccacgttggtcaacttgaccaaaacctcactcttcactcaaacaggctctgagaatggacttagtggccaattctaggtacatgagcacttcctgtatcccagttttgggaataaactggctgtatttatagaatgtgctttttttttttcaatttctcactctctctcctatctctagcaagtctcaggcaagatctttgatt

SEQ ID NO: 30 - protein, 1336 aa, Homo sapiens
MTLDRPGEGATMLKTFTVLLFCIRMSLGMTSIVMDPQPELWIESNYPQAPWENITLWCRSPSRISSKFLLLKDKTQMTWIRPSHKTFQVSFLIGALTESNAGLYRCCYWKETGWSKPSKVLELEAPGQLPKPIFWIQAETPALPGCNVNILCHGWLQDLVFMLFKEGYAEPVDYQVPTGTMAIFSIDNLTPEDEGVYICRTHIQMLPTLWSEPSNPLKLVVAGLYPKPTLTAHPGPIMAPGESLNLRCQGPIYGMTFALMRVEDLEKSFYHKKTIKNEANFFFQSLKIQDTGHYLCFYYDASYRGSLLSDVLKIWVTDTFPKTWLLARPSAVVQMGQNVSLRCRGPVDGVGLALYKKGEDKPLQFLDATSIDDNTSFFLNNVTYSDTGIYSCHYLLTWKTSIRMPSHNTVELMVVDKPPKPSLSAWPSTVFKLGKAITLQCRVSHPVLEFSLEWEERETFQKFSVNGDFIISNVDGKGTGTYSCSYRVETHPNIWSHRSEPLKLMGPAGYLTWNYVLNEAIRLSLIMQLVALLLVVLWIRWKCRRLRIREAWLLGTAQGVTMLFIVTALLCCGLCNGVLIEETEIVMPTPKPELWAETNFPLAPWKNLTLWCRSPSGSTKEFVLLKDGTGWIATRPASEQVRAAFPLGALTQSHTGSYHCHSWEEMAVSEPSEALELVGTDILPKPVISASPTIRGQELQLRCKGWLAGMGFALYKEGEQEPVQQLGAVGREAFFTIQRMEDKDEGNYSCRTHTEKRPFKWSEPSEPLELVIKEMYPKPFFKTWASPVVTPGARVTFNCSTPHQHMSFILYKDGSEIASSDRSWASPGASAAHFLIISVGIGDGGNYSCRYYDFSIWSEPSDPVELVVTEFYPKPTLLAQPGPVVFPGKSVILRCQGTFQGMRFALLQEGAHVPLQFRSVSGNSADFLLHTVGAEDSGNYSCIYYETTMSNRGSYLSMPLMIWVTDTFPKPWLFAEPSSVVPMGQNVTLWCRGPVHGVGYILHKEGEATSMQLWGSTSNDGAFPITNISGTSMGRYSCCYHPDWTSSIKIQPSNTLELLVTGLLPKPSLLAQPGPMVAPGENMTLQCQGELPDSTFVLLKEGAQEPLEQQRPSGYRADFWMPAVRGEDSGIYSCVYYLDSTPFAASNHSDSLEIWVTDKPPKPSLSAWPSTMFKLGKDITLQCRGPLPGVEFVLEHDGEEAPQQFSEDGDFVINNVEGKGIGNYSCSYRLQAYPDIWSEPSDPLELVGAAGPVAQECTVGNIVRSSLIVVVVVALGVVLAIEWKKWPRLRTRGSETDGRDQTIALEECNQEGEPGTPANSPSSTSQRISVELPVPI

SEQ ID NO: 31 - DNA, 3859 bp, Homo sapiens
aatctatcagggaacggcggtggccggtgcggcgtgttcggtgcgctctggccgctcaggccgtgcggctgggtgagcgcacgcgaggcggcgaggcggcaagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaagcaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcagttcatgaaattatatgaagaaaaaaccgggaacgcttggcactccaaaaatttcacgaagtatcccaaaaagttttaccccctggagattgactatggccaggatgaagaggcagtgaagaagctcacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacagtgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactaccatacgtctcagggagacccaataggcttaatcctgttgggagaagttgcccttggaaacatgtatgaactgaagcacgcttcacatatcagcaggttacccaagggcaagcacagtgtcaaaggtttgggcaaaactacccctgatccttcagctaacattagtctggatggtgtagacgttcctcttgggaccgggatttcatctggtgtgatagacacctctctactatataacgagtacattgtctatgatattgctcaggtaaatctgaagtatctgctgaaactgaaattcaattttaagacctccctgtggtaattgggagaggtagccgagtcacacccggtggctgtggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaaagcttcgttagaatgtctgccttactggtttccccagggaaggaaaaatacacttccacccttttttctaagtgttcgtctttagttttgattttggaaagatgttaagcatttatttttagttaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttg

SEQ ID NO: 32 - protein, 9 aa, Homo sapiens
SLLKFLAKV

SEQ ID NO: 33 - protein, 9 aa, Homo sapiens
MLLVFGIDV

SEQ ID NO: 34 - protein, 9 aa, Homo sapiens
KVTDLVQFL

SEQ ID NO: 35 - protein, 10 aa, Homo sapiens
GLYDGMMEHL

SEQ ID NO: 36 - protein, 9 aa, Homo sapiens
ILILSIIFI

SEQ ID NO: 37 - protein, 9 aa, Homo sapiens
FLWGPRAHA

SEQ ID NO: 38 - protein, 9 aa, Homo sapiens
VIWEALNMM

SEQ ID NO: 39 - protein, 9 aa, Homo sapiens
KMSILKFLA

SEQ ID NO: 40 - protein, 9 aa, Homo sapiens
KNYEDHFPL

SEQ ID NO: 41 - protein, 9 aa, Homo sapiens
FVLVTSLGL

SEQ ID NO: 42 - protein, 9 aa, Homo sapiens
ILFSEASEC

SEQ ID NO: 43 - protein, 9 aa, Homo sapiens
GMLSDVQSM

SEQ ID NO: 44 - protein, 9 aa, Homo sapiens
ILILILSII

SEQ ID NO: 45 - protein, 9 aa, Homo sapiens
GILILILSI

SEQ ID NO: 46 - protein, 9 aa, Homo sapiens
NMMGLYDGM

SEQ ID NO: 47 - protein, 9 aa, Homo sapiens
QIACSSPSV

SEQ ID NO: 48 - protein, 9 aa, Homo sapiens
LIPSTPEEV

SEQ ID NO: 49 - protein, 9 aa, Homo sapiens
SMPKTGILI

SEQ ID NO: 50 - protein, 9 aa, Homo sapiens
IIFIEGYCT

SEQ ID NO: 51 - protein, 8 aa, Homo sapiens
WEALNMGL

SEQ ID NO: 52 - DNA, 50 bp, Homo sapiens
ccctgcaagtcagccccatctgctgttccttggtctctaatcacctgagc

SEQ ID NO: 53 - DNA, 50 bp, Homo sapiens
accttggtgctcgccctgacagtgattctggagctgacgtgaaggcaccc

SEQ ID NO: 54 - DNA, 50 bp, Homo sapiens
gcagcacctggttatgcctcctttcatctcaaagccaaagagctgccagg

SEQ ID NO: 55 - DNA, 50 bp, Homo sapiens
gtacagttttgctcaggtcacgccaacagggaaacctcaagtgtaggtct

SEQ ID NO: 56 - DNA, 50 bp, Homo sapiens
gctcccactagttcctcgggatctccaataggctctcctacaaccacccc

SEQ ID NO: 57 - DNA, 50 bp, Homo sapiens
ccctgcagcctacgggtctgttttctgtgtgtgcccatttccttgacagc

SEQ ID NO: 58 - DNA, 50 bp, Homo sapiens
cagcccctcgcacagagacggccaaaatgtatgctgtagacacaagggtg

SEQ ID NO: 59 - DNA, 50 bp, Homo sapiens
gcaggtggaagtgtgatcccgtcgaccaatgccaggattcagagactggg

SEQ ID NO: 60 - DNA, 50 bp, Homo sapiens
agtggacttagcccctgtttgctcctccgataactggggtgaccttggtt

SEQ ID NO: 61 - DNA, 50 bp, Homo sapiens
ggtcatggccctggactacctgcagaaccagcgcatcattcacagggata

SEQ ID NO: 62 - DNA, 50 bp, Homo sapiens
agacttcccagaaataactggttagctgtttcctgtcatagaatggagtc

SEQ ID NO: 63 - DNA, 50 bp, Homo sapiens
ctctggctgcagttaaatggtcttttgcattttgctctggctttcaggcc

SEQ ID NO: 64 - DNA, 50 bp, Homo sapiens
cagagtctccgggccttggtaattcctagaccacagcaccatgcattagg

SEQ ID NO: 65 - DNA, 50 bp, Homo sapiens
atctaggaaaggaggagtgggtgtagccgtgcagcaagattggggcctcc

SEQ ID NO: 66 - DNA, 50 bp, Homo sapiens
tctggcttacaggggaacacaactattccacaagtggcctttagtgctct

SEQ ID NO: 67 - DNA, 50 bp, Homo sapiens
gctggctcctgcttatgtgtcagtctgtctcctcctcttgtgtccaaggg

SEQ ID NO: 68 - DNA, 50 bp, Homo sapiens
gggatggggctgtggggatagtgaggcatcgcaatgtaagactcgggatt

SEQ ID NO: 69 - DNA, 50 bp, Homo sapiens
gatttaggatctgtggtgcagggcaatgtttcaaagtttagtcacagctt

SEQ ID NO: 70 - DNA, 22 bp, Homo sapiens
gggccttcaactaccatcccac

SEQ ID NO: 71 - DNA, 21 bp, Homo sapiens
ccctgtctaggtagctggcac

SEQ ID NO: 72 - DNA, 22 bp, Homo sapiens
agagaaacctgacgcagggagc

SEQ ID NO: 73 - DNA, 21 bp, Homo sapiens
tccaccagcggagcacaggcc

SEQ ID NO: 74 - DNA, 20 bp, Homo sapiens
ctctgtggcccaggcactgc

SEQ ID NO: 75 - DNA, 28 bp, Homo sapiens
tcttttctgtccattgattctcagcctc

SEQ ID NO: 76 - DNA, 19 bp, Homo sapiens
cggtgtagaggcggcggac

SEQ ID NO: 77 - DNA, 20 bp, Homo sapiens
cacaaccccgaccgcaggac

SEQ ID NO: 78 - DNA, 28 bp, Homo sapiens
ttatggccggtactcctataactgtgtc

SEQ ID NO: 79 - DNA, 23 bp, Homo sapiens
ggcaccaaagcgtgatgttctcc

SEQ ID NO: 80 - DNA, 21 bp, Homo sapiens
tatgcagagcagcactggagc

SEQ ID NO: 81 - DNA, 25 bp, Homo sapiens
gctatgattgatggcagaaaccagc

SEQ ID NO: 82 - DNA, 24 bp, Homo sapiens
tgccttcctatctcagcaggggac

SEQ ID NO: 83 - DNA, 24 bp, Homo sapiens
acactcttggcataggtcatcggc

SEQ ID NO: 84 - DNA, 24 bp, Homo sapiens
ctctcatgcaggtgaggaacgagc

SEQ ID NO: 85 - DNA, 29 bp, Homo sapiens
actgatggaaaaagcctcagactttgatc

SEQ ID NO: 86 - DNA, 23 bp, Homo sapiens
acaggaaatgtctggccacgagt

SEQ ID NO: 87 - DNA, 27 bp, Homo sapiens
tctgtggctgatggaggtggtttatac

SEQ ID NO: 88 - DNA, 50 bp, Homo sapiens
gctgcagctcgttcctcacctgcatgagagaagaatgaagagattcagag

SEQ ID NO: 89 - DNA, 50 bp, Homo sapiens; identical to SEQ ID NO: 55

SEQ ID NO: 90 - DNA, 50 bp, Homo sapiens
atggcgaccaagacaccttgaagggcctaatgcacgcactaaagcactca

SEQ ID NO: 91 - DNA, 50 bp, Homo sapiens
ccaagtcagcagtcctagccccaaaccagcccagagcagggtctctctaa

SEQ ID NO: 92 - DNA, 50 bp, Homo sapiens
acccagcttttgtccacaaggtgactgtaactcagaatggaaagtgggct

SEQ ID NO: 93 - DNA, 50 bp, Homo sapiens
gcttttgtgcctgcagcagagcctgcagaagctaatacaagggacactgg

SEQ ID NO: 94 - DNA, 50 bp, Homo sapiens
cacactgtggcccctgagtcccctaatgtacacgctgcagccagaatgca

SEQ ID NO: 95 - DNA, 50 bp, Homo sapiens
gcatagcacctttgcaagcctgcggcgatttgggtgccagcatcctgcaa

SEQ ID NO: 96 - DNA, 1283 bp, Homo sapiens
ctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc